SEQUENCE LISTING (275 sequences)

SEQ ID NO: 1 - protein, 244 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGLPEGKFDYWGQGTLVTVSSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIKR

SEQ ID NO: 2 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVSSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKR

SEQ ID NO: 3 - protein, 244 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKR

SEQ ID NO: 4 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYSYNNKNYLAWYQQKPGQPPKLLMYWASTRESGVPDRFSGSGSGAEFTLTISSLQAEDVAIYYCQQYYSTLTFGGGTKVEIKR

SEQ ID NO: 5 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYSWNNKNYLAWYQQKPGQPPKLLMYWASTRESGVPDRFSGSGSGAEFTLTISSLQAEDVAIYYCQQYYSTLTFGGGTKVEIKR

SEQ ID NO: 6 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIKR

SEQ ID NO: 7 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIKR

SEQ ID NO: 8 - protein, 244 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVSSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIKR

SEQ ID NO: 9 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIKR

SEQ ID NO: 10 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIKR

SEQ ID NO: 11 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGLPEGKIDYWGQGTLVTVSSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIKR

SEQ ID NO: 12 - protein, 244 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPGLPEGKFDYWGQGTLVTVSSDIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIKR

SEQ ID NO: 13 - protein, 244 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGLPEGKFDYWGQGTLVTVSSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIKR

SEQ ID NO: 14 - protein, 244 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVSSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIKR

SEQ ID NO: 15 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 16 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYASNNKNYLAWYQQKPGQPPKLLMYWASTRESGVPDRFSGSGSGAEFTLTISSLQAEDVAIYYCQQYYSTLTFGGGTKVEIK

SEQ ID NO: 17 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSANNKNYLAWYQQKPGQPPKLLMYWASTRESGVPDRFSGSGSGAEFTLTISSLQAEDVAIYYCQQYYSTLTFGGGTKVEIK

SEQ ID NO: 18 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSSNNKNYLAWYQQKPGQPPKLLMYWASERESGVPDRFSGSGSGAEFTLTISSLQAEDVAIYYCQQYYSTLTFGGGTKVEIK

SEQ ID NO: 19 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSSNNKNYLAWYQQKPGQPPKLLMYWASHRESGVPDRFSGSGSGAEFTLTISSLQAEDVAIYYCQQYYSTLTFGGGTKVEIK

SEQ ID NO: 20 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 21 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 22 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 23 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPEVPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 24 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYAYNNKNYLAWYQQKPGQPPKLLMYWASERESGVPDRFSGSGSGAEFTLTISSLQAEDVAIYYCQQYYSTLTFGGGTKVEIK

SEQ ID NO: 25 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPEVPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 26 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPEVPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 27 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 28 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 29 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPEVPEGKFDYWGQGTLVTVSDIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 30 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYAYNNKNYLAWYQQKPGQPPKLLMYWASERESGVPDRFSGSGSGAEFTLTISSLQAEDVAIYYCQQYYSTLTFGGGTKVEIK

SEQ ID NO: 31 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYDDTVGPGLPEGKIDYWGQGTLVTVSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 32 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYDDTVGPGLPEGKIDYWGQGTLVTVSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 33 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTRGPGLPEGKIDYWGQGTLVTVSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 34 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPELPEGKIDYWGQGTLVTVSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 35 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGVPEGKIDYWGQGTLVTVSDIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 36 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGLPEGKIDYWGQGTLVTVSDIVMTQSPDSLAVSLGERATINCKSSQSVLYSVNNKNYLAWYQQKPGQPPKLLMNWASERESGVPDRFSGSGSGAEFTLTISSLQAEDVAIYYCQQYYSTLTFGGGTKVEIK

SEQ ID NO: 37 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGLPEGKIDYWGQGTLVTVSDIVMTQSPDSLAVSLGERATINCKSSQSVLYSVNNKNYLAWYQQKPGQPPKLLMNWASHRESGVPDRFSGSGSGAEFTLTISSLQAEDVAIYYCQQYYSTLTFGGGTKVEIK

SEQ ID NO: 38 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVLPGLPEGKFDYWGQGTLVTVSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 39 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVLPGLPEGKFDYWGQGTLVTVSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 40 - protein, 242 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTRLPGLPEGKFDYWGQGTLVTVSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 41 - protein, 242 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPELPEGKFDYWGQGTLVTVSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 42 - protein, 242 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGVPEGKFDYWGQGTLVTVSDIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 43 - protein, 242 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGLPEGKFDYWGQGTLVTVSDIVMTQSPDSLAVSLGERATINCKSSQSVLYSYNNKNYLAWYQQKPGQPPKLLMEWASHRESGVPDRFSGSGSGAEFTLTISSLQAEDVAIYYCQQYYSTLTFGGGTKVEIK

SEQ ID NO: 44 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTRGPGLPEGKFDYWGQGTLVTVSDIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIK

SEQ ID NO: 45 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPELPEGKFDYWGQGTLVTVSDIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIK

SEQ ID NO: 46 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTRGPELPEGKFDYWGQGTLVTVSDIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIK

SEQ ID NO: 47 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTRGPGLPEGKFDYWGQGTLVTVSDIVMTQSPDSLAVSLGERATINCKSSQSVLYRSNNLNYLAWYQQKPGQPPKLLMYWASERESGVPDRFSGSGSGAEFTLTISSLQAEDVAIYYCQQYYSTLTFGGGTKVEIK

SEQ ID NO: 48 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPGLPEGKFDYWGQGTLVTVSDIVMTQSPDSLAVSLGERATINCKSSQSVLYRSNNLNYLAWYQQKPGQPPKLLMYWASERESGVPDRFSGSGSGAEFTLTISSLQAEDVAIYYCQQYYSTLTFGGGTKVEIK

SEQ ID NO: 49 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYHDTVGPGLPEGKFDYWGQGTLVTVSDIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIK

SEQ ID NO: 50 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 51 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 52 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 53 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 54 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGVPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 55 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 56 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSANNKNYLAWYQQKPGQPPKLLMYWASERESGVPDRFSGSGSGAEFTLTISSLQAEDVAIYYCQQYYSTLTFGGGTKVEIK

SEQ ID NO: 57 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSWNNKNYLAWYQQKPGQPPKLLMYWASERESGVPDRFSGSGSGAEFTLTISSLQAEDVAIYYCQQYYSTLTFGGGTKVEIK

SEQ ID NO: 58 - protein, 242 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYSYNNKNYLAWYQQKPGQPPKLLMYWASERESGVPDRFSGSGSGAEFTLTISSLQAEDVAIYYCQQYYSTLTFGGGTKVEIK

SEQ ID NO: 59 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYFDTVGPGLPEGKFDYWGQGTLVTVSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 60 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTRGPGLPEGKFDYWGQGTLVTVSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 61 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPELPEGKFDYWGQGTLVTVSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 62 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGVPEGKFDYWGQGTLVTVSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 63 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTRGPELPEGKFDYWGQGTLVTVSDIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 64 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGLPEGKFDYWGQGTLVTVSDIVMTQSPDSLAVSLGERATINCKSSQSVLYWANNKNYLAWYQQKPGQPPKLLMYWASERESGVPDRFSGSGSGAEFTLTISSLQAEDVAIYYCQQYYSTLTFGGGTKVEIK

SEQ ID NO: 65 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 66 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 67 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 68 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 69 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGVPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 70 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 71 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 72 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 73 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 74 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYWANNKNYLAWYQQKPGQPPKLLMYWASERESGVPDRFSGSGSGAEFTLTISSLQAEDVAIYYCQQYYSTLTFGGGTKVEIK

SEQ ID NO: 75 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 76 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 77 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 78 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 79 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 80 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 81 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPEVPEGKFDYWGQGTLVTVSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 82 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYYSNNYNYLAWYQQKPGQPPKLLMYWASTRESGVPDRFSGSGSGAEFTLTISSLQAEDVAIYYCQQYYSTLTFGGGTKVEIK

SEQ ID NO: 83 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYYANNYNYLAWYQQKPGQPPKLLMYWASTRESGVPDRFSGSGSGAEFTLTISSLQAEDVAIYYCQQYYSTLTFGGGTKVEIK

SEQ ID NO: 84 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 85 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYYVNNYNYLAWYQQKPGQPPKLLMYWASERESGVPDRFSGSGSGAEFTLTISSLQAEDVAIYYCQQYYSTLTFGGGTKVEIK

SEQ ID NO: 86 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 87 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 88 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 89 - protein, 242 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGVPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 90 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 91 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPGLPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 92 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 93 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPELPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 94 - protein, 242 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 95 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 96 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYQYNNKNYLAWYQQKPGQPPKLLMYWASERESGVPDRFSGSGSGAEFTLTISSLQAEDVAIYYCQQYYSTLTFGGGTKVEIK

SEQ ID NO: 97 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYSWNNKNYLAWYQQKPGQPPKLLMYWASTRESGVPDRFSGSGSGAEFTLTISSLQAEDVAIYYCQQYYSTLTFGGGTKVEIK

SEQ ID NO: 98 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYSYNNKNYLAWYQQKPGQPPKLLMYWASTRESGVPDRFSGSGSGAEFTLTISSLQAEDVAIYYCQQYYSTLTFGGGTKVEIK

SEQ ID NO: 99 - protein, 242 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVSDIVMTQSPDSLAVSLGERATINCKSSQSVLYSANNKNYLAWYQQKPGQPPKLLMYWASTRESGVPDRFSGSGSGAEFTLTISSLQAEDVAIYYCQQYYSTLTFGGGTKVEIK

SEQ ID NO: 100 - protein, 131 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGLPEGKFDYWGQGTLVTVSS

SEQ ID NO: 101 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVSS

SEQ ID NO: 102 - protein, 131 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSS

SEQ ID NO: 103 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSS

SEQ ID NO: 104 - protein, 131 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVSS

SEQ ID NO: 105 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVSS

SEQ ID NO: 106 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGLPEGKIDYWGQGTLVTVSS

SEQ ID NO: 107 - protein, 131 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPGLPEGKFDYWGQGTLVTVSS

SEQ ID NO: 108 - protein, 131 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGLPEGKFDYWGQGTLVTVSS

SEQ ID NO: 109 - protein, 131 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVSS

SEQ ID NO: 110 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPELPEGKFDYWGQGTLVTVS

SEQ ID NO: 111 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVS

SEQ ID NO: 112 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPGLPEGKFDYWGQGTLVTVS

SEQ ID NO: 113 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPELPEGKFDYWGQGTLVTVS

SEQ ID NO: 114 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPELPEGKFDYWGQGTLVTVS

SEQ ID NO: 115 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPEVPEGKFDYWGQGTLVTVS

SEQ ID NO: 116 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPELPEGKFDYWGQGTLVTVS

SEQ ID NO: 117 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPEVPEGKFDYWGQGTLVTVS

SEQ ID NO: 118 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPEVPEGKFDYWGQGTLVTVS

SEQ ID NO: 119 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPELPEGKFDYWGQGTLVTVS

SEQ ID NO: 120 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVGPELPEGKFDYWGQGTLVTVS

SEQ ID NO: 121 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPEVPEGKFDYWGQGTLVTVS

SEQ ID NO: 122 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYDDTVGPGLPEGKIDYWGQGTLVTVS

SEQ ID NO: 123 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYDDTVGPGLPEGKIDYWGQGTLVTVS

SEQ ID NO: 124 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTRGPGLPEGKIDYWGQGTLVTVS

SEQ ID NO: 125 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPELPEGKIDYWGQGTLVTVS

SEQ ID NO: 126 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGVPEGKIDYWGQGTLVTVS

SEQ ID NO: 127 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYDDTVGPGLPEGKIDYWGQGTLVTVS

SEQ ID NO: 128 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTVLPGLPEGKFDYWGQGTLVTVS

SEQ ID NO: 129 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVLPGLPEGKFDYWGQGTLVTVS

SEQ ID NO: 130 - protein, 130 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTRLPGLPEGKFDYWGQGTLVTVS

SEQ ID NO: 131 - protein, 130 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPELPEGKFDYWGQGTLVTVS

SEQ ID NO: 132 - protein, 130 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGVPEGKFDYWGQGTLVTVS

SEQ ID NO: 133 - protein, 130 aa, synthetic construct
EVQLVESGGGLVKPGGSLRLSCAASGFTFRDVWMSWVRQAPGKGLEWVGRIKSKHDGGTTDYAAPVKGRFTISRDDSKNTLYLQMNSLKTEDTAVYYCTTAGSYYYDTVLPGLPEGKFDYWGQGTLVTVS

SEQ ID NO: 134 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTRGPGLPEGKFDYWGQGTLVTVS

SEQ ID NO: 135 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPELPEGKFDYWGQGTLVTVS

SEQ ID NO: 136 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTRGPELPEGKFDYWGQGTLVTVS

SEQ ID NO: 137 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYHDTVGPGLPEGKFDYWGQGTLVTVS

SEQ ID NO: 138 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYHDTVGPGLPEGKFDYWGQGTLVTVS

SEQ ID NO: 139 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVS

SEQ ID NO: 140 - protein, 130 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTRGPGLPEGKFDYWGQGTLVTVS

SEQ ID NO: 141 - protein, 130 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPELPEGKFDYWGQGTLVTVS

SEQ ID NO: 142 - protein, 130 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGVPEGKFDYWGQGTLVTVS

SEQ ID NO: 143 - protein, 130 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTRGPELPEGKFDYWGQGTLVTVS

SEQ ID NO: 144 - protein, 130 aa, synthetic construct
EVQLVESGGGLVKPGGSLRLSCAASGFTFRDVWMSWVRQAPGKGLEWVGRIKSKDDGGTTDYAAPVKGRFTISRDDSKNTLYLQMNSLKTEDTAVYYCTTAGSYYYDTVGPGLPEGKFDYWGQGTLVTVS

SEQ ID NO: 145 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYFDTVGPGLPEGKFDYWGQGTLVTVS

SEQ ID NO: 146 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTRGPGLPEGKFDYWGQGTLVTVS

SEQ ID NO: 147 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPELPEGKFDYWGQGTLVTVS

SEQ ID NO: 148 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGVPEGKFDYWGQGTLVTVS

SEQ ID NO: 149 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTRGPELPEGKFDYWGQGTLVTVS

SEQ ID NO: 150 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGLPEGKFDYWGQGTLVTVS

SEQ ID NO: 151 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVS

SEQ ID NO: 152 - protein, 130 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGVPEGKFDYWGQGTLVTVS

SEQ ID NO: 153 - protein, 130 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTRGPGLPEGKFDYWGQGTLVTVS

SEQ ID NO: 154 - protein, 130 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYYDTRGPGLPEGKFDYWGQGTLVTVS

SEQ ID NO: 155 - protein, 113 aa, synthetic construct
DIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIKR

SEQ ID NO: 156 - protein, 113 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKR

SEQ ID NO: 157 - protein, 113 aa, synthetic construct
DIVMTQSPDSLAVSLGERATINCKSSQSVLYSYNNKNYLAWYQQKPGQPPKLLMYWASTRESGVPDRFSGSGSGAEFTLTISSLQAEDVAIYYCQQYYSTLTFGGGTKVEIKR

SEQ ID NO: 158 - protein, 113 aa, synthetic construct
DIVMTQSPDSLAVSLGERATINCKSSQSVLYSWNNKNYLAWYQQKPGQPPKLLMYWASTRESGVPDRFSGSGSGAEFTLTISSLQAEDVAIYYCQQYYSTLTFGGGTKVEIKR

SEQ ID NO: 159 - protein, 113 aa, synthetic construct
DIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIKR

SEQ ID NO: 160 - protein, 113 aa, synthetic construct
DIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIKR

SEQ ID NO: 161 - protein, 113 aa, synthetic construct
DIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIKR

SEQ ID NO: 162 - protein, 113 aa, synthetic construct
DIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIKR

SEQ ID NO: 163 - protein, 113 aa, synthetic construct
DIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIKR

SEQ ID NO: 164 - protein, 113 aa, synthetic construct
DIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIKR

SEQ ID NO: 165 - protein, 113 aa, synthetic construct
DIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIKR

SEQ ID NO: 166 - protein, 113 aa, synthetic construct
DIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIKR

SEQ ID NO: 167 - protein, 112 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIK

SEQ ID NO: 168 - protein, 112 aa, synthetic construct
DIVMTQSPDSLAVSLGERATINCKSSQSVLYASNNKNYLAWYQQKPGQPPKLLMYWASTRESGVPDRFSGSGSGAEFTLTISSLQAEDVAIYYCQQYYSTLTFGGGTKVEIK

SEQ ID NO: 169 - protein, 112 aa, synthetic construct
DIVMTQSPDSLAVSLGERATINCKSSQSVLYSANNKNYLAWYQQKPGQPPKLLMYWASTRESGVPDRFSGSGSGAEFTLTISSLQAEDVAIYYCQQYYSTLTFGGGTKVEIK

SEQ ID NO: 170 - protein, 112 aa, synthetic construct
DIVMTQSPDSLAVSLGERATINCKSSQSVLYSSNNKNYLAWYQQKPGQPPKLLMYWASERESGVPDRFSGSGSGAEFTLTISSLQAEDVAIYYCQQYYSTLTFGGGTKVEIK

SEQ ID NO: 171 - protein, 112 aa, synthetic construct
DIVMTQSPDSLAVSLGERATINCKSSQSVLYSSNNKNYLAWYQQKPGQPPKLLMYWASHRESGVPDRFSGSGSGAEFTLTISSLQAEDVAIYYCQQYYSTLTFGGGTKVEIK

SEQ ID NO: 172 - protein, 112 aa, synthetic construct
DIVMTQSPDSLAVSLGERATINCKSSQSVLYAANNKNYLAWYQQKPGQPPKLLMYWASERESGVPDRFSGSGSGAEFTLTISSLQAEDVAIYYCQQYYSTLTFGGGTKVEIK

SEQ ID NO: 173 - protein, 112 aa, synthetic construct
DIVMTQSPDSLAVSLGERATINCKSSQSVLYAYNNKNYLAWYQQKPGQPPKLLMYWASERESGVPDRFSGSGSGAEFTLTISSLQAEDVAIYYCQQYYSTLTFGGGTKVEIK

SEQ ID NO: 174 - protein, 112 aa, synthetic construct
DIVMTQSPDSLAVSLGERATINCKSSQSVLYSVNNKNYLAWYQQKPGQPPKLLMNWASTRESGVPDRFSGSGSGAEFTLTISSLQAEDVAIYYCQQYYSTLTFGGGTKVEIK

SEQ ID NO: 175 - protein, 112 aa, synthetic construct
DIVMTQSPDSLAVSLGERATINCKSSQSVLYSVNNKNYLAWYQQKPGQPPKLLMNWASERESGVPDRFSGSGSGAEFTLTISSLQAEDVAIYYCQQYYSTLTFGGGTKVEIK

SEQ ID NO: 176 - protein, 112 aa, synthetic construct
DIVMTQSPDSLAVSLGERATINCKSSQSVLYSVNNKNYLAWYQQKPGQPPKLLMNWASHRESGVPDRFSGSGSGAEFTLTISSLQAEDVAIYYCQQYYSTLTFGGGTKVEIK

SEQ ID NO: 177 - protein, 112 aa, synthetic construct
DIVMTQSPDSLAVSLGERATINCKSSQSVLYSYNNKNYLAWYQQKPGQPPKLLMEWASTRESGVPDRFSGSGSGAEFTLTISSLQAEDVAIYYCQQYYSTLTFGGGTKVEIK

SEQ ID NO: 178 - protein, 112 aa, synthetic construct
DIVMTQSPDSLAVSLGERATINCKSSQSVLYSYNNKNYLAWYQQKPGQPPKLLMEWASHRESGVPDRFSGSGSGAEFTLTISSLQAEDVAIYYCQQYYSTLTFGGGTKVEIK

SEQ ID NO: 179 - protein, 112 aa, synthetic construct
DIVMTQSPDSLAVSLGERATINCKSSQSVLYRSNNLNYLAWYQQKPGQPPKLLMYWASTRESGVPDRFSGSGSGAEFTLTISSLQAEDVAIYYCQQYYSTLTFGGGTKVEIK

SEQ ID NO: 180 - protein, 112 aa, synthetic construct
DIVMTQSPDSLAVSLGERATINCKSSQSVLYRSNNLNYLAWYQQKPGQPPKLLMYWASERESGVPDRFSGSGSGAEFTLTISSLQAEDVAIYYCQQYYSTLTFGGGTKVEIK

SEQ ID NO: 181 - protein, 112 aa, synthetic construct
DIVMTQSPDSLAVSLGERATINCKSSQSVLYSFNNKNYLAWYQQKPGQPPKLLMYWASERESGVPDRFSGSGSGAEFTLTISSLQAEDVAIYYCQQYYSTLTFGGGTKVEIK

SEQ ID NO: 182 - protein, 112 aa, synthetic construct
DIVMTQSPDSLAVSLGERATINCKSSQSVLYSANNKNYLAWYQQKPGQPPKLLMYWASERESGVPDRFSGSGSGAEFTLTISSLQAEDVAIYYCQQYYSTLTFGGGTKVEIK

SEQ ID NO: 183 - protein, 112 aa, synthetic construct
DIVMTQSPDSLAVSLGERATINCKSSQSVLYSWNNKNYLAWYQQKPGQPPKLLMYWASERESGVPDRFSGSGSGAEFTLTISSLQAEDVAIYYCQQYYSTLTFGGGTKVEIK

SEQ ID NO: 184 - protein, 112 aa, synthetic construct
DIVMTQSPDSLAVSLGERATINCKSSQSVLYSYNNKNYLAWYQQKPGQPPKLLMYWASERESGVPDRFSGSGSGAEFTLTISSLQAEDVAIYYCQQYYSTLTFGGGTKVEIK

SEQ ID NO: 185 - protein, 112 aa, synthetic construct
DIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIK

SEQ ID NO: 186 - protein, 112 aa, synthetic construct
DIVMTQSPDSLAVSLGERATINCKSSQSVLYWANNKNYLAWYQQKPGQPPKLLMYWASERESGVPDRFSGSGSGAEFTLTISSLQAEDVAIYYCQQYYSTLTFGGGTKVEIK

SEQ ID NO: 187 - protein, 112 aa, synthetic construct
DIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIK

SEQ ID NO: 188 - protein, 112 aa, synthetic construct
DIVMTQSPDSLAVSLGERATINCKSSQSVLYYVNNYNYLAWYQQKPGQPPKLLMYWASTRESGVPDRFSGSGSGAEFTLTISSLQAEDVAIYYCQQYYSTLTFGGGTKVEIK

SEQ ID NO: 189 - protein, 112 aa, synthetic construct
DIVMTQSPDSLAVSLGERATINCKSSQSVLYYSNNYNYLAWYQQKPGQPPKLLMYWASTRESGVPDRFSGSGSGAEFTLTISSLQAEDVAIYYCQQYYSTLTFGGGTKVEIK

SEQ ID NO: 190 - protein, 112 aa, synthetic construct
DIVMTQSPDSLAVSLGERATINCKSSQSVLYYANNYNYLAWYQQKPGQPPKLLMYWASTRESGVPDRFSGSGSGAEFTLTISSLQAEDVAIYYCQQYYSTLTFGGGTKVEIK

SEQ ID NO: 191 - protein, 112 aa, synthetic construct
DIVMTQSPDSLAVSLGERATINCKSSQSVLYYVNNYNYLAWYQQKPGQPPKLLMYWASERESGVPDRFSGSGSGAEFTLTISSLQAEDVAIYYCQQYYSTLTFGGGTKVEIK

SEQ ID NO: 192 - protein, 112 aa, synthetic construct
DIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIK

SEQ ID NO: 193 - protein, 112 aa, synthetic construct
DIVMTQSPDSLAVSLGERATINCKSSQSVLYQYNNKNYLAWYQQKPGQPPKLLMYWASERESGVPDRFSGSGSGAEFTLTISSLQAEDVAIYYCQQYYSTLTFGGGTKVEIK

SEQ ID NO: 194 - protein, 112 aa, synthetic construct
DIVMTQSPDSLAVSLGERATINCKSSQSVLYSWNNKNYLAWYQQKPGQPPKLLMYWASTRESGVPDRFSGSGSGAEFTLTISSLQAEDVAIYYCQQYYSTLTFGGGTKVEIK

SEQ ID NO: 195 - protein, 112 aa, synthetic construct
DIVMTQSPDSLAVSLGERATINCKSSQSVLYSYNNKNYLAWYQQKPGQPPKLLMYWASTRESGVPDRFSGSGSGAEFTLTISSLQAEDVAIYYCQQYYSTLTFGGGTKVEIK

SEQ ID NO: 196 - protein, 8 aa, synthetic construct
GFTFRDVW

SEQ ID NO: 197 - protein, 10 aa, synthetic construct
IKSKEDGGTT

SEQ ID NO: 198 - protein, 10 aa, synthetic construct
IKSKIDGGTT

SEQ ID NO: 199 - protein, 10 aa, synthetic construct
IKSKWDGGTT

SEQ ID NO: 200 - protein, 10 aa, synthetic construct
IKSKDDGGTT

SEQ ID NO: 201 - protein, 10 aa, synthetic construct
IKSKHDGGTT

SEQ ID NO: 202 - protein, 22 aa, synthetic construct
TTAGSYYFDTVGPGLPEGKFDY

SEQ ID NO: 203 - protein, 22 aa, synthetic construct
TTAGSYYYDTRGPGLPEGKFDY

SEQ ID NO: 204 - protein, 22 aa, synthetic construct
TTAGSYYYDTVGPGLPEGKFDY

SEQ ID NO: 205 - protein, 22 aa, synthetic construct
TTAGSYYYDTVGPELPEGKFDY

SEQ ID NO: 206 - protein, 22 aa, synthetic construct
TTAGSYYDDTVGPGLPEGKIDY

SEQ ID NO: 207 - protein, 22 aa, synthetic construct
TTAGSYYHDTVGPGLPEGKFDY

SEQ ID NO: 208 - protein, 22 aa, synthetic construct
TTAGSYYYDTVLPGLPEGKFDY

SEQ ID NO: 209 - protein, 22 aa, synthetic construct
TTAGSYYYDTRGPELPEGKFDY

SEQ ID NO: 210 - protein, 22 aa, synthetic construct
TTAGSYYYDTRGPEVPEGKFDY

SEQ ID NO: 211 - protein, 22 aa, synthetic construct
TTAGSYYYDTVGPEVPEGKFDY

SEQ ID NO: 212 - protein, 22 aa, synthetic construct
TTAGSYYDDTRGPGLPEGKIDY

SEQ ID NO: 213 - protein, 22 aa, synthetic construct
TTAGSYYDDTVGPELPEGKIDY

SEQ ID NO: 214 - protein, 22 aa, synthetic construct
TTAGSYYDDTVGPGVPEGKIDY

SEQ ID NO: 215 - protein, 22 aa, synthetic construct
TTAGSYYYDTRLPGLPEGKFDY

SEQ ID NO: 216 - protein, 22 aa, synthetic construct
TTAGSYYYDTVLPELPEGKFDY

SEQ ID NO: 217 - protein, 22 aa, synthetic construct
TTAGSYYYDTVLPGVPEGKFDY

SEQ ID NO: 218 - protein, 22 aa, synthetic construct
TTAGSYYHDTRGPGLPEGKFDY

SEQ ID NO: 219 - protein, 22 aa, synthetic construct
TTAGSYYHDTVGPELPEGKFDY

SEQ ID NO: 220 - protein, 22 aa, synthetic construct
TTAGSYYHDTRGPELPEGKFDY

SEQ ID NO: 221 - protein, 22 aa, synthetic construct
TTAGSYYYDTVGPGVPEGKFDY

SEQ ID NO: 222 - protein, 22 aa, synthetic construct
TTAGSYYFDTRGPGLPEGKFDY

SEQ ID NO: 223 - protein, 22 aa, synthetic construct
TTAGSYYFDTVGPELPEGKFDY

SEQ ID NO: 224 - protein, 22 aa, synthetic construct
TTAGSYYFDTVGPGVPEGKFDY

SEQ ID NO: 225 - protein, 22 aa, synthetic construct
TTAGSYYFDTRGPELPEGKFDY

SEQ ID NO: 226 - protein, 12 aa, synthetic construct
QSVLYWANNKNY

SEQ ID NO: 227 - protein, 12 aa, synthetic construct
QSVLYSSNNKNY

SEQ ID NO: 228 - protein, 12 aa, synthetic construct
QSVLYSYNNKNY

SEQ ID NO: 229 - protein, 12 aa, synthetic construct
QSVLYSWNNKNY

SEQ ID NO: 230 - protein, 12 aa, synthetic construct
QSVLYQYNNKNY

SEQ ID NO: 231 - protein, 12 aa, synthetic construct
QSVLYWSNNKNY

SEQ ID NO: 232 - protein, 12 aa, synthetic construct
QSVLYSFNNKNY

SEQ ID NO: 233 - protein, 12 aa, synthetic construct
QSVLYYVNNYNY

SEQ ID NO: 234 - protein, 12 aa, synthetic construct
QSVLYAANNKNY

SEQ ID NO: 235 - protein, 12 aa, synthetic construct
QSVLYSVNNKNY

SEQ ID NO: 236 - protein, 12 aa, synthetic construct
QSVLYRSNNLNY

SEQ ID NO: 237 - protein, 12 aa, synthetic construct
QSVLYASNNKNY

SEQ ID NO: 238 - protein, 12 aa, synthetic construct
QSVLYSANNKNY

SEQ ID NO: 239 - protein, 12 aa, synthetic construct
QSVLYAYNNKNY

SEQ ID NO: 240 - protein, 12 aa, synthetic construct
QSVLYYSNNYNY

SEQ ID NO: 241 - protein, 12 aa, synthetic construct
QSVLYYANNYNY

SEQ ID NO: 242 - skipped

SEQ ID NO: 243 - protein, 6 aa, synthetic construct
YWASTR

SEQ ID NO: 244 - protein, 6 aa, synthetic construct
YWASHR

SEQ ID NO: 245 - protein, 6 aa, synthetic construct
YWASER

SEQ ID NO: 246 - protein, 6 aa, synthetic construct
NWASTR

SEQ ID NO: 247 - protein, 6 aa, synthetic construct
EWASTR

SEQ ID NO: 248 - protein, 6 aa, synthetic construct
NWASER

SEQ ID NO: 249 - protein, 6 aa, synthetic construct
NWASHR

SEQ ID NO: 250 - protein, 6 aa, synthetic construct
EWASHR

SEQ ID NO: 251 - protein, 8 aa, synthetic construct
QQYYSTLT

SEQ ID NO: 252 - protein, 384 aa, Homo sapiens
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 253 - protein, 330 aa, Homo sapiens [sequence omitted]

SEQ ID NO: 254 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 255 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 256 - protein, 452 aa, Homo sapiens
GSASAPTLFPLVSCENSPSDTSSVAVGCLAQDFLPDSITLSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVAHEALPNRVTERTVDKSTGKPTLYNVSLVMSDTAGTCY

SEQ ID NO: 257 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 258 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 259 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 260 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 261 - protein, 461 aa, synthetic construct
EVQLVESGGGLVKPGGSLRLSCAASGFTFRDVWMSWVRQAPGKGLEWVGRIKSKEDGGTTDYAAPVKGRFTISRDDSKNTLYLQMNSLKTEDTAVYYCTTAGSYYFDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 461 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTRGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 461 aa, synthetic construct
EVQLVESGGGLVKPGGSLRLSCAASGFTFRDVWMSWVRQAPGKGLEWVGRIKSKWDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 461 aa, synthetic construct
EVQLVESGGGLVKPGGSLRLSCAASGFTFRDVWMSWVRQAPGKGLEWVGRIKSKIDGGTTDYAAPVKGRFTISRDDSKNTLYLQMNSLKTEDTAVYYCTTAGSYYYDTVGPGLPEGKFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 219 aa, synthetic construct
DIVMTQSPDSLAVSLGERATINCKSSQSVLYWANNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 219 aa, synthetic construct
DIVMTQSPDSLAVSLGERATINCKSSQSVLYSSNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 267 - protein, 219 aa, synthetic construct
DIVMTQSPDSLAVSLGERATINCKSSQSVLYSYNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 219 aa, synthetic construct
DIVMTQSPDSLAVSLGERATINCKSSQSVLYSWNNKNYLAWYQQKPGQPPKLLMYWASTRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 269 - protein, 219 aa, synthetic construct
DIVMTQSPDSLAVSLGERATINCKSSQSVLYQYNNKNYLAWYQQKPGQPPKLLMYWASHRESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 219 aa, synthetic construct
DIVMTQSPDSLAVSLGERATINCKSSQSVLYWSNNKNYLAWYQQKPGQPPKLLMYWASERESGVPDRFSGSGSGAEFTLTISSLQAEDVAIYYCQQYYSTLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 271 - protein, 259 aa, unidentified
MGILPSPGMPALLSLVSLLSVLLMGCVAETGTRFPNITNLCPFGEVFNATRFASVYAWNRKRISNCVADFSVLYNSASFSTFKCYGVSPTKLNDLCWTNIYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGLNDIFEAQKIEWHEHHHHHHHH

SEQ ID NO: 272 - protein, 259 aa, unidentified
MGILPSPGMPALLSLVSLLSVLLMGCVAETGTRFPNITNLCPFDEVFNATRFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGLNDIFEAQKIEWHEHHHHHHHH

SEQ ID NO: 273 - protein, 259 aa, unidentified
MGILPSPGMPALLSLVSLLSVLLMGCVAETGTRFPNITNLCPFDEVFNATKFASVYAWNRKRISNCVADFSVLYNLAPFFTFKCYGVSPTKLNDLCWTNIYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTGLNDIFEAQKIEWHEHHHHHHHH

SEQ ID NO: 274 - protein, 131 aa, unidentified; identical to SEQ ID NO: 103

SEQ ID NO: 275 - protein, 113 aa, unidentified; identical to SEQ ID NO: 156

(19) United States
(12) Patent Application Publication
(10) Pub. No.: US 2023/0365658 A1
(43) Pub. Date: Nov. 16, 2023

(54) REPAIRED THERAPEUTIC AND PROPHYLACTIC ANTIBODIES AGAINST SARS-CoV-2 VARIANTS

(71) Applicants: Lawrence Livermore National Security, LLC, Livermore, CA (US); Vanderbilt University, Nashvile, TN (US); Triad National Security, LLC, Los Alamos, NM (US)

(72) Inventors: Edmond Y. Lau, Dublin, CA (US); Daniel M. Faissol, Livermore, CA (US); Thomas Anthony Desautels, Brentwood, CA (US); Fangqiang Zhu, Pleasanton, CA (US); Adam T. Zemla, Brentwood, CA (US); Robert Herschel Carnahan, JR., Nashville, CA (US); Antonietta Maria Lillo, Los Alamos, NM (US); James E. Crowe, JR., Nashville, TN (US); Kathryn Arrildt, Livermore, CA (US)

(21) Appl. No.: 18/122,658

(22) Filed: Mar. 16, 2023

Related U.S. Application Data
(60) Provisional application No. 63/342,591, filed on May 16, 2022.
(60) Provisional application No. 63/380,060, filed on Oct. 18, 2022.
(60) Provisional application No. 63/439,038, filed on Jan. 13, 2023.

(51) Int. Cl. C07K 16/10; A61P 31/14

(52) U.S. Cl. CPC C07K 16/10; A61P 31/14; C07K 2317/92; C07K 2317/565

(57) ABSTRACT

Provided herein are novel monoclonal antibodies and antibody fragments with improved binding to and/or neutralization against multiple SARS-CoV-2 variants, e.g., Omicron lineages. Also provided are compositions and biosensors comprising the monoclonal antibodies and antibody fragments and methods of using the novel monoclonal antibodies and antibody fragments for treating, preventing, and diagnosing COVID-19 and detecting SARS-CoV-2.

### CROSS-REFERENCE TO RELATED APPLICATION

This application claims the benefit under 35 U.S.C. § 119(e) and the Paris Convention U.S. provisional applications, 63/342,591, filed May 16, 2022, U.S. provisional application 63/380,060, filed Oct. 18, 2022, and U.S. provisional application 63/439,038, filed Jan. 13, 2023, the contents of each of which are incorporated herein by reference in their entirety.

### STATEMENT OF GOVERNMENT SUPPORT

The United States Government has rights in this invention pursuant to Contract No. DEAC52-07NA27344 between the U.S. Department of Energy and Lawrence Livermore National Security, LLC, for the operation of Lawrence Livermore National Laboratory. This invention also was made with government support under grant number HR0011-18-2-0001 awarded by the Department of Defense. The government has certain rights in the invention.

### SEQUENCE LISTING

The instant application contains a Sequence Listing which has been submitted electronically in XML format and is hereby incorporated by reference in its entirety. Said XML copy, created Apr. 7, 2023, is named 093866-9303.xml and is 280,240 bytes in size.

### TECHNICAL FIELD

The present disclosure relates generally to the fields of infectious disease and immunology. Specifically, the disclosure relates to engineered human antibodies against coronaviruses that cause Severe Acute Respiratory Syndrome (SARS), such as SARS-CoV-2 and its variants.

### BACKGROUND

Coronaviruses are a family of viruses that can cause illnesses such as the common cold, pneumonia, severe acute respiratory syndrome (SARS) and Middle East respiratory syndrome (MERS). In 2019, a new coronavirus was identified as the causative agent for a severe acute respiratory syndrome (SARS) outbreak that originated in Wuhan, China. The virus is known as severe acute respiratory syndrome coronavirus 2 (SARS-CoV-2). The disease it causes is called coronavirus disease 2019 (COVID-19).

Vaccination against SARS-CoV-2 reduces the burden of COVID-19. However, vaccination is not safe for some persons, including immunocompromised persons and those who cannot be vaccinated. Accordingly, a potential option for COVID-19 immunoprophylaxis in those who cannot be vaccinated includes monoclonal antibodies, which protect against disease irrespective of immune system status and provide rapid protection. Such prophylactics can provide crucial immune protection, especially for immunocompromised individuals. Failure of such a prophylactic could result in illness or death of those who cannot be vaccinated.

Some combinations of monoclonal antibodies are already in use through the U.S. Food and Drug Administration (FDA)-approved Emergency or temporary Use Authorizations for preexposure (e.g., Ronapreve™ (casirivimab and imdevimab) 120 mg/mL solution for injection or infusion), postexposure (e.g., REGEN-COV™ (casirivimab and imdevimab)), prophylaxis against COVID-19, or treatment of mild-to-moderate disease (e.g., Bamlanivimab and etesevimab; sotrovimab; or Evusheld (AZD7442)). ZD7442 is a combination of two fully human, SARS-CoV-2-neutralizing monoclonal antibodies (tixagevimab and cilgavimab). The antibody COV2-2130 (Cilgavimab; AZD1061), which after further engineering, is one of two constituents of ZD7442. During the course of the COVID-19 pandemic, the SARS-CoV-2 virus, like most viruses, evolved gradually into new strains that evaded the protection offered by the human immune system and available medical countermeasures, including therapeutic or prophylactic monoclonal antibodies and aforementioned FDA-authorized countermeasures, and other candidates.

Accordingly, there is a need for monoclonal antibodies for use as immunoprophylaxis for preexposure, postexposure, prophylaxis, or treatment of mild-to-moderate pneumonia or severe acute respiratory syndrome caused by a coronavirus. This disclosure addresses these needs.

### SUMMARY OF THE DISCLOSURE

One aspect of the present disclosure provides an isolated antibody or antibody fragment thereof that binds to a SARS-CoV-2 surface protein. In one aspect, the antibody or fragments thereof have enhanced binding and/or neutralization characteristics against all known SARS-CoV-2 strains when compared to antibodies previously described in the prior art, e.g., without limitation a monoclonal antibody selected from sotrovimab (S309), COV2-2381, cilgavimab (COV2-2130 or AZD1061), and/or tixagevimab (COV2-2196 or AZD8895). In some embodiments, the SARS-CoV-2 surface protein is a spike (S) glycoprotein. In some embodiments, the monoclonal antibody or antibody fragment binds to and/or neutralizes SARS-CoV-2 Omicron (B.1.1.529, BA.1, BA.1.1, BA.2, BA.3, BA.4 and BA.5 lineages).

In some embodiments, the antibody or antibody fragment comprises, or consists essentially of, or consists of: (a) a heavy chain complementary determining region 1 (CDRH1) comprising, or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 202, and a light chain complementary determining region 1 (CDRL1) comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (b) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (c) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (d) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (e) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 229, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (f) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251; (g) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251; (h) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251; (i) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (j) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251; (k) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251; (l) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 207, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251; (m) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251; or (n) a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises, or consists essentially of, or consists of: (a) a heavy chain variable sequence having at least 70%, 80%, 90%, 95%, 96%, 97%, 98%, or 99% identity to the amino acid sequence of SEQ ID NO: 100, 101, 102, 103, 104, 105, 106, 107, 108, or 109; and/or (b) a light chain variable sequences having at least 70%, 80%, 90%, 95%, 96%, 97%, 98%, or 99% identity to the amino acid sequence of SEQ ID NO: 155, 156, 157, 158, 159, 160, 161, 162, 163, 164, 165, or 166.

In some embodiments, the antibody or antibody fragment comprises, or consists essentially of, or consists of a heavy chain variable sequence and a light chain variable sequence having at least 96% identity to an amino acid sequence comprising, or consisting essentially of, or consisting of an amino acid sequence selected from: (a) the amino acid sequence of SEQ ID NO: 100 and the amino acid sequence of SEQ ID NO: 155; (b) the amino acid sequence of SEQ ID NO: 101 and the amino acid sequence of SEQ ID NO: 156; (c) the amino acid sequence of SEQ ID NO: 102 and the amino acid sequence of SEQ ID NO: 156; (d) the amino acid sequence of SEQ ID NO: 103 and the amino acid sequence of SEQ ID NO: 157; (e) the amino acid sequence of SEQ ID NO: 103 and the amino acid sequence of SEQ ID NO: 158; (f) the amino acid sequence of SEQ ID NO: 103 and the amino acid sequence of SEQ ID NO: 159; (g) the amino acid sequence of SEQ ID NO: 103 and the amino acid sequence of SEQ ID NO: 160; (h) the amino acid sequence of SEQ ID NO: 104 and the amino acid sequence of SEQ ID NO: 161; (i) the amino acid sequence of SEQ ID NO: 105 and the amino acid sequence of SEQ ID NO: 162; (j) the amino acid sequence of SEQ ID NO: 105 and the amino acid sequence of SEQ ID NO: 163; (k) the amino acid sequence of SEQ ID NO: 106 and the amino acid sequence of SEQ ID NO: 164; (l) the amino acid sequence of SEQ ID NO: 107 and the amino acid sequence of SEQ ID NO: 165; (m) the amino acid sequence of SEQ ID NO: 108 and the amino acid sequence of SEQ ID NO: 166; or (n) the amino acid sequence of SEQ ID NO: 109 and the amino acid sequence of SEQ ID NO: 163. In one aspect the sequence that is at least 96% identical has a CDR sequence that is 100% identical to the CDR of the reference polypeptide. The reference CDRs are provided above, and further within this document. Stated another way, the amino acid substitutions and modifications are in regions outside of the CDR regions.

In some embodiments, the antibody fragment is a recombinant antibody variable domain fragment (Fv fragment) and/or the antibody fragment comprises, or consists essentially of, or consists of the amino acid sequence of SEQ ID NOs: 1-14 or an equivalent of each thereof. In one aspect the equivalent has at least 96% identity to any one of SEQ ID NOS: 1-14 and further in one embodiment has a CDR sequence that is 100% identical to the CDR of the reference polypeptide. The reference CDRs are provided above, and further within this document. Stated another way, the amino acid substitutions and modifications are in regions outside of the CDR regions.

In some embodiments, the antibody or antibody fragment comprises a heavy chain complementary determining region 1 (CDRH1) comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 237, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises, or consisting essentially of, or consisting of a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 239, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of g the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 211, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 239, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197 and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 212, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 213, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 214, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 248, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 249, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 215, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 216, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 217, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 250, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 218, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 219, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 220, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 218, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 207, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 207, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 221, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 229, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 202, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 222, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 223, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 224, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 225, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 202, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising the amino acid sequence of SEQ ID NO: 221, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 211, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 240, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 241, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 221, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 229, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment disclosed herein comprises, or consists essentially of, or consists of a heavy chain variable sequence and a light chain variable sequence having at least 96%, at least 97%, at least 98%, at least 99% or 100% identity to an amino acid sequence selected from: the amino acid sequence of SEQ ID NO: 110 and the amino acid sequence of SEQ ID NO: 167; the amino acid sequence of SEQ ID NO: 111 and the amino acid sequence of SEQ ID NO: 168; the amino acid sequence of SEQ ID NO: 111 and the amino acid sequence of SEQ ID NO: 169; the amino acid sequence of SEQ ID NO: 111 and the amino acid sequence of SEQ ID NO: 170; the amino acid sequence of SEQ ID NO: 111 and the amino acid sequence of SEQ ID NO: 171; the amino acid sequence of SEQ ID NO: 112 and the amino acid sequence of SEQ ID NO: 173; the amino acid sequence of SEQ ID NO: 113 and the amino acid sequence of SEQ ID NO: 172; the amino acid sequence of SEQ ID NO: 114 and the amino acid sequence of SEQ ID NO: 172; the amino acid sequence of SEQ ID NO: 115 and the amino acid sequence of SEQ ID NO: 172; the amino acid sequence of SEQ ID NO: 116 and the amino acid sequence of SEQ ID NO: 173; the amino acid sequence of SEQ ID NO: 117 and the amino acid sequence of SEQ ID NO: 172; the amino acid sequence of SEQ ID NO: 118 and the amino acid sequence of SEQ ID NO: 172; the amino acid sequence of SEQ ID NO: 119 and the amino acid sequence of SEQ ID NO: 172; the amino acid sequence of SEQ ID NO: 120 and the amino acid sequence of SEQ ID NO: 172; the amino acid sequence of SEQ ID NO: 121 and the amino acid sequence of SEQ ID NO: 172; the amino acid sequence of SEQ ID NO: 110 and the amino acid sequence of SEQ ID NO: 173; the amino acid sequence of SEQ ID NO: 122 and the amino acid sequence of SEQ ID NO: 174; the amino acid sequence of SEQ ID NO: 123 and the amino acid sequence of SEQ ID NO: 174; the amino acid sequence of SEQ ID NO: 124 and the amino acid sequence of SEQ ID NO: 174; the amino acid sequence of SEQ ID NO: 125 and the amino acid sequence of SEQ ID NO: 174; the amino acid sequence of SEQ ID NO: 126 and the amino acid sequence of SEQ ID NO: 174; the amino acid sequence of SEQ ID NO: 127 and the amino acid sequence of SEQ ID NO: 175; the amino acid sequence of SEQ ID NO: 127 and the amino acid sequence of SEQ ID NO: 176; the amino acid sequence of SEQ ID NO: 128 and the amino acid sequence of SEQ ID NO: 177; the amino acid sequence of SEQ ID NO: 129 and the amino acid sequence of SEQ ID NO: 177; the amino acid sequence of SEQ ID NO: 130 and the amino acid sequence of SEQ ID NO: 177; the amino acid sequence of SEQ ID NO: 131 and the amino acid sequence of SEQ ID NO: 177; the amino acid sequence of SEQ ID NO: 132 and the amino acid sequence of SEQ ID NO: 177; the amino acid sequence of SEQ ID NO: 133 and the amino acid sequence of SEQ ID NO: 178; the amino acid sequence of SEQ ID NO: 134 and the amino acid sequence of SEQ ID NO: 179; the amino acid sequence of SEQ ID NO: 135 and the amino acid sequence of SEQ ID NO: 179; the amino acid sequence of SEQ ID NO: 136 and the amino acid sequence of SEQ ID NO: 179; the amino acid sequence of SEQ ID NO: 134 and the amino acid sequence of SEQ ID NO: 180; the amino acid sequence of SEQ ID NO: 137 and the amino acid sequence of SEQ ID NO: 180; the amino acid sequence of SEQ ID NO: 138 and the amino acid sequence of SEQ ID NO: 179; the amino acid sequence of SEQ ID NO: 139 and the amino acid sequence of SEQ ID NO: 181; the amino acid sequence of SEQ ID NO: 112 and the amino acid sequence of SEQ ID NO: 181; the amino acid sequence of SEQ ID NO: 140 and the amino acid sequence of SEQ ID NO: 181; the amino acid sequence of SEQ ID NO: 141 and the amino acid sequence of SEQ ID NO: 181; the amino acid sequence of SEQ ID NO: 142 and the amino acid sequence of SEQ ID NO: 181; the amino acid sequence of SEQ ID NO: 143 and the amino acid sequence of SEQ ID NO: 181; the amino acid sequence of SEQ ID NO: 144 and the amino acid sequence of SEQ ID NO: 182; the amino acid sequence of SEQ ID NO: 144 and the amino acid sequence of SEQ ID NO: 183; the amino acid sequence of SEQ ID NO: 144 and the amino acid sequence of SEQ ID NO: 184; the amino acid sequence of SEQ ID NO: 145 and the amino acid sequence of SEQ ID NO: 185; the amino acid sequence of SEQ ID NO: 146 and the amino acid sequence of SEQ ID NO: 185; the amino acid sequence of SEQ ID NO: 147 and the amino acid sequence of SEQ ID NO: 185; the amino acid sequence of SEQ ID NO: 148 and the amino acid sequence of SEQ ID NO: 185; the amino acid sequence of SEQ ID NO: 149 and the amino acid sequence of SEQ ID NO: 185; the amino acid sequence of SEQ ID NO: 150 and the amino acid sequence of SEQ ID NO: 186; the amino acid sequence of SEQ ID NO: 139 and the amino acid sequence of SEQ ID NO: 187; the amino acid sequence of SEQ ID NO: 112 and the amino acid sequence of SEQ ID NO: 187; the amino acid sequence of SEQ ID NO: 151 and the amino acid sequence of SEQ ID NO: 187; the amino acid sequence of SEQ ID NO: 110 and the amino acid sequence of SEQ ID NO: 187; the amino acid sequence of SEQ ID NO: 152 and the amino acid sequence of SEQ ID NO: 187; the amino acid sequence of SEQ ID NO: 153 and the amino acid sequence of SEQ ID NO: 187; the amino acid sequence of SEQ ID NO: 119 and the amino acid sequence of SEQ ID NO: 187; the amino acid sequence of SEQ ID NO: 154 and the amino acid sequence of SEQ ID NO: 187; the amino acid sequence of SEQ ID NO: 120 and the amino acid sequence of SEQ ID NO: 187; the amino acid sequence of SEQ ID NO: 111 and the amino acid sequence of SEQ ID NO: 186; the amino acid sequence of SEQ ID NO: 116 and the amino acid sequence of SEQ ID NO: 187; the amino acid sequence of SEQ ID NO: 113 and the amino acid sequence of SEQ ID NO: 187; the amino acid sequence of SEQ ID NO: 114 and the amino acid sequence of SEQ ID NO: 187; the amino acid sequence of SEQ ID NO: 119 and the amino acid sequence of SEQ ID NO: 188; the amino acid sequence of SEQ ID NO: 120 and the amino acid sequence of SEQ ID NO: 188; the amino acid sequence of SEQ ID NO: 116 and the amino acid sequence of SEQ ID NO: 188; the amino acid sequence of SEQ ID NO: 121 and the amino acid sequence of SEQ ID NO: 188; the amino acid sequence of SEQ ID NO: 110 and the amino acid sequence of SEQ ID NO: 189; the amino acid sequence of SEQ ID NO: 110 and the amino acid sequence of SEQ ID NO: 190; the amino acid sequence of SEQ ID NO: 113 and the amino acid sequence of SEQ ID NO: 188; the amino acid sequence of SEQ ID NO: 113 and the amino acid sequence of SEQ ID NO: 191; the amino acid sequence of SEQ ID NO: 139 and the amino acid sequence of SEQ ID NO: 192; the amino acid sequence of SEQ ID NO: 151 and the amino acid sequence of SEQ ID NO: 192; the amino acid sequence of SEQ ID NO: 110 and the amino acid sequence of SEQ ID NO: 192; the amino acid sequence of SEQ ID NO: 152 and the amino acid sequence of SEQ ID NO: 192; the amino acid sequence of SEQ ID NO: 154 and the amino acid sequence of SEQ ID NO: 192; the amino acid sequence of SEQ ID NO: 153 and the amino acid sequence of SEQ ID NO: 192; the amino acid sequence of SEQ ID NO: 120 and the amino acid sequence of SEQ ID NO: 192; the amino acid sequence of SEQ ID NO: 119 and the amino acid sequence of SEQ ID NO: 192; the amino acid sequence of SEQ ID NO: 114 and the amino acid sequence of SEQ ID NO: 192; the amino acid sequence of SEQ ID NO: 113 and the amino acid sequence of SEQ ID NO: 192; the amino acid sequence of SEQ ID NO: 113 and the amino acid sequence of SEQ ID NO: 193; the amino acid sequence of SEQ ID NO: 113 and the amino acid sequence of SEQ ID NO: 194; the amino acid sequence of SEQ ID NO: 113 and the amino acid sequence of SEQ ID NO: 195; or the amino acid sequence of SEQ ID NO: 113 and the amino acid sequence of SEQ ID NO: 169.

In some embodiments, the antibody or antibody fragment disclosed herein comprises, or consists essentially of, or consists of a heavy chain variable sequence having at least 96%, at least 97%, at least 98% or at least 99% identity to a heavy chain variable amino acid sequence in Table 14 and/or a light chain variable sequence having at least 96%, at least 97%, at least 98% or at least 99% identity to a light chain variable amino acid sequence in Table 14, with the proviso that the amino acid mutations are not altered in the equivalent from those mutations identified in Table 13.

In another aspect, the present disclosure provides an isolated nucleic acid molecule comprising, or consisting essentially of, or consisting of a nucleotide sequence encoding the CDR, heavy chain, light chain, scFV, antibody or antibody fragment and equivalents as disclosed herein that are optionally detectably labeled. In some embodiments, the nucleic acid comprises a nucleotide sequence encoding an Fv fragment comprising an amino acid sequence selected from any one of SEQ ID NO: 15-99; or an amino acid sequence disclosed in Table 13 or an equivalent thereof with the proviso that the amino acid mutations are not altered in the equivalent from those mutations identified in Table 13.

Another aspect of the present disclosure provides an isolated nucleic acid comprising, or consisting essentially of, or consisting of a nucleotide sequence encoding the antibody or antibody fragment disclosed herein. In some embodiments, the nucleic acid comprises, or consists essentially of, or consists of the nucleotide sequence encoding: (a) an antibody fragment comprising an amino acid sequence selected from SEQ ID NO: 1-14 or an equivalent thereof; or (b) a heavy chain variable sequence comprising an amino acid sequence selected from SEQ ID NO: 100, 101, 102, 103, 104, 105, 106, 107, 108, or 109; or an equivalent thereof and/or (b) a light chain variable sequence comprising an amino acid sequence selected from SEQ ID NO: 155, 156, 157, 158, 159, 160, 161, 162, 163, 164, 165, or 166 or an equivalent thereof. In one aspect the equivalent nucleic acid encodes a polypeptide that has at least 96% identity to any one of the reference polypeptides and further in one embodiment has a CDR sequence that is 100% identical to the CDR of the reference polypeptide. The reference CDRs are provided above, and further within this document. Stated another way, the amino acid substitutions and modifications are in regions outside of the CDR regions.

In some embodiments, the nucleic acid comprises a nucleotide sequence encoding a heavy chain variable sequence comprising an amino acid sequence selected from any one of SEQ ID NOs: 110-154. In some embodiments, the nucleic acid comprises a nucleotide sequence encoding a heavy chain variable sequence comprising a heavy chain variable amino acid sequence disclosed in Table 14. In some embodiments, the nucleic acid comprises a nucleotide sequence encoding a heavy chain variable sequence comprising an amino acid sequence having at least 70%, 80%, 90%, 95%, 96%, 97%, 98%, or 99% identity a heavy chain variable amino acid sequence disclosed in Table 14, with the proviso that the amino acid mutations are not altered in the equivalent from those mutations identified in Table 14.

In some embodiments, the nucleic acid comprises a nucleotide sequence encoding a light chain variable sequence comprising an amino acid sequence selected from SEQ ID NO: 167, 168, 169, 170, 171, 172, 173, 174, 175, 176, 177, 178, 179, 180, 181, 182, 183, 184, 185, 186, 187, 188, 189, 190, 191, 192, 193, 194, or 195. In some embodiments, the nucleic acid comprises a nucleotide sequence encoding a light chain variable amino acid sequence comprising a light chain variable amino acid sequence disclosed in Table 14. In some embodiments, the nucleic acid comprises a nucleotide sequence encoding a light chain variable sequence comprising a light chain variable amino acid sequence having at least 70%, 80%, 90%, 95%, 96%, 97%, 98%, or 99% identity to the amino acid sequence disclosed in Table 14, with the proviso that the amino acid mutations are not altered in the equivalent from those mutations identified in Table 13.

Another aspect of the present disclosure provides a vector comprising, or consisting essentially of, or consisting of an isolated nucleic acid disclosed herein.

Another aspect of the present disclosure provides an engineered cell comprising, or consisting essentially of, or consisting of the isolated nucleic acid disclosed herein; or the vector disclosed herein.

Another aspect of the present disclosure provides a composition comprising, or consisting essentially of, or consisting of one or more antibodies or antibody fragments disclosed herein, the isolated nucleic acid disclosed herein, or the vector disclosed herein and a carrier. In some embodiments, the composition is a pharmaceutically acceptable composition.

Another aspect of the present disclosure provides a method of one or more of: treating a subject infected with SARS-CoV-2; reducing the likelihood of infection with SARS-CoV-2 in a subject at risk of infection; reducing the likelihood of symptomatic infection with SARS-CoV-2, treating a subject at risk of contracting SARS-CoV-2, the method comprising, or consisting essentially of, or consisting of administering to the subject the antibody or antibody fragment disclosed herein.

Another aspect of the present disclosure provides a method of one or more of: treating a subject infected with SARS-CoV-2; reducing the likelihood of infection of a subject at risk of infection with SARS-CoV-2; reducing the likelihood of symptomatic infection with SARS-CoV-2; treating a subject at risk of contracting SARS-CoV-2, the method comprising, or consisting essentially of, or consisting of administering to the subject a first antibody or antibody fragment and a second antibody or antibody fragment. In some embodiments, the first and second antibodies or antibody fragments synergistically neutralize the SARS-CoV-2 virus. In some embodiments, the first antibody or antibody fragment and the second antibody or antibody fragment bind to non-overlapping sites on the S glycoprotein. In some embodiments, the first antibody or antibody fragment and the second antibody or antibody fragment have a synergy score of about 10, about 12, about 14, about 15, about 17, about 18, about 20 or more. In some embodiments, the synergy score is based on experimental combination response in a focus reduction neutralization test (FRNT) and/or by synergy-scoring models.

Another aspect of the present disclosure provides a vaccine formulation comprising, or consisting essentially of, or consisting of one or more antibodies or antibody fragments disclosed herein. In some embodiments, the vaccine formulation further comprises, or consists essentially of, or consists of a second antibody or antibody fragment that binds to a SARS-CoV-2 surface spike protein. In some embodiments, the second antibody or antibody fragment is an antibody or antibody fragment disclosed herein. In some embodiments, the second antibody or antibody fragment is an antibody or antibody fragment selected from sotrovimab (S309), COV2-2381, cilgavimab (COV2-2130 or AZD1061), or tixagevimab (COV2-2196 or AZD8895). The vaccine compositions can optionally comprise adjuvants, stabilizers and/or preservatives.

Another aspect of the present disclosure provides a vaccine formulation comprising, or consisting essentially of, or consisting of one or more expression vectors. In some embodiments, the one or more expression vectors comprise at least one nucleic acid encoding an antibody or antibody fragment disclosed herein. In some embodiments, the nucleic acid is DNA, RNA, or mRNA. The vaccine compositions can optionally comprise adjuvants, stabilizers and/or preservatives.

Another aspect of the present disclosure provides a method of detecting infection with SARS-CoV-2 in a subject the method comprising, or consisting essentially of, or consisting of (a) contacting a sample isolated from the subject with the antibody or antibody fragment disclosed herein; and (b) detecting SARS-CoV-2 in the sample by detecting the binding of the antibody or antibody fragment to a SARS-CoV-2 antigen in the sample.

Another aspect of the present disclosure provides a method of determining a SARS-CoV-2 virus variant antigenic profile the method comprising, or consisting essentially of, or consisting of: (a) contacting a sample comprising the SARS-CoV-2 virus variant with the antibody or antibody fragment disclosed herein; and (b) determining the binding of the antibody or antibody fragment to the SARS-CoV-2 virus variant, wherein absence of binding indicates a conformational change and/or a mutation in the SARS-CoV-2 surface spike protein.

Another aspect of the present disclosure provides a biosensor for detecting the presence of a SARS-CoV-2 antigen in a sample, the biosensor comprising, or consisting essentially of, or consisting of (a) a transducer component comprising an electrode operatively connected to a microprocessor, the microprocessor being adapted to receive, process and transmit a signal; (b) a receptor component having a sensing element capable of detecting and binding to the SARS-CoV-2 antigen; and (c) the transducer component and the receptor component are capable of being brought into direct contact with the sample in situ. In some embodiments, the sensing element comprises the antibody or antibody fragment disclosed herein.

Another aspect of the present disclosure provides a kit comprising, or consisting essentially of, or consisting of the antibody or antibody fragment disclosed herein and instructions for using the first antibody or antibody fragment.

Another aspect of the present disclosure provides a kit comprising, or consisting essentially of, or consisting of a first antibody or antibody fragment that binds to a SARS-CoV-2 surface spike protein and a second antibody or antibody fragment that binds to a SAR-CoV-2 surface spike protein. In some embodiments, the first and/or second antibody and antibody fragment is antibody or antibody fragment disclosed herein. In some embodiments, at least one of the first and/or second antibody and antibody fragment is antibody or antibody fragment disclosed herein. In some embodiments, the kit further comprises, consists essentially of, or consists of instructions for using the first antibody or antibody fragment and the second antibody or antibody fragment for treating a subject infected with SARS-CoV-2 or for reducing the likelihood of infection of a subject at risk of contracting SARS-CoV-2.

### BRIEF DESCRIPTION OF THE DRAWINGS

FIG. 1: shows a table demonstrating the improved binding affinity (EC₅₀) of the isolated monoclonal antibodies disclosed herein against the SARS-CoV-2 Wuhan and Omicron strains when compared to monoclonal antibodies known in the art.

FIG. 2: shows a table demonstrating the estimated binding affinity (K_{D}) of the isolated monoclonal antibodies of the present disclosure.

FIG. 3: shows a table demonstrating the enhanced neutralization effects (IC₅₀) of the isolated monoclonal antibodies disclosed herein against SARS-CoV-2 Omicron strains when compared to the COV2-2130 monoclonal antibody

FIG. 4: discloses the amino acid sequences of the variable domain fragments (Fv fragments) of the novel class of anti-SARS-CoV-2 monoclonal antibodies disclosed herein.

FIGS. 5A-5E: show binding and thermostability characteristics of the disclosed IgG antibodies. FIG. 5A shows the parental COV2-2130 (pink circles) and antibodies (2130-1-0114-112 (also referred to herein as LLNL199) highlighted in orange asterisks; remainder in black) were assayed for thermal shift (n=3, technical replicates). Bars indicate the mean and error bars indicate standard deviation. FIGS. 5B-5E show the parental COV2-2130 antibody and the disclosed antibodies (represented by the same symbols as in FIG. 5A) and cross-reactive positive control antibody s309 (blue squares), which were analyzed for relative binding against four SARS-CoV-2 Spike-RBD variants in Gyrolab immunoassay: Wild-type (FIG. 5B), Delta (FIG. 5C), Omicron BA.1 (FIG. 5D) and Omicron BA.1.1 (FIG. 5E). Lines represent 4-parameter logistic regression model fit using GraphPad Prism to each titration, executed without technical replicates.

FIGS. 6A-6E: show pseudoviral neutralization. The parental COV2-2130 antibody (circles), cross-reactive positive control antibody 5309 (squares), and down-selected by the disclosed antibodies (2130-1-0114-112 (also referred to herein as LLNL199) highlighted in orange asterisks; others in black) were assayed for neutralization of lentivirus pseudotyped with Spike variants D614G (FIG. 6A), Omicron BA.1 (FIG. 6B), BA.1.1 (FIG. 6C), BA.2 (FIG. 6D), and BA.4 (FIG. 6E). Symbols indicate the mean and standard deviation of two technical replicates; curves are 4-parameter logistic regression models fit using GraphPad Prism.

FIGS. 7A-7G: show authentic virus neutralization by focus reduction neutralization test in Vero-TMPRSS2 cells. 2130-1-0114-112 (also referred to herein as LNLL199) potently neutralizes (FIG. 7A) WA1/2020 D614G (FIG. 7B) Omicron BA.1, (FIG. 7C) Omicron BA.1.1, (FIG. 7D) Omicron BA.2, (FIG. 7E) Omicron BA.2.12.1, (FIG. 7F) Omicron BA.4, and (FIG. 7G) Omicron BA.5 authentic viruses in focus reduction neutralization assays in Vero-TMPRSS2 cells. All experiments were performed in technical duplicate, symbols represent the mean of the duplicates, and fits are four-parameter logistic curves. All analysis performed in GraphPad Prism.

FIG. 8: show in vivo prophylaxis by 2130-1-0114-112 (also referred to herein as LLNL199) against SARS-CoV-2 variants. Eight-week-old female K18-hACE2 mice were administered 100 μg (about 5 mg/kg) of the indicated mAb treatment by intraperitoneal injection one day before intranasal inoculation with 104 FFU of WA1/2020 D614G (Left), BA.1.1 (Center) or BA.5 (Right). Tissues were collected four days after inoculation and viral RNA levels in the lungs (Top), nasal turbinates (Center), and nasal washes (Bottom) were determined by RT-qPCR (lines indicate median±SEM.; n=9-10 mice per group, two experiments; Kruskal-Wallis test with Dunn's multiple comparisons test; ns, not significant; *P<0.05, **P<0.01, ***P<0.001, ****P<0.0001). All analysis conducted in GraphPad Prism.

FIGS. 9A-9E: show Cryo-EM structure of neutralizing antibodies 2130-1-0114-112 (also referred to herein as LLNL199) in complex with Cov2 BA.2 RBD. FIG. 9A shows Cryo-EM map and model of the RBD-Fab complex. The map is transparent and colored by chain with RBD red, 2130-1-0114-112 (also referred to herein as LNLL199) HC yellow and 2130-1-0114-112 (also referred to herein as LNLL199) LC green. FIG. 9B shows atomic model of the RBD-Fab complex, which were labeled as in FIG. 9A, with hydrogen bond shown in dashed line; BA.2 RBD mutation in orange, and The heavy chain (HC) and light chain variable regions of the 2130-1-0114-112 (also referred to herein as also referred to herein as LLNL199) mutation shown in cyan and blue respectively. FIG. 9C shows detail illustrating the interaction between the heavy chain complementary-determining region (HCDR) and Light chain CDR (LCDR) of 2130-1-0114-112 (also referred to herein as LLNL199) modified residues and the Cov2 BA.2 RBD. Left, HCDR3 Glu112. Middle, LCDR1 Ala32 and Ala33 hydrophobic network. Right, LCDR2 Glu59 salt bridge with Arg498. Orange and green dashed lines indicate H-bonds and hydrophobic interactions, respectively; yellow dashed lines are labeled with distances. FIG. 9D shows the HCDR3 shown as in (FIG. 9C) with surface color by electrostatic potential, showing the positive and negative charges of Lys444 and Glu112; and A32 and A33 in the LCDR1 with the nearby RBD surface colored by hydrophobicity (orange to cyan hydrophobic to hydrophilic). FIG. 9E shows a 2D diagram of Fab 2130-1-0114-112 (also referred to herein as LLNL199) paratope and epitope residues involved in hydrogen bonding (dashed lines) and hydrophobic interactions. Residues involved in hydrophobic interactions are shown as curved lines with rays, where atoms are shown as circles, with oxygen red, carbon black, and nitrogen blue, and interacting residues that belong to the CDR loops are colored in different shade. Asterisks correspond to mutated residues. Image created with Ligplot+[Laskowski 2011].

FIGS. 10A-10D: show optimized, single-concentration screening data in Gyrolab immunoassays, which allowed for the selection of candidates from Set 1 (n=230) for down-stream characterization. Parental mAb COV2-2130 (magenta circles) and positive control mAb 5309 (blue squares, [Pinto 2020]) served as references for disclosed mAbs (gray diamonds) in single-concentration immunoassays. Target antigens were Wild Type (FIG. 10A), Omicron BA.1 (FIG. 10B), Delta (FIG. 10C), and Omicron BA.1.1 (FIG. 10D). Each screened antibody and antigen combination was evaluated in two replicate assays; each of the controls was replicated in two replicate assays for each of three groups of antibodies, resulting in six replicate assays for each point on the control curves. All error bars indicate standard deviation.

FIGS. 11A-11D: show line graphs demonstrating results of ELISA screening, which allowed for down selection of candidates from Set 2 (n=204), for down-stream characterization. Parental mAb COV-22130 (red circles; 3 technical replicates) and positive control mAb 5309 (blue squares; 3 technical replicates [pinto2020]) serve as references for disclosed mAbs (black curves). Orange squares are 2130-1-0114-112 (also referred to herein as also referred to herein as LLNL199). Each designed antibody had a single measurement (n=1) at each concentration. All curves were 4-parameter logistic fits, produced in GraphPad Prism. Target antigens are Wild Type (FIG. 11A), Omicron (Acro) (FIG. 11B), Omicron BA.1 (FIG. 11C), and Omicron BA.1.1 (FIG. 11D).

FIGS. 12A-12G: show normalized pseudoviral neutralization of recently emerged VOCs by 2130-1-0114-112 (also referred to herein as LLNL199). FIG. 12A provides a legend for antibodies tested. FIG. 12B shows that COV2-2130 and 2130-1-0114-112 (also referred to herein as LLNL199) potently neutralize D614G. FIG. 12C shows that only 2130-1-0114-112 (also referred to herein as LLNL199) potently neutralized BA.1, consistent with other pseudoviral neutralization assays. FIG. 12D shows 2130-1-0114-112 (also referred to herein as LLNL199) potently neutralizes BA.2.75, outperforming COV2-2130 by 90-fold—12E-12F show that 2130-1-0114-112 (also referred to herein as LLNL199) loses substantial potency in the context of BA.4.6 and artificially-produced BA.2.75+R346T (here listed as “BA.2.76”) but retains measurable neutralization, demonstrating mitigation of this important weakness of COV2-2130. FIGS. 12B-12F show the mean of two technical replicates and curves show a fitted four-parameter logistic curve with fixed minimum and maximum values. FIG. 12G shows neutralization IC50 values in ng/ml. “>” indicates IC50 greater than 10,000 ng/mL. All analysis conducted in GraphPad Prism.

FIGS. 13A-13F: show authentic virus neutralization by focus reduction neutralization test in Vero-TMPRSS2 cells. 2130-1-0114-112 (also referred to herein as LLNL199) potently neutralizes (FIG. 13A) Delta (B.1.617.2), (FIG. 13B) Omicron BA.1, (FIG. 13C) Omicron BA.1.1, (FIG. 13D) Omicron BA.4, and (FIG. 13E) Omicron BA.5.5 authentic viruses in focus reduction neutralization assays in Vero-TMPRSS2 cells. All experiments were performed in technical duplicate, symbols represent the mean of the duplicates, and fits are four-parameter logistic curves. FIG. 13F shows IC₅₀ values in μg/ml. >indicates greater than 10 μg/ml. All analysis performed in GraphPad Prism.

FIGS. 14A-14D: show authentic virus neutralization in plaque assays using Vero E6-TMPRSS2-T2A-ACE2 (VAT) cells. All SARS-CoV-2 viral stocks as well as VAT cells were obtained through BEI Resources, NIAID, NIH. FIG. 14A shows Delta variant isolate hCoV-19/USA/MD-HP05647/2021, lineage B.1.617.2; NR-55672. FIG. 14B shows BA.1 isolate hCoV-19/USA/GA-EHC-2811C/2021, lineage B.1.1.529; NR-56481). FIG. 14C shows BA1.1 (isolate hCoV-19/USA/HI-CDC-4359259-001/2021, lineage B.1.1.529; NR-56475). Viral stocks were amplified in Vero E6 cells (Delta variant) or VAT cells (Omicron variants). Serial dilutions of mAbs were incubated with virus at a concentration of 400 PFU/mL at 37° C. with 5% CO2 for 30 min. Antibody-virus complexes were then added to VAT cells in 12-well plates and incubated for 30 min, then overlaid with 2 mL per well of 0.6% microcrystalline cellulose (Sigma) in minimal essential media (ThermoFisher) supplemented with 0.3% bovine serum albumin (Sigma), and 1% penicillin/streptomycin (ThermoFisher). After 72 hours incubation, plaques were visualized by incubation in 0.25% crystal violet in 100% methanol for 10 minutes. Data are represented as the normalized infection of mAb-treated virus to virus treated with control human IgG (Invitrogen). For S309, each point shows the mean of four technical replicates; all other points are means of two technical replicates; error bars show standard error. Curves are two-parameter (IC50, hill-slope) logistic fits to normalized response. FIG. 14D shows IC₅₀ values (μg/ml) illustrating that 2130-1-0104-024 (also referred to herein as LLNL193), while having only two mutations from COV2-2130, remained strongly potent against BA.1 and suffered a 20-times loss in potency against BA.1.1. 2130-1-0114-112 (LLNL199) was strongly potent against all three tested variants. All analysis performed in GraphPad Prism.

FIGS. 15A-15E: show Cryo-EM structure of neutralizing antibodies 2130-1-0114-112 (also referred to herein as LLNL199) in complex with Cov2 BA.2 RBD. FIG. 15A shows RBD residues in 7 Å distance form 2130-1-0114-112 (also referred to herein as LLNL199). RBD in red, with BA.2 mutated residues in orange, and 2130-1-0114-112 (also referred to herein as LLNL199) in yellow/green. FIG. 15B shows Left, 3D representation of the interaction plot between RBD and 2130-1-0114-112 (also referred to herein as LLNL199) HC. 2130-1-0114-112 (also referred to herein as LLNL199) is shown as stick (yellow) and RBD as gray sphere with the contact residues in red. Contact residues labelled and numbered. Right, 3D representation of the interaction plot between RBD and 2130-1-0114-112 (also referred to herein as LLNL199) LC. 2130-1-0114-112 (also referred to herein as LLNL199) is shown as stick (green) and RBD as gray sphere with the contact residues in red. Contact residues are labelled and numbered. FIG. 15C shows Glu112 and Lys440 with the EM map and distance between the sidechains. FIG. 15D shows Fab COV-2130 paratope and epitope residues involved in hydrogen bonding (dashed lines) and hydrophobic interactions. Hydrophobic interactions residues are shown as curved lines with rays. Atoms shown as circles, with oxygen red, carbon black, and nitrogen blue. Image was created with Ligplot+. FIG. 15E Representative micrograph.

FIG. 16: shows CryoEM workflow.

FIGS. 17A-17D: show full-curve Gyros binding of strain (FIG. 17A) Wuhan; (FIG. 17B) Delta; (FIG. 17C) Omicron BA.1; (FIG. 17D) Omicron BA.1.1.

FIGS. 18A-18D: show the neutralization of strain (FIG. 18A) BA1; (FIG. 18B) BA1.1; (FIG. 18C) BA1.1 (bottom); (FIG. 18D) BA2.

FIGS. 19A-19E: show authentic virus neutralization by focus reduction neutralization test in Vero-TMPRSS2 cells. 2130-1-0114-112 (also referred to herein as LLNL199) and 2130-1-0121-354 (also referred to herein as B-133) potently neutralizes (FIG. 19A) B.1.617.2; (FIG. 19B) BA.1; (FIG. 19C) BA1.1; (FIG. 19D) BA.4; (FIG. 19E) BA.5.5 authentic viruses in focus reduction neutralization assays in Vero-TMPRSS2 cells. All experiments were performed in technical duplicate, symbols represent the mean of the duplicates, and fits are four-parameter logistic curves.

FIGS. 20A-20F: Show designed antibodies improve pseudoviral neutralization over COV2-2130. The parental COV2-2130 antibody (orange circles), cross-reactive positive control antibody rS2K146 (magenta squares), and down-selected computationally designed antibodies were assayed by neutralization with lentiviruses pseudotyped with spike variants of Wuhan-1 D614G (FIG. 20A), Omicron BA.1 (FIG. 20B), BA.1.1 (FIG. 20C), BA.2 (FIG. 20D), and BA.4 (FIG. 20E). FIG. 20F shows a key for the symbols of FIGS. 20A-20E. Symbols indicate the mean and standard deviation of two technical replicates; curves are 4-parameter logistic regression models fit using GraphPad Prism.

### DETAILED DESCRIPTION

The COVID-19 pandemic has highlighted how viral variants that escape monoclonal antibody drugs can undermine drug development efforts. The substantially mutated SARS-CoV-2 variants Omicron BA.1 and BA.1.1 escaped many clinical antibody drug products, including AZD1061 [VanBlargan 2022]. Rapidly modifying existing antibody-based drugs products to restore efficacy to emerging variants is a potential mitigation strategy. To that end, the design of a novel antibody derived from COV2-2130, the progenitor of AZD1061, that could potently neutralize BA.1 and BA.1.1 while maintaining efficacy against the Delta variant were sought. Accordingly, the present disclosure provides disclosed derivatives of COV2-2130 that achieve this goal. Notably, the antibody designs described herein achieved potent neutralization of Delta, BA.1, BA.1.1, and BA.2, and also potently neutralized more recent BA.4 and BA.5.5 Omicron strains, where the parental COV2-2130 again suffers significant losses in potency. This work provides a novel computational system for rapid antibody adaptation addressing escape variants. The present disclosure also provides a platform that combines high-performance computing-enabled simulation, machine learning, and structural bioinformatics to optimize multiple antibody properties simultaneously. Combined with knowledge of potential escape liabilities, the approach described herein could support pre-emptive design of broadly protective monoclonal antibodies that are robust across a wide range of possible antigenic variants.

Viruses evolve gradually or suddenly, which can result in evasion of the protection offered by medical countermeasures, including therapeutic or prophylactic monoclonal antibodies. During the course of the COVID-19 pandemic, new variants of the SARS-CoV-2 virus have emerged that evade FDA-approved countermeasures, candidates, and the human immune system, posing new dangers to the public.

AZD7442 (Evusheld), an FDA Emergency Authorized monoclonal antibody, is a combination of two fully human, SARS-CoV-2-neutralizing monoclonal antibodies (tixagevimab and cilgavimab) that are derived from antibodies isolated from B cells obtained from persons infected with SARS-CoV-2. Cilgavimab (AZD1061) is a derivative of the monoclonal antibody COV2-2130. Another derivative of COV2-2130 is also used in the antibody product being produced by Ology Bioservices (e.g., COV2-2130 and COV2-2381) under contract with the Department of Defense (DOD). While the combination of Tixagevimab and cilgavimab is fully active against the Delta variant of SARS-CoV-2 (B.1.617.2 and AY lineages), the combination of Tixagevimab and cilgavimab has decreased neutralizing activity in vitro against the Omicron variants. See e.g., JAMA, 327(4):384-385 (2022). Specifically, the antibody COV-2130 fails to neutralize the BA.1 and BA.1.1 (“Omicron”) strains of SARS-CoV-2 in vitro. Given the continuous evolution of the virus that leads to SARS-CoV-2 variants and the constant developments in our understanding of the impacts of these variants, there is a need for novel prophylactic monoclonal antibodies that can bind to and neutralize old and emerging strains of SARS-CoV-2.

One aspect of the present disclosure provides a novel class of anti-SARS-CoV-2 antibodies that exhibited dramatically improved in vitro binding and/or neutralization effects against all known SARS-CoV-2 variants, including but not limited to the “Omicron” variants BA.1 and BA.1.1 (e.g, B.1.1.529, BA.1, BA.1.1, BA.2, BA.3, BA.4 and BA.5 lineages). The novel class anti-SARS-CoV-2 antibodies showed enhanced binding and/or neutralization of older SARS-CoV strains (e.g., Alpha (B.1.1.7 and Q lineages); Beta (B.1.351 and descendent lineages); Gamma (P.1 and descendent lineages); Delta (B.1.617.2 and AY lineages); Epsilon (B.1.427 and B.1.429); Eta (B.1.525); Iota (B.1.526); Kappa (B.1.617.1); 1.617.3; Mu (B.1.621, B.1.621.1); Zeta (P.2)). In particular, the novel class anti-SARS-CoV-2 antibodies showed enhanced binding and/or neutralization of the “Omicron” variants such as B.1.1.529, BA.1, BA.1.1, BA.2, BA.3, BA.4 and BA.5 lineages. This novel class antibodies disclosed herein were derived from the COV-2130 monoclonal antibody and were re-engineered specifically for improved and enhanced binding and neutralization characteristics against several SARS-CoV-2 strains (i.e., old and emerging strains).

The novel antibodies of the present disclosure can be used as part of a therapeutic or prophylactic drug substance or drug product. They also can be used in diagnostics, detection and as personalized therapies. In some embodiments, the novel antibodies are used as: (1) a “drop-in” replacement of existing AZD1061/COV-2130 prophylactic, (2) a parallel prophylactic (delivered prior to infection) or therapeutic (delivered after infection) products, or (3) in combination with other anti-SARS-CoV-2 antibodies or therapeutics. In some embodiments, the novel antibodies of the present disclosure are used as part of a diagnostic or biosensor for identifying which strain or strains of coronavirus, severe acute respiratory syndrome coronavirus, or SARS-CoV-2 is or are present in a sample. In some embodiments, the novel antibodies are used to predict the efficacy of Evusheld, Cilgavimab, Resilience or related therapeutics, and/or prophylactics against a patient's individual COVID-19 case. In some embodiments, the novel antibodies are used as reagents in laboratory processes, or for similar functions.

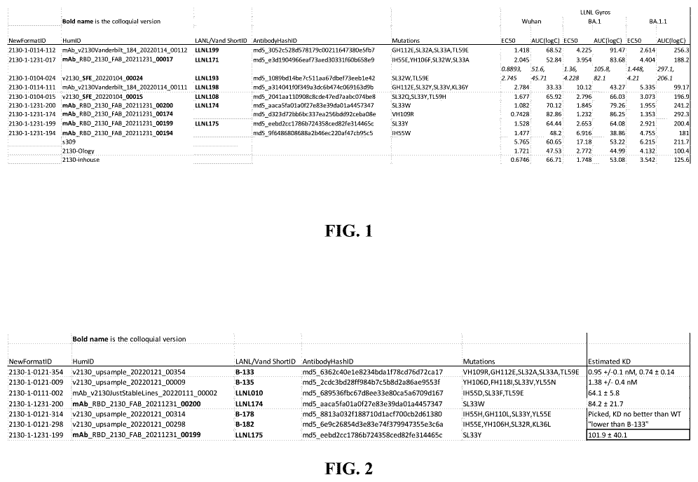

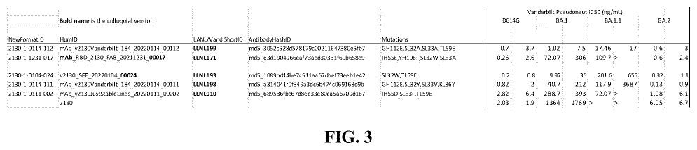

The state of the art method for dealing with the emergence of novel SARS-CoV-2 strains is to abandon or substantially modify the drug product or drug substance, resulting in large delays and/or total loss of the product line. The antibodies of this disclosure and the methods of making them (1) can avoid this outcome for the deployed Evusheld/Cilgavimab product, and (2) are far less expensive and have a less uncertain path to minimal, robust repair of approved and deployed antibody or protein drug substances and products. In the context of the COVID-19 pandemic in particular, better protection against BA.1 and BA.1.1 and future related strains could save lives and substantial sums of money, depending on how the pandemic continues to evolve. Because the antibodies of the present disclosure introduced targeted new chemistries to the antibody/antigen interface, they showed excellent binding and neutralization characteristics against old SARS-CoV-2 strains, while also achieving the critical goal of improving/restoring efficacy against new emerging variants, such as BA.1 and BA.1.1 and other Omicron lineages and related strains (e.g, B.1.1.529, BA.1, BA.1.1, BA.2, BA.3, BA.4, and BA.5 lineages). Indeed, FIG. 1 and Table 15 shows the antibodies of the present disclosure show improved affinity (EC₅₀) against either the BA1 variant and the Wuhan variant compared to the parental antibody or the BA.1.1 variant (e.g., EC₅₀=1.353, 1.955 or 2.614) than known COV2-2130 variants (e.g., EC₅₀=3.542 or 4.132). Moreover, as shown in FIG. 3, the neutralization characteristics (IC₅₀) of the antibodies of the present disclosure are also superior to those of the classic COV2-2130 antibody. FIG. 3 shows that the IC₅₀ of the antibodies of the present disclosure ranges from 0.1-1.1 ng/ml vs 6.1 ng/ml for COV2-2130; 17 ng/ml vs>3687 ng/ml for COV2-2130; or 7.5 ng/ml v. 1769 ng/ml for COV2-2130.

The monoclonal antibodies of the present disclosure are an improvement over previously described anti-SARS-CoV-2 monoclonal antibodies particularly because of their enhancements in binding and neutralization of known SARS-CoV-2 strains when compared to the COV-2130 antibody, even for some strains that COV-2130 binds and neutralizes well. The novel class of antibodies of the present disclosure have unique mutations in the complementary determining regions of the heavy (V_{H}) and light chain (V_{L}) variable domains. These mutations were surprising because they alter residues in the COV2-2130 monoclonal antibody that offered surprising, and across-the-board improvements for binding old and emerging strains of SARS-CoV-2, including the Omicron variants (e.g, B.1.1.529, BA.1, BA.1.1, BA.2, BA.3, BA.4, and BA.5 lineages). For example, in the CDR2 of the heavy chain variable domain, the isoleucine residue at position 55 of the COV2-2130 sequence was mutated to either histidine (I55H; SEQ ID NO: 108), glutamic acid (I55E; SEQ ID NO: 100 or 107), aspartate (I55D; SEQ ID NO: 104) or tryptophan (I55W; SEQ ID NO: 102). In the CDR3 of the heavy chain variable domain, the glycine residue at position 112 of the COV2-2130 sequence was mutated to glutamic acid (G112E), which altered the characteristics of the H3 loop. Other mutations were surprising because they delivered large empirical effects from chemically modest mutant residues. For example, in the CDR1 of the light chain variable domain of the COV2-2130 sequence, the serine residues at positions 32 and 33 of the COV2-2130 sequence were mutated to alanine (e.g., S32A, S33A; SEQ ID NO: 163). See Table 13 for the list of specific mutations in the disclosed antibodies.

In addition, the antibodies provided by the present disclosure repair a clinically deployed prophylactic product (e.g., COV2-2130; Cilgavimab (AZD1061)) rendered dramatically less effective by the emergence of novel SARS-CoV-2 strains, such as the current BA.1, BA.1.1, and related “Omicron” strains.

Accordingly, in one aspect, the present disclosure provides an isolated antibody or antibody fragment that binds to a SARS-CoV-2 surface protein (e.g., a spike (S) glycoprotein) having improved binding and/or neutralization characteristics against all known SARS-CoV-2 strains when compared to a monoclonal antibody selected from sotrovimab (S309), COV2-2381, cilgavimab (COV2-2130 or AZD1061), and/or tixagevimab (COV2-2196 or AZD8895). The SARS-CoV-2 variant is selected from Alpha (B.1.1.7 and Q lineages); Beta (B.1.351 and descendent lineages); Gamma (P.1 and descendent lineages); Delta (B.1.617.2 and AY lineages); Epsilon (B.1.427 and B.1.429); Eta (B.1.525); Iota (B.1.526); Kappa (B.1.617.1); 1.617.3; Mu (B.1.621, B.1.621.1); Zeta (P.2); or Omicron (B.1.1.529, BA.1, BA.1.1, BA.2, BA.3, BA.4 and BA.5 lineages). In particular, the SARS-CoV-2 strain is an “Omicron” strain or variants thereof, such as B.1.1.529, BA.1, BA.1.1, BA.2, BA.3, BA.4 and BA.5 lineages. In some embodiments, the isolated antibody or antibody fragment comprises, or consists essentially of, or consists of heavy chain complementary determining regions 1-3 (CDRH1-3) sequences; and a light chain complementary determining regions 1-3 (CDRL1-3) sequences disclosed in Tables 1˜4 or Table 13. In some embodiments, the isolated antibody or antibody fragment comprises a heavy chain variable sequence and a light chain variable sequence disclosed in Table 4. The antibody fragment is a recombinant antibody variable domain fragment (Fv fragment) and/or comprises the amino acid sequence of SEQ ID NOs: 1-14 or 15-99, or an amino acid sequence SEQ ID NO disclosed in Table 4, Table 13 or Table 14.

Another aspect of the present disclosure provides an isolated nucleic acid comprising, or consisting essentially of, or consisting of a nucleotide sequence encoding the antibody or antibody fragment disclosed herein. Another aspect of the present disclosure provides a vector comprising, or consisting essentially of, or consisting of an isolated nucleic acid comprising a nucleotide sequence encoding the antibody or antibody fragment disclosed herein. Another aspect of the present disclosure provides an engineered cell comprising, or consisting essentially of, or consisting of the isolated nucleic acid comprising a nucleotide sequence encoding the antibody or antibody fragment disclosed herein. Another aspect of the present disclosure provides a composition comprising, or consisting essentially of, or consisting of one or more of the antibodies or antibody fragments disclosed herein, the isolated nucleic acid, or the vector disclosed herein. Another aspect of the present disclosure provides a vaccine formulation comprising, or consisting essentially of, or consisting of one or more antibodies or antibody fragments disclosed herein.

Another aspect of the present disclosure provides a method of treating an infectious disease, e.g., viral infection, coronavirus infection, SARS-CoV-2 infection. In one aspect, the present disclosure provides a method of treating a subject infected with SARS-CoV-2 or reducing the likelihood of infection of a subject at risk of contracting SARS-CoV-2, comprising, or consisting essentially of, or consisting of delivering to the subject the antibody or antibody fragment disclosed herein. Another aspect provides a method of treating a subject infected with SARS-CoV-2 or reducing the likelihood of infection of a subject at risk of contracting SARS-CoV-2, comprising, or consisting essentially of, or consisting of delivering to the subject a first antibody or antibody fragment and a second antibody or antibody fragment. In some embodiments, the first and second antibodies or antibody fragments synergistically neutralize the SARS-CoV-2 virus.

Another aspect of the present disclosure provides a method of detecting an infectious agent in a subject suspected of being infected by a coronavirus. In one aspect, the disclosure provides a method of detecting infection with SARS-CoV-2 in a subject comprising, or consisting essentially of, or consisting of contacting a sample isolated from the subject with the antibody or antibody fragment disclosed herein; and detecting SARS-CoV-2 in the sample by assessing the binding of the antibody or antibody fragment disclosed herein to a SARS-CoV-2 antigen in the sample.

Another aspect of the present disclosure provides a method of determining a SARS-CoV-2 virus variant antigenic profile comprising, or consisting essentially of, or consisting of: contacting a sample comprising the SARS-CoV-2 virus variant with the antibody or antibody fragment disclosed herein; and determining the binding of the antibody or antibody fragment to the SARS-CoV-2 virus variant, wherein absence of binding indicates a conformational change and/or a mutation in the SARS-CoV-2 surface spike protein.

Another aspect of the present disclosure provides a biosensor for detecting the presence of a SARS-CoV-2 antigen in a sample, the biosensor comprising a transducer component comprising, or consisting essentially of, or consisting of an electrode operatively connected to a microprocessor, the microprocessor being adapted to receive, process and transmit a signal; a receptor component having a sensing element capable of detecting and binding to the SARS-CoV-2 antigen; and the transducer component and the receptor component are capable of being brought into direct contact with the sample in situ. For example, the sensing element comprises the antibody or antibody fragment disclosed herein.

Another aspect of the present disclosure provides a kit comprising, or consisting essentially of, or consisting of the antibody or antibody fragment disclosed herein and instructions for using the antibody or antibody fragment.

It will be understood that monoclonal antibodies binding to SARS-CoV-2 will have several applications. These include the production of diagnostic kits for use in detecting and diagnosing SARS-CoV-2 infection, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art (see, e.g., Antibodies: A Laboratory Manual, Cold Spring Harbor Laboratory, 1988; U.S. Pat. No. 4,196,265).

### Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, the singular forms “a”, “an,” and “the” include plural referents unless the context clearly indicates otherwise. For example, the term “a cell” includes a plurality of cells, including mixtures thereof, and means one cell or more than one cell

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (—) (±) 20%, 15%, 10%, 5%, 3%, 2%, or 1%. Preferably ±5%, more preferably ±1%, and still more preferably ±0.1% from the specified value, as such variations are appropriate to perform the disclosed methods.

Administration or treatment in “combination” refers to administering two agents such that their pharmacological effects are manifest at the same time. Combination does not require administration at the same time or substantially the same time, although combination can include such administrations.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “Antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response.

As used herein, the term “antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, variable domain fragment (Fv), Fab and F(ab)2, as well as single chain antibodies (scFv) and humanized antibodies In some embodiments, antibody refers to such assemblies (e.g., intact antibody molecules, immunoadhesins, or variants thereof) which have significant known specific immunoreactive activity to an antigen of interest (e.g., SARS-CoV-2 protein). Antibodies and immunoglobulins comprise light and heavy chains, with or without an interchain covalent linkage between them. Basic immunoglobulin structures in vertebrate systems are relatively well understood.

In some embodiments, an antibody is a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules. In one embodiment, the antibody or antibody molecule comprises, e.g., consists of, an antibody fragment.

The term “antibody fragment” refers to a portion of an intact antibody and refers to the antigenic determining variable regions of an intact antibody. In some embodiments, the term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, Fv fragments, scFv antibody fragments, linear antibodies, single domain antibodies such as sdAb (either V_{L} or V_{H}), camelid VHH domains, and multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3)(see U.S. Pat. No. 6,703,199, which describes fibronectin polypeptide minibodies). “Fab” means a monovalent antigen-binding fragment of an immunoglobulin that is composed of the light chain and part of the heavy chain. F(ab′)2 means a bivalent antigen-binding fragment of an immunoglobulin that contains both light chains and part of both heavy chains.

As used herein, the term “Fv fragment” or “variable domain fragment” refers to a VH domain and a VL domain of an antibody specifically binding to an antigen, both domains forming together a Fv fragment. In some embodiment, Fv fragments means an antibody fragment comprising the V_{H} and V_{L} domains of an antibody, wherein these domains are present in a single polypeptide chain as disclosed in Table 4 herein. Generally, the Fv fragment polypeptide further comprises a polypeptide linker between the V_{H} and V_{L} domains polypeptide that enables the scFv to form.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

As used herein, the term “antibody heavy chain” refers to the larger of the two types of polypeptide chains present in all antibody molecules in their naturally occurring conformations.

As used herein, the term “antibody light chain” refers to the smaller of the two types of polypeptide chains present in all antibody molecules in their naturally occurring conformations. Kappa (κ) and lambda (λ) light chains refer to the two major antibody light chain isotypes.

As used herein, the term “synthetic antibody” means an antibody, which is generated using recombinant DNA technology, such as, for example, an antibody expressed by a bacteriophage. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bispecific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “antigen” or “Ag” is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

As used herein, the term “complementarity determining region” or “CDR” refers to the sequences of amino acids within antibody variable regions which confer antigen specificity and binding affinity. For example, in general, there are three CDRs in each heavy chain variable region (e.g., CDRH1, CDRH2, and CDRH3) and three CDRs in each light chain variable region (CDRL1, CDRL2, and CDRL3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof, North et al., J. Molecular Biology, 406 (2): 228-256 (2011) and; Lefranc et al. Nucl. Acids Res. 27:209-212 (1999) or Ruiz et al. Nucl. Acids Res. 28:219-221 (2000)) (IMGT numbering scheme).

Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). Under the IMGT numbering scheme, in some embodiments, the CDR amino acids in the VH are numbered 27-38 (HCDR1), 56-65 (HCDR2), and 105-120 (HCDR3); and the CDR amino acid residues in the VL are numbered 27-38 (LCDR1), 56-65 (LCDR2), and 105-120 (LCDR3).

In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

As used herein, the term “disease” refers to a state of health of an animal wherein the animal cannot maintain homeostasis, and wherein if the disease is not ameliorated then the animal's health continues to deteriorate. In contrast, the term “disorder” in an animal refers to a state of health in which the animal is able to maintain homeostasis, but in which the animal's state of health is less favorable than it would be in the absence of the disorder. Left untreated, a disorder does not necessarily cause a further decrease in the animal's state of health.

A “composition” as used herein, refers to an active agent, such as a compound as disclosed herein and a carrier, inert or active. In some embodiments, a carrier can be, without limitation, solid such as a bead or resin, or liquid, such as phosphate buffered saline. In some embodiments, a carrier also includes pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein, the term “encoding” refers to the inherent property of specific sequences of nucleotides in a polynucleotide (such as a gene, a cDNA, or an mRNA), to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

As used herein, the term “recombinant host cell,” “recombinant cell,” “engineered host cell,” or “engineered cell,” means a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” or “cell” as used herein. Host cells include progeny of a single host cell, and the progeny may not necessarily be completely identical (in morphology or in total DNA complement) to the original parent cell due to natural, accidental, or deliberate mutation and/or change. A host cell includes a cell transfected or infected in vivo or in vitro with a recombinant vector, an expression vector, or a nucleic acid encoding an antibody of the present disclosure. A host cell, which comprises a recombinant vector, expression vector, or a nucleic acid encoding an antibody disclosed herein, may also be referred to as a “recombinant host cell,” “engineered host cell,” or “engineered cell”.

As used herein, the term “host cell” refers to a cell, which may be used in a process for purifying an immunogenic protein or recombinant antibody in accordance with the present disclosure. Such host cell expresses the protein of interest (the antibody disclosed herein). A host cell may also be referred to as a protein-expressing cell. “Host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell, according to the present disclosure, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsellia sp., Lactobacillus sp. or Bacillus subtilis. In some embodiments, the host cell is a yeast cell. In that embodiment, the yeast host cell is selected from the group consisting of Saccharomyces cerevisiae, Hansenula polymorpha, and Pichia pastoris.

“Eukaryotic cells” comprise all of the life kingdoms except Monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

“Effective amount” or “therapeutically effective amount” as used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

In some embodiments, an “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. Provided herein in the experimental examples are effective amounts determined in a murine or rat animal model, which can be converted to an effective dose by converting the reported μg of particle per g of the mouse. A typical adult mouse is from about 15 to about 35 g for a female mouse and about 20 g to about 30 g for a male mouse. In general, one will desire to administer an amount of the compound that is effective to achieve a serum level commensurate with the concentrations found to be effective in vivo. These considerations, as well as effective formulations and administration procedures are well known in the art and are described in standard textbooks.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “ex vivo,” refers to cells that have been removed from a living organism, (e.g., a human) and propagated outside the organism (e.g., in a culture dish, test tube, or bioreactor).

As used herein, the term “humanized antibodies” refers to human forms of non-human (e.g., murine) antibodies, and are chimeric immunoglobulins, immunoglobulin chains or fragments thereof (such as Fv, Fab, Fab′, F(ab′)2 or other antigen-binding subsequences of antibodies), which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues.

Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321: 522-525 (1986); Reichmann et al., Nature 332: 323-329 (1988); Presta, Curr. Op. Struct. Biol. 2: 593-596 (1992).

As used herein, the term “fully human antibody” refers to an immunoglobulin, such as an antibody, where the whole molecule is of human origin or consists of an amino acid sequence identical to a human form of the antibody. In some embodiments, the antibodies disclosed herein are fully human antibodies. In some embodiments, the antibodies are humanized antibodies.

As used herein, the term “sequence identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” as used herein is defined as a cellular response to an antigen that occurs when lymphocytes identify antigenic molecules as foreign and induce the formation of antibodies and/or activate lymphocytes to remove the antigen.

As used herein, the term “immune effector cell,” refers to a cell that is involved in an immune response, e.g., in the promotion of an immune effector response. Examples of immune effector cells include T cells (e.g., alpha/beta T cells and gamma/delta T cells), B cells, natural killer (NK) cells, natural killer T (NKT) cells, mast cells, and myeloid-derived phagocytes.

As used herein, the term “immune effector function or immune effector response,” refers to a function or response that enhances or promotes an immune attack of a target cell. In some embodiment, an immune effector function or response refers to a property of a T or NK cell that promotes the killing or the inhibition of growth or proliferation, of a target cell. In the case of a T cell, primary stimulation and co-stimulation are examples of immune effector function or response.

As used herein, the term “instructional material” includes a publication, a recording, a diagram, or any other medium of expression which can be used to communicate the usefulness of the compositions and methods of the disclosure. The instructional material of the kit of the invention may, for example, be affixed to a container which contains the nucleic acid, peptide, and/or composition of the disclosure or be shipped together with a container which contains the nucleic acid, peptide, and/or composition. Alternatively, the instructional material may be shipped separately from the container with the intention that the instructional material and the compound be used cooperatively by the recipient.

As used herein, the term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “lentivirus” refers to a genus of the retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, the term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3. n=4, n=5 and n=6, n=7, n=8, n=9 and n=10. In one embodiment, the flexible polypeptide linkers include, but are not limited to, (Gly4 Ser)4 or (Gly4 Ser)3. In another embodiment, the linkers include multiple repeats of (Gly2Ser), (GlySer) or (Gly3Ser).

As used herein, the term “modified” means a changed state or structure of a molecule or cell of the invention. Molecules may be modified in many ways, including chemically, structurally, and functionally. Cells may be modified through the introduction of nucleic acids.

As used herein the term, “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

As used herein, the term “neutralization” refers to the ability of an antibody by itself to inhibit infection of susceptible cells or, in the case of some extracellular organisms, to inhibit an initial pathogenic step. In some embodiments, neutralization of infectivity in vitro means that antibodies are capable of blocking the infectivity or pathogenesis of viruses, bacteria, parasites, and fungi. Neutralization generally occurs as a result of interfering with an organism's attachment to host tissues. Several mechanisms account for the neutralization of a given organism. In addition, a single antibody or antibodies with different specificities can neutralize a given organism, at least in vitro, through multiple mechanisms.

“Neutralization” refers to the function of the antibody disclosed herein. An antibody function refers to the biological effect that the antibody has on a pathogen or its toxin. Additional examples of antibody functions include phagocytosis, antibody-dependent cellular cytotoxicity (ADCC), and complement-mediated lysis of pathogens or of infected cells. See e.g., Forthal, Microbiol Spectr. 2(4): 1-17 (2014).

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “parenteral administration” of an immunogenic composition includes, e.g., subcutaneous (s.c.), intravenous (i.v.), intramuscular (i.m.), or intrasternal injection, or infusion techniques.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “promoter” is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “sample” and “biological sample” and “agricultural sample” are used interchangeably, referring to sample material derived from a subject. Biological samples may include tissues, cells, protein or membrane extracts of cells, and biological fluids (e.g., ascites fluid or cerebrospinal fluid (CSF)) isolated from a subject, as well as tissues, cells and fluids present within a subject. Biological samples may include, but are not limited to, samples taken from breast tissue, renal tissue, the uterine cervix, the endometrium, the head or neck, the gallbladder, parotid tissue, the prostate, the brain, the pituitary gland, kidney tissue, muscle, the esophagus, the stomach, the small intestine, the colon, the liver, the spleen, the pancreas, thyroid tissue, heart tissue, lung tissue, the bladder, adipose tissue, lymph node tissue, the uterus, ovarian tissue, adrenal tissue, testis tissue, the tonsils, thymus, blood, hair, buccal, skin, serum, plasma, CSF, semen, prostate fluid, seminal fluid, urine, feces, sweat, saliva, sputum, mucus, bone marrow, lymph, and tears. Agricultural samples include soil, foliage or any plant tissue or surface or other sample suspected of harboring virus. In addition, the sample can include industrial samples, such as those isolated from surfaces and the environment.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

In some embodiments, the samples include fluid from a subject, including, without limitation, blood or a blood product (e.g., serum, plasma, or the like), umbilical cord blood, amniotic fluid, cerebrospinal fluid, spinal fluid, lavage fluid (e.g., bronchoalveolar, gastric, peritoneal, ductal, ear, arthroscopic), washings of female reproductive tract, urine, feces, sputum, saliva, nasal mucous, prostate fluid, lavage, semen, lymphatic fluid, bile, tears, sweat, breast milk, breast fluid, the like or combinations thereof. In some embodiments, a liquid biological sample is a blood plasma or serum sample. The term “blood” as used herein refers to a blood sample or preparation from a subject. The term encompasses whole blood, blood product or any fraction of blood, such as serum, plasma, buffy coat, or the like as conventionally defined. In some embodiments, the term “blood” refers to peripheral blood. Blood plasma refers to the fraction of whole blood resulting from centrifugation of blood treated with anticoagulants. Blood serum refers to the watery portion of fluid remaining after a blood sample has coagulated. Fluid samples often are collected in accordance with standard protocols hospitals or clinics generally follow. For blood, an appropriate amount of peripheral blood (e.g., between 3-40 milliliters) often is collected and can be stored according to standard procedures prior to or after preparation.

As used herein, the term “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

As used herein, the term “single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, Science 242:423-442 (1988); Huston et al., Proc. Natl. Acad. Sci. USA 85:5879-5883 (1988); Ward et al., Nature 334:54454 (1989); Skerra et al., Science 242:1038-1041 (1988).

As used herein, the term “specificity” refers to the ability to specifically bind (e.g., immunoreact with) a given target antigen (e.g., a human target antigen). A chimeric antigen receptor may be monospecific and contain one or more binding sites, which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

As used herein, the term “specifically binds,” with respect to an antibody, means an antibody or binding fragment thereof (e.g., Fv fragment or scFv) which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. Any living organism in which an immune response can be elicited may be a subject or patient. In certain exemplary embodiments, a subject is a human.

As used herein to “treat” a disease as the term is used herein, means to reduce the frequency or severity of at least one sign or symptom of a disease or disorder experienced by a subject. In one aspect, the term “treat” excludes propylaxis.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

As used herein, the term “therapy” refers to any protocol, method and/or agent (e.g., prophylactic, therapeutic, adjuvant or vaccine) that can be used in the prevention, management, treatment and/or amelioration of a disease or a symptom related thereto.

As used herein, the term “vector” is a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Isolated Antibodies

One aspect of the present disclosure provides an isolated antibody or antibody fragment thereof that binds to a SARS-CoV-2 surface protein. In one aspect, the isolated antibody or fragment thereof has improved binding and/or neutralization characteristics against a broad range of SARS-CoV-2 strains when compared to a monoclonal antibody selected from sotrovimab (S309), COV2-2381, cilgavimab (COV2-2130 or AZD1061), and/or tixagevimab (COV2-2196 or AZD8895). In some embodiments, the SARS-CoV-2 surface protein is a spike (S) glycoprotein.

### Coronaviruses

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

In some embodiments, the coronavirus as used herein refers to a severe acute respiratory syndrome (SARS) associated coronavirus (SARS-CoV). In some embodiments, the coronavirus is either or both of SARS-CoV-1 and SARS-CoV-2. In some embodiments, the coronavirus comprises a virus selected from the group consisting of an Alphacoronavirus; a Colacovirus such as Bat coronavirus CDPHE15; a Decacovirus such as Bat coronavirus HKU10 or Rhinolophus ferrumequinum alphacoronavirus HuB-2013; a Duvinacovirus such as Human coronavirus 229E; a Luchacovirus such as Lucheng Rn rat coronavirus; a Minacovirus such as a Ferret coronavirus or Mink coronavirus 1; a Minunacovirus such as Miniopterus bat coronavirus 1 or Miniopterus bat coronavirus HKU8; a Myotacovirus such as Myotis ricketti alphacoronavirus Sax-2011; a nyctacovirus such as Nyctalus velutinus alphacoronavirus SC-2013; a Pedacovirus such as Porcine epidemic diarrhea virus or Scotophilus bat coronavirus 512; a Rhinacovirus such as Rhinolophus bat coronavirus HKU2; a Setracovirus such as Human coronavirus NL63 or NL63-related bat coronavirus strain BtKYNL63-9b; a Tegacovirus such as Alphacoronavirus 1; a Betacoronavirus; a Embecovirus such as Betacoronavirus 1, Human coronavirus OC43, China Rattus coronavirus HKU24, Human coronavirus HKU1 or Murine coronavirus; a Hibecovirus such as Bat Hp-betacoronavirus Zhejiang2013; a Merbecovirus such as Hedgehog coronavirus 1, Middle East respiratory syndrome-related coronavirus (MERS-CoV), Pipistrellus bat coronavirus HKU5 or Tylonycteris bat coronavirus HKU4; a Nobecovirus such as Rousettus bat coronavirus GCCDC1 or Rousettus bat coronavirus HKU9, a Sarbecovirus such as a Severe acute respiratory syndrome-related coronavirus, Severe acute respiratory syndrome coronavirus (SARS-CoV) or Severe acute respiratory syndrome coronavirus 2 (SARS-CoV-2, COVID-19); a Deltacoronavirus; an Andecovirus such as Wigeon coronavirus HKU20; a Buldecovirus such as Bulbul coronavirus HKU11, Porcine coronavirus HKU15, Munia coronavirus HKU13 or White-eye coronavirus HKU16; a Herdecovirus such as Night Heron coronavirus HKU19; a Moordecovirus such as Common moorhen coronavirus HKU21; a Gammacoronavirus; a Cegacovirus such as Beluga Whale coronavirus SW1; and an Igacovirus such as Avian coronavirus.

Symptoms of a coronavirus infection include, but are not limited to, mild symptoms, such as fatigues, tingling, tingling or numbness in the hands and feet, dizziness, confusion, brain fog, body ache, chills, loss of appetite, nausea, vomiting, abdominal pain or discomfort, loss of smell, inability to taste, muscle weakness, photophobia, adenopathy, headaches, cough, dry cough, shortness of breath, sore throat, lower extremity weakness/numbness, diarrhea, low blood O2, sneezing, runny nose or post-nasal drip; severe symptoms, such as ventilatory use, high fever, severe cough, delirium, seizures, stroke, systematic inflammation, cytokine storm; and other symptoms, such as fever, swollen adenoids, pneumonia, bronchitis, and Dyspnea.

### SARS-CoV-2

SARS-CoV-2 is a contagious virus that causes the acute respiratory disease designated coronavirus disease 2019 (COVID-19). All viruses, including SARS-CoV-2, change over time. Most changes have little to no impact on the virus' properties. However, some changes may affect the virus's properties, such as how easily it spreads, the associated disease severity, or the performance of vaccines, therapeutic medicines, diagnostic tools, or other public health and social measures. During the ongoing COVID-19 outbreak (December 2019 to present), SARS-CoV-2 has continuously evolved into more dangerous strains. During late 2020, the emergence of variants that posed an increased risk to global public health prompted the characterization of specific Variants of Interest (VOIs) and Variants of Concern (VOCs). See e.g., Center for Disease Control and Prevention, cdc.gov/coronavirus/2019-ncov/variants/variant-classifications.html#anchor_1632154493691 (Updated Apr. 26, 2022); World Health Organization, who.int/en/activities/tracking-SARS-CoV-2-variants (updated on 25 Apr. 2022).

A SARS-CoV-2 variant that meets the definition of a VOI (see below) and, through a comparative assessment, has been demonstrated to be associated with one or more of the following changes at a degree of global public health significance: (1) increase in transmissibility or detrimental change in COVID-19 epidemiology; (2) increase in virulence or change in clinical disease presentation; and/or decrease in effectiveness of public health and social measures or available diagnostics, vaccines, therapeutics. Variants that do not otherwise meet all these criteria may be designated as VOCs/VOIs/VUMs, and those posing a diminishing risk relative to other circulating variants may be reclassified.

As of On Apr. 14, 2022, the U.S. government SARS-CoV-2 Interagency Group (SIG) recognizes the following defined four classes of SARS-CoV-2 variants: (1) Variant Being Monitored (VBM); Variant of Interest (VOI); Variant of Concern (VOC); and Variant of High Consequence (VOHC). As of that date, no SARS-CoV-2 variants are designated as VOI or VOHC. See e.g., Center for Disease Control and Prevention, cdc.gov/coronavirus/2019-ncov/variants/variant-classifications.html#anchor_1632154493691 (Updated Apr. 26, 2022).

The Variant Being Monitored (VBM) are selected from Alpha (B.1.1.7 and Q lineages), Beta (B.1.351 and descendent lineages), Gamma (P.1 and descendent lineages), Delta (B.1.617.2 and AY lineages), Epsilon (B.1.427 and B.1.429), Eta (B.1.525), Iota (B.1.526), Kappa (B.1.617.1), 1.617.3, Mu (B.1.621, B.1.621.1), Zeta (P.2). See e.g., Center for Disease Control and Prevention, cdc.gov/coronavirus/2019-ncov/variants/variant-classifications.html#anchor_1632154493691 (Updated Apr. 26, 2022).

The Variant of Concern (VOC) is Omicron (B.1.1.529, BA.1, BA.1.1, BA.2, BA.3, BA.4 and BA.5 lineages). The Omicron variant, which was first identified in South Africa shows evidence of an increase in transmissibility, more severe disease (for example, increased hospitalizations or deaths), significant reduction in neutralization by antibodies generated during previous infection or vaccination, reduced effectiveness of treatments or vaccines, or diagnostic detection failures.

The Omicron variant and its lineages comprise mutations selected from deletions, insertions and substitutions in the SARS-CoV-2's Spike Protein. The substitutions are selected from A67V, T95I, Y145D, L212I, G339D, S371L, S373P, S375F, K417N, N440K, G4465, S477N, T478K, E484A, Q493R, G496S, Q498R, N501Y, Y505H, T547K, D614G, H655Y, N679K, P681H, N764K, D796Y, N856K, Q954H, N969K, and/or L981F. The deletions are selected from de169-70, de1142-144, and/or de1211; and the insertion is ins214EPE. See e.g., Center for Disease Control and Prevention, cdc.gov/coronavirus/2019-ncov/variants/variant-classifications.html#anchor_1632154493691 (Updated Apr. 26, 2022)

Given the continuous evolution of the virus that leads to SARS-CoV-2 and the constant developments in our understanding of the impacts of variants, there is a need for novel prophylactic monoclonal antibodies that can bind to and neutralize old and emerging strains of SARS-CoV-2, such as the Omicron variants.

In some embodiments, the antibody or antibody fragment disclosed herein binds a SARS-CoV-2 variant selected from Alpha (B.1.1.7 and Q lineages); Beta (B.1.351 and descendent lineages); Gamma (P.1 and descendent lineages); Delta (B.1.617.2 and AY lineages); Epsilon (B.1.427 and B.1.429); Eta (B.1.525); Iota (B.1.526); Kappa (B.1.617.1); 1.617.3; Mu (B.1.621, B.1.621.1); Zeta (P.2); or Omicron (B.1.1.529, BA.1, BA.1.1, BA.2, BA.3, BA.4 and BA.5 lineages). In some embodiments, the monoclonal antibody or antibody fragment binds to and neutralizes SARS-CoV-2 Omicron (B.1.1.529, BA.1, BA.1.1, BA.2, BA.3, BA.4 and BA.5 lineages).

In some embodiments, the antibody or antibody fragment disclosed herein binds a SARS-CoV-2 variant comprising a mutation in the spike (S) glycoprotein. In one embodiment, the mutation is a substitution selected from D614G, G339D, S371L, S373P, S375F, K417N, N440K, G4465, S477N, T478K, E484A, Q493R, G496S, Q498R, N501Y, Y505H, T547K, H655Y, N764K, D796Y, N856K, Q954H, N969K, L981F, or a combination thereof. In one embodiment, the substitution is selected from A67V, T95I, Y145D, L212I, G339D, S371L, S373P, S375F, K417N, N440K, G4465, S477N, T478K, E484A, Q493R, G496S, Q498R, N501Y, Y505H, T547K, D614G, H655Y, N679K, P681H, N764K, D796Y, N856K, Q954H, N969K, and/or L981F. In some embodiments, the SARS-CoV-2 variant comprises a D614G substitution. In some embodiments, the mutation comprises deletion of amino acids 143-145 (143-145del). In some embodiments, the mutation is in the receptor-binding domain (RBD) or the N-terminal domain of the spike glycoprotein.

In some embodiments, the antibody or antibody fragment disclosed herein is capable of neutralizing from about 80 to about 100% of the SARS-CoV-2 virus with a concentration of about 0.5 ng/ml to about 0.01 ng/ml, 0.75 ng/ml to about 0.01 ng/ml, 1 ng/ml to about 0.01 ng/ml, 1.6 ng/ml to about 0.01 ng/ml, 2 ng/ml to about 0.01 ng/ml, 5 ng/ml to about 0.01 ng/ml, 8 ng/ml to about 0.01 ng/ml, 10 ng/ml to about 0.01 ng/ml, 12 ng/ml to about 0.01 ng/ml, 15 ng/ml to about 0.01 ng/ml, 16 ng/ml to about 0.01 ng/ml, about 20 ng/ml to about 0.01 ng/ml, about 30 ng/ml to about 0.01 ng/ml, about 40 ng/ml to about 0.01 ng/ml, about 50 ng/ml to about 0.01 ng/ml, about 75 ng/ml to about 0.01 ng/ml, about 100 ng/ml to about 0.01 ng/ml, about 125 ng/ml to about 0.01 ng/ml, 150 ng/ml to about 0.01 ng/ml, about 175 ng/ml to about 0.01 ng/ml, about 190 ng/ml to about 0.01 ng/ml, about 200 ng/ml to about 0.01 ng/ml, about 220 ng/ml to about 0.01 ng/ml, about 235 ng/ml to about 0.01 ng/ml, about 240 ng/ml to about 0.01 ng/ml, or about 250 ng/ml to about 0.01 ng/ml.

In some embodiments, the antibody or antibody fragment disclosed herein is capable of neutralizing from about 80 to about 100% of the SARS-CoV-2 virus with a concentration from about 0.01 ng/ml to about 1.0 ng/ml; from about 0.5 ng/ml to about 1.5 ng/ml; from about 1 ng/ml to about 2.0 ng/ml; from about 0.01 ng/ml to about 1.9 ng/ml; from about 1 ng/ml to about 5.0 ng/ml; or from about 5.0 ng/ml to about 10.0 ng/ml.

In some embodiments, the antibody or antibody fragment inhibits or blocks the binding of the SARS-CoV-2 virus to a human receptor angiotensin converting enzyme 2 (hACE2). In some embodiments, the antibody or antibody fragment inhibits or blocks the binding to hACE2 with an IC₅₀ of about 0.1 ng/ml, 0.5 ng/ml to about 0.1 ng/ml, 0.8 ng/ml to about 0.1 ng/ml, 1 ng/ml to about 0.1 ng/ml, 2 ng/ml to about 0.1 ng/ml, 5 ng/ml to about 0.1 ng/ml, 10 ng/ml to about 0.1 ng/ml, 15 ng/ml to about 0.1 ng/ml, about 20 ng/ml to about 0.1 ng/ml, about 30 ng/ml to about 0.1 ng/ml, about 40 ng/ml to about 0.1 ng/ml, about 50 ng/ml to about 0.1 ng/ml, about 75 ng/ml to about 0.1 ng/ml, about 100 ng/ml to about 0.1 ng/ml, about 110 ng/ml to about 0.1 ng/ml, about 120 ng/ml to about 0.1 ng/ml, about 130 ng/ml to about 0.1 ng/ml, about 140 ng/ml to about 0.1 ng/ml, or about 150 ng/ml to about 0.1 ng/ml, as measured by Biacore™ assay systems, or any methods known to those of skill in the art. See e.g., Casper et al., Anal Biochem, 325(1):126-36 (2004); Jason-Moller et al., Curr Protoc Protein Sci, Chapter 19: Unit 19.13 (2006); giffordbioscience.com/spr-biacore-services/; Zost et al., Nature 584: 443-449 (2020). In some embodiments, the antibody or antibody fragment reduces (e.g., blocks) S protein binding to a cell that expresses hACE2 with an IC₅₀ of less than about 1.5 nM, or about 1 nM, or about 0.8 nM, or about 0.6 nM, or about 0.4 nM, or about 0.2 nM, or about 0.1 nM, e.g., between about 0.2 nM and about 0.1 nM, e.g., about 0.15 nM to about 0.1 nM, e.g., about 0.145 nM. In some embodiments, the antibody or antibody fragment reduces (e.g., blocks) Spike protein binding to a cell that expresses hACE2 with an IC₅₀ of less than about 2 nM, or less than about 1.5 nM, or less than about 1 nM, or less than about 0.5 nM, or less than about 0.2 nM, e.g., between about 0.5 nM and about 0.01 nM, or about 0.2 nM to 0.05 nM, e.g., about 0.1 nM.

In some embodiments, the antibody or antibody fragment binds to a target antigen (e.g., SARS-CoV-2 spike protein receptor binding domain) with a binding affinity (K_{D}) of about 0.01 nM to about 0.1 nM, about 0.03 nM to about 0.1 nM, about 0.05 nM to about 0.1 nM, about 0.07 nM to about 0.1 nM, about 0.1 nM to about 0.5 nM, about 0.25 nM to about 0.5 nM, about 0.5 nM to about 0.5 nM, about 0.75 nM to about 0.5 nM, about 1.0 nM to about 0.5 nM, about 1.25 nM to about 0.5 nM, about 1.5 nM to about 0.5 nM, about 1.75 nM to about 0.5 nM, about 2.0 nM to about 0.5 nM, about 2.5 nM to about 0.5 nM, from about 10 nM to about 120 nM, from 30 nM to about 120 nM, from 50 nM to about 120 nM, from about 60 nM to about 120 nM, from about 80 nM to about 105 nM, from about 63 nM to about 85 nM, or from about 50 nM to about 85 nM, as measured by Biacore™ assay systems or any methods known to those of skill in the art. See e.g., Casper et al., Anal Biochem, 325(1):126-36 (2004); Jason-Moller et al., Curr Protoc Protein Sci, Chapter 19: Unit 19.13 (2006); giffordbioscience.com/spr-biacore-services/; Zost et al., Nature 584: 443-449 (2020).

In some embodiments, the antibody or antibody fragment binds to a target antigen (e.g., SARS-CoV-2 spike protein receptor binding domain) with a K_{D} slower than 5×10⁻⁴, 1×10⁻⁴, 5×10⁻⁵, 1×10⁻⁵, 5×10⁻⁶ or 1×10⁻⁶ s⁻¹, e.g., about 6.33×10⁻⁵ s⁻¹, as measured by Biacore™ assay systems. In some embodiments, the antibody or antibody fragment binds to a target antigen (e.g., hACE2) with a KA faster than 1×10⁴, 5×10⁴, 1×10⁵, 5×10⁵ or 1×10⁶ M⁻¹ s⁻¹, e.g., about 3.07×10⁴ M⁻¹ s⁻¹, as measured by Biacore™ assay systems, or any methods known to those of skill in the art. See e.g., Casper et al., Anal Biochem, 325(1):126-36 (2004); Jason-Moller et al., Curr Protoc Protein Sci, Chapter 19: Unit 19.13 (2006); giffordbioscience.com/spr-biacore-services/; Zost et al., Nature 584: 443-449 (2020).

In some embodiments, the antibody or antibody fragment (e.g., an isolated or recombinant antibody molecule) binds to a trimeric spike protein ectodomain and/or spike protein receptor binding domain (RBD) with high affinity. For example, the antibody or antibody fragment (e.g., an isolated or recombinant antibody molecule) binds to a trimeric spike protein ectodomain and/or spike protein receptor binding domain (RBD) with an affinity constant of at least about 10⁷ M⁻¹, typically about 10⁸ M⁻¹, and more typically, about 10⁹ M⁻¹ to 10¹⁰ M⁻¹ or stronger. In some embodiments, the antibody or antibody fragment binds specifically to an epitope on hACE2. In some embodiments, the antibody or antibody fragment binds specifically to the same or similar epitope as the epitope recognized by the monoclonal antibody COV2-2130 or a chimeric antibody thereof. In some embodiments, the antibody or antibody fragment binds specifically to the same or similar epitope as the epitope recognized by the monoclonal antibody COV2-2381 or a chimeric antibody thereof. In some embodiments, the antibody or antibody fragment binds specifically to the same or similar epitope as the epitope recognized by the monoclonal antibody COV2-2196 or a chimeric antibody thereof. In some embodiments, the antibody or antibody fragment binds to non-overlapping epitopes on the receptor-binding domain of SARS-CoV-2 spike protein when compared to the epitope bound by the monoclonal antibody or antibody fragment selected from sotrovimab (S309), COV2-2381, cilgavimab (COV2-2130 or AZD1061), or tixagevimab (COV2-2196 or AZD8895).

In some embodiments, the antibody or antibody fragment binds: a trimeric spike protein ectodomain, and/or a monomeric spike protein binding domain (RBD). In some embodiments, the binding affinity of the antibody or antibody fragment to the trimeric spike protein ectodomain and/or the monomeric spike protein RBD has an EC₅₀ of about 1.0 pg/ml to 0.1 ng/ml, 1.0 pg/ml to 0.25 ng/ml, 1.0 pg/ml to 0.75 ng/ml, 1.0 pg/ml to 1.0 ng/ml, 1.0 pg/ml to 1.25 ng/ml, 1.0 pg/ml to 1.50 ng/ml, 1.0 pg/ml to 1.75 ng/ml, 1.0 pg/ml to about 2 ng/mL, 1.0 pg/ml to about 2.5 ng/mL, 1.0 pg/ml to about 3 ng/mL, 1.0 pg/ml to about 3.5 ng/mL, 1.0 pg/ml to about 4.0 ng/mL, 1.0 pg/ml to about 4.5 ng/mL, 1.0 pg/ml to about 5 ng/mL, or 1.0 pg/ml to about 10 ng/mL.

In some embodiments, the antibody or antibody fragment comprises a heavy chain complementary determining region 1 (CDRH1) comprising, or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 202, and a light chain complementary determining region 1 (CDRL1) comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises, or consisting essentially of, or consisting of a CDRH1 comprising, or consisting essentially of, or consisting of the amino acid sequence of SEQ ID NO: 196, a CDRH2 comprising or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 204, and a CDRL1 comprising or consisting essentially of, or consisting of the amino acid sequence of SEQ ID NO: 228, a CDRL2 comprising or consisting essentially of, or consisting of the amino acid sequence of SEQ ID NO: 242 or 243, and a CDRL3 comprising or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 204, and a CDRL1 comprising or consisting essentially of, or consisting of the amino acid sequence of SEQ ID NO: 229, a CDRL2 comprising or consisting essentially of, or consisting of the amino acid sequence of SEQ ID NO: 242 or 243, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 204, and a CDRL1 comprising or consisting essentially of, or consisting of the amino acid sequence of SEQ ID NO: 230, a CDRL2 comprising or consisting essentially of, or consisting of the amino acid sequence of SEQ ID NO: 242 or 244, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 204, and a CDRL1 comprising or consisting essentially of, or consisting of the amino acid sequence of SEQ ID NO: 231, a CDRL2 comprising or consisting essentially of, or consisting of the amino acid sequence of SEQ ID NO: 242 or 245, and a CDRL3 comprising or consisting essentially of, or consisting of the amino acid sequence of SEQ ID NO: 251.

In some embodiments, the antibody or antibody fragment comprises or consists essentially of, or consists of a CDRH1 comprising or consisting essentially of, or consisting of the amino acid sequence of SEQ ID NO: 196, a CDRH2 comprising or consisting essentially of, or consisting of the amino acid sequence of SEQ ID NO: 200, and a CDRH3 comprising or consisting essentially of, or consisting of the amino acid sequence of SEQ ID NO: 204, and a CDRL1 comprising or consisting essentially of, or consisting of the amino acid sequence of SEQ ID NO:232, a CDRL2 comprising or consisting essentially of, or consisting of the amino acid sequence of SEQ ID NO: 242 or 245, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 205, and a CDRL1 comprising or consisting essentially of, or consisting of the amino acid sequence of SEQ ID NO: 233, a CDRL2 comprising or consisting essentially of, or consisting of the amino acid sequence of SEQ ID NO: 242 or 243, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of S SEQ ID NO: 205, and a CDRL1 comprising or consisting essentially of, or consisting of the amino acid sequence of SEQ ID NO: 234, a CDRL2 comprising or consisting essentially of, or consisting of the amino acid sequence of SEQ ID NO: 242 or 245, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of g the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 206, and a CDRL1 comprising or consisting essentially of, or consisting of the amino acid sequence of SEQ ID NO: 235, a CDRL2 comprising or consisting essentially of, or consisting of the amino acid sequence of SEQ ID NO: 242 or 246, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 197, and a CDRH3 comprising or consisting essentially of, or consisting of the amino acid sequence of SEQ ID NO: 207, and a CDRL1 comprising or consisting essentially of, or consisting of the amino acid sequence of SEQ ID NO: 236, a CDRL2 comprising or consisting essentially of, or consisting of the amino acid sequence of SEQ ID NO: 242 or 243, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 201, and a CDRH3 comprising or consisting essentially of, or consisting of the amino acid sequence of SEQ ID NO: 208, and a CDRL1 comprising or consisting essentially of, or consisting of the amino acid sequence of SEQ ID NO: 228, a CDRL2 comprising or consisting essentially of, or consisting of the amino acid sequence of SEQ ID NO: 242 or 247, and a CDRL3 comprising or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising or consisting essentially of, or consisting of the amino acid sequence of SEQ ID NO: 198, and a CDRH3 comprising or consisting essentially of, or consisting of the amino acid sequence of SEQ ID NO: 209, and a CDRL1 comprising or consisting essentially of, or consisting of the amino acid sequence of SEQ ID NO: 234, a CDRL2 comprising or consisting essentially of, or consisting of the amino acid sequence of SEQ ID NO: 242 or 245, and a CDRL3 comprising or consisting essentially of, or consisting of the amino acid sequence of SEQ ID NO: 251.

In some embodiments, the CDRs of the heavy chain variable domain or the light chain variable domain of the antibody or antibody fragment disclosed herein comprises the amino acid sequence disclosed in Tables 1, 2, or 3.

In some embodiments, the antibody or antibody fragment disclosed herein comprises: a heavy chain variable sequence having at least 70%, 80%, 90%, 95%, 96%, 97%, 98%, or 99% identity to the amino acid sequence of SEQ ID NO: 100, 101, 102, 103, 104, 105, 106, 107, 108, or 109; and/or a light chain variable sequences having at least 70%, 80%, 90%, 95%, 96%, 97%, 98%, or 99% identity to the amino acid sequence of SEQ ID NO: 155, 156, 157, 158, 159, 160, 161, 162, 163, 164, 165, or 166. In one embodiment, the CDRs of the heavy chain variable sequence or the light chain variable sequences are 100% identical to a CDR disclosed in Tables 1-4.

In some embodiments, the antibody or antibody fragment disclosed herein comprises: a heavy chain variable sequence having at least 70%, 80%, 90%, 95%, 96%, 97%, 98%, or 99% identity to the amino acid sequence disclosed in Table 4; and/or a light chain variable sequences having at least 70%, 80%, 90%, 95%, 96%, 97%, 98%, or 99% identity to the amino acid sequence disclosed in Table 5. In some embodiments, the CDRs of the heavy chain variable sequence or the light chain variable sequences are 100% identical to a CDR disclosed in Tables 1, 2, or 3.

In some embodiments, the antibody or antibody fragment disclosed herein comprises, or consists essentially of, or consists of a heavy chain variable sequence and a light chain variable sequence having at least 96% identity to an amino acid sequence selected from: the amino acid sequence of SEQ ID NO: 100 and the amino acid sequence of SEQ ID NO: 155; the amino acid sequence of SEQ ID NO: 101 and the amino acid sequence of SEQ ID NO: 156; the amino acid sequence of SEQ ID NO: 102 and the amino acid sequence of SEQ ID NO: 156; the amino acid sequence of SEQ ID NO: 103 and the amino acid sequence of SEQ ID NO: 157; the amino acid sequence of SEQ ID NO: 103 and the amino acid sequence of SEQ ID NO: 158; the amino acid sequence of SEQ ID NO: 103 and the amino acid sequence of SEQ ID NO: 159; the amino acid sequence of SEQ ID NO: 103 and the amino acid sequence of SEQ ID NO: 160; the amino acid sequence of SEQ ID NO: 104 and the amino acid sequence of SEQ ID NO: 161; the amino acid sequence of SEQ ID NO: 105 and the amino acid sequence of SEQ ID NO: 162; the amino acid sequence of SEQ ID NO: 105 and the amino acid sequence of SEQ ID NO: 163; the amino acid sequence of SEQ ID NO: 106 and the amino acid sequence of SEQ ID NO: 164; the amino acid sequence of SEQ ID NO: 107 and the amino acid sequence of SEQ ID NO: 165; the amino acid sequence of SEQ ID NO: 108 and the amino acid sequence of SEQ ID NO: 166; or the amino acid sequence of SEQ ID NO: 109 and the amino acid sequence of SEQ ID NO: 163.

In some embodiments, the antibody or antibody fragment disclosed herein comprises, or consists essentially of, or consists of a heavy chain variable sequence and a light chain variable sequence having at least 96% identity to an amino acid sequence in Table 4.

In some embodiments, the antibody or antibody fragment comprises a heavy chain complementary determining region 1 (CDRH1) comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 237, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises, or consisting essentially of, or consisting of a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising, or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 227, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 239, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of g the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 210, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 211, and a CDRL1 comprising, or consisting essentially of, or consisting of the amino acid sequence of SEQ ID NO: 234, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 239, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 212, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 213, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 214, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 246, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 248, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 206, and a CDRL1 comprising, or consisting essentially of, or consisting of the amino acid sequence of SEQ ID NO: 235, a CDRL2 comprising, or consisting essentially of, or consisting of the amino acid sequence of SEQ ID NO: 242 or 249, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 215, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 216, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 217, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 247, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 201, and a CDRH3 comprising, or consisting essentially of, or consisting of the amino acid sequence of SEQ ID NO: 208, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 250, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 218, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 219, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 220, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 218, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 207, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 207, and a CDRL1 comprising, or consisting essentially of, or consisting of the amino acid sequence of SEQ ID NO: 236, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 221, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 232, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 229, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 200, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 202, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 222, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 223, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 224, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 225, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 202, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising the amino acid sequence of SEQ ID NO: 221, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 226, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 231, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 211, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 240, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 241, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 233, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 204, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 198, and a CDRH3 comprising, or consisting essentially of, or consisting of the amino acid sequence of SEQ ID NO: 221, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 203, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 205, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 197, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 244, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 230, a CDRL2 comprising, or consisting essentially of, or consisting of the amino acid sequence of SEQ ID NO: 242 or 245, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 229, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 228, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the antibody or antibody fragment comprises a CDRH1 comprising or consisting essentially of, or consisting of the amino acid sequence of SEQ ID NO: 196, a CDRH2 comprising, or consisting essentially of, or consisting of the amino acid sequence of SEQ ID NO: 199, and a CDRH3 comprising, or consisting essentially of, or consisting of the amino acid sequence of SEQ ID NO: 209, and a CDRL1 comprising, or consisting essentially of, or consisting of the amino acid sequence of SEQ ID NO: 238, a CDRL2 comprising, or consisting essentially of, or consisting of the amino acid sequence of SEQ ID NO: 242 or 243, and a CDRL3 comprising, or consisting essentially of, or consisting of the amino acid sequence of SEQ ID NO: 251.

In some embodiments, the CDRs of the heavy chain variable domain or the light chain variable domain of the antibody or antibody fragment disclosed herein comprises any of the CDR amino acid sequences as shown by SEQ ID NOs disclosed in Table 13.

In some embodiments, the antibody or antibody fragment disclosed herein comprises: a heavy chain variable sequence having at least 70%, 80%, 90%, 95%, 96%, 97%, 98%, or 99% identity to the amino acid sequence of SEQ ID NO: 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, or 154; and/or a light chain variable sequences having at least 70%, 80%, 90%, 95%, 96%, 97%, 98%, or 99% identity to the amino acid sequence of SEQ ID NO: 67. C 170, 171, 172, 173, 174, 175, 176, 177, 178, 179, 180, 181, 182, 183, 184, 185, 186, 187, 188, 189, 190, 191, 192, 193, 194, or 195, with the proviso that the amino acid mutations are not altered in the equivalent from those mutations identified in Table 13. In one embodiment, the CDRs of the heavy chain variable sequence or the light chain variable sequences are 100% identical to a CDR disclosed in Table 13.

In some embodiments, the antibody or antibody fragment disclosed herein comprises: a heavy chain variable sequence having at least 70%, 80%, 90%, 95%, 96%, 97%, 98%, or 99% identity to the amino acid sequence disclosed in Table 14; and/or a light chain variable sequences having at least 70%, 80%, 90%, 95%, 96%, 97%, 98%, or 99% identity to the amino acid sequence disclosed in Table 14, with the proviso that the amino acid mutations are not altered in the equivalent from those mutations identified in Table 13. In some embodiments, the CDRs of the heavy chain variable sequence or the light chain variable sequences are 100% identical to a CDR disclosed in Table 13.

In some embodiments, the antibody or antibody fragment disclosed herein comprises, or consists essentially of, or consists of a heavy chain variable sequence and a light chain variable sequence having at least 96%, at least 97%, at least 98%, at least 99% or 100% identity to an amino acid sequence selected from: the amino acid sequence of SEQ ID NO: 110 and the amino acid sequence of SEQ ID NO: 167; the amino acid sequence of SEQ ID NO: 111 and the amino acid sequence of SEQ ID NO: 168; the amino acid sequence of SEQ ID NO: 111 and the amino acid sequence of SEQ ID NO: 169; the amino acid sequence of SEQ ID NO: 111 and the amino acid sequence of SEQ ID NO: 170; the amino acid sequence of SEQ ID NO: 111 and the amino acid sequence of SEQ ID NO: 171; the amino acid sequence of SEQ ID NO: 112 and the amino acid sequence of SEQ ID NO: 173; the amino acid sequence of SEQ ID NO: 113 and the amino acid sequence of SEQ ID NO: 172; the amino acid sequence of SEQ ID NO: 114 and the amino acid sequence of SEQ ID NO: 172; the amino acid sequence of SEQ ID NO: 115 and the amino acid sequence of SEQ ID NO: 172; the amino acid sequence of SEQ ID NO: 116 and the amino acid sequence of SEQ ID NO: 173; the amino acid sequence of SEQ ID NO: 117 and the amino acid sequence of SEQ ID NO: 172; the amino acid sequence of SEQ ID NO: 118 and the amino acid sequence of SEQ ID NO: 172; the amino acid sequence of SEQ ID NO: 119 and the amino acid sequence of SEQ ID NO: 172; the amino acid sequence of SEQ ID NO: 120 and the amino acid sequence of SEQ ID NO: 172; the amino acid sequence of SEQ ID NO: 121 and the amino acid sequence of SEQ ID NO: 172; the amino acid sequence of SEQ ID NO: 110 and the amino acid sequence of SEQ ID NO: 173; the amino acid sequence of SEQ ID NO: 122 and the amino acid sequence of SEQ ID NO: 174; the amino acid sequence of SEQ ID NO: 123 and the amino acid sequence of SEQ ID NO: 174; the amino acid sequence of SEQ ID NO: 124 and the amino acid sequence of SEQ ID NO: 174; the amino acid sequence of SEQ ID NO: 125 and the amino acid sequence of SEQ ID NO: 174; the amino acid sequence of SEQ ID NO: 126 and the amino acid sequence of SEQ ID NO: 174; the amino acid sequence of SEQ ID NO: 127 and the amino acid sequence of SEQ ID NO: 175; the amino acid sequence of SEQ ID NO: 127 and the amino acid sequence of SEQ ID NO: 176; the amino acid sequence of SEQ ID NO: 128 and the amino acid sequence of SEQ ID NO: 177; the amino acid sequence of SEQ ID NO: 129 and the amino acid sequence of SEQ ID NO: 177; the amino acid sequence of SEQ ID NO: 130 and the amino acid sequence of SEQ ID NO: 177; the amino acid sequence of SEQ ID NO: 131 and the amino acid sequence of SEQ ID NO: 177; the amino acid sequence of SEQ ID NO: 132 and the amino acid sequence of SEQ ID NO: 177; the amino acid sequence of SEQ ID NO: 133 and the amino acid sequence of SEQ ID NO: 178; the amino acid sequence of SEQ ID NO: 134 and the amino acid sequence of SEQ ID NO: 179; the amino acid sequence of SEQ ID NO: 135 and the amino acid sequence of SEQ ID NO: 179; the amino acid sequence of SEQ ID NO: 136 and the amino acid sequence of SEQ ID NO: 179; the amino acid sequence of SEQ ID NO: 134 and the amino acid sequence of SEQ ID NO: 180; the amino acid sequence of SEQ ID NO: 137 and the amino acid sequence of SEQ ID NO: 180; the amino acid sequence of SEQ ID NO: 138 and the amino acid sequence of SEQ ID NO: 179; the amino acid sequence of SEQ ID NO: 139 and the amino acid sequence of SEQ ID NO: 181; the amino acid sequence of SEQ ID NO: 112 and the amino acid sequence of SEQ ID NO: 181; the amino acid sequence of SEQ ID NO: 140 and the amino acid sequence of SEQ ID NO: 181; the amino acid sequence of SEQ ID NO: 141 and the amino acid sequence of SEQ ID NO: 181; the amino acid sequence of SEQ ID NO: 142 and the amino acid sequence of SEQ ID NO: 181; the amino acid sequence of SEQ ID NO: 143 and the amino acid sequence of SEQ ID NO: 181; the amino acid sequence of SEQ ID NO: 144 and the amino acid sequence of SEQ ID NO: 182; the amino acid sequence of SEQ ID NO: 144 and the amino acid sequence of SEQ ID NO: 183; the amino acid sequence of SEQ ID NO: 144 and the amino acid sequence of SEQ ID NO: 184; the amino acid sequence of SEQ ID NO: 145 and the amino acid sequence of SEQ ID NO: 185; the amino acid sequence of SEQ ID NO: 146 and the amino acid sequence of SEQ ID NO: 185; the amino acid sequence of SEQ ID NO: 147 and the amino acid sequence of SEQ ID NO: 185; the amino acid sequence of SEQ ID NO: 148 and the amino acid sequence of SEQ ID NO: 185; the amino acid sequence of SEQ ID NO: 149 and the amino acid sequence of SEQ ID NO: 185; the amino acid sequence of SEQ ID NO: 150 and the amino acid sequence of SEQ ID NO: 186; the amino acid sequence of SEQ ID NO: 139 and the amino acid sequence of SEQ ID NO: 187; the amino acid sequence of SEQ ID NO: 112 and the amino acid sequence of SEQ ID NO: 187; the amino acid sequence of SEQ ID NO: 151 and the amino acid sequence of SEQ ID NO: 187; the amino acid sequence of SEQ ID NO: 110 and the amino acid sequence of SEQ ID NO: 187; the amino acid sequence of SEQ ID NO: 152 and the amino acid sequence of SEQ ID NO: 187; the amino acid sequence of SEQ ID NO: 153 and the amino acid sequence of SEQ ID NO: 187; the amino acid sequence of SEQ ID NO: 119 and the amino acid sequence of SEQ ID NO: 187; the amino acid sequence of SEQ ID NO: 154 and the amino acid sequence of SEQ ID NO: 187; the amino acid sequence of SEQ ID NO: 120 and the amino acid sequence of SEQ ID NO: 187; the amino acid sequence of SEQ ID NO: 111 and the amino acid sequence of SEQ ID NO: 186; the amino acid sequence of SEQ ID NO: 116 and the amino acid sequence of SEQ ID NO: 187; the amino acid sequence of SEQ ID NO: 113 and the amino acid sequence of SEQ ID NO: 187; the amino acid sequence of SEQ ID NO: 114 and the amino acid sequence of SEQ ID NO: 187; the amino acid sequence of SEQ ID NO: 119 and the amino acid sequence of SEQ ID NO: 188; the amino acid sequence of SEQ ID NO: 120 and the amino acid sequence of SEQ ID NO: 188; the amino acid sequence of SEQ ID NO: 116 and the amino acid sequence of SEQ ID NO: 188; the amino acid sequence of SEQ ID NO: 121 and the amino acid sequence of SEQ ID NO: 188; the amino acid sequence of SEQ ID NO: 110 and the amino acid sequence of SEQ ID NO: 189; the amino acid sequence of SEQ ID NO: 110 and the amino acid sequence of SEQ ID NO: 190; the amino acid sequence of SEQ ID NO: 113 and the amino acid sequence of SEQ ID NO: 188; the amino acid sequence of SEQ ID NO: 113 and the amino acid sequence of SEQ ID NO: 191; the amino acid sequence of SEQ ID NO: 139 and the amino acid sequence of SEQ ID NO: 192; the amino acid sequence of SEQ ID NO: 151 and the amino acid sequence of SEQ ID NO: 192; the amino acid sequence of SEQ ID NO: 110 and the amino acid sequence of SEQ ID NO: 192; the amino acid sequence of SEQ ID NO: 152 and the amino acid sequence of SEQ ID NO: 192; the amino acid sequence of SEQ ID NO: 154 and the amino acid sequence of SEQ ID NO: 192; the amino acid sequence of SEQ ID NO: 153 and the amino acid sequence of SEQ ID NO: 192; the amino acid sequence of SEQ ID NO: 120 and the amino acid sequence of SEQ ID NO: 192; the amino acid sequence of SEQ ID NO: 119 and the amino acid sequence of SEQ ID NO: 192; the amino acid sequence of SEQ ID NO: 114 and the amino acid sequence of SEQ ID NO: 192; the amino acid sequence of SEQ ID NO: 113 and the amino acid sequence of SEQ ID NO: 192; the amino acid sequence of SEQ ID NO: 113 and the amino acid sequence of SEQ ID NO: 193; the amino acid sequence of SEQ ID NO: 113 and the amino acid sequence of SEQ ID NO: 194; the amino acid sequence of SEQ ID NO: 113 and the amino acid sequence of SEQ ID NO: 195; or the amino acid sequence of SEQ ID NO: 113 and the amino acid sequence of SEQ ID NO: 169.

In some embodiments, the antibody or antibody fragment disclosed herein comprises, or consists essentially of, or consists of a heavy chain variable sequence having at least 96%, at least 97%, at least 98% or at least 99% identity to a heavy chain variable amino acid sequence in Table 14 and/or a light chain variable sequence having at least 96%, at least 97%, at least 98% or at least 99% identity to a light chain variable amino acid sequence in Table 14, with the proviso that the amino acid mutations are not altered in the equivalent from those mutations identified in Table 13.

When comparing polynucleotide and polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogeny pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One particular example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example, with the parameters described herein, to determine percent sequence identity for the polynucleotides and polypeptides of the disclosure. Software for performing BLAST analyses is publicly available through the National Center for Biotechnology Information. The rearranged nature of an antibody sequence and the variable length of each gene requires multiple rounds of BLAST searches for a single antibody sequence. Also, manual assembly of different genes is difficult and error-prone. The sequence analysis tool IgBLAST (world-wide-web at ncbi.nlm.nih.gov/igblast/) identifies matches to the germline V, D and J genes, details at rearrangement junctions, the delineation of Ig V domain framework regions and complementarity determining regions. IgBLAST can analyze nucleotide or protein sequences and can process sequences in batches and allows searches against the germline gene databases and other sequence databases simultaneously to minimize the chance of missing possibly the best matching germline V gene.

In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one approach, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residues occur in both sequences to yield the number of matched positions, dividing the number of matched positions by the total number of positions in the reference sequence (i.e., the window size) and multiplying the results by 100 to yield the percentage of sequence identity.

In some embodiments, the antibody or antibody fragment disclosed herein comprises, or consists essentially of, or consists of: the amino acid sequence of SEQ ID NO: 100 and the amino acid sequence of SEQ ID NO: 155; the amino acid sequence of SEQ ID NO: 101 and the amino acid sequence of SEQ ID NO: 156; the amino acid sequence of SEQ ID NO: 102 and the amino acid sequence of SEQ ID NO: 156; the amino acid sequence of SEQ ID NO: 103 and the amino acid sequence of SEQ ID NO: 157; the amino acid sequence of SEQ ID NO: 103 and the amino acid sequence of SEQ ID NO: 158; the amino acid sequence of SEQ ID NO: 103 and the amino acid sequence of SEQ ID NO: 159; the amino acid sequence of SEQ ID NO: 103 and the amino acid sequence of SEQ ID NO: 160; the amino acid sequence of SEQ ID NO: 104 and the amino acid sequence of SEQ ID NO: 161; the amino acid sequence of SEQ ID NO: 105 and the amino acid sequence of SEQ ID NO: 162; the amino acid sequence of SEQ ID NO: 105 and the amino acid sequence of SEQ ID NO: 163; the amino acid sequence of SEQ ID NO: 106 and the amino acid sequence of SEQ ID NO: 164; the amino acid sequence of SEQ ID NO: 107 and the amino acid sequence of SEQ ID NO: 165; the amino acid sequence of SEQ ID NO: 108 and the amino acid sequence of SEQ ID NO: 166; or the amino acid sequence of SEQ ID NO: 109 and the amino acid sequence of SEQ ID NO: 163.

In some embodiments, the antibody or antibody fragment disclosed herein comprises, or consists essentially of, or consists of a heavy chain variable sequence and a light chain variable sequence having an amino acid sequence disclosed in Table 4. In some embodiments, the antibody or antibody fragment is a monoclonal antibody or a human monoclonal antibody. In some embodiments, the antibody fragment is a recombinant scFv (single chain fragment variable) antibody, a Fab fragment, a F(ab′)2 fragment, or a variable domain fragment (Fv fragment).

In some embodiments, the antibody fragment is a recombinant scFv (single chain fragment variable) antibody. In some embodiments, the antibody fragment is a recombinant antibody variable domain fragment (Fv fragment). In some embodiments, the Fv fragment retains the specificity of the intact antibody from which it is derived. In some embodiments, the antibody fragment (e.g., Fv fragment) comprises the amino acid sequence of SEQ ID NOs: 1-14 or SEQ ID NOs: 15-99 or an amino acid sequence disclosed in Table 4 or FIG. 4 or Table 13. In some embodiments, the antibody fragment (e.g., scFv or Fv fragment) comprises an amino acid sequence substantially identical (e.g., at least 80%, 85%, 90%, 92%, 95%, 97%, 98%, 99% or higher identical) to any of the amino acid sequences disclosed in Table 4 or FIG. 4 or Table 13, with the proviso that the amino acid mutations are not altered in the equivalent from those mutations identified in Table 13.

In some embodiments, the Fv fragment further comprises a linker domain to generate a single chain variable fragment (scFv). In one embodiment, the linker domain is operably linked to the heavy chain variable domain and the light chain variable domain. In some embodiments, the light and heavy chain variable regions are contiguously linked via a short flexible polypeptide linker, and capable of being expressed as a single chain polypeptide, and the scFv retains the specificity of the intact antibody from which it is derived. In one embodiment, the flexible polypeptide linker includes, but are not limited to, (Gly4 Ser)4 or (Gly4 Ser)3. In another embodiment, the linker includes multiple repeats of (Gly2Ser), (GlySer) or (Gly3Ser).

### Fc Mutations

In some embodiments, the antibody or fragment thereof, or nucleic acid encoding same is conjugated or bound to a label or therapeutic agent. In one aspect, the antibody is an IgG antibody, conjugated to a label, and/or conjugated to a therapeutic agent. In some embodiments, the antibody is an IgG. In some embodiments, the antibody is a recombinant IgG antibody. In some embodiments, the antibody is an antibody fragment comprising an Fc portion mutated to alter (eliminate or enhance) FcR interactions. In one embodiment, the mutated Fc increases the half-life and/or increase the therapeutic efficacy of the antibody disclosed herein. In one embodiment, the mutated Fc portion comprises a LALA, LALA PG, N297, GASD/ALIE, DHS, YTE or LS mutation. In some embodiments, the Fc portion is glycan modified to alter, eliminate, or enhance FcR interactions, such as enzymatic or chemical addition or removal of glycans or expression in a cell line engineered with a defined glycosylating pattern. In some embodiments, the antibody comprises a YTE mutation.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced. Amino acid modifications that alter C1q and/or modify its complement dependent cytotoxicity function are described in WO 2000/0042072.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, (2011)), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

In some embodiments, the antibody or antibody fragment further comprises a label. In some embodiments, the antibody is a chimeric antibody, a bispecific antibody, humanized antibody, a human antibody, or a fully human antibody.

### Antibody Derivatives

One aspect of the present disclosure provides a “derivative” of an antibody or antigen fragment described herein or disclosed in Table 4. The term “derivative” refers to an antibody or antigen-binding fragment thereof that immunospecifically binds to an antigen but which comprises, one, two, three, four, five or more amino acid substitutions, additions, deletions or modifications relative to a “parental” (or wild-type) molecule. Such amino acid substitutions or additions may introduce naturally occurring (i.e., DNA-encoded) or non-naturally occurring amino acid residues. The term “derivative” encompasses, for example, as variants having altered CH1, hinge, CH2, CH3 or CH4 regions, so as to form, for example, antibodies, etc., having variant Fc regions that exhibit enhanced or impaired effector or binding characteristics. The term “derivative” additionally encompasses non-amino acid modifications, for example, amino acids that may be glycosylated (e.g., have altered mannose, 2-N-acetylglucosamine, galactose, fucose, glucose, sialic acid, 5-N-acetylneuraminic acid, 5-glycolneuraminic acid, etc. content), acetylated, pegylated, phosphorylated, amidated, derivatized by known protecting/blocking groups, proteolytic cleavage, linked to a cellular ligand or other protein, etc.

In some embodiments, the altered carbohydrate modifications modulate one or more of the following: solubilization of the antibody, facilitation of subcellular transport and secretion of the antibody, promotion of antibody assembly, conformational integrity, and antibody-mediated effector function. In a specific embodiment, the altered carbohydrate modifications enhance antibody mediated effector function relative to the antibody lacking the carbohydrate modification. Carbohydrate modifications that lead to altered antibody mediated effector function are well known in the art. See e.g., Shields et al., J. Biol. Chem. 277(30): 26733-26740 (2002); Davies J. et al., Biotechnology & Bioengineering 74(4): 288-294 (2001). Methods of altering carbohydrate contents are also known to those skilled in the art. See, e.g., Wallick et al. J. Exp. Med. 168(3): 1099-1109 (1988); Tao et al. J. Immunol. 143(8): 2595-2601 (1989); Routledge et al., Transplantation 60(8):847-53 (1995); Elliott et al., Nature Biotechnol. 21:414-21 (2003); Shield et al. J. Biol. Chem. 277(30): 26733-26740 (2002).

A derivative antibody or antibody fragment can be generated with an engineered sequence or glycosylation state to confer preferred levels of activity in antibody dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

A derivative antibody or antibody fragment may be modified by chemical modifications using techniques known to those of skill in the art, including, but not limited to, specific chemical cleavage, acetylation, formulation, metabolic synthesis of tunicamycin, etc. In one embodiment, an antibody derivative will possess a similar or identical function as the parental antibody. In another embodiment, an antibody derivative will exhibit an altered activity relative to the parental antibody. For example, a derivative antibody (or fragment thereof) can bind to its epitope more tightly or be more resistant to proteolysis than the parental antibody.

### Antibody Conjugates

In some embodiments, the antibody or antibody fragment of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules which have been attached to antibodies include toxins, anti-tumor agents, therapeutic enzymes, radionuclides, antiviral agents, chelating agents, cytokines, growth factors, and oligo- or polynucleotides. By contrast, a reporter molecule is defined as any moiety which may be detected using an assay. Non-limiting examples of reporter molecules, which have been conjugated to antibodies include enzymes, radiolabels, haptens, fluorescent labels, phosphorescent molecules, chemiluminescent molecules, chromophores, photoaffinity molecules, colored particles or ligands, such as biotin.

### Labeled Antibodies

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies. See e.g., U.S. Pat. Nos. 5,021,236, 4,938,948, and 4,472,509. The imaging moieties used can be paramagnetic ions, radioactive isotopes, fluorochromes, NMR-detectable substances, and X-ray imaging agents. In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine211, 14carbon, 51chromium, 36chlorine, 57cobalt, 58cobalt, copper67, 152Eu, gallium67, 3hydrogen, iodine123, iodine125, iodine131, indium111, 59iron, 32phosphorus, rhenium186, rhenium188, 75selenium, 35sulphur, technicium99m and/or yttrium90. 1251 is often being preferred for use in certain embodiments, and technicium99m and/or indium111 are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium99m by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNC12, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### Antibody-Dependent Cell-Mediated Cytotoxicity (ADCC)

In some embodiments, the antibody or antibody fragment of the present disclosure is an antibody-dependent cell-mediated cytotoxicity (ADCC). An ADCC is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

### Complement-Dependent Cytotoxicity (CDC)

In some embodiments, the antibody or antibody fragment of the present disclosure is a Complement-dependent cytotoxicity (CDC). A CDC is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC. It is one of the mechanisms by which antibodies or antibody fragments have an anti-viral effect.

### Cross-Reactivity

Two main categories of SARS-CoV-2 antigens are the surface spike (S) protein and the internal proteins, especially the nucleocapsid (N) protein. Antibodies to the S protein will be useful for prophylaxis, or therapy, or diagnostics, or for characterizing vaccines. S protein antibodies will have additional binding specificity with that protein, with particular antibodies binding to the full-length ectodomain of the SARS-CoV-2 S protein (presented as a monomer or oligomer such as a timer; with our without conformation stabilizing mutations such as introduction of prolines at critical sites (“2P mutation”)) and (a) anti-S protein antibodies that binds to the receptor binding domain (RBD), (b) anti-S protein antibodies that bind to domains other than the RBD. Some of the subset that bind to domains other than the RBD bind to the N terminal domain (NTD), while others bind to an epitope other than the NTD or RBD), and (c) S protein antibodies may further be found to neutralize SARS-CoV-2 by blocking binding of the SARS-CoV-2 S protein to its receptor, human angiotensin-converting enzyme 2 (hACE2), with others that neutralize but do not block receptor binding. Finally, antibodies can cross-react with both SARS-CoV-2 S protein and the S protein of other coronaviruses such as SARS-CoV, MERS-CoV, HCoV-229E, HCoV-OC43, HCoV-NL63 and/or HCoV-HKU1, as well as cross-neutralize both SARS-CoV-2 and these other coronaviruses.

Another specificity will be antibodies that bind to N antibodies (or other internal targets) that will have primarily diagnostics uses. For example, antibodies to N or other internal proteins of SARS-CoV-2 that specifically recognize SARS-CoV-2 or that cross-reactively recognize SARS-CoV-2 and other coronaviruses such as SARS-CoV, MERS-CoV, HCoV-229E, HCoV-OC43, HCoV-NL63 and/or HCoV-HKU1.

### Constant Region of an Antibody

In one aspect, the present technology provides an antibody or an antigen binding fragment thereof, comprising a heavy chain immunoglobulin variable domain (V_{H}) and a light chain immunoglobulin variable domain (V_{L}) as disclosed herein, and a Fc domain of any isotype, e.g., but are not limited to, IgG (including IgG1, IgG2, IgG3, and IgG4), IgA (including IgA₁ and IgA₂), IgD, IgE, or IgM, and IgY. Non-limiting examples of constant region sequences include:

The constant regions of antibodies can also be varied. For example, antibodies are provided with Fc regions of any isotype: IgA (IgA1, IgA2), IgD, IgE, IgG (IgG1, IgG2, IgG3, IgG4) or IgM. Non-limiting examples of constant region sequences include:

Human IgD constant region, Uniprot: P01880
(SEQ ID NO: 252)
APTKAPDVFPIISGCRHPKDNSPVVLACLITGYHPTSVTVTWYMGTQSQP
QRTFPEIQRRDSYYMTSSQLSTPLQQWRQGEYKCVVQHTASKSKKEIFRW
PESPKAQASSVPTAQPQAEGSLAKATTAPATTRNTGRGGEEKKKEKEKEE
QEERETKTPECPSHTQPLGVYLLTPAVQDLWLRDKATFTCFVVGSDLKDA
HLTWEVAGKVPTGGVEEGLLERHSNGSQSQHSRLTLPRSLWNAGTSVTCT
LNHPSLPPQRLMALREPAAQAPVKLSLNLLASSDPPEAASWLLCEVSGFS
PPNILLMWLEDQREVNTSGFAPARPPPQPGSTTFWAWSVLRVPAPPSPQP
ATYTCVVSHEDSRTLLNASRSLEVSYVTDHGPMK, and
equivalents thereof.
Human IgG1 constant region, Uniprot: P01857
(SEQ ID NO: 253)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK, and equivalents
thereof.
Human IgG2 constant region, Uniprot: P01859
(SEQ ID NO: 254)
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK, and equivalents
thereof.
Human IgG3 constant region, Uniprot: P01860
(SEQ ID NO: 255)
ASTKGPSVFPLAPCSRSTSGGTAALGCLVKDYFPEPVTVSWNSGALTSGV
HTFPAVLQSSGLYSLSSVVTVPSSSLGTQTYTCNVNHKPSNTKVDKRVEL
KTPLGDTTHTCPRCPEPKSCDTPPPCPRCPEPKSCDTPPPCPRCPEPKSC
DTPPPCPRCPAPELLGGPSVFLFPPKPKDTLMISRTPEVTCVVVDVSHED
PEVQFKWYVDGVEVHNAKTKPREEQYNSTFRVVSVLTVLHQDWLNGKEYK
CKVSNKALPAPIEKTISKTKGQPREPQVYTLPPSREEMTKNQVSLTCLVK
GFYPSDIAVEWESSGQPENNYNTTPPMLDSDGSFFLYSKLTVDKSRWQQG
NIFSCSVMHEALHNRFTQKSLSLSPGK, and equivalents
thereof.
Human IgM constant region, Uniprot: P01871
(SEQ ID NO: 256)
GSASAPTLFPLVSCENSPSDTSSVAVGCLAQDFLPDSITLSWKYKNNSDI
SSTRGFPSVLRGGKYAATSQVLLPSKDVMQGTDEHVVCKVQHPNGNKEKN
VPLPVIAELPPKVSVFVPPRDGFFGNPRKSKLICQATGFSPRQIQVSWLR
EGKQVGSGVTTDQVQAEAKESGPTTYKVTSTLTIKESDWLGQSMFTCRVD
HRGLTFQQNASSMCVPDQDTAIRVFAIPPSFASIFLTKSTKLTCLVTDLT
TYDSVTISWTRQNGEAVKTHTNISESHPNATFSAVGEASICEDDWNSGER
FTCTVTHTDLPSPLKQTISRPKGVALHRPDVYLLPPAREQLNLRESATIT
CLVTGFSPADVFVQWMQRGQPLSPEKYVTSAPMPEPQAPGRYFAHSILTV
SEEEWNTGETYTCVAHEALPNRVTERTVDKSTGKPTLYNVSLVMSDTAGT
CY, and equivalents thereof.
Human IgG4 constant region, Uniprot: P01861
(SEQ ID NO: 257)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK, and equivalents
thereof.
Human IgA1 constant region, Uniprot: P01876
(SEQ ID NO: 258)
ASPTSPKVFPLSLCSTQPDGNVVIACLVQGFFPQEPLSVTWSESGQGVTA
RNFPPSQDASGDLYTTSSQLTLPATQCLAGKSVTCHVKHYTNPSQDVTVP
CPVPSTPPTPSPSTPPTPSPSCCHPRLSLHRPALEDLLLGSEANLTCTLT
GLRDASGVTFTWTPSSGKSAVQGPPERDLCGCYSVSSVLPGCAEPWNHGK
TFTCTAAYPESKTPLTATLSKSGNTFRPEVHLLPPPSEELALNELVTLTC
LARGFSPKDVLVRWLQGSQELPREKYLTWASRQEPSQGTTTFAVTSILRV
AAEDWKKGDTFSCMVGHEALPLAFTQKTIDRLAGKPTHVNVSVVMAEVDG
TCY, and equivalents thereof.
Human IgA2 constant region, Uniprot: P01877
(SEQ ID NO: 259)
ASPTSPKVFPLSLDSTPQDGNVVVACLVQGFFPQEPLSVTWSESGQNVTA
RNFPPSQDASGDLYTTSSQLTLPATQCPDGKSVTCHVKHYTNPSQDVTVP
CPVPPPPPCCHPRLSLHRPALEDLLLGSEANLTCTLTGLRDASGATFTWT
PSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVR
WLQGSQELPREKYLTWASRQEPSQGTTTFAVTSILRVAAEDWKKGDTFSC
MVGHEALPLAFTQKTIDRMAGKPTHVNVSVVMAEVDGTCY, and
equivalents thereof.
Human Ig kappa constant region, Uniprot: P01834
(SEQ ID NO: 260)
TVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKS
FNRGEC, and equivalents thereof.

Additional exemplary constant regions are disclosed in Table 5.

In some embodiments, the antibody disclosed herein comprises a heavy chain constant region that is at least 80% identical to an amino acid sequence disclosed in Table 5. In some embodiments, the antibody disclosed herein comprises a heavy chain constant region that is at least 80% identical to any one of SEQ ID NO: 261-264. In some embodiments, the antibody disclosed herein comprises a heavy chain constant region having the amino acid sequence of SEQ ID NO: 261-264.

In some embodiments, the antibodies comprise a light chain constant region that is at least 80% identical to an amino acid sequence disclosed in Table 5. In some embodiments, the antibody disclosed herein comprises a heavy chain constant region that is at least 80% identical to an amino acid sequence disclosed in Table 5. In some embodiments, the antibody disclosed herein comprises a light chain constant region that is at least 80% identical to any one of SEQ ID NO: 265-270 or 252-254. In some embodiments, the antibody disclosed herein comprises a light chain constant region having the amino acid sequence of SEQ ID NO: 265-270.

The constant regions of antibodies can also be varied. For example, antibodies are provided with Fc regions of any isotype: IgA (IgA1, IgA2), IgD, IgE, IgG (IgG1, IgG2, IgG3, IgG4) or IgM. Non-limiting examples of constant region sequences include: Human IgD constant region, Uniprot: P01880 (e.g., as show in SEQ ID NO: 252), and equivalents thereof; Human IgG1 constant region (Uniprot: P01857 or as show in SEQ ID NO: 253), and equivalents thereof; Human IgG2 constant region (Uniprot: P01859 or as show in SEQ ID NO: 254), and equivalents thereof; Human IgG3 constant region (Uniprot: P01860 or as show in SEQ ID NO: 255), and equivalents thereof; Human IgM constant region (Uniprot: P01871 or as show in SEQ ID NO: 256), and equivalents thereof; Human IgG4 constant region (Uniprot: P01861 or as show in SEQ ID NO: 257), and equivalents thereof; Human IgA1 constant region (Uniprot: P01876 or as show in SEQ ID NO: 258), and equivalents thereof; Human IgA2 constant region (Uniprot: P01877 or as show in SEQ ID NO: 259), and equivalents thereof; and Human Ig kappa constant region (Uniprot: P01834 or as show in SEQ ID NO: 260), and equivalents thereof.

In some embodiments, the antibody or antigen binding fragment thereof, comprising a heavy chain immunoglobulin variable domain (VH) and a light chain immunoglobulin variable domain (VL) as disclosed herein, and a constant region selected from SEQ ID NOs: 252-260. In a specific embodiment, the VH domain is linked to SEQ ID NO: 253. In a specific embodiment, the VL domain is linked to SEQ ID NO: 260.

TABLE 5
Amino acid sequences of the constant region of selected anti-SARS-
CoV2 antibodies disclosed herein.
Table 5: Amino acid sequences of the constant region of selected anti-SARS-CoV2
antibodies

| | | Antibody | |
|---|---|---|---|
| SEQ | Protein | MD5 Hash | |
| ID NO | Name | ID | Variable Region Sequences (VH/VL) |
| 1 | mAb_RBD_ | md5_e3d1904 | EVQLVESGGGLVKPGGSLRLSCAASGFTFRDVW |
| | 2130_ | 966eaf73aed3 | MSWVRQAPGKGLEWVGRIKSKEDGGTTDYAA |
| | FAB_ | 0331f60b658e | PVKGRFTISRDDSKNTLYLQMNSLKTEDTAVYY |
| | 20211231_ | 9 | CTTAGSYYFDTVGPGLPEGKFDYWGQGTLVTV |
| | 00017 | | SS/DIVMTQSPDSLAVSLGERATINCKSSQSVLY |
| | | | WANNKNYLAWYQQKPGQPPKLLMYWASTRES |
| | | | GVPDRFSGSGSGAEFTLTISSLQAEDVAIYYCQQ |
| | | | YYSTLTFGGGTKVEIKR |
| 2 | mAb_RBD_ | md5_d323d72 | EVQLVESGGGLVKPGGSLRLSCAASGFTFRDVW |
| | 2130_ | bb6bc337ea25 | MSWVRQAPGKGLEWVGRIKSKIDGGTTDYAAP |
| | FAB_ | 6bdd92ceba08 | VKGRFTISRDDSKNTLYLQMNSLKTEDTAVYYC |
| | 20211231_ | e | TTAGSYYYDTRGPGLPEGKFDYWGQGTLVTVS |
| | 00174 | | S/DIVMTQSPDSLAVSLGERATINCKSSQSVLYSS |
| | | | NNKNYLAWYQQKPGQPPKLLMYWASTRESGV |
| | | | PDRFSGSGSGAEFTLTISSLQAEDVAIYYCQQYY |
| | | | STLTFGGGTKVEIKR |
| 3 | mAb_RBD_ | md5_9f64868 | EVQLVESGGGLVKPGGSLRLSCAASGFTFRDVW |
| | 2130_ | 08688a2b46ec | MSWVRQAPGKGLEWVGRIKSKWDGGTTDYAA |
| | FAB_ | 220af47cb95c | PVKGRFTISRDDSKNTLYLQMNSLKTEDTAVYY |
| | 20211231_ | 5 | CTTAGSYYYDTVGPGLPEGKFDYWGQGTLVTV |
| | 00194 | | SS/DIVMTQSPDSLAVSLGERATINCKSSQSVLYS |
| | | | SNNKNYLAWYQQKPGQPPKLLMYWASTRESG |
| | | | VPDRFSGSGSGAEFTLTISSLQAEDVAIYYCQQY |
| | | | YSTLTFGGGTKVEIKR |
| 4 | mAb_RBD_ | md5_eebd2cc | EVQLVESGGGLVKPGGSLRLSCAASGFTFRDVW |
| | 2130_ | 17866724358 | MSWVRQAPGKGLEWVGRIKSKIDGGTTDYAAP |
| | FAB_ | ced82fe31446 | VKGRFTISRDDSKNTLYLQMNSLKTEDTAVYYC |
| | 20211231_ | 5c | TTAGSYYYDTVGPGLPEGKFDYWGQGTLVTVS |
| | 00199 | | S/DIVMTQSPDSLAVSLGERATINCKSSQSVLYSY |
| | | | NNKNYLAWYQQKPGQPPKLLMYWASTRESGV |
| | | | PDRFSGSGSGAEFTLTISSLQAEDVAIYYCQQYY |
| | | | STLTFGGGTKVEIKR |
| 5 | mAb_RBD_ | md5_aaca5fa0 | EVQLVESGGGLVKPGGSLRLSCAASGFTFRDVW |
| | 2130_ | 1a0f27e83e39 | MSWVRQAPGKGLEWVGRIKSKIDGGTTDYAAP |
| | FAB_ | da01a445734 | VKGRFTISRDDSKNTLYLQMNSLKTEDTAVYYC |
| | 20211231_ | 7 | TTAGSYYYDTVGPGLPEGKFDYWGQGTLVTVS |
| | 00200 | | S/DIVMTQSPDSLAVSLGERATINCKSSQSVLYS |
| | | | WNNKNYLAWYQQKPGQPPKLLMYWASTRESG |
| | | | VPDRFSGSGSGAEFTLTISSLQAEDVAIYYCQQY |
| | | | YSTLTFGGGTKVEIKR |
| 6 | v2130_ | md5_2041aa1 | EVQLVESGGGLVKPGGSLRLSCAASGFTFRDVW |
| | SFE_ | 10908c8cde47 | MSWVRQAPGKGLEWVGRIKSKIDGGTTDYAAP |
| | 02022014_ | ed7aabc074be | VKGRFTISRDDSKNTLYLQMNSLKTEDTAVYYC |
| | 00015 | 8 | TTAGSYYYDTVGPGLPEGKFDYWGQGTLVTVS |
| | | | S/DIVMTQSPDSLAVSLGERATINCKSSQSVLYQ |
| | | | YNNKNYLAWYQQKPGQPPKLLMYWASHRESG |
| | | | VPDRFSGSGSGAEFTLTISSLQAEDVAIYYCQQY |
| | | | YSTLTFGGGTKVEIKR |
| 7 | v2130_ | md5_1089bd1 | EVQLVESGGGLVKPGGSLRLSCAASGFTFRDVW |
| | SFE_ | 4be7c511aa67 | MSWVRQAPGKGLEWVGRIKSKIDGGTTDYAAP |
| | 20220104_ | dbef73eeb1e4 | VKGRFTISRDDSKNTLYLQMNSLKTEDTAVYYC |
| | 00024 | 2 | TTAGSYYYDTVGPGLPEGKFDYWGQGTLVTVS |
| | | | S/DIVMTQSPDSLAVSLGERATINCKSSQSVLYW |
| | | | SNNKNYLAWYQQKPGQPPKLLMYWASERESG |
| | | | VPDRFSGSGSGAEFTLTISSLQAEDVAIYYCQQY |
| | | | YSTLTFGGGTKVEIKR |
| | | Antibody | |
| | Protein | MD5 Hash | |
| | Name | ID | Full Heavy Chain Sequence (VH + Constant) |
| 261 | mAb_RBD_ | md5_e3d1904 | EVQLVESGGGLVKPGGSLRLSCAASGFTFRDVW |
| | 2130_ | 966eaf73aed3 | MSWVRQAPGKGLEWVGRIKSKEDGGTTDYAA |
| | FAB_ | 0331f60b658e | PVKGRFTISRDDSKNTLYLQMNSLKTEDTAVYY |
| | 20211231_ | 9 | CTTAGSYYFDTVGPGLPEGKFDYWGQGTLVTV |
| | 00017 | | SSASTKGPSVFPLAPSSKSTSGGTAALGCLVKDY |
| | | | FPEPVTVSWNSGALTSGVHTFPAVLQSSGLYSLS |
| | | | SVVTVPSSSLGTQTYICNVNHKPSNTKVDKKVE |
| | | | PKSCDKTHTCPPCPAPELLGGPSVFLFPPKPKDT |
| | | | LMISRTPEVTCVVVDVSHEDPEVKFNWYVDGV |
| | | | EVHNAKTKPREEQYNSTYRVVSVLTVLHQDWL |
| | | | NGKEYKCKVSNKALPAPIEKTISKAKGQPREPQ |
| | | | VYTLPPSRDELTKNQVSLTCLVKGFYPSDIAVE |
| | | | WESNGQPENNYKTTPPVLDSDGSFFLYSKLTVD |
| | | | KSRWQQGNVFSCSVMHEALHNHYTQKSLSLSP |
| | | | GK* |
| 262 | mAb_RBD_ | md5_d323d72 | EVQLVESGGGLVKPGGSLRLSCAASGFTFRDVW |
| | 2130_ | bb6bc337ea25 | MSWVRQAPGKGLEWVGRIKSKIDGGTTDYAAP |
| | FAB_ | 6bdd92ceba08 | VKGRFTISRDDSKNTLYLQMNSLKTEDTAVYYC |
| | 20211231_ | e | TTAGSYYYDTRGPGLPEGKFDYWGQGTLVTVS |
| | 00174 | | SASTKGPSVFPLAPSSKSTSGGTAALGCLVKDYF |
| | | | PEPVTVSWNSGALTSGVHTFPAVLQSSGLYSLSS |
| | | | VVTVPSSSLGTQTYICNVNHKPSNTKVDKKVEP |
| | | | KSCDKTHTCPPCPAPELLGGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALPAPIEKTISKAKGQPREPQV |
| | | | YTLPPSRDELTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPG |
| | | | K* |
| 263 | mAb_RBD_ | md5_9f64868 | EVQLVESGGGLVKPGGSLRLSCAASGFTFRDVW |
| | 2130_ | 08688a2b46ec | MSWVRQAPGKGLEWVGRIKSKWDGGTTDYAA |
| | FAB_ | 220af47cb95c | PVKGRFTISRDDSKNTLYLQMNSLKTEDTAVYY |
| | 20211231_ | 5 | CTTAGSYYYDTVGPGLPEGKFDYWGQGTLVTV |
| | 00194 | | SSASTKGPSVFPLAPSSKSTSGGTAALGCLVKDY |
| | | | FPEPVTVSWNSGALTSGVHTFPAVLQSSGLYSLS |
| | | | SVVTVPSSSLGTQTYICNVNHKPSNTKVDKKVE |
| | | | PKSCDKTHTCPPCPAPELLGGPSVFLFPPKPKDT |
| | | | LMISRTPEVTCVVVDVSHEDPEVKFNWYVDGV |
| | | | EVHNAKTKPREEQYNSTYRVVSVLTVLHQDWL |
| | | | NGKEYKCKVSNKALPAPIEKTISKAKGQPREPQ |
| | | | VYTLPPSRDELTKNQVSLTCLVKGFYPSDIAVE |
| | | | WESNGQPENNYKTTPPVLDSDGSFFLYSKLTVD |
| | | | KSRWQQGNVFSCSVMHEALHNHYTQKSLSLSP |
| | | | GK* |
| 264 | mAb_RBD_ | md5_eebd2cc | EVQLVESGGGLVKPGGSLRLSCAASGFTFRDVW |
| | 2130_ | 1786b724358 | MSWVRQAPGKGLEWVGRIKSKIDGGTTDYAAP |
| | FAB_ | ced82fe31446 | VKGRFTISRDDSKNTLYLQMNSLKTEDTAVYYC |
| | 20211231_ | 5c | TTAGSYYYDTVGPGLPEGKFDYWGQGTLVTVS |
| | 00199 | | SASTKGPSVFPLAPSSKSTSGGTAALGCLVKDYF |
| | | | PEPVTVSWNSGALTSGVHTFPAVLQSSGLYSLSS |
| | | | VVTVPSSSLGTQTYICNVNHKPSNTKVDKKVEP |
| | | | KSCDKTHTCPPCPAPELLGGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALPAPIEKTISKAKGQPREPQV |
| | | | YTLPPSRDELTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPG |
| | | | K* |
| 264 | mAb_RBD_ | md5_aaca5fa0 | EVQLVESGGGLVKPGGSLRLSCAASGFTFRDVW |
| | 2130_ | 1a0f27e83e39 | MSWVRQAPGKGLEWVGRIKSKIDGGTTDYAAP |
| | FAB_ | da01a445734 | VKGRFTISRDDSKNTLYLQMNSLKTEDTAVYYC |
| | 20211231_ | 7 | TTAGSYYYDTVGPGLPEGKFDYWGQGTLVTVS |
| | 00200 | | SASTKGPSVFPLAPSSKSTSGGTAALGCLVKDYF |
| | | | PEPVTVSWNSGALTSGVHTFPAVLQSSGLYSLSS |
| | | | VVTVPSSSLGTQTYICNVNHKPSNTKVDKKVEP |
| | | | KSCDKTHTCPPCPAPELLGGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALPAPIEKTISKAKGQPREPQV |
| | | | YTLPPSRDELTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPG |
| | | | K* |
| 264 | v2130_SFE_ | md5_2041aa1 | EVQLVESGGGLVKPGGSLRLSCAASGFTFRDVW |
| | 20220104_ | 10908c8cde47 | MSWVRQAPGKGLEWVGRIKSKIDGGTTDYAAP |
| | 00015 | ed7aabc074be | VKGRFTISRDDSKNTLYLQMNSLKTEDTAVYYC |
| | | 8 | TTAGSYYYDTVGPGLPEGKFDYWGQGTLVTVS |
| | | | SASTKGPSVFPLAPSSKSTSGGTAALGCLVKDYF |
| | | | PEPVTVSWNSGALTSGVHTFPAVLQSSGLYSLSS |
| | | | VVTVPSSSLGTQTYICNVNHKPSNTKVDKKVEP |
| | | | KSCDKTHTCPPCPAPELLGGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALPAPIEKTISKAKGQPREPQV |
| | | | YTLPPSRDELTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPG |
| | | | K* |
| 264 | v2130_SFE_ | md5_1089bd1 | EVQLVESGGGLVKPGGSLRLSCAASGFTFRDVW |
| | 20220104_ | 4be7c511aa67 | MSWVRQAPGKGLEWVGRIKSKIDGGTTDYAAP |
| | 00024 | dbef73eeb1e4 | VKGRFTISRDDSKNTLYLQMNSLKTEDTAVYYC |
| | | 2 | TTAGSYYYDTVGPGLPEGKFDYWGQGTLVTVS |
| | | | SASTKGPSVFPLAPSSKSTSGGTAALGCLVKDYF |
| | | | PEPVTVSWNSGALTSGVHTFPAVLQSSGLYSLSS |
| | | | VVTVPSSSLGTQTYICNVNHKPSNTKVDKKVEP |
| | | | KSCDKTHTCPPCPAPELLGGPSVFLFPPKPKDTL |
| | | | MISRTPEVTCVVVDVSHEDPEVKFNWYVDGVE |
| | | | VHNAKTKPREEQYNSTYRVVSVLTVLHQDWLN |
| | | | GKEYKCKVSNKALPAPIEKTISKAKGQPREPQV |
| | | | YTLPPSRDELTKNQVSLTCLVKGFYPSDIAVEW |
| | | | ESNGQPENNYKTTPPVLDSDGSFFLYSKLTVDK |
| | | | SRWQQGNVFSCSVMHEALHNHYTQKSLSLSPG |
| | | | K* |
| | | Antibody | |
| | Protein | MD5 Hash | |
| | Name | ID | Full Light Chain Sequence (VL + Constant) |
| 265 | mAb_RBD_ | md5_e3d1904 | DIVMTQSPDSLAVSLGERATINCKSSQSVLYWA |
| | 2130_FAB_ | 966eaf73aed3 | NNKNYLAWYQQKPGQPPKLLMYWASTRESGV |
| | 20211231_ | 0331f60b658e | PDRFSGSGSGAEFTLTISSLQAEDVAIYYCQQYY |
| | 00017 | 9 | STLTFGGGTKVEIKRTVAAPSVFIFPPSDEQLKSG |
| | | | TASVVCLLNNFYPREAKVQWKVDNALQSGNSQ |
| | | | ESVTEQDSKDSTYSLSSTLTLSKADYEKHKVYA |
| | | | CEVTHQGLSSPVTKSFNRGEC* |
| 266 | mAb_RBD_ | md5_d323d72 | DIVMTQSPDSLAVSLGERATINCKSSQSVLYSSN |
| | 2130_FAB_ | bb6bc337ea25 | NKNYLAWYQQKPGQPPKLLMYWASTRESGVP |
| | 20211231_ | 6bdd92ceba08 | DRFSGSGSGAEFTLTISSLQAEDVAIYYCQQYYS |
| | 00174 | e | TLTFGGGTKVEIKRTVAAPSVFIFPPSDEQLKSGT |
| | | | ASVVCLLNNFYPREAKVQWKVDNALQSGNSQE |
| | | | SVTEQDSKDSTYSLSSTLTLSKADYEKHKVYAC |
| | | | EVTHQGLSSPVTKSFNRGEC* |
| 266 | mAb_RBD_ | md5_9f64868 | DIVMTQSPDSLAVSLGERATINCKSSQSVLYSSN |
| | 2130_FAB_ | 08688a2b46ec | NKNYLAWYQQKPGQPPKLLMYWASTRESGVP |
| | 20211231_ | 220af47cb95c | DRFSGSGSGAEFTLTISSLQAEDVAIYYCQQYYS |
| | 00194 | 5 | TLTFGGGTKVEIKRTVAAPSVFIFPPSDEQLKSGT |
| | | | ASVVCLLNNFYPREAKVQWKVDNALQSGNSQE |
| | | | SVTEQDSKDSTYSLSSTLTLSKADYEKHKVYAC |
| | | | EVTHQGLSSPVTKSFNRGEC* |
| 267 | mAb_RBD_ | md5_eebd2cc | DIVMTQSPDSLAVSLGERATINCKSSQSVLYSYN |
| | 2130_FAB_ | 17866724358 | NKNYLAWYQQKPGQPPKLLMYWASTRESGVP |
| | 20211231_ | ced82fe31446 | DRFSGSGSGAEFTLTISSLQAEDVAIYYCQQYYS |
| | 00199 | 5c | TLTFGGGTKVEIKRTVAAPSVFIFPPSDEQLKSGT |
| | | | ASVVCLLNNFYPREAKVQWKVDNALQSGNSQE |
| | | | SVTEQDSKDSTYSLSSTLTLSKADYEKHKVYAC |
| | | | EVTHQGLSSPVTKSFNRGEC* |
| 268 | mAb_RBD_ | md5_aaca5fa0 | DIVMTQSPDSLAVSLGERATINCKSSQSVLYSW |
| | 2130_FAB_ | 1a0f27e83e39 | NNKNYLAWYQQKPGQPPKLLMYWASTRESGV |
| | 20211231_ | da01a445734 | PDRFSGSGSGAEFTLTISSLQAEDVAIYYCQQYY |
| | 00200 | 7 | STLTFGGGTKVEIKRTVAAPSVFIFPPSDEQLKSG |
| | | | TASVVCLLNNFYPREAKVQWKVDNALQSGNSQ |
| | | | ESVTEQDSKDSTYSLSSTLTLSKADYEKHKVYA |
| | | | CEVTHQGLSSPVTKSFNRGEC* |
| 269 | v2130_SFE_ | md5_2041aa1 | DIVMTQSPDSLAVSLGERATINCKSSQSVLYQY |
| | 20220104_ | 10908c8cde47 | NNKNYLAWYQQKPGQPPKLLMYWASHRESGV |
| | 00015 | ed7aabc074be | PDRFSGSGSGAEFTLTISSLQAEDVAIYYCQQYY |
| | | 8 | STLTFGGGTKVEIKRTVAAPSVFIFPPSDEQLKSG |
| | | | TASVVCLLNNFYPREAKVQWKVDNALQSGNSQ |
| | | | ESVTEQDSKDSTYSLSSTLTLSKADYEKHKVYA |
| | | | CEVTHQGLSSPVTKSFNRGEC* |
| 270 | v2130_SFE_ | md5_1089bd1 | DIVMTQSPDSLAVSLGERATINCKSSQSVLYWS |
| | 20220104_ | 4be7c511aa67 | NNKNYLAWYQQKPGQPPKLLMYWASERESGV |
| | 00024 | dbef73eeb1e4 | PDRFSGSGSGAEFTLTISSLQAEDVAIYYCQQYY |
| | | 2 | STLTFGGGTKVEIKRTVAAPSVFIFPPSDEQLKSG |
| | | | TASVVCLLNNFYPREAKVQWKVDNALQSGNSQ |
| | | | ESVTEQDSKDSTYSLSSTLTLSKADYEKHKVYA |
| | | | CEVTHQGLSSPVTKSFNRGEC* |

### Nucleic Acids

In another aspect, the present disclosure provides an isolated nucleic acid molecule comprising, or consisting essentially of, or consisting of a nucleotide sequence encoding the CDR, heavy chain, light chain, scFV, antibody or antibody fragment as disclosed herein that are optionally detectably labeled. In some embodiments, the nucleic acid comprises a nucleotide sequence encoding an Fv fragment comprising an amino acid sequence selected from SEQ ID NO: 1-14 or 15-99; or an amino acid sequence disclosed in Table 4 or Table 13.

In some embodiments, the nucleic acid comprises a nucleotide sequence encoding a heavy chain variable sequence comprising an amino acid sequence selected from SEQ ID NOs: 100, 101, 102, 103, 104, 105, 106, 107, 108, or 109. In some embodiments, the nucleic acid comprises a nucleotide sequence encoding a heavy chain variable sequence comprising an amino acid sequence disclosed in Table 4. In some embodiments, the nucleic acid comprises a nucleotide sequence encoding a heavy chain variable sequence comprising an amino acid sequence having at least 70%, 80%, 90%, 95%, 96%, 97%, 98%, or 99% identity to the amino acid sequence disclosed in Table 4.

In some embodiments, the nucleic acid comprises a nucleotide sequence encoding a light chain variable sequence comprising an amino acid sequence selected from SEQ ID NO: 155, 156, 157, 158, 159, 160, 161, 162, 163, 164, 165, or 166. In some embodiments, the nucleic acid comprises a nucleotide sequence encoding a light chain variable sequence comprising an amino acid sequence disclosed in Table 4. In some embodiments, the nucleic acid comprises a nucleotide sequence encoding a light chain variable sequence comprising an amino acid sequence having at least 70%, 80%, 90%, 95%, 96%, 97%, 98%, or 99% identity to the amino acid sequence disclosed in Table 4. In some embodiments, the nucleic acid comprises a nucleotide sequence encoding a heavy chain variable sequence comprising an amino acid sequence selected from any one of SEQ ID NOs: 110-154. In some embodiments, the nucleic acid comprises a nucleotide sequence encoding a heavy chain variable sequence comprising a heavy chain variable amino acid sequence disclosed in Table 14. In some embodiments, the nucleic acid comprises a nucleotide sequence encoding a heavy chain variable sequence comprising an amino acid sequence having at least 70%, 80%, 90%, 95%, 96%, 97%, 98%, or 99% identity a heavy chain variable amino acid sequence disclosed in Table 14, with the proviso that the amino acid mutations are not altered in the equivalent from those mutations identified in Table 14.

In some embodiments, the nucleic acid comprises a nucleotide sequence encoding a light chain variable sequence comprising an amino acid sequence selected from SEQ ID NO: 167, 168, 169, 170, 171, 172, 173, 174, 175, 176, 177, 178, 179, 180, 181, 182, 183, 184, 185, 186, 187, 188, 189, 190, 191, 192, 193, 194, or 195. In some embodiments, the nucleic acid comprises a nucleotide sequence encoding a light chain variable amino acid sequence comprising a light chain variable amino acid sequence disclosed in Table 14. In some embodiments, the nucleic acid comprises a nucleotide sequence encoding a light chain variable sequence comprising a light chain variable amino acid sequence having at least 70%, 80%, 90%, 95%, 96%, 97%, 98%, or 99% identity to the amino acid sequence disclosed in Table 14, with the proviso that the amino acid mutations are not altered in the equivalent from those mutations identified in Table 13.

In one aspect, the present disclosure provides an isolated polynucleotide comprising a nucleic acid sequence encoding the heavy chain variable region of the antibody or antibody fragment disclosed herein and/or a nucleic acid sequence encoding the light chain variable region of the antibody or antibody fragment disclosed herein. In some embodiments, the isolated polynucleotide comprises a nucleic acid sequence encoding the heavy chain variable region of the antibody or antibody fragment disclosed in Table 4 or Table 14 and/or a nucleic acid sequence encoding the light chain variable region of the antibody or antibody fragment disclosed in Table 4 or Table 14. In some embodiments, the isolated polynucleotide comprises a nucleic acid sequence having at least 70%, 80%, 90%, 95%, 96%, 97%, 98%, or 99% identity to a nucleic acid sequence encoding the amino acid sequence of a heavy chain variable region of the antibody or antibody fragment disclosed in Table 4 or Table 14 and/or a nucleic acid sequence having at least 70%, 80%, 90%, 95%, 96%, 97%, 98%, or 99% identity to a nucleic acid sequence encoding the amino acid sequence of a light chain variable region of an antibody or antibody fragment disclosed in Table 4 or Table 14, with the proviso that the amino acid mutations are not altered in the equivalent from those mutations identified in Table 13.

In some embodiments, the nucleic acid comprises a nucleotide sequence encoding a heavy chain complementarity determining region (CHRH1, CDRH2, and/or CDRH3) comprising an amino acid sequence disclosed in Table 13. In some embodiments, the nucleic acid comprises a nucleotide sequence encoding a heavy chain complementarity determining region comprising an amino acid sequence having at least 70%, 80%, 90%, 95%, 96%, 97%, 98%, or 99% identity to a heavy chain complementarity determining region amino acid sequence disclosed in Table 13 with the proviso that the amino acid mutations are not altered in the equivalent from those mutations identified in Table 13.

In some embodiments, the nucleic acid comprises a nucleotide sequence encoding a light chain complementarity determining region (CHRL1, CDRL2, and/or CDRL3) comprising an amino acid sequence disclosed in Table 13. In some embodiments, the nucleic acid comprises a nucleotide sequence encoding a light chain complementarity determining region comprising an amino acid sequence having at least 70%, 80%, 90%, 95%, 96%, 97%, 98%, or 99% identity to a light chain complementarity determining region amino acid sequence disclosed in Table 13, with the proviso that the amino acid mutations are not altered in the equivalent from those mutations identified in Table 13.

The polynucleotides can be DNA or RNA, and can be operably linked to expression elements for transcription, translation or replication of the polynucleotides. Such include, for example promoters and enhancer elements, as are known in the art. The polynucleotides can be used for recombinant production of the polynucleotides or antibodies and fragments thereof as disclosed herein.

### Vectors

In another aspect, the present disclosure provides a vector comprising the isolated nucleic acid molecule comprising, or consisting essentially of, or consisting of a nucleotide sequence encoding the antibody or antibody fragment disclosed herein. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

In one embodiment, the vector comprising the nucleic acid encoding the antibody or antibody fragment disclosed herein is a DNA, a RNA, a plasmid, an adenoviral vector, a lentivirus vector, or a retrovirus vector. A retroviral vector may also be, e.g., a gammaretroviral vector. A gammaretroviral vector may include, e.g., a promoter, a packaging signal (w), a primer binding site (PBS), one or more (e.g., two) long terminal repeats (LTR), and a transgene of interest (e.g., a gene encoding the antibody or antibody fragment disclosed herein). A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., Viruses 3(6): 677-713 (2011).

In another embodiment, the vector comprising the nucleic acid encoding the antibody or antibody fragment disclosed herein is an adenoviral vector (A5/35). In another embodiment, the expression of nucleic acids encoding the antibody or antibody fragment disclosed herein can be accomplished using of transposons such as sleeping beauty, CRISPR, CAS9, and zinc finger nucleases. See, e.g., June et al. Nature Reviews Immunology 9(10): 704-716 (2009). In brief summary, the expression of natural or synthetic nucleic acids encoding the antibody or antibody fragment disclosed herein is typically achieved by operably linking a nucleic acid encoding the antibody or antibody fragment polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence. The expression constructs of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors. Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. See e.g., WO 2001/096584; WO 2001/029058; and U.S. Pat. No. 6,326,193.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, such as enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. A vector may also include a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of the antibody or antibody fragment, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### Methods of Making an Antibody

### Engineered Cell

On aspect of the present disclosure provides an engineered cell comprising an isolated nucleic acid molecule comprising, or consisting essentially of, or consisting of a nucleotide sequence encoding the antibody or antibody fragment disclosed herein or a vector comprising the isolated nucleic acid molecule comprising a nucleotide sequence encoding the antibody or antibody fragment disclosed herein. In some embodiments, the engineered cell produces the antibody or antibody fragment disclosed herein. In some embodiments, the antibody or antibody fragment disclosed herein binds to a SARS-CoV-2 spike protein. In some embodiments, a host cell or engineered cell comprises the vector disclosed herein. In one aspect the cell is a prokaryotic cell. In another aspect it is a eukaryotic cell, such as for example an HEK 293 cell.

### Generating an Antibody

One aspect of the present disclosure provides a method of making an antibody or antibody fragment comprising, or consisting essentially of, or consisting of culturing the engineered cell disclosed herein; and isolating the antibody or antibody fragment thereof from the cultured cell. The “monoclonal antibodies” may also be isolated from phage antibody libraries using the techniques described in Clackson et al., Nature, 352:624-628 (1991) and Marks et al., J. Mol. Biol., 222:581-597 (1991), for example.

The methods for generating monoclonal antibodies generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce SARS-CoV-2-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells identified as responding to infection or vaccination because of plasmablast or activated B cell markers, or memory B cells labelled with the antigen of interest, can be sorted physically using paramagnetic bead selection or flow cytometric sorting, then RNA can be isolated from the single cells and antibody genes amplified by RT-PCR. Various single-cell RNA-seq methods are available to obtain antibody variable genes from single cells. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes from single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 104 times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

DNA encoding the antibody can be employed to produce the antibodies. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

Expression of Recombinant Antibodies. As noted above, the antibodies of the present technology can be produced through the application of recombinant DNA technology. Recombinant polynucleotide constructs an antibody of the present technology typically include an expression control sequence operably-linked to the coding sequences of the antibody chains, including naturally-associated or heterologous promoter regions. As such, another aspect of the technology includes vectors containing one or more nucleic acid sequences encoding the antibody of the present technology. For recombinant expression of one or more of the polypeptides of the present technology, the nucleic acid containing all or a portion of the nucleotide sequence encoding the antibody is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160 and 6,680,192.

In general, expression vectors useful in recombinant DNA techniques are often in the form of plasmids. In the present disclosure, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression of a construct in that subject. In some embodiments, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the antibody, and the collection and purification of the antibody, e.g., cross-reacting antibodies. See generally, U.S. 2002/0199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a protein with binding properties of the antibodies and fragments thereof as disclosed herein in a form suitable for expression of the nucleic acid in a host cell, which means that the recombinant expression vectors include one or more regulatory sequences, selected on the basis of the host cells to be used for expression that is operably-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (i.e., antibody or fragment thereof), include, e.g., but are not limited to, promoters of 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding the antibody of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., antibody, etc.).

Another aspect of the present technology pertains to an antibody-expressing host cells, which contain a nucleic acid encoding one or more antibodies or fragments thereof. The recombinant expression vectors of the present technology can be designed for expression of an antibody in prokaryotic or eukaryotic cells. For example, an antibody can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g., using T7 promoter regulatory sequences and T7 polymerase. Methods useful for the preparation and screening of polypeptides having a predetermined property, e.g., antibody, via expression of stochastically generated polynucleotide sequences has been previously described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., an anti-L1-CAM antibody, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (See, e.g., Wada, et al., 1992. Nucl. Acids Res. 20: 2111-2118). Such alteration of nucleic acid sequences of the present technology can be carried out by standard DNA synthesis techniques.

In another embodiment, the antibody expression vector is a yeast expression vector. Examples of vectors for expression in yeast Saccharomyces cerevisiae include pYepSec1 (Baldari, et al., 1987. EMBO J. 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif), and picZ (Invitrogen Corp, San Diego, Calif). Alternatively, an anti-L1-CAM antibody can be expressed in insect cells using baculovirus expression vectors. Baculovirus vectors available for expression of polypeptides, e.g., anti-L1-CAM antibody, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding an antibody or fragment thereof of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO J. 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells that are useful for expression of the anti-L1-CAM antibody of the present technology, see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid in a particular cell type (e.g., tissue-specific regulatory elements). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

Another aspect of the present methods pertains to host cells into which a recombinant expression vector of the present technology has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an antibody or fragment thereof can be expressed in bacterial cells such as E. coli, insect cells, yeast or mammalian cells. Mammalian cells are a suitable host for expressing nucleotide segments encoding immunoglobulins or fragments thereof. See Winnacker, From Genes To Clones, (VCH Publishers, NY, 1987). A number of suitable host cell lines capable of secreting intact heterologous proteins have been developed in the art, and include Chinese hamster ovary (CHO) cell lines, various COS cell lines, HeLa cells, L cells and myeloma cell lines. In some embodiments, the cells are non-human. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Illustrative expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, electroporation, biolistics or viral-based transfection. Other methods used to transform mammalian cells include the use of polybrene, protoplast fusion, liposomes, electroporation, and microinjection (See generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well-known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the antibody or fragment thereof or can be introduced on a separate vector. Cells stably transfected with the introduced nucleic acid can be identified by drug selection (e.g., cells that have incorporated the selectable marker gene will survive, while the other cells die).

A host cell that includes an antibody or fragment thereof of the present technology, such as a prokaryotic or eukaryotic host cell in culture, can be used to produce (i.e., express) recombinant antibody or fragment thereof. In one embodiment, the method comprises culturing the host cell (into which a recombinant expression vector encoding the antibody or fragment thereof has been introduced) in a suitable medium such that the antibody is produced. In another embodiment, the method further comprises the step of isolating the antibody from the medium or the host cell. Once expressed, collections of the antibody, e.g., the antibodies or the antibody-related polypeptides are purified from culture media and host cells. The antibody can be purified according to standard procedures of the art, including HPLC purification, column chromatography, gel electrophoresis and the like. In one embodiment, the antibody is produced in a host organism by the method of Boss et al., U.S. Pat. No. 4,816,397. Usually, antibody chains are expressed with signal sequences and are thus released to the culture media. However, if the antibody chains are not naturally secreted by host cells, the antibody chains can be released by treatment with mild detergent. Purification of recombinant polypeptides is well known in the art and includes ammonium sulfate precipitation, affinity chromatography purification technique, column chromatography, ion exchange purification technique, gel electrophoresis and the like (See generally Scopes, Protein Purification (Springer-Verlag, N.Y., 1982).

Polynucleotides encoding antibodies, e.g., the antibody coding sequences, can be incorporated in transgenes for introduction into the genome of a transgenic animal and subsequent expression in the milk of the transgenic animal. See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Single-Chain Antibodies. In one embodiment, the antibody of the present technology is a single-chain antibody. According to the present technology, techniques can be adapted for the production of single-chain antibodies specific to a SARs protein (See, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric Antibodies. In one embodiment, the antibody of the present technology is a chimeric antibody. In one embodiment of the present technology, the donor and acceptor antibodies are monoclonal antibodies from different species. For example, the acceptor antibody is a murine antibody (to minimize its antigenicity in a murine animal model).

Recombinant antibodies, such as chimeric monoclonal antibodies, comprising both human and non-human portions, can be made using standard recombinant DNA techniques, and are within the scope of the present technology. For some uses, including in vivo use of the antibody of the present technology in humans or non-human animals as well as use of these agents in in vitro detection assays, it is possible to use chimeric antibodies. Such chimeric monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187; European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559; Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be made chimerice using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding an monoclonal antibody is digested with a restriction enzyme selected specifically to remove the sequence encoding the Fc constant region, and the equivalent portion of a cDNA encoding a different Fc constant region is substituted (See Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc. Natl. Acad. Sci. USA 84: 3439-3443; Liu et al. (1987) J Immunol 139: 3521-3526; Sun et al. (1987) Proc. Natl. Acad. Sci. USA 84: 214-218; Nishimura et al. (1987) Cancer Res 47: 999-1005; Wood et al. (1985) Nature 314: 446-449; and Shaw et al. (1988) J. Natl. Cancer Inst. 80: 1553-1559; U.S. Pat. Nos. 6,180,370; 6,300,064; 6,696,248; 6,706,484; 6,828,422.

CDR Antibodies. Generally the donor and acceptor antibodies used to generate the antibody are monoclonal antibodies from different species; typically the acceptor antibody is a non-human antibody (to minimize its antigenicity in a non-human animal). The graft may be of a single CDR (or even a portion of a single CDR) within a single V_{H} or V_{L} of the acceptor antibody, or can be of multiple CDRs (or portions thereof) within one or both of the V_{H} and V_{L}. Frequently, all three CDRs in all variable domains of the acceptor antibody will be replaced with the corresponding donor CDRs, though one need replace only as many as necessary to permit adequate binding of the resulting CDR-grafted antibody. Methods for generating CDR-grafted antibodies are taught by Queen et al. U.S. Pat. Nos. 5,585,089; 5,693,761; 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; and EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting CDR-grafted antibody by replacing certain residues of a given FR to make the FR more similar to the corresponding FR of the donor antibody. Suitable locations of the substitutions include amino acid residues adjacent to the CDR, or which are capable of interacting with a CDR (See, e.g., U.S. Pat. No. 5,585,089, especially columns 12-16). Techniques for making these modifications are known in the art. Particularly if the resulting FR fits a human consensus FR for that position, or is at least 90% or more identical to such a consensus FR, doing so may not increase the antigenicity of the resulting modified anti-CDR-grafted antibody significantly compared to the same antibody with a fully human FR.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)2) that are produced, for example, by the proteolytic cleavage of the monoclonal antibodies, or single-chain immunoglobulins producible, for example, via recombinant means. F(ab′) antibody derivatives are monovalent, while F(ab′)2 antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 3 or more (e.g., 3, 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke, Methods Mol. Biol. 248: 443-63, 2004), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer Prot. Sci. 9: 487-496, 2000). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring, Analytical Biochemistry 267: 252-259 (1999), Engen and Smith, Anal. Chem. 73: 256A-265A (2001). When the antibody neutralizes SARS-CoV-2, antibody escape mutant variant organisms can be isolated by propagating SARS-CoV-2 in vitro or in animal models in the presence of high concentrations of the antibody. Sequence analysis of the SARS-CoV-2 gene encoding the antigen targeted by the antibody reveals the mutation(s) conferring antibody escape, indicating residues in the epitope or that affect the structure of the epitope allosterically.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see U.S. Patent Publication 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma. clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-SARS-CoV-2 antibody, the above-described binding methodology is performed in two orientations: to a first orientation, the reference antibody is allowed to bind to the SARS-CoV-2 antigen under saturating conditions followed by assessment of binding of the test antibody to the SARS-CoV-2 molecule. In a second orientation, the test antibody is allowed to bind to the SARS-CoV-2 antigen molecule under saturating conditions followed by assessment of binding of the reference antibody to the SARS-CoV-2 molecule. If, in both orientations, only the first (saturating) antibody is capable of binding to SARS-CoV-2, then it is concluded that the test antibody and the reference antibody compete for binding to SARS-CoV-2. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et at., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other. In some aspects an antibody or antibody fragment that binds to the same or overlapping epitope as COV2-2196, COV2-2130, COV2-2381 is used in combination with an antibody or antibody fragment that binds to the same or overlapping epitope as COV2-2130, COV2-2196, or COV2-2381.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

In another aspect, there are provided monoclonal antibodies having clone-paired CDRs from the heavy and light chains as illustrated in Tables 1, 2 or 3 or Tables 13 or 14. Such antibodies may be produced by the clones disclosed in the Examples section using methods described herein.

In another aspect, the antibodies may be defined by their variable sequence, which include additional “framework” regions. Furthermore, the antibodies sequences may vary from these sequences, optionally using methods discussed in greater detail below. For example, nucleic acid sequences may vary from those set out above in that (a) the variable regions may be segregated away from the constant domains of the light and heavy chains, (b) the nucleic acids may vary from those set out above while not affecting the residues encoded thereby, (c) the nucleic acids may vary from those set out above by a given percentage, e.g., 70%, 75%, 80%, 85%, 90%, 91%, 92%, 93%, 94%, 95%, 96%, 97%, 98% or 99% homology, (d) the nucleic acids may vary from those set out above by virtue of the ability to hybridize under high stringency conditions, as exemplified by low salt and/or high temperature conditions, such as provided by about 0.02 M to about 0.15 M NaCl at temperatures of about 50° C. to about 70° C., (e) the amino acids may vary from those set out above by a given percentage, e.g., 80%, 85%, 90%, 91%, 92%, 93%, 94%, 95%, 96%, 97%, 98% or 99% homology, or (f) the amino acids may vary from those set out above by permitting conservative substitutions (discussed below). Each of the foregoing applies to the nucleic acid sequences and the amino acid sequences.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

### Derivatized Antibodies

Derivatized antibodies may be used to alter the half-lives (e.g., serum half-lives) of parental antibodies in a mammal, particularly a human. Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb but were completely devoid of enhancing activity. LALA mutations of this nature are therefore contemplated in the context of the presently disclosed antibodies.

### Altered Glycosylation

A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a monoclonal antibody with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic monoclonal antibodies. The disclosure is in line with a recent study that shows increased anti-lentivirus cell-mediated viral inhibition of a fucose free anti-HIV monoclonal antibody in vitro. This embodiment of the present disclosure with homogenous glycans lacking a core fucose, showed increased protection against specific viruses by a factor greater than two-fold. Elimination of core fucose dramatically improves the ADCC activity of monoclonal antibodies mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a K_{D} of 1×10⁻⁸ M or less and from Fc gamma RIII with a K_{D} of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation sites found in the polypeptide, and/or adding one or more glycosylation sites that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO 2003/011878, U.S. Pat. Pub. 2003/0003097A1, and Umana et al., Nature Biotechnology, 17:176-180 (1999). Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in HEK 293 or CHO cells used to express recombinant monoclonal antibodies.

It is possible to eliminate protein sequence liabilities of monoclonal antibodies. For instance, it is possible to engineer the antibody variable gene sequences obtained from human B cells to enhance their manufacturability and safety. Potential protein sequence liabilities can be identified by searching for sequence motifs associated with sites containing: 1) Unpaired Cys residues, 2) N-linked glycosylation, 3) Asn deamidation, 4) Asp isomerization, 5) SYE truncation, 6) Met oxidation, 7) Trp oxidation, 8) N-terminal glutamate, 9) Integrin binding, 10) CD11c/CD18 binding, or 11) Fragmentation. Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306 (2004); Chennamsetty et al., PNAS 106: (29), 11937-11942 (2009); Voynov et al., Biocon. Chem., 21:(2), 385-392, (2010). Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

### Stability

Antibodies can be engineered for enhanced biophysical properties. One can use elevated temperature to unfold antibodies to determine relative stability, using average apparent melting temperatures. Differential Scanning calorimetry (DSC) measures the heat capacity, Cp, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, CH2, and CH3 domains). Typically unfolding of the Fab domain produces the strongest peak. The DSC profiles and relative stability of the Fc portion show characteristic differences for the human IgG1, IgG2, IgG3, and IgG4 subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min. One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

### Solubility

One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 (heavy chain CDR3; CDRH3) of each antibody fragment, such as a scFv or a Fv fragment, can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

### Auto-Reactivity

Generally, it is thought that autoreactive clones should be eliminated during ontogeny by negative selection, however it has become clear that many naturally occurring human antibodies with autoreactive properties persist in adult mature repertoires, and the autoreactivity may enhance the antiviral function of many antibodies to pathogens. It has been noted that HCDR3 (heavy chain CDR3; CDRH3)loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293 S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

### Compositions

Another aspect of the present disclosure provides a composition comprising, or consisting essentially of, or consisting of one or more antibodies or antibody fragments disclosed herein; the isolated nucleic acid, or the vector described herein. In some embodiments, the composition is a pharmaceutically acceptable composition, and optionally can comprise other therapeutic agents for combination therapy.

The present disclosure provides pharmaceutical compositions comprising, or consisting essentially of, or consisting of anti-SARS-CoV-2 virus antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, buccal, systemic, nasal, intraarterial, intrabuccal, intranasal, ocular, nebulized, injection, infusion, bronchial inhalation, inhalation, insufflation, intra-rectal, transdermal, rectal, vaginal, topical or delivered by mechanical ventilation.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions can further comprise an additional agent for the augmentation of the antibody response or treatment.

### Mode of Administration

The pharmaceutical compositions for the administration of a component or a combination as disclosed herein can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each component provided herein is included in an amount sufficient to produce the desired effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the component or the combination can be formulated as solutions, gels, ointments, creams, suspensions, etc., as is well-known in the art. Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the component or the combination provided herein can be dried by any art-known technique, such as lyophilization, and reconstituted prior to use. For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### Dosages and Dosing Regimens

The appropriate amount and dosing regimen of the vaccine, component or the combination, when present to be administered to the subject according to any of the methods disclosed herein, may be determined by one of ordinary skill in the art.

In some embodiments, the vaccine, component or the combination as disclosed herein, may be administered to a subject in need thereof, either alone or as part of a pharmaceutically acceptable formulation, once a week, once a day, twice a day, three times a day, or four times a day, or even more frequently.

Administration of the vaccine, component or the combination as disclosed herein may be effected by any method that enables delivery of the component or the combination to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present disclosure.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration of the vaccine, composition or formulation are well-known in the relevant art and would be understood to be encompassed by the skilled artisan once provided the teachings disclosed herein.

### Methods of Treatment

One aspect of the present disclosure provides a method of treating a subject infected with SARS-CoV-2 or reducing the likelihood of infection of a subject at risk of contracting SARS-CoV-2, comprising, or consisting essentially of, or consisting of administering to the subject the antibody or antibody fragment described herein.

In some embodiments, the subject is age 60 or older. In some embodiments, the subject is immunocompromised, and/or suffering from a respiratory and/or cardiovascular disorder. In some embodiments, the antibody or antibody fragment reduces the viral burden and/or level of inflammation in the lung of the subject. In some embodiments, the antibody or antibody fragment reduces viral infection, optionally SARS-CoV-2 infection.

In some embodiments, the method of treating a subject infected with SARS-CoV-2 or reducing the likelihood of infection of a subject at risk of contracting SARS-CoV-2 further comprises administering to the subject a second antibody or antibody fragment. In some embodiments, the second antibody or antibody fragment is an antibody or antibody fragment described herein or disclosed in Table 4 or Table 13.

One aspect of the present disclosure provides a method of treating a subject infected with SARS-CoV-2 or reducing the likelihood of infection of a subject at risk of contracting SARS-CoV-2, comprising, or consisting essentially of, or consisting of administering to the subject a first antibody or antibody fragment and a second antibody or antibody fragment, wherein the first and second antibodies or antibody fragments synergistically neutralize the SARS-CoV-2 virus.

As used herein, the term “synergy” means a higher neutralizing activity mediated by a cocktail of two monoclonal antibodies or antibody fragments disclosed herein when compared to that mediated by the antibody or antibody fragment at the same total concentration (in vitro) or dose (in vivo) of antibodies. To assess if two antibodies synergize in a cocktail to neutralize SARS-CoV-2, a skilled artisan may use previously reported approach to quantitate synergy. See e.g., Ianevski et al., Bioinformatics. 33, 2413-2415 (2017). To evaluate the significance of the beneficial effect from combining monoclonal antibodies, the observed combination responses (dose-response matrix) may be compared with the expected responses calculated by means of synergy scoring models as disclosed by Ianevski et al., Bioinformatics. 33, 2413-2415 (2017). Virus neutralization may be measured in a conventional focus reduction neutralization test (FRNT) assay using wild-type SARS-CoV-2 and Vero-E2 cell culture monolayers. Individual monoclonal antibodies disclosed in Table 4 or Table 13 are mixed at different concentrations to assess neutralizing activity of different monoclonal antibody ratios in the cocktail. Specifically, each of seven-fold dilutions of one monoclonal antibody disclosed in Table 4 or Table 13 (starting from 500 ng/mL) are mixed with each of nine dilutions of a different monoclonal antibody disclosed in Table 4 or Table 13 (starting from 500 ng/mL) in total volume 50 μL of per each condition and then incubated with 50 μL of live SARS-CoV-2 in cell culture medium (RPMI-1640 medium supplemented with 2% FBS) before applying to confluent Vero-E2 cells grown in 96-well plates. The control values included those for dose-response of the neutralizing activity measured separately for individual monoclonal antibodies are assessed at the same doses as in the cocktail). Each measurement can be performed in duplicate. A skilled person then calculates the percent virus neutralization for each condition and then derives the synergy score value, which defined interaction between these two monoclonal antibodies in the cocktail as synergistic. A synergy score of less than −10 generally indicates antagonism, a score from −10 to 10 indicates an additive effect, and a score greater than 10 indicates a synergistic effect. In some embodiments, the synergy score value of any two monoclonal antibodies disclosed in Table 4 or Table 13 is about 17.4.

In some embodiments, the first antibody or antibody fragment and the second antibody or antibody fragment bind to non-overlapping sites on the S glycoprotein. In some embodiments, the first antibody or antibody fragment and the second antibody or antibody fragment have a synergy score of about 10, about 12, about 14, about 15, about 17, about 18, about 20 or more. In some embodiments, the synergy score is based on experimental combination response in a focus reduction neutralization test (FRNT) and/or by synergy-scoring models as described in Zost et al., Nature 584: 443-449 (2020) and/or Ianevski et al., Bioinformatics. 33, 2413-2415 (2017).

In some embodiment, the first antibody or antibody fragment comprises a CDRH1 comprising the amino acid sequence of SEQ ID NO: 196, a CDRH2 comprising the amino acid sequence of SEQ ID NO: 198, a CDRH3 comprising the amino acid sequence of SEQ ID NO: 209, a CDRL1 comprising the amino acid sequence of SEQ ID NO: 234, a CDRL2 comprising the amino acid sequence of SEQ ID NO: 242 or 245, a CDRL3 comprising the amino acid sequence of SEQ ID NO: 251; and/or the second antibody or antibody fragment comprises a CDRH1 comprises the amino acid sequence of SEQ ID NO: 196, a CDRH2 comprising the amino acid sequence of SEQ ID NO: 198, a CDRH3 comprising the amino acid sequence of SEQ ID NO: 203, a CDRL1 comprising the amino acid sequence of SEQ ID NO: 227, a CDRL2 comprising the amino acid sequence of SEQ ID NO: 242 or 243, a CDRL3 comprising the amino acid sequence of SEQ ID NO: 251. In some embodiments, the second antibody or antibody fragment comprises a CDRH1 comprising the amino acid sequence of SEQ ID NO: 196, a CDRH2 comprising the amino acid sequence of SEQ ID NO: 198, a CDRH3 comprising the amino acid sequence of SEQ ID NO: 204, a CDRL1 comprising the amino acid sequence of SEQ ID NO: 229, a CDRL2 comprising the amino acid sequence of SEQ ID NO: 242 or 243, a CDRL3 comprising the amino acid sequence of SEQ ID NO: 251. In some embodiments, the second antibody is sotrovimab (S309), COV2-2381, cilgavimab (COV2-2130 or AZD1061), or tixagevimab (COV2-2196 or AZD8895). In some embodiments, the second antibody is an antibody that binds to a non-overlapping epitopes on the receptor-binding domain of SARS-CoV-2 spike protein.

In some embodiments, the first antibody or antibody fragment comprises a heavy chain variable sequence comprising the amino acid sequence of SEQ ID NO: 109 and a light chain variable sequence comprising the amino acid sequence of SEQ ID NO: 163; and/or the second antibody or antibody fragment comprises a heavy chain variable sequence comprising the amino acid sequence of SEQ ID NO: 103 and a light chain variable sequence comprising the amino acid sequence of SEQ ID NO: 158. In some embodiments, the second antibody or antibody fragment comprises a heavy chain variable sequence comprising the amino acid sequence of SEQ ID NO: 101 and a light chain variable sequence comprising the amino acid sequence of SEQ ID NO: 156.

In some embodiment, the first antibody or antibody fragment comprises a sequence selected from SEQ ID NOs: 15-99; and/or the second antibody or antibody fragment comprises a different sequence selected from SEQ ID NOs: 15-99. In some embodiments, the second antibody or antibody fragment comprises a CDRH1 comprising the amino acid sequence of SEQ ID NO: 196, a CDRH2 comprising an amino acid sequence selected from SEQ ID NOs: 197-201, a CDRH3 comprising an amino acid sequence selected from SEQ ID NOs: 202-225, a CDRL1 comprising an amino acid sequence selected from SEQ ID NOs: 226-241, a CDRL2 comprising an amino acid sequence selected from SEQ ID NOs: 243-250, a CDRL3 comprising the amino acid sequence of SEQ ID NO: 251. In some embodiments, the second antibody or antibody fragment comprises a specific CDR combination as shown in Table 13. In some embodiments, the second antibody is sotrovimab (S309), COV2-2381, cilgavimab (COV2-2130 or AZD1061), or tixagevimab (COV2-2196 or AZD8895). In some embodiments, the second antibody is an antibody that binds to a non-overlapping epitopes on the receptor-binding domain of SARS-CoV-2 spike protein.

### Vaccine Formulations

One aspect of the present disclosure provides a vaccine formulation comprising, or consisting essentially of, or consisting of one or more antibodies or antibody fragments disclosed herein. In some embodiments, the vaccine formulation described herein further comprises a second antibody or antibody fragment that binds to a SARS-CoV-2 surface spike protein. In some embodiments, the second antibody or antibody fragment is an antibody or antibody fragment described herein. In some embodiments, the second antibody or antibody fragment is an antibody or antibody fragment selected from sotrovimab (S309), COV2-2381, cilgavimab (COV2-2130 or AZD1061), or tixagevimab (COV2-2196 or AZD8895).

In another aspect, the present disclosure provides a vaccine formulation comprising one or more expression vectors described herein. In some embodiments, the one or more expression vectors comprise at least one nucleic acid encoding an antibody or antibody fragment disclosed herein. In some embodiments, the nucleic acid is DNA, RNA, or mRNA. In some embodiments, the one or more expression vectors are a Sindbis virus or a Venezuelan equine encephalitis (VEE) vector.

In some embodiments, the vaccine formulation disclosed herein further comprises one or more expression vectors encoding a second antibody or antibody fragment that binds to a SARS-CoV-2 surface spike protein. In some embodiments, the second antibody or antibody fragment is an antibody or antibody fragment described herein or disclosed in Table 4 or Table 13. In some embodiments, the second antibody or antibody fragment is different from the first antibody or antibody fragment. In some embodiments, the first and second antibodies or antibody fragments synergistically neutralize the SARS-CoV-2 virus.

The present disclosure also contemplates the use of antibodies and antibody fragments as described herein for use in assessing the antigenic profile of a viral antigen in a sample. Biological medicinal products like vaccines differ from chemical drugs in that they cannot normally be characterized molecularly; antibodies are large molecules of significant complexity and have the capacity to vary widely from preparation to preparation. They are also administered to healthy individuals, including children at the start of their lives, and thus a strong emphasis must be placed on their quality to ensure, to the greatest extent possible, that they are efficacious in preventing or treating life-threatening disease, without themselves causing harm.

The quality control processes may begin with preparing a sample for an immunoassay that identifies binding of an antibody or fragment disclosed herein to a viral antigen. Such immunoassays are described herein, and any of these may be used to assess the structural/antigenic profile of the antigen. Standards for finding the sample to contain acceptable amounts of antigenically correct and intact antigen may be established by regulatory agencies.

Another important embodiment where antigen integrity is assessed is in determining shelf-life and storage stability. Most medicines, including vaccines, can deteriorate over time. Therefore, it is critical to determine whether, over time, the degree to which an antigen, such as in a vaccine, degrades or destabilizes such that is it no longer antigenic and/or capable of generating an immune response when administered to a subject. Again, standards for finding the sample to contain acceptable amounts of antigenically intact antigen may be established by regulatory agencies.

In certain embodiments, viral antigens may contain more than one protective epitope. In these cases, it may prove useful to employ assays that look at the binding of more than one antibody, such as 2, 3, 4, 5 or even more antibodies. These antibodies bind to closely related epitopes, such that they are adjacent or even overlap each other. On the other hand, they may represent distinct epitopes from disparate parts of the antigen. By examining the integrity of multiple epitopes, a more complete picture of the antigen's overall integrity, and hence ability to generate a protective immune response, may be determined.

Active vaccines are also envisioned where antibodies like those disclosed are produced in vivo in a subject at risk of SARS-CoV-2 infection. Such vaccines can be formulated for parenteral administration, e.g., formulated for injection via the intradermal, intravenous, intramuscular, subcutaneous, or even intraperitoneal routes. Administration by intradermal and intramuscular routes are contemplated. The vaccine could alternatively be administered by a topical route directly to the mucosa, for example, by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

### Detection Methods

One aspect of the present disclosure provides a method of detecting COVID-19 virus in vitro or an infection in a subject, the method comprising, or consisting essentially of, or consisting of contacting a sample isolated from the subject with the antibody or antibody fragment disclosed herein; and detecting SARS-CoV-2 in the sample by detecting the binding of the antibody or antibody fragment to a SARS-CoV-2 antigen in the sample. In some embodiments, the sample is a body fluid. In some embodiments, the sample is blood, sputum, tears, saliva, mucous or serum, semen, cervical or vaginal secretions, amniotic fluid, placental tissues, urine, exudate, transudate, tissue scrapings or feces.

Another aspect of the present disclosure provides a method of determining a SARS-CoV-2 virus variant antigenic profile comprising, or consisting essentially of, or consisting of: contacting a sample comprising the SARS-CoV-2 virus variant with the antibody or antibody fragment as described herein; and determining the binding of the antibody or antibody fragment to the SARS-CoV-2 virus variant, wherein absence of binding indicates a conformational change and/or a mutation in the SARS-CoV-2 surface spike protein.

In some embodiments, the sample comprises recombinantly produced SARS-CoV-2 surface spike protein variants. In some embodiments, the sample comprises a vaccine formulation or vaccine production batch described herein. In some embodiments, the detection comprises ELISA, RIA, western blot, a biosensor using surface plasmon resonance or biolayer interferometry, or flow cytometric staining. In some embodiments, steps (a) and (b) of the method of determining a SARS-CoV-2 virus variant antigenic profile as described herein are repeated at least two, three, or four times to determine the antigenic stability of the SARS-CoV-2 surface spike protein over time.

In another aspect, the present disclosure provides a method of detecting SARS-CoV-2 antigen in a sample, comprising contacting the sample the antibody or antibody fragment described herein; and detecting SARS-CoV-2 in the sample by detecting the binding of the antibody or antibody fragment to a SARS-CoV-2 antigen in the sample.

In still further embodiments, the present disclosure provides immunodetection methods for binding, purifying, removing, quantifying and otherwise generally detecting SARS-CoV-2 and its associated antigens. While such methods can be applied in a traditional sense, another use will be in quality control and monitoring of vaccine and other virus stocks, where antibodies according to the present disclosure can be used to assess the amount or integrity (i.e., long term stability) of antigens in viruses. Alternatively, the methods may be used to screen various antibodies for appropriate/desired reactivity profiles.

Other immunodetection methods include specific assays for determining the presence of SARS-CoV-2 in a subject. A wide variety of assay formats are contemplated, but specifically those that would be used to detect SARS-CoV-2 in a fluid obtained from a subject, such as saliva, blood, plasma, sputum, semen or urine. In particular, semen has been demonstrated as a viable sample for detecting SARS-CoV-2 (Purpura et al., 2016; Mansuy et al., 2016; Barzon et al., 2016; Gornet et al., 2016; Duffy et al., 2009; CDC, 2016; Halfon et al., 2010; Elder et al. 2005). The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

In some embodiments, the detection comprises enzyme linked immunosorbent assay (ELISA), radioimmunoassay (RIA), lateral flow assay or western blot. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of SARS-CoV-2 antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing SARS-CoV-2, and contacting the sample with a first antibody in accordance with the present disclosure, as the case may be, under conditions effective to allow the formation of immunocomplexes.

These methods include methods for purifying SARS-CoV-2 or related antigens from a sample. The antibody will preferably be linked to a solid support, such as in the form of a column matrix, and the sample suspected of containing the SARS-CoV-2 or antigenic component will be applied to the immobilized antibody. The unwanted components will be washed from the column, leaving the SARS-CoV-2 antigen immunocomplexed to the immobilized antibody, which is then collected by removing the organism or antigen from the column.

The immunobinding methods also include methods for detecting and quantifying the amount of SARS-CoV-2 or related components in a sample and the detection and quantification of any immune complexes formed during the binding process. Here, one would obtain a sample suspected of containing SARS-CoV-2 or its antigens and contact the sample with an antibody that binds SARS-CoV-2 or components thereof, followed by detecting and quantifying the amount of immune complexes formed under the specific conditions. In terms of antigen detection, the biological sample analyzed may be any sample that is suspected of containing SARS-CoV-2 or SARS-CoV-2 antigen, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to SARS-CoV-2 or antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, for example, with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

### ELISAs

Immunoassays, in their most simple and direct sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the SARS-CoV-2 or SARS-CoV-2 antigen is added to the wells. After binding and washing to remove non-specifically bound immune complexes, the bound antigen may be detected. Detection may be achieved by the addition of another anti-SARS-CoV-2 antibody that is linked to a detectable label. This type of ELISA is a simple “sandwich ELISA.” Detection may also be achieved by the addition of a second anti-SARS-CoV-2 antibody, followed by the addition of a third antibody that has binding affinity for the second antibody, with the third antibody being linked to a detectable label.

In another exemplary ELISA, the samples suspected of containing the SARS-CoV-2 or SARS-CoV-2 antigen are immobilized onto the well surface and then contacted with the anti-SARS-CoV-2 antibodies of the disclosure. After binding and washing to remove non-specifically bound immune complexes, the bound anti-SARS-CoV-2 antibodies are detected. Where the initial anti-SARS-CoV-2 antibodies are linked to a detectable label, the immune complexes may be detected directly. Again, the immune complexes may be detected using a second antibody that has binding affinity for the first anti-SARS-CoV-2 antibody, with the second antibody being linked to a detectable label.

Irrespective of the format employed, ELISAs have certain features in common, such as coating, incubating and binding, washing to remove non-specifically bound species, and detecting the bound immune complexes. These are described below. In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H2O2, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of SARS-CoV-2 antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

Here, the inventor proposes the use of labeled SARS-CoV-2 monoclonal antibodies to determine the amount of SARS-CoV-2 antibodies in a sample. The basic format would include contacting a known amount of SARS-CoV-2 monoclonal antibody (linked to a detectable label) with SARS-CoV-2 antigen or particle. The SARS-CoV-2 antigen or organism is preferably attached to a support. After binding of the labeled monoclonal antibody to the support, the sample is added and incubated under conditions permitting any unlabeled antibody in the sample to compete with, and hence displace, the labeled monoclonal antibody. By measuring either the lost label or the label remaining (and subtracting that from the original amount of bound label), one can determine how much non-labeled antibody is bound to the support, and thus how much antibody was present in the sample.

### Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

### Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

### Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

### Biosensors

One aspect of the present disclosure provides a biosensor for detecting the presence of a SARS-CoV-2 antigen in a sample comprising a transducer component comprising, or consisting essentially of, or consisting of an electrode operatively connected to a microprocessor, the microprocessor being adapted to receive, process and transmit a signal; a receptor component having a sensing element capable of detecting and binding to the SARS-CoV-2 antigen; and (c) the transducer component and the receptor component are capable of being brought into direct contact with the sample in situ. In some embodiments, the sensing element comprises the antibody or antibody fragment disclosed herein. Biosensors for use in analyzing the presence or absence of an antigen in a biological sample are known to those skilled in the art. See e.g., U.S. Pat. No. 10,849,540.

### Kits

Antibodies and fragments thereof as described in the present disclosure may also be used in a kit for monitoring the efficacy of vaccination procedures by detecting the presence of protective SARS-CoV-2 antibodies. Antibodies, antibody fragment, or variants and derivatives thereof, as described in the present disclosure may also be used in a kit for monitoring vaccine manufacture with the desired immunogenicity.

In one aspect, the present disclosure provides a kit comprising the antibody or antibody fragment disclosed herein and instructions for using the first antibody or antibody fragment. In another aspect, the present disclosure provides a kit comprising a first antibody or antibody fragment that binds to a SARS-CoV-2 surface spike protein and a second antibody or antibody fragment that binds to a SAR-CoV-2 surface spike protein. In some embodiments, the first and/or second antibody and antibody fragment is antibody or antibody fragment of described herein or disclosed in Table 4 or Table 13. In some embodiments, at least one of the first and/or second antibody and antibody fragment is antibody or antibody fragment described herein or disclosed in Table 4 or Table 13. In some embodiments, the kit further comprises instructions for using the first antibody or antibody fragment and the second antibody or antibody fragment for treating a subject infected with SARS-CoV-2 or for reducing the likelihood of infection of a subject at risk of contracting SARS-CoV-2.

In some embodiments, the first antibody or antibody fragment and the second antibody or antibody fragment have a synergy score of 17.4. The synergy score is determined by the method disclosed in Zost et al., Nature 584: 443-449 (2020). In some embodiments, the first antibody or antibody fragment and the second antibody or antibody fragment are in separate containers.

In still further embodiments, the present disclosure provides an immunodetection kit for use with the immunodetection methods described herein. As the antibodies may be used to detect SARS-CoV-2 or SARS-CoV-2 antigens, the antibodies may be included in the kit. The immunodetection kit comprises, in suitable container means, a first antibody that binds to SARS-CoV-2 or SARS-CoV-2 antigen, and optionally an immunodetection reagent.

In certain embodiments, the SARS-CoV-2 antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of the SARS-CoV-2 or SARS-CoV-2 antigens, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

In some embodiments, the kits of the present disclosure comprises a means for containing the antibody, antigen, and any other reagent containers in close confinement for commercial sale. The container means of the kits will generally include at least one vial, test tube, flask, bottle, syringe or other container means, into which the antibody may be placed, or preferably, suitably aliquoted. Such containers may include injection or blow-molded plastic containers into which the desired vials are retained.

TABLE 2
Amino acid sequences for the heavy chain (H) CDR1, CDR2, and CDR3
regions of the anti-SARS_CoV_2 monoclonal antibodies

| | | SEQ | | SEQ | | SEQ |
|---|---|---|---|---|---|---|
| | | ID | | ID | | ID |
| Protein Name | CDRH1 | NO | CDRH2 | NO | CDRH3 | NO |
| mAb_RBD_2130_ | gftfrdvw | 196 | ikskEdggtt | 197 | ttagsyyFdtvgpglpegkfdy | 202 |
| FAB_20211231_ | | | | | | |
| 00017 | | | | | | |
| mAb_RBD_2130_ | gftfrdvw | 196 | ikskidggtt | 198 | ttagsyyydtRgpglpegkfdy | 203 |
| FAB_20211231_ | | | | | | |
| 00174 | | | | | | |
| mAb_RBD_2130_ | gftfrdvw | 196 | ikskWdggtt | 199 | ttagsyyydtvgpglpegkfdy | 204 |
| FAB_20211231_ | | | | | | |
| 00194 | | | | | | |
| mAb_RBD_2130_ | gftfrdvw | 196 | ikskidggtt | 198 | ttagsyyydtvgpglpegkfdy | 204 |
| FAB_20211231_ | | | | | | |
| 00199 | | | | | | |
| mAb_RBD_2130_ | gftfrdvw | 196 | ikskidggtt | 198 | ttagsyyydtvgpglpegkfdy | 204 |
| FAB_20211231_ | | | | | | |
| 00200 | | | | | | |
| v2130_SFE_ | gftfrdvw | 196 | ikskidggtt | 198 | ttagsyyydtvgpglpegkfdy | 204 |
| 20220104_00015 | | | | | | |
| v2130_SFE_ | gftfrdvw | 196 | ikskidggtt | 198 | ttagsyyydtvgpglpegkfdy | 204 |
| 20220104_00024 | | | | | | |
| mAb_v2130Just- | gftfrdvw | 196 | ikskDdggtt | 200 | ttagsyyydtvgpglpegkfdy | 204 |
| StableLines_ | | | | | | |
| 20220111_00002 | | | | | | |
| mAb_v2130Van- | gftfrdvw | 196 | ikskidggtt | 198 | ttagsyyydtvgpElpegkfdy | 205 |
| derbilt_184_ | | | | | | |
| 20220114_00111 | | | | | | |
| mAb_v2130Van- | gftfrdvw | 196 | ikskidggtt | 198 | ttagsyyydtvgpElpegkfdy | 205 |
| derbilt_184_ | | | | | | |
| 20220114_00112 | | | | | | |
| v2130_upsample_ | gftfrdvw | 196 | ikskidggtt | 198 | ttagsyyDdtvgpglpegkIdy | 206 |
| 20220121_00009 | | | | | | |
| v2130_upsample | gftfrdvw | 196 | ikskEdggtt | 197 | ttagsyyHdtvgpglpegkfdy | 207 |
| 20220121_00298_ | | | | | | |
| v2130_upsample_ | gftfrdvw | 196 | ikskHdggtt | 201 | ttagsyyydtvLpglpegkfdy | 208 |
| 20220121_00314 | | | | | | |
| v2130_upsample_ | gftfrdvw | 196 | ikskidggtt | 198 | ttagsyyydtRgpElpegkfdy | 209 |
| 20220121_00354 | | | | | | |

TABLE 3
Amino acid sequences for the light chain (L) CDR1, CDR2, and CDR3
regions of the novel anti-SARS_CoV_2 monoclonal antibodies

| | | SEQ | | SEQ | | |
|---|---|---|---|---|---|---|
| | | ID | | ID | | SEQ ID |
| Protein Name | CDRL1 | NO | CDRL2 | NO | CDRL3 | NO |
| mAb_RBD_2130_ | qsvlyWAnnkny | 226 | was | 242 | qqyystlt | 251 |
| FAB_20211231_ | | | | | | |
| 00017 | | | | | | |
| mAb_RBD_2130_ | qsvlyssnnkny | 227 | was | 242 | qqyystlt | 251 |
| FAB_20211231_ | | | | | | |
| 00174 | | | | | | |
| mAb_RBD_2130_ | qsvlyssnnkny | 227 | was | 242 | qqyystlt | 251 |
| FAB_20211231_ | | | | | | |
| 00194 | | | | | | |
| mAb_RBD_2130_ | qsvlysYnnkny | 228 | was | 242 | qqyystlt | 251 |
| FAB_20211231_ | | | | | | |
| 00199 | | | | | | |
| mAb_RBD_2130_ | qsvlysWnnkny | 229 | was | 242 | qqyystlt | 251 |
| FAB_20211231_ | | | | | | |
| 00200 | | | | | | |
| v2130_SFE_ | qsvlyQYnnkny | 230 | was | 242 | qqyystlt | 251 |
| 20220104_00015 | | | | | | |
| v2130_SFE_ | qsvlyWsnnkny | 231 | was | 242 | qqyystlt | 251 |
| 20220104_00024 | | | | | | |
| mAb_v2130Just- | qsvlysFnnkny | 232 | was | 242 | qqyystlt | 251 |
| StableLines_ | | | | | | |
| 20220111_00002 | | | | | | |
| mAb_v2130Van- | qsvlyYVnnYny | 233 | was | 242 | qqyystlt | 251 |
| derbilt_184_ | | | | | | |
| 20220114_00111 | | | | | | |
| mAb_v2130Van- | qsvlyAAnnkny | 234 | was | 242 | qqyystlt | 251 |
| derbilt_184_ | | | | | | |
| 20220114_00112 | | | | | | |
| v2130_upsample_ | qsvlysVnnkny | 235 | was | 242 | qqyystlt | 251 |
| 20220121_ | | | | | | |
| 00009 | | | | | | |
| v2130_upsample_ | qsvlyRsnnLny | 236 | was | 242 | qqyystlt | 251 |
| 20220121_ | | | | | | |
| 00298 | | | | | | |
| v2130_upsample_ | qsvlysYnnkny | 228 | was | 242 | qqyystlt | 251 |
| 20220121_ | | | | | | |
| 00314 | | | | | | |
| v2130_upsample_ | qsvlyAAnnkny | 234 | was | 242 | qqyystlt | 251 |
| 20220121_ | | | | | | |
| 00354 | | | | | | |
| mAb_RBD_2130_ | qsvlyWAnnkny | 226 | ywastr | 243 | qqyystlt | 251 |
| FAB_20211231_ | | | | | | |
| 00017 | | | | | | |
| mAb_RBD_2130_ | qsvlyssnnkny | 227 | ywastr | 243 | qqyystlt | 251 |
| FAB_20211231_ | | | | | | |
| 00174 | | | | | | |
| mAb_RBD_2130_ | qsvlyssnnkny | 227 | ywastr | 243 | qqyystlt | 251 |
| FAB_20211231_ | | | | | | |
| 00194 | | | | | | |
| mAb_RBD_2130_ | qsvlysYnnkny | 228 | ywastr | 243 | qqyystlt | 251 |
| FAB_20211231_ | | | | | | |
| 00199 | | | | | | |
| mAb_RBD_2130_ | qsvlysWnnkny | 229 | ywastr | 243 | qqyystlt | 251 |
| FAB_20211231_ | | | | | | |
| 00200 | | | | | | |
| v2130_SFE_ | qsvlyQYnnkny | 230 | ywasHr | 244 | qqyystlt | 251 |
| 20220104_00015 | | | | | | |
| v2130_SFE_ | qsvlyWsnnkny | 231 | ywasEr | 245 | qqyystlt | 251 |
| 20220104_00024 | | | | | | |
| mAb_v2130Just- | qsvlysFnnkny | 232 | ywasEr | 245 | qqyystlt | 251 |
| StableLines_ | | | | | | |
| 20220111_00002 | | | | | | |
| mAb_v2130Van- | qsvlyYVnnYny | 233 | ywastr | 243 | qqyystlt | 251 |
| derbilt_184_ | | | | | | |
| 20220114_00111 | | | | | | |
| mAb_v2130Van- | qsvlyAAnnkny | 234 | ywasEr | 245 | qqyystlt | 251 |
| derbilt_184_ | | | | | | |
| 20220114_00112 | | | | | | |
| v2130_upsample_ | qsvlysVnnkny | 235 | Nwastr | 246 | qqyystlt | 251 |
| 20220121_ | | | | | | |
| 00009 | | | | | | |
| v2130_upsample_ | qsvlyRsnnLny | 236 | ywastr | 243 | qqyystlt | 251 |
| 20220121_ | | | | | | |
| 00298 | | | | | | |
| v2130_upsample_ | qsvlysYnnkny | 228 | Ewastr | 247 | qqyystlt | 251 |
| 20220121_ | | | | | | |
| 00314 | | | | | | |
| v2130_upsample_ | qsvlyAAnnkny | 234 | ywasEr | 245 | qqyystlt | 251 |
| 20220121_ | | | | | | |
| 00354 | | | | | | |

TABLE 4
Amino acid sequences of the novel anti-SARS_CoV_2 monoclonal
antibodies

| SEQ | | |
|---|---|---|
| ID | | |
| NO: | DESCRIPTION | SEQUENCE |
| 1 | >md5_e3d1904966eaf73aed | EVQLVESGGGLVKPGGSLRLSCAASGFTFRDVW |
| | 30331f60b658e9 | MSWVRQAPGKGLEWVGRIKSKEDGGTTDYAAP |
| | Mutant_number: | VKGRFTISRDDSKNTLYLQMNSLKTEDTAVYYC |
| | md5_e3d1904966eaf73aed3 | TTAGSYYFDTVGPGLPEGKFDYWGQGTLVTVSS/ |
| | 0331f60b658e9 HumID: | DIVMTQSPDSLAVSLGERATINCKSSQSVLYWA |
| | mAb_RBD_2130_FAB_ | NNKNYLAWYQQKPGQPPKLLMYWASTRESGV |
| | 20211231_00017 Master: | PDRFSGSGSGAEFTLTISSLQAEDVAIYYCQQYY |
| | RBD_2130_FAB | STLTFGGGTKVEIKR |
| | Mutations: IH55E, | |
| | YH106F, SL32W, SL33A | |
| | Fv Fragment- no linker | |
| 100 | Heavy chain (V_{H}) | EVQLVESGGGLVKPGGSLRLSCAASGFTFRDVW |
| | | MSWVRQAPGKGLEWVGRIKSKEDGGTTDYAAP |
| | | VKGRFTISRDDSKNTLYLQMNSLKTEDTAVYYC |
| | | TTAGSYYFDTVGPGLPEGKFDYWGQGTLVTVSS |
| 196 | CDRH1 | GFTFRDVW |
| 197 | CDRH2 | IKSKEDGGTT |
| 202 | CDRH3 | TTAGSYYFDTVGPGLPEGKFDY |
| 155 | Light chain (V_{L}) | DIVMTQSPDSLAVSLGERATINCKSSQSVLYWA |
| | | NNKNYLAWYQQKPGQPPKLLMYWASTRESGV |
| | | PDRFSGSGSGAEFTLTISSLQAEDVAIYYCQQYY |
| | | STLTFGGGTKVEIKR |
| 226 | CDRL1 | QSVLYWANNKNY |
| 242 | CDRL2 | WAS |
| 243 | CDRL2 | YWASTR |
| 251 | CDRL3 | QQYYSTLT |
| 2 | >md5_d323d72bb6bc337ea | EVQLVESGGGLVKPGGSLRLSCAASGFTFRDVW |
| | 256bdd92ceba08e | MSWVRQAPGKGLEWVGRIKSKIDGGTTDYAAP |
| | Mutant_number: | VKGRFTISRDDSKNTLYLQMNSLKTEDTAVYYC |
| | md5_d323d72bb6bc337ea2 | TTAGSYYYDTRGPGLPEGKFDYWGQGTLVTVSS/ |
| | 56bdd92ceba08e HumID: | DIVMTQSPDSLAVSLGERATINCKSSQSVLYSSN |
| | mAb_RBD_2130_FAB_ | NKNYLAWYQQKPGQPPKLLMYWASTRESGVPD |
| | 20211231_00174 Master: | RFSGSGSGAEFTLTISSLQAEDVAIYYCQQYYSTL |
| | RBD_2130_FAB | TFGGGTKVEIKR |
| | Mutations: VH109R | |
| | Fv Fragment- no linker | |
| 101 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTRGPGLPEGKFDYWGQGTLVTVSS |
| 196 | CDRH1 | GFTFRDVW |
| 198 | CDRH2 | IKSKIDGGTT |
| 203 | CDRH3 | TTAGSYYYDTRGPGLPEGKFDY |
| 156 | Light chain (V_{L}) | DIVMTQSPDSLAVSLGERATINCKSSQSVLYSSN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 227 | CDRL1 | QSVLYSSNNKNY |
| 242 | CDRL2 | WAS |
| 243 | CDRL2 | YWASTR |
| 251 | CDRL3 | QQYYSTLT |
| 3 | >md5_9f6486808688a2b46 | EVQLVESGGGLVKPGGSLRLSCAASGFTFRDVW |
| | ec220af47cb95c5 | MSWVRQAPGKGLEWVGRIKSKWDGGTTDYAA |
| | Mutant_number: | PVKGRFTISRDDSKNTLYLQMNSLKTEDTAVYY |
| | md5_9f6486808688a2b46e | CTTAGSYYYDTVGPGLPEGKFDYWGQGTLVTV |
| | c220af47cb95c5 HumID: | SS/DIVMTQSPDSLAVSLGERATINCKSSQSVLYS |
| | mAb_RBD_2130_FAB_ | SNNKNYLAWYQQKPGQPPKLLMYWASTRESG |
| | 20211231_00194 Master: | VPDRFSGSGSGAEFTLTISSLQAEDVAIYYCQQY |
| | RBD_2130_FAB | YSTLTFGGGTKVEIKR |
| | Mutations: IH55W | |
| | Fv Fragment- no linker | |
| 102 | Heavy chain (V_{H}) | EVQLVESGGGLVKPGGSLRLSCAASGFTFRDVW |
| | | MSWVRQAPGKGLEWVGRIKSKWDGGTTDYAA |
| | | PVKGRFTISRDDSKNTLYLQMNSLKTEDTAVYY |
| | | CTTAGSYYYDTVGPGLPEGKFDYWGQGTLVTVS |
| | | S |
| 196 | CDRH1 | GFTFRDVW |
| 199 | CDRH2 | IKSKWDGGTT |
| 204 | CDRH3 | TTAGSYYYDTVGPGLPEGKFDY |
| 156 | Light chain (V_{L}) | DIVMTQSPDSLAVSLGERATINCKSSQSVLYSSN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 227 | CDRL1 | QSVLYSSNNKNY |
| 242 | CDRL2 | WAS |
| 243 | CDRL2 | YWASTR |
| 251 | CDRL3 | QQYYSTLT |
| 4 | >md5_eebd2cc1786b72435 | EVQLVESGGGLVKPGGSLRLSCAASGFTFRDVW |
| | 8ced82fe314465c | MSWVRQAPGKGLEWVGRIKSKIDGGTTDYAAP |
| | Mutant_number: | VKGRFTISRDDSKNTLYLQMNSLKTEDTAVYYC |
| | md5_eebd2cc1786b724358 | TTAGSYYYDTVGPGLPEGKFDYWGQGTLVTVSS/ |
| | ced82fe314465c HumID: | DIVMTQSPDSLAVSLGERATINCKSSQSVLYSYN |
| | mAb_RBD_2130_FAB_ | NKNYLAWYQQKPGQPPKLLMYWASTRESGVPD |
| | 20211231_00199 Master: | RFSGSGSGAEFTLTISSLQAEDVAIYYCQQYYSTL |
| | RBD_2130_FAB | TFGGGTKVEIKR |
| | Mutations: SL33Y | |
| | Fv Fragment- no linker | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 157 | Light chain (V_{L}) | DIVMTQSPDSLAVSLGERATINCKSSQSVLYSYN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 228 | CDRL1 | QSVLYSYNNKNY |
| 242 | CDRL2 | WAS |
| 243 | CDRL2 | YWASTR |
| 251 | CDRL3 | QQYYSTLT |
| 5 | >md5_aaca5fa01a0f27e83e | EVQLVESGGGLVKPGGSLRLSCAASGFTFRDVW |
| | 39da01a4457347 | MSWVRQAPGKGLEWVGRIKSKIDGGTTDYAAP |
| | Mutant_number: | VKGRFTISRDDSKNTLYLQMNSLKTEDTAVYYC |
| | md5_aaca5fa01a0f27e83e3 | TTAGSYYYDTVGPGLPEGKFDYWGQGTLVTVSS/ |
| | 9da01a4457347 HumID: | DIVMTQSPDSLAVSLGERATINCKSSQSVLYSW |
| | mAb_RBD_2130_FAB_ | NNKNYLAWYQQKPGQPPKLLMYWASTRESGVP |
| | 20211231_00200 Master: | DRFSGSGSGAEFTLTISSLQAEDVAIYYCQQYYST |
| | RBD_2130_FAB | LTFGGGTKVEIKR |
| | Mutations: SL33W | |
| | Fv Fragment- no linker | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 158 | Light chain (V_{L}) | DIVMTQSPDSLAVSLGERATINCKSSQSVLYSWN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 229 | CDRL1 | QSVLYSWNNKNY |
| 242 | CDRL2 | WAS |
| 243 | CDRL2 | YWASTR |
| 251 | CDRL3 | QQYYSTLT |
| 6 | >md5_2041aa110908c8cde | EVQLVESGGGLVKPGGSLRLSCAASGFTFRDVW |
| | 47ed7aabc074be8 | MSWVRQAPGKGLEWVGRIKSKIDGGTTDYAAP |
| | Mutant_number: | VKGRFTISRDDSKNTLYLQMNSLKTEDTAVYYC |
| | md5_2041aa110908c8cde4 | TTAGSYYYDTVGPGLPEGKFDYWGQGTLVTVSS/ |
| | 7ed7aabc074be8 HumID: | DIVMTQSPDSLAVSLGERATINCKSSQSVLYQYN |
| | v2130 SFE_20220104_ | NKNYLAWYQQKPGQPPKLLMYWASHRESGVPD |
| | 00015 Master: | RFSGSGSGAEFTLTISSLQAEDVAIYYCQQYYSTL |
| | RBD_2130_FAB | TFGGGTKVEIKR |
| | Mutations: SL32Q, SL33Y, | |
| | TL59H | |
| | Fv Fragment- no linker | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 159 | Light chain (V_{L}) | DIVMTQSPDSLAVSLGERATINCKSSQSVLYQYN |
| | | NKNYLAWYQQKPGQPPKLLMYWASHRESGVPD |
| | | RFSGSGSGAEFTLTISSLQAEDVAIYYCQQYYSTL |
| | | TFGGGTKVEIKR |
| 230 | CDRL1 | QSVLYQYNNKNY |
| 242 | CDRL2 | WAS |
| 244 | CDRL2 | YWASHR |
| 251 | CDRL3 | QQYYSTLT |
| 7 | >md5_1089bd14be7c511aa | EVQLVESGGGLVKPGGSLRLSCAASGFTFRDVW |
| | 67dbef73eeb1e42 | MSWVRQAPGKGLEWVGRIKSKIDGGTTDYAAP |
| | Mutant_number: | VKGRFTISRDDSKNTLYLQMNSLKTEDTAVYYC |
| | md5_1089bd14be7c511aa6 | TTAGSYYYDTVGPGLPEGKFDYWGQGTLVTVSS |
| | 7dbef73eeb1e42 HumID: | /DIVMTQSPDSLAVSLGERATINCKSSQSVLYWS |
| | v2130 SFE 20220104 | NNKNYLAWYQQKPGQPPKLLMYWASERESGVP |
| | 00024 Master: | DRFSGSGSGAEFTLTISSLQAEDVAIYYCQQYYST |
| | RBD_2130_FAB | LTFGGGTKVEIKR |
| | Mutations: SL32W, TL59E | |
| | Fv Fragment- no linker | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 160 | Light chain (V_{L}) | DIVMTQSPDSLAVSLGERATINCKSSQSVLYWSN |
| | | NKNYLAWYQQKPGQPPKLLMYWASERESGVPD |
| | | RFSGSGSGAEFTLTISSLQAEDVAIYYCQQYYSTL |
| | | TFGGGTKVEIKR |
| 231 | CDRL1 | QSVLYWSNNKNY |
| 242 | CDRL2 | WAS |
| 245 | CDRL2 | YWASER |
| 251 | CDRL3 | QQYYSTLT |
| 8 | >md5_689536fbc67d8ee33 | EVQLVESGGGLVKPGGSLRLSCAASGFTFRDVW |
| | e80ca5a6709d167 | MSWVRQAPGKGLEWVGRIKSKDDGGTTDYAAP |
| | Mutant_number: | VKGRFTISRDDSKNTLYLQMNSLKTEDTAVYYC |
| | md5_689536fbc67d8ee33e8 | TTAGSYYYDTVGPGLPEGKFDYWGQGTLVTVSS/ |
| | 0ca5a6709d167 HumID: | DIVMTQSPDSLAVSLGERATINCKSSQSVLYSFN |
| | mAb_v2130JustStableLines_ | NKNYLAWYQQKPGQPPKLLMYWASERESGVPD |
| | 20220111_00002 Master: | RFSGSGSGAEFTLTISSLQAEDVAIYYCQQYYSTL |
| | RBD_2130_FAB | TFGGGTKVEIKR |
| | Mutations: IH55D, SL33F, | |
| | TL59E | |
| | Fv Fragment- no linker | |
| 104 | Heavy chain (V_{H}) | EVQLVESGGGLVKPGGSLRLSCAASGFTFRDVW |
| | | MSWVRQAPGKGLEWVGRIKSKDDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 200 | CDRH2 | IKSKDDGGTT |
| 204 | CDRH3 | TTAGSYYYDTVGPGLPEGKFDY |
| 161 | Light chain (V_{L}) | DIVMTQSPDSLAVSLGERATINCKSSQSVLYSFN |
| | | NKNYLAWYQQKPGQPPKLLMYWASERESGVPD |
| | | RFSGSGSGAEFTLTISSLQAEDVAIYYCQQYYSTL |
| | | TFGGGTKVEIKR |
| 232 | CDRL1 | QSVLYSFNNKNY |
| 242 | CDRL2 | WAS |
| 245 | CDRL2 | YWASER |
| 251 | CDRL3 | QQYYSTLT |
| 9 | >md5_a314041f0f349a3dc6 | EVQLVESGGGLVKPGGSLRLSCAASGFTFRDVW |
| | b474c069163d9b | MSWVRQAPGKGLEWVGRIKSKIDGGTTDYAAP |
| | Mutant_number: | VKGRFTISRDDSKNTLYLQMNSLKTEDTAVYYC |
| | md5_a314041f0f349a3dc6b | TTAGSYYYDTVGPELPEGKFDYWGQGTLVTVSS/ |
| | 474c069163d9b HumID: | DIVMTQSPDSLAVSLGERATINCKSSQSVLYYVN |
| | mAb_v2130Vanderbilt_184_ | NYNYLAWYQQKPGQPPKLLMYWASTRESGVPD |
| | 20220114_00111 Master: | RFSGSGSGAEFTLTISSLQAEDVAIYYCQQYYSTL |
| | RBD_2130_FAB | TFGGGTKVEIKR |
| | Mutations: GH112E, | |
| | SL32Y, SL33V, KL36Y | |
| | Fv Fragment- no linker | |
| 105 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPELPEGKFDYWGQGTLVTVSS |
| 196 | CDRH1 | GFTFRDVW |
| 198 | CDRH2 | IKSKIDGGTT |
| 205 | CDRH3 | TTAGSYYYDTVGPELPEGKFDY |
| 162 | Light chain (V_{L}) | DIVMTQSPDSLAVSLGERATINCKSSQSVLYYVN |
| | | NYNYLAWYQQKPGQPPKLLMYWASTRESGVPD |
| | | RFSGSGSGAEFTLTISSLQAEDVAIYYCQQYYSTL |
| | | TFGGGTKVEIKR |
| 233 | CDRL1 | QSVLYYVNNYNY |
| 242 | CDRL2 | WAS |
| 243 | CDRL2 | YWASTR |
| 251 | CDRL3 | QQYYSTLT |
| 10 | >md5_3052c528d578179c0 | EVQLVESGGGLVKPGGSLRLSCAASGFTFRDVW |
| | 0211647380e5fb7 | MSWVRQAPGKGLEWVGRIKSKIDGGTTDYAAP |
| | Mutant_number: | VKGRFTISRDDSKNTLYLQMNSLKTEDTAVYYC |
| | md5_3052c528d578179c00 | TTAGSYYYDTVGPELPEGKFDYWGQGTLVTVSS/ |
| | 211647380e5fb7 HumID: | DIVMTQSPDSLAVSLGERATINCKSSQSVLYAAN |
| | mAb_v2130Vanderbilt_184_ | NKNYLAWYQQKPGQPPKLLMYWASERESGVPD |
| | 20220114_00112 Master: | RFSGSGSGAEFTLTISSLQAEDVAIYYCQQYYSTL |
| | RBD_2130_FAB | TFGGGTKVEIKR |
| | Mutations: GH112E, | |
| | SL32A, SL33A, TL59E | |
| | Fv Fragment- no linker | |
| 105 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPELPEGKFDYWGQGTLVTVS |
| | | S |
| 196 | CDRH1 | GFTFRDVW |
| 198 | CDRH2 | IKSKIDGGTT |
| 205 | CDRH3 | TTAGSYYYDTVGPELPEGKFDY |
| 163 | Light chain (V_{L}) | DIVMTQSPDSLAVSLGERATINCKSSQSVLYAAN |
| | | NKNYLAWYQQKPGQPPKLLMYWASERESGVP |
| | | DRFSGSGSGAEFTLTISSLQAEDVAIYYCQQYYS |
| | | TLTFGGGTKVEIKR |
| 234 | CDRL1 | QSVLYAANNKNY |
| 242 | CDRL2 | WAS |
| 245 | CDRL2 | YWASER |
| 251 | CDRL3 | QQYYSTLT |
| 11 | >md5_2cdc3bd28ff984b7c5 | EVQLVESGGGLVKPGGSLRLSCAASGFTFRDVW |
| | b8d2a86ae9553f | MSWVRQAPGKGLEWVGRIKSKIDGGTTDYAAP |
| | Mutant_number: | VKGRFTISRDDSKNTLYLQMNSLKTEDTAVYYC |
| | md5_2cdc3bd28ff984b7c5b | TTAGSYYDDTVGPGLPEGKIDYWGQGTLVTVSS/ |
| | 8d2a86ae9553f HumID: | DIVMTQSPDSLAVSLGERATINCKSSQSVLYSVN |
| | v2130_upsample_20220121_ | NKNYLAWYQQKPGQPPKLLMNWASTRESGVPD |
| | 00009 Master: | RFSGSGSGAEFTLTISSLQAEDVAIYYCQQYYSTL |
| | RBD_2130_FAB | TFGGGTKVEIKR |
| | Mutations: YH106D, | |
| | FH118I, SL33V, YL55N | |
| | Fv Fragment- no linker | |
| 106 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYDDTVGPGLPEGKIDYWGQGTLVTVSS |
| 196 | CDRH1 | GFTFRDVW |
| 198 | CDRH2 | IKSKIDGGTT |
| 206 | CDRH3 | TTAGSYYDDTVGPGLPEGKIDY |
| 164 | Light chain (V_{L}) | DIVMTQSPDSLAVSLGERATINCKSSQSVLYSVN |
| | | NKNYLAWYQQKPGQPPKLLMNWASTRESGVP |
| | | DRFSGSGSGAEFTLTISSLQAEDVAIYYCQQYYS |
| | | TLTFGGGTKVEIKR |
| 235 | CDRL1 | QSVLYSVNNKNY |
| 242 | CDRL2 | WAS |
| 246 | CDRL2 | NWASTR |
| 251 | CDRL3 | QQYYSTLT |
| 12 | >md5_6e9c26854d3e83e74 | EVQLVESGGGLVKPGGSLRLSCAASGFTFRDVW |
| | f379947355e3c6a | MSWVRQAPGKGLEWVGRIKSKEDGGTTDYAAP |
| | Mutant_number: | VKGRFTISRDDSKNTLYLQMNSLKTEDTAVYYC |
| | md5_6e9c26854d3e83e74f3 | TTAGSYYHDTVGPGLPEGKFDYWGQGTLVTVSS/ |
| | 79947355e3c6a HumID: | DIVMTQSPDSLAVSLGERATINCKSSQSVLYRSN |
| | v2130_upsample_20220121_ | NLNYLAWYQQKPGQPPKLLMYWASTRESGVPD |
| | 00298 Master: | RFSGSGSGAEFTLTISSLQAEDVAIYYCQQYYSTL |
| | RBD_2130_FAB | TFGGGTKVEIKR |
| | Mutations: IH55E, | |
| | YH106H, SL32R, KL36L | |
| | Fv Fragment- no linker | |
| 107 | Heavy chain (V_{H}) | EVQLVESGGGLVKPGGSLRLSCAASGFTFRDVW |
| | | MSWVRQAPGKGLEWVGRIKSKEDGGTTDYAAP |
| | | VKGRFTISRDDSKNTLYLQMNSLKTEDTAVYYC |
| | | TTAGSYYHDTVGPGLPEGKFDYWGQGTLVTVS |
| | | S |
| 196 | CDRH1 | GFTFRDVW |
| 197 | CDRH2 | IKSKEDGGTT |
| 207 | CDRH3 | TTAGSYYHDTVGPGLPEGKFDY |
| 165 | Light chain (V_{L}) | DIVMTQSPDSLAVSLGERATINCKSSQSVLYRSN |
| | | NLNYLAWYQQKPGQPPKLLMYWASTRESGVPD |
| | | RFSGSGSGAEFTLTISSLQAEDVAIYYCQQYYST |
| | | LTFGGGTKVEIKR |
| 236 | CDRL1 | QSVLYRSNNLNY |
| 242 | CDRL2 | WAS |
| 243 | CDRL2 | YWASTR |
| 251 | CDRL3 | QQYYSTLT |
| 13 | >md5_8813a032f188710d1 | EVQLVESGGGLVKPGGSLRLSCAASGFTFRDVW |
| | acf700cb2d61380 | MSWVRQAPGKGLEWVGRIKSKHDGGTTDYAAP |
| | Mutant_number: | VKGRFTISRDDSKNTLYLQMNSLKTEDTAVYYC |
| | md5_8813a032f188710dla | TTAGSYYYDTVLPGLPEGKFDYWGQGTLVTVSS/ |
| | cf700cb2d61380 HumID: | DIVMTQSPDSLAVSLGERATINCKSSQSVLYSYN |
| | v2130_upsample_20220121_ | NKNYLAWYQQKPGQPPKLLMEWASTRESGVPD |
| | 00314 Master: | RFSGSGSGAEFTLTISSLQAEDVAIYYCQQYYSTL |
| | RBD_2130_FAB | TFGGGTKVEIKR |
| | Mutations: IH55H, | |
| | GH110L, SL33Y, YL55E | |
| | Fv Fragment- no linker | |
| 108 | Heavy chain (V_{H}) | EVQLVESGGGLVKPGGSLRLSCAASGFTFRDVW |
| | | MSWVRQAPGKGLEWVGRIKSKHDGGTTDYAA |
| | | PVKGRFTISRDDSKNTLYLQMNSLKTEDTAVYY |
| | | CTTAGSYYYDTVLPGLPEGKFDYWGQGTLVTV |
| | | SS |
| 196 | CDRH1 | GFTFRDVW |
| 201 | CDRH2 | IKSKHDGGTT |
| 208 | CDRH3 | TTAGSYYYDTVLPGLPEGKFDY |
| 166 | Light chain (V_{L}) | DIVMTQSPDSLAVSLGERATINCKSSQSVLYSYN |
| | | NKNYLAWYQQKPGQPPKLLMEWASTRESGVPD |
| | | RFSGSGSGAEFTLTISSLQAEDVAIYYCQQYYST |
| | | LTFGGGTKVEIKR |
| 228 | CDRL1 | QSVLYSYNNKNY |
| 242 | CDRL2 | WAS |
| 247 | CDRL2 | EWASTR |
| 251 | CDRL3 | QQYYSTLT |
| 14 | >md5_6362c40ele8234bda | EVQLVESGGGLVKPGGSLRLSCAASGFTFRDVW |
| | 1f78cd76d72ca17 | MSWVRQAPGKGLEWVGRIKSKIDGGTTDYAAP |
| | Mutant_number: | VKGRFTISRDDSKNTLYLQMNSLKTEDTAVYYC |
| | md5_6362c40ele8234bdalf | TTAGSYYYDTRGPELPEGKFDYWGQGTLVTVSS/ |
| | 78cd76d72ca17 HumID: | DIVMTQSPDSLAVSLGERATINCKSSQSVLYAAN |
| | v2130_upsample_20220121_ | NKNYLAWYQQKPGQPPKLLMYWASERESGVPD |
| | 00354 Master: | RFSGSGSGAEFTLTISSLQAEDVAIYYCQQYYSTL |
| | RBD_2130_FAB | TFGGGTKVEIKR |
| | Mutations: VH109R, | |
| | GH112E, SL32A, SL33A, | |
| | TL59E | |
| | Fv Fragment- no linker | |
| 109 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTRGPELPEGKFDYWGQGTLVTVSS |
| 196 | CDRH1 | GFTFRDVW |
| 198 | CDRH2 | IKSKIDGGTT |
| 209 | CDRH3 | TTAGSYYYDTRGPELPEGKFDY |
| 163 | Light chain (V_{L}) | DIVMTQSPDSLAVSLGERATINCKSSQSVLYAAN |
| | | NKNYLAWYQQKPGQPPKLLMYWASERESGVP |
| | | DRFSGSGSGAEFTLTISSLQAEDVAIYYCQQYYS |
| | | TLTFGGGTKVEIKR |
| 234 | CDRL1 | QSVLYAANNKNY |
| 242 | CDRL2 | WAS |
| 245 | CDRL2 | YWASER |
| 251 | CDRL3 | QQYYSTLT |
| 271 | COV2-RBD (rpk9) Wuhan- | MGILPSPGMPALLSLVSLLSVLLMGCVAETGTRF |
| | Hu-1 (WT) | PNITNLCPFGEVFNATRFASVYAWNRKRISNCVA |
| | | DFSVLYNSASFSTFKCYGVSPTKLNDLCWTNIYA |
| | | DSFVIRGDEVRQIAPGQTGKIADYNYKLPDDFTG |
| | | CVIAWNSNNLDSKVGGNYNYLYRLFRKSNLKPF |
| | | ERDISTEIYQAGSTPCNGVEGFNCYFPLQSYGFQP |
| | | TNGVGYQPYRVVVLSFELLHAPATVCGPKKSTG |
| | | LNDIFEAQKIEWHEHHHHHHHH* |
| 272 | COV2-RBD (rpk9) | MGILPSPGMPALLSLVSLLSVLLMGCVAETGTRF |
| | B.1.1.529 (BA.1) | PNITNLCPFDEVFNATRFASVYAWNRKRISNCVA |
| | | DFSVLYNLAPFFTFKCYGVSPTKLNDLCWTNIYA |
| | | DSFVIRGDEVRQIAPGQTGNIADYNYKLPDDFTG |
| | | CVIAWNSNKLDSKVSGNYNYLYRLFRKSNLKPF |
| | | ERDISTEIYQAGNKPCNGVAGFNCYFPLRSYSFRP |
| | | TYGVGHQPYRVVVLSFELLHAPATVCGPKKSTG |
| | | LNDIFEAQKIEWHEHHHHHHHH* |
| 273 | COV2-RBD (rpk9) | MGILPSPGMPALLSLVSLLSVLLMGCVAETGTRF |
| | B.1.1.529 R346K (BA.1.1) | PNITNLCPFDEVFNATKFASVYAWNRKRISNCVA |
| | | DFSVLYNLAPFFTFKCYGVSPTKLNDLCWTNIYA |
| | | DSFVIRGDEVRQIAPGQTGNIADYNYKLPDDFTG |
| | | CVIAWNSNKLDSKVSGNYNYLYRLFRKSNLKPF |
| | | ERDISTEIYQAGNKPCNGVAGFNCYFPLRSYSFRP |
| | | TYGVGHQPYRVVVLSFELLHAPATVCGPKKSTG |
| | | LNDIFEAQKIEWHEHHHHHHHH* |
| 274 | COV2-2130 Heavy chain | EVQLVESGGGLVKPGGSLRLSCAASGFTFRDVW |
| | variable domain | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 275 | COV2-2130 Light chain | DIVMTQSPDSLAVSLGERATINCKSSQSVLYSSN |
| | variable domain | NKNYLAWYQQKPGQPPKLLMYWASTRESGVPD |
| | | RFSGSGSGAEFTLTISSLQAEDVAIYYCQQYYSTL |
| | | TFGGGTKVEIKR |

### EXAMPLES

These examples are provided for illustrative purposes only and not to limit the scope of the claims provided herein. Specifically, these examples illustrate the identification and characterization of novel monoclonal antibodies against emerging SARS-CoV-2 strains.

### Example 1: Generation of Monoclonal Antibodies Against Emerging SARS-CoV-2 Strains

Using a novel computational software and the COV2-2130 monoclonal antibody as an input, over 700 putative novel anti-SARS-CoV-2 monoclonal antibodies were designed. A computer modeling predicted that these novel antibodies would be efficacious against emerging SARS-CoV-2 strains based on identified mutations in the Omicron strain and its lineages. The computer modeling also predicted that these putative novel antibodies would be manufacturable.

### Example 2: Functional Characterization of the Novel Monoclonal Antibodies Against Emerging SARS-CoV-2 Strains

### Gyros Screening Procedure to Identified and Prioritized Antibodies

To characterize the putative novel anti-SARS-CoV-2 monoclonal antibodies, the amino acid sequences of the scFv were generated for each of the 700 putative antibodies. Candidate monoclonal antibodies were evaluated at different concentrations for their interactions with the SARS-CoV-2 antigen. The binding response was measured optically. The derived binding affinity (EC₅₀) was compared with standard curves (concentration, response) derived from two control antibodies (s230, and COV2-2130). The comparison was concentration dependent. Each concentration of the candidate antibody was compared with the standard curves at the appropriate, individual concentration to determine if the candidate antibody showed performance characteristics that were superior to the parental COV2-2130 antibody. (FIG. 1 or Table 15). The antigens used to determine the affinity of the candidate antibodies comprise the amino acid selected from SEQ ID NO: 271, 272 or 273.

COV2-RBD_(rpk9) Wuhan-Hu-1 (WT)
SEQ ID NO: 271)
MGILPSPGMPALLSLVSLLSVLLMGCVAETGTRFPNITNLCPFGEVFNAT
RFASVYAWNRKRISNCVADFSVLYNSASFSTFKCYGVSPTKLNDLCWTNI
YADSFVIRGDEVRQIAPGQTGKIADYNYKLPDDFTGCVIAWNSNNLDSKV
GGNYNYLYRLFRKSNLKPFERDISTEIYQAGSTPCNGVEGFNCYFPLQSY
GFQPTNGVGYQPYRVVVLSFELLHAPATVCGPKKSTGLNDIFEAQKIEWH
EHHHHHHHH*
COV2-RBD_(rpk9) B.1.1.529 (BA.1)
(SEQ ID NO: 272)
MGILPSPGMPALLSLVSLLSVLLMGCVAETGTRFPNITNLCPFDEVFNAT
RFASVYAWNRKRISNCVADFSVLYNLAPFFTFKCYGVSPTKLNDLCWTNI
YADSFVIRGDEVRQIAPGQTGNIADYNYKLPDDFTGCVIAWNSNKLDSKV
SGNYNYLYRLFRKSNLKPFERDISTEIYQAGNKPCNGVAGFNCYFPLRSY
SFRPTYGVGHQPYRVVVLSFELLHAPATVCGPKKSTGLNDIFEAQKIEWH
EHHHHHHHH*
COV2-RBD_(rpk9) B.1.1.529 R346K (BA.1.1)
(SEQ ID NO: 273)
MGILPSPGMPALLSLVSLLSVLLMGCVAETGTRFPNITNLCPFDEVFNAT
KFASVYAWNRKRISNCVADFSVLYNLAPFFTFKCYGVSPTKLNDLCWTNI
YADSFVIRGDEVRQIAPGQTGNIADYNYKLPDDFTGCVIAWNSNKLDSKV
SGNYNYLYRLFRKSNLKPFERDISTEIYQAGNKPCNGVAGFNCYFPLRSY
SFRPTYGVGHQPYRVVVLSFELLHAPATVCGPKKSTGLNDIFEAQKIEWH
EHHHHHHHH*

### Full-Curve Procedure to Rank Candidate Antibodies.

To rank the candidate antibodies by performance, a full curve analysis of the binding affinity was performed to determine the Area under the curve (AUC) for each binding response. As above, standard curves of concentration and response to three selected antigens (SARS-CoV-2 Wuhan, BA.1 and BA.1.1) were prepared for control and parental reference antibodies (s309, COV2-2130 Ology, and an in-house manufactured COV2-2130). Analogous full curves described above were prepared for all candidate antibodies, and the curves were compared in terms of their EC₅₀ (μg/ml) and AUC (integral of normalized response units d log(concentraiton [μg/ml])) vs. the parental IgG). (FIG. 1).

### Antibody Screening Procedure to Isolate the Best Candidates

To further characterize the putative novel anti-SARS-CoV-2 monoclonal antibodies, yeast cells were transformed with each identified nucleic acid encoding the scFv of the desired antibodies. Yeast cell expressing the scFv were population sorted by FACS based on the overall level of expression and specific binding to selected antigens. The fractions with high overall expression and specific binding (top fraction of FACS-sorted yeast cells) were selected, grown, picked colonies, and sequenced.

### Kinetic Assay to Determine the Estimated K_{D} Values

To determine the estimated binding affinity for the antibody for the SARS-CoV-2 antigens, selected yeast colonies displaying the desired scFv were incubated with a range of antigen concentrations. Initial binding of the antigen to the yeast cells were measured by flow cytometry and quantified by yeast-associated APC fluorescence derived from the interaction of stretpavidin-APC with yeast-bound biotinylated antigen. APC-streptavidin conjugate assays for flow cytometry are known to those skilled in the art. See e.g., thermofisher.com/order/catalog/product/SA1005; bdbiosciences.com/en-eu/products/reagents/flow-cytometry-reagents/research-reagents/single-color-antibodies-ruo/apc-streptavidin.554067. Binding and antigen concentrations were then fitted to the Michaelis-Menten equation to derive the estimated K_{D} (FIG. 2). See, perkinelmer.com/product/surelighttm-binding-assay-apc-streptavid-ad0201; aatbio.com/products/apc-streptavidin-conjugate?adwords=1&gclid=EAIaIQobChMIqczU8cjJ9wIVFiyGCh18cAxMEAAYAyAAE gLCyfD_BwE; or biolegend.com/de-at/products/apc-streptavidin-1470.

Live Virus neutralization Assay

To determine whether the candidate antibodies could neutralize a live virus, a neutralization assay was conducted. One day prior to the assay, HEK 293T cells stably expressing human ACE2 (293T-hACE2 cells) were seeded onto 96-well tissue culture plates coated with poly-D-lysine. The day of the assay, serial dilutions of the monoclonal antibodies in duplicate were prepared in a 96-well microtiter plate and pre-incubated with pseudovirus for 1 h at 37° C., before the pseudovirus-mAb mixtures were added to HEK 293T-hACE2 monolayers. Plates were returned to the 37° C. incubator, and incubated for 48-60 h. After the 48-60 h incubation period, the luciferase activity was measured on a CLARIOStar® plate reader (BMG LabTech) using the Bright-Glo™ Luciferase Assay System (Promega). Percent inhibition of pseudovirus infection was calculated relative to pseudovirus-only control. IC₅₀ values were determined by nonlinear regression using Prism v.8.1.0 (GraphPad). Each neutralization assay was repeated at least twice.

Additional methods for characterizing the antibodies of the present disclosure can be found in Crawford et al., Protocol and Reagents for Pseudotyping Lentiviral Particles with SARS-CoV-2 Spike Protein for Neutralization Assays, Viruses 12 (5):513-(2020).

### Results

Following in vitro characterization of these over 700 putative antibodies, a subset of those antibodies (14 novel antibodies) were shown to bind to a recombinant receptor binding domain (RBD) construct (SRBD) of the viral spike (S) glycoprotein. Each of the 14 novel antibodies also exhibited neutralizing activity in a rapid screening assay with the Wuhan, original, SARS-CoV-2 strain and the Omicron strains (BA.1, BA.1.1 and BA. 2) (FIG. 3).

The estimated binding affinity for a subset of these 14 monoclonal antibodies was shown to be far superior to the binding affinity of the parental COV2-2130 antibody (FIG. 2). In addition, many of the 14 selected antibodies were shown to have higher neutralizing potencies when compared to the COV2-2130 (IC₅₀<150 ng/ml; FIG. 3). Such neutralization potencies imply likely competitive inhibition of human ACE2.

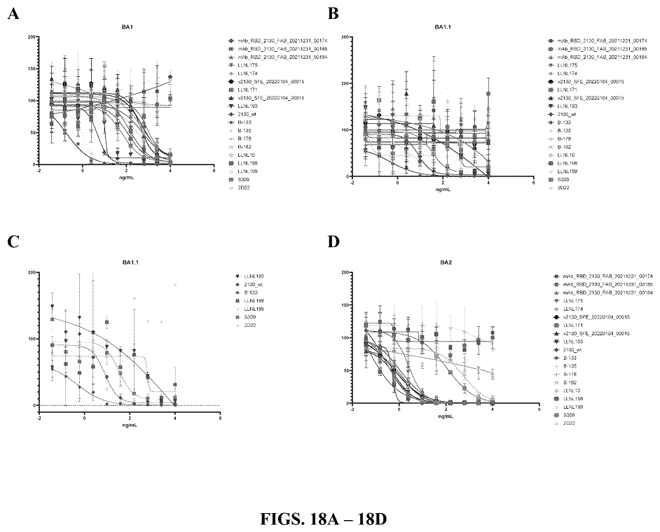

To further confirm the potent neutralization of these 14 selected antibodies, a pseudovirus neutralization assay was conducted using a recombinant RBD construct of the viral spike (S) glycoprotein comprising a D614G mutation or derived from the Omicron strains (BA.1, BA.1.1 and BA. 2). As shown in FIG. 3, 5 of these 14 novel antibodies exhibited half-maximum inhibitory concentration (IC₅₀) values that ranged from 0.1 to over 3,000 ng ml⁻¹ virus. Surprisingly, the majority of observed IC₅₀ values against emerging SARS-CoV-2 strains were less than 1.0 ng ml⁻¹ virus compared to more than 6.0 ng ml⁻¹ virus for the parental COV2-2130. Accordingly, the neutralization characteristics of the disclosed novel antibodies were superior to those of the parent COV2-2130 antibody. Peseudovirus neutralization of the other 9 novel antibodies described herein is shown in FIGS. 18A-18D.

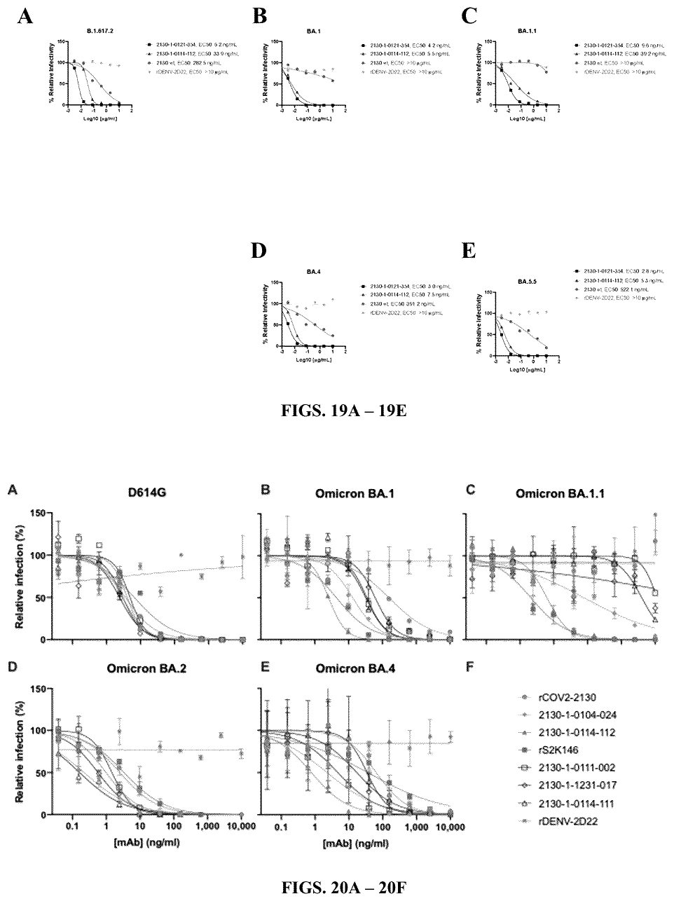

FIGS. 20A-20F show how designed antibodies improved pseudoviral neutralization over COV2-2130. The parental COV2-2130 antibody (orange circles), cross-reactive positive control antibody rS2K146 (magenta squares), and down-selected computationally designed antibodies were assayed by neutralization with lentiviruses pseudotyped with spike variants of Wuhan-1 D614G (FIG. 20A), Omicron BA.1 (FIG. 20B), BA.1.1 (FIG. 20C), BA.2 (FIG. 20D), and BA.4 (FIG. 20E). FIG. 20F shows a key for the symbols of FIGS. 20A-20E. rDENV-2D22 was used as a negative control (grey crosses). Example 3—Antigen production.

To express the RBD subdomain of the SARS-CoV-2 S protein, residues 328-531 were cloned into a mammalian expression vector downstream of an mu-phosphatase signal peptide and upstream of an AviTag and a 8×His tag. Three previously identified stabilizing mutations (Y365F, F392W, V395I) were included in the RBD to enhance stability and yield. For RBD constructs corresponding to the Omicron subvariants, mutations present in each subvariant were introduced into the context of the stabilized, wild-type RBD construct. RBD constructs were transfected into Expi293F cells (ThermoFisher Scientific), and expressed protein was isolated by metal affinity chromatography on HisTrap Excel columns (Cytiva). Purified proteins were analyzed by SDS-PAGE to ensure purity and appropriate molecular weights.

Nucleotide sequences encoding the designed heavy and light chain sequences for each antibody in a first set of 230 antibody candidates were synthesized, cloned into an hIgG1 framework, and used to produce mAbs via transient transfection of HEK293 cells at ATUM (Newark, CA, USA).

For second set of 204 antibody candidates, monoclonal antibody sequences were synthesized (Twist Bioscience) and cloned into an IgG1 monocistronic expression vector (designated as pVVC-mCisK hG1) [Chng 2015] and expressed either at microscale in transiently transfected ExpiCHO cells [Zost 2020a] for screening or at a larger-scale for down-stream assays. Sequences in this group all contain an additional arginine at the beginning of the light chain constant region with respect to sequences expressed in the first set. Larger-scale monoclonal antibody expression was performed by transfecting (30 ml per antibody) CHO cell cultures using the Gibco ExpiCHO Expression System and protocol for 125 ml flasks (Corning) as described by the vendor. Culture supernatants were purified using HiTrap MabSelect SuRe (Cytiva, formerly GE Healthcare Life Sciences) on a 24-column parallel protein chromatography system (Protein BioSolutions). Purified monoclonal antibodies were buffer-exchanged into PBS, and stored at 4° C. until use.

### Binding Screening and Characterization

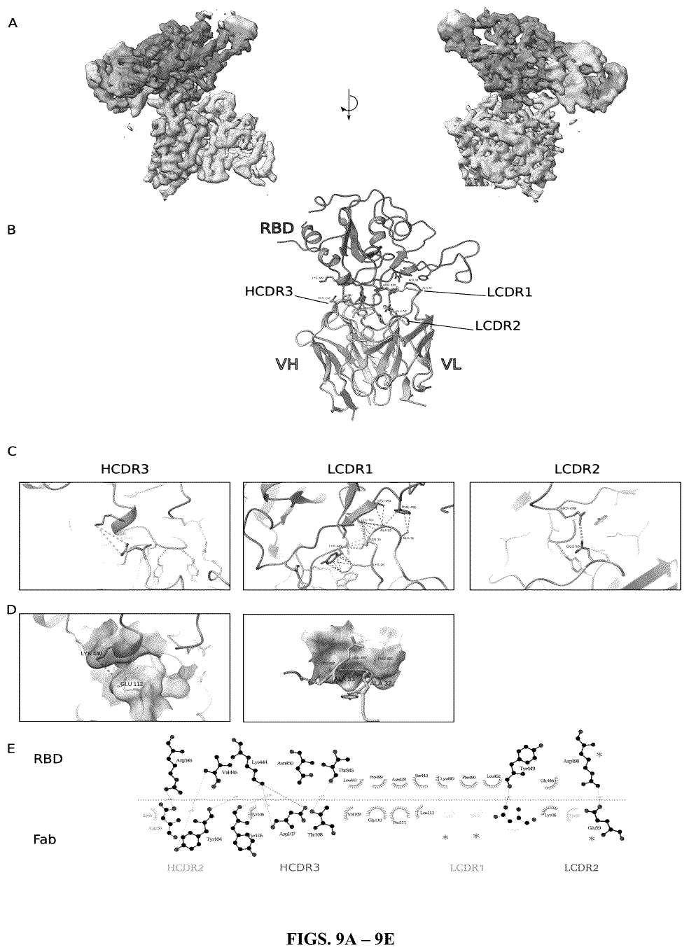

Immunoassays for screening the first set of 230 antibody candidates (FIGS. 10A-10D) and later characterization were performed on the Gyrolab xPlore instrument (Gyros Protein Technologies) using the Bioaffy 200 discs (Gyros Protein Technologies). The standard manufacturer's immunoassay automated protocol was executed with fluorescence detection set to 0.1% PMT. Assay column washes were performed with in PBS+0.02% Tween 20 (PBST). Capture antigens were applied to the assay columns at 0.5 to 2.0 μM in PBS. Analyte mAbs were applied to the assay columns diluted in PBST at 1:200 for single-concentration screening or as a serial dilution from 1,000 nM to 0.25 nM for characterization of down-selected candidate antibodies. A secondary detection antibody served as a fluorescent reporter: Alexa Fluor 647 AffiniPure Fab Fragment Goat Anti-Human IgG, Fcγ fragment specific (Jackson ImmunoResearch) diluted to 50-100 nM in RexxipF buffer (Gyros Protein Technologies). Resulting values were fit to a 4PL model or calculated as area under the curve (AUC) using GraphPad Prism software.

### Dose-Response ELISA Binding Assays

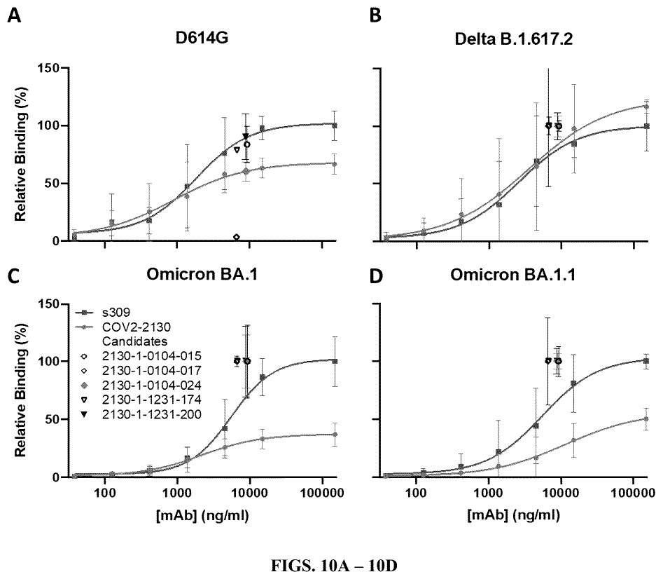

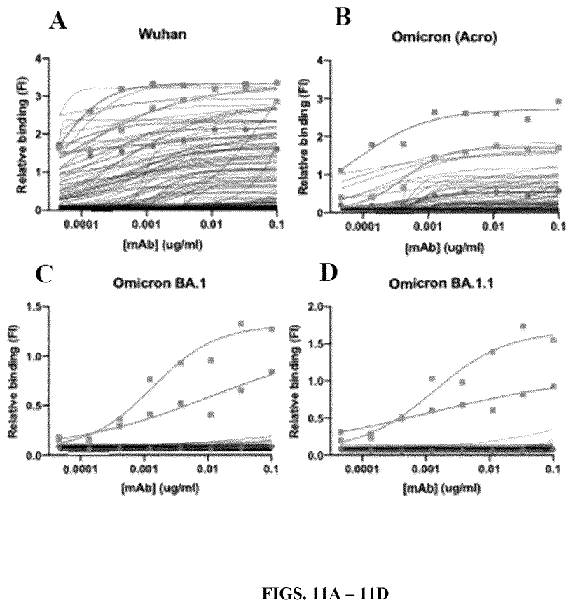

For screening and characterizing the second set of 204 antibody candidates (FIGS. 11A-11D), wells of 384-well microtiter plates were coated with purified recombinant SARS-CoV-2 RBD proteins at 4° C. overnight at a concentration of 2 mg/mL of antigen. Plates were washed with Dulbecco's phosphate-buffered saline (DPBS) containing 0.05% Tween-20 (DPBS-T) and blocked with 2% bovine serum albumin and 2% normal goat serum in DPBS-T (blocking buffer) for 1 h. mAbs were diluted in 12 three-fold serial dilutions in blocking buffer at a starting concentration of 10 mg/mL. Plates were then washed and mAb dilutions were added and incubated for 1 h. Plates were washed and a goat anti-human IgG conjugated with horseradish peroxidase (HRP) (Southern Biotech, cat. 2014-05, lot L2118-VG00B, 1:5,000 dilution in blocking buffer) and incubated for 1 h. After plates were washed, signal was developed with a 3,3′,5,5′-tetramethylbenzidine (TMB) substrate (Thermo Fisher Scientific). Color development was monitored, 1M hydrochloric acid was added to stop the reaction, and the absorbance was measured at 450 nm using a spectrophotometer (Biotek). Dose-response ELISAs were performed in technical triplicate with at least two independent experimental replicates.

### Thermal Shift Protein Assays (Melt-Curve Assays)

Antibody concentrations were determined using the Qubit Protein Assay Kit (ThermoFisher). The GloMelt™ Thermal Shift Protein Stability Kit (Biotum) was utilized to determine the thermal stability of the antibodies, following the manufacturer's suggested protocols. The analysis was performed using a melt-curve program on an ABI 7500 Fast Dx Real-Time PCR instrument. Each assay was done in triplicates, using 5 ug of mAb per well. The raw melt curve data was imported into and analyzed via Protein Thermal Shift™ software version 1.4 (ThermoFisher) to generate the melting temperature and fit data.

### Pseudovirus Neutralization

Pseudovirus neutralization assays were or are carried out according to the protocol of Crawford et al. [VPneut1]. One day prior to the assay, 293T cells stably expressing human ACE2 (293T-hACE2 cells) were or are seeded onto 96-well tissue culture plates coated with poly-D-lysine. The day of the assay, serial dilutions of monoclonal antibodies in duplicate were or are prepared in a 96-well microtiter plate and pre-incubated with pseudovirus for 1 h at 37° C. in the presence of a final concentration of 5 mg/mL polybrene (EMD Millipore), before the pseudovirus-mAb mixtures were added to 293T-hACE2 monolayers. Plates were or are returned to the 37° C. incubator, and then 48-60 h later luciferase activity was measured on a CLARIOStar plate reader (BMG LabTech) using the Bright-Glo Luciferase Assay System (Promega). Percent inhibition of pseudovirus infection was or is calculated relative to pseudovirus-only control. IC50 values were or are determined by nonlinear regression using Prism v.8.1.0 (GraphPad). Each neutralization assay was or is repeated at least twice.

### Focus Reduction Neutralization Test

Serial dilutions of sera were or are incubated with 10² focus-forming units (FFU) of WA1/2020 D614G, B.1.617.2, BA.1, BA.1.1, BA.2, BA.2.12.1, BA.4, BA.5, or BA.5.5 for 1 h at 37° C. Antibody-virus complexes were or are added to Vero-TMPRSS2 cell monolayers in 96-well plates and incubated at 37° C. for 1 h. Subsequently, cells were or are overlaid with 1% (w/v) methylcellulose in MEM. Plates were or are harvested 30 h (WA1/2020 D614G and B.1.617.2) or 70 h (BA.1, BA.1.1, BA.2, BA.2.12.1, BA.4, BA.5, and BA.5.5) later by removing overlays and fixed with 4% PFA in PBS for 20 min at room temperature. Plates were or are washed and sequentially incubated with a pool (SARS2-02, -08, -09, -10, -11, -13, -14, -17, -20, -26, -27, -28, -31, -38, -41, -42, -44, -49, -57, -62, -64, -65, -67, and -71 [VanBlargan2021]) of anti-S murine antibodies (including cross-reactive mAbs to SARS-CoV) and HRP-conjugated goat anti-mouse IgG (Sigma Cat #A8924, RRID: AB 258426) in PBS supplemented with 0.1% saponin and 0.1% bovine serum albumin. SARS-CoV-2-infected cell foci were or are visualized using TrueBlue peroxidase substrate (KPL) and quantitated on an ImmunoSpot microanalyzer (Cellular Technologies).

### In Vivo Studies: Viruses

The WA1/2020 recombinant strain with D614G substitution and B.1.617.2 was described previously [Plante2020][Ying2021]. The BA.1 isolate was obtained from an individual in Wisconsin as a mid-turbinate nasal swab [Halfmann2022]. The BA.1.1 and BA.2 strains were obtained from nasopharyngeal isolates. The BA.2.12.1, BA.4, BA.5, and BA.5.5 isolates were generous gifts from M. Suthar (Emory University), A. Pekosz (Johns Hopkins University), and R. Webby (St. Jude Children's Research Hospital). All viruses were passaged once on Vero-TMPRSS2 cells and subjected to next-generation sequencing [Chen2021] to confirm the introduction and stability of substitutions. All virus experiments were performed in an approved biosafety level 3 (BSL-3) facility.

### In Vivo Studies—Mice.

Animal studies were carried out in accordance with the recommendations in the Guide for the Care and Use of Laboratory Animals of the National Institutes of Health. The protocols were approved by the Institutional Animal Care and Use Committee at the Washington University School of Medicine (assurance number A3381-01). Virus inoculations were performed under anesthesia that was induced and maintained with ketamine hydrochloride and xylazine, and all efforts were made to minimize animal suffering. Heterozygous K18-hACE2 C57BL/6J mice (strain: 2B6.Cg-Tg(K18-ACE2)2Prlmn/J, Cat #34860) were obtained from The Jackson Laboratory. Animals were housed in groups and fed standard chow diets.

### In Vivo Studies-Experiments.

Eight-week-old female K18-hACE2 C57BL/6 mice were administered 100 μg of 2130-1-0114-112 (also referred to herein as LLNL199), parental 2130, or isotype control anti-West Nile hE16 mAb [Oliphant 2005] by intraperitoneal injection one day before intranasal inoculation with 104 focus-forming units (FFU) of WA1/2020 D614G, BA.1.1 or BA.5. Animals were euthanized at 4 days post-infection and tissues were harvested for virological analysis.

### In Vivo Studies: Measurement of Viral RNA.

Tissues were weighed and homogenized with zirconia beads in a MagNA Lyser instrument (Roche Life Science) in 1 ml of DMEM medium supplemented with 2% heat-inactivated FBS. Tissue homogenates were clarified by centrifugation at 10,000 rpm for 5 min and stored at −80° C. RNA was extracted using the MagMax mirVana Total RNA isolation kit (Thermo Fisher Scientific) on the Kingfisher Flex extraction robot (Thermo Fisher Scientific). RNA was reverse transcribed and amplified using the TaqMan RNA-to-CT 1-Step Kit (Thermo Fisher Scientific). Reverse transcription was carried out at 48° C. for 15 min followed by 2 min at 95° C. Amplification was accomplished over 50 cycles as follows: 95° C. for 15 s and 60° C. for 1 min. Copies of SARS-CoV-2 N gene RNA in samples were determined using a published assay [Case 2020].

### Cryo-EM Sample Preparation and Data Collection.

The Fab 2130-1-0114-112 (also referred to herein as LLNL199) and Cov2 BA.2 were expressed recombinantly and combined in a molar ration of 1:4 (Ag:Fab). The mixture was incubated over-night at 4° C. and purified by gel filtration. 2.2 μl of the purified mixture at concentration of 0.5 mg/mL was applied to glow discharged (30 s at 25 mA) grid (300 mesh 1.2/1.3, Quantifoil). The grids were blotted for 3.5 s before plunging into liquid ethane using Vitrobot MK4 (TFS) at 20° C. and 100% RH. Grids were screened on a Glacios (TFS) microscope and imaged on Krios operated at 300 keV equipped with a K3 and GIF (Gatan) DED detector using counting mode. Movies were collected at nominal magnification of 130,000×, pixel size of 0.647 Å/pixel and defocus range of 0.8 to 1.8 μm. Grids were exposed at ˜1.09 e⁻/Å²/frame resulting in total dose of ˜52.2 e⁻/Å² (Table 7).

### Cryo-EM Data Processing.

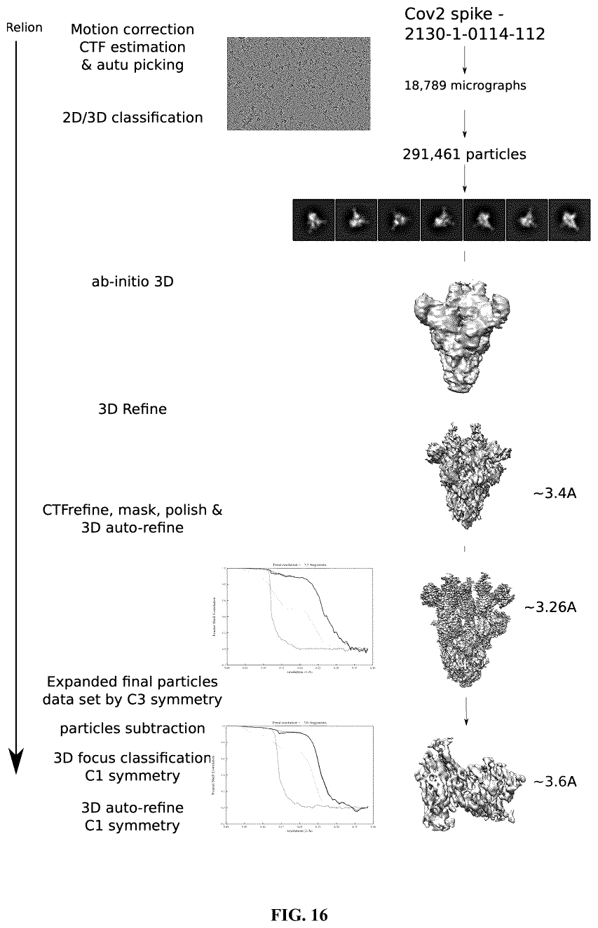

Data processing was performed with Relion 4.0 beta2 [Kimanius2021]. Movies were preprocessed with Relion Motioncor2 [Zheng2017] and CTFFind4 [Rohou2015]. Micrographs with low resolution, high astigmatism and defocus were removed from the data set. The data set was first manual pick to generate 2D images and then autopicked by Relion template picker [Fernandez-Leiro2017] and was subject to 2D and 3D classification. Good classes were selected and used for another round of autopicking with Topaz training and Topaz picking [Bepler2020][Kimanus2021]. The particles were extracted in a box size of 600 pixel and binned to 96 pixels (pixel size of 4.04 Å/pixel). The particles were subjected to multiple rounds of 2D class averages, 3D initial map and 3D classification without symmetry to obtain a clean homogeneous particle set. This set was re-extracted at a pixel size of 1.516 Å/pixel and was subjected to 3D autorefinement. The data were further re-extracted at a pixel size of 1.29 Å/pixel and processed with CTFrefine, polished [Zivanov2018][Scheres2018] and subjected to final 3D autorefinement and postprocessing resulting in ˜3.26 Å map. To better resolve the area of interaction between Cov2-RBD/2130-1-0114-112 (also referred to herein as LLNL199), a focused refinement was performed by particles expansion (C3 symmetry) and signal subtraction with masking around the RBD/2130-1-0114-112 (also referred to herein as LLNL199). The subtracted particles were subjected to 3D classification without alignment and selected particles were subjected to 3D autorefinement and postprocessing resulting in ˜3.7 Å map. Detailed statistics are provided in FIG. 16 and Table 7 described herein.

### Example 4

### Antibodies Improved Binding to Omicron Variants and Preserved Thermostability

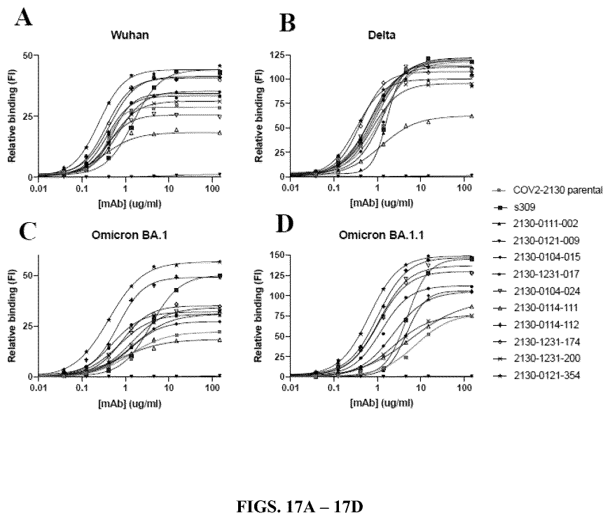

All antibody candidates antibodies were screened for binding by a single-concentration immunoassay (Gyrolab xPlore) in the contexts of BA.1, BA.1.1, and wild type RBDs (set 1; FIGS. 10A-10D for later optimization of this assay) or a multi-concentration immunoassay (ELISA; FIGS. 11A-11D), respectively, in the context of wild type, BA.1 or BA.1.1 RBDs (set 2) in comparison with a broadly cross-reactive control antibody 5309 [pinto2020] and the parental COV2-2130 antibody. As intended, the majority of the antibody designs had altered binding profiles, i.e., the designed mutations were consequential. Thus, only approximately 11% of the first set of 230 designs retained wild type antigen binding at the measured concentration; roughly 6% improved binding against BA.1 and 5% against BA.1.1. Following this initial screen, both sets of antibody designs were down-selected to those with improved binding to Omicron variants BA.1 and BA.1.1. A subset of antibody candidates were also evaluated as compared to COV2-2130 and s309 using full-curve Gyros binding to strain (FIG. 17A) Wuhan; (FIG. 17B) Delta; (FIG. 17C) Omicron BA.1; (FIG. 17D) Omicron BA.1.1.

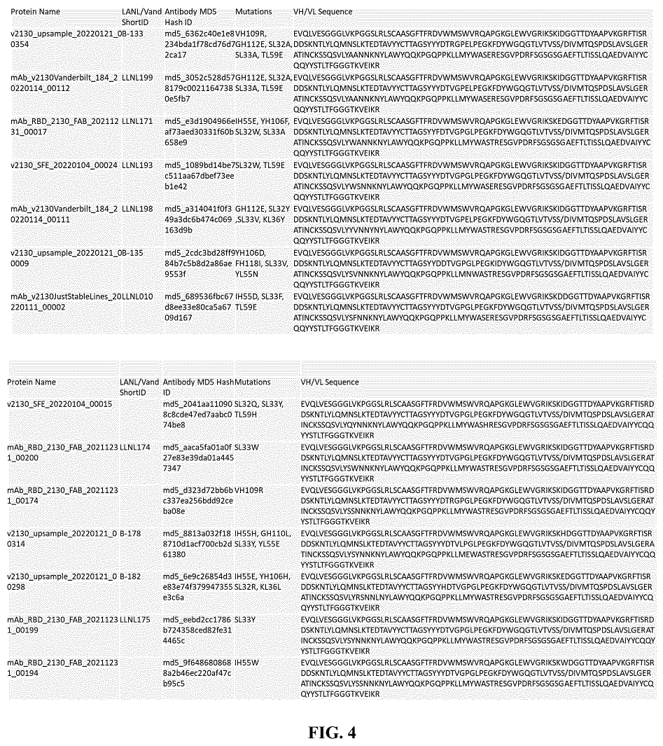

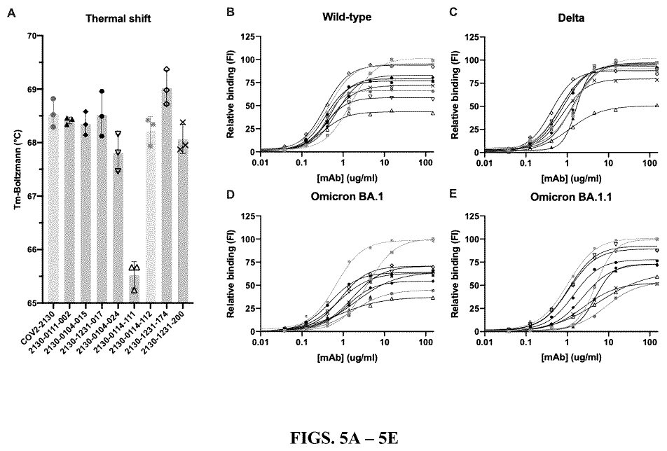

These down-selected antibodies were remanufactured at larger scale. The resulting antibodies were characterized IgG antibodies by immunoassay and thermal shift (melt temperature) assessments. Seven of the eight top-performing antibodies preserved comparable binding to wild type and Delta and improved over the parental COV2-2130 antibody in their binding to Omicron BA.1 and BA.1.1 RBDs (FIGS. 5A-5E). Further, seven of the eight antibodies had melting temperatures and expression properties comparable to those of COV2-2130. One antibody, 2130-1-0114-111 (also referred to herein as LLNL198) had reduced melting temperature. (Table 6). Antibody 2130-1-0114-112 (also referred to herein as LLNL199) displayed best-in-class binding across all RBD variants and had no significant difference in thermal stability compared to the parental COV2-2130 antibody.

### Antibodies Restored Neutralization to Omicron Variants in Pseudoviral Neutralization Assays

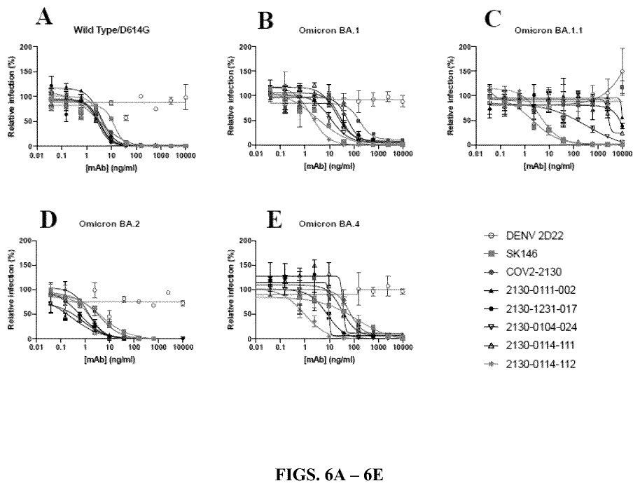

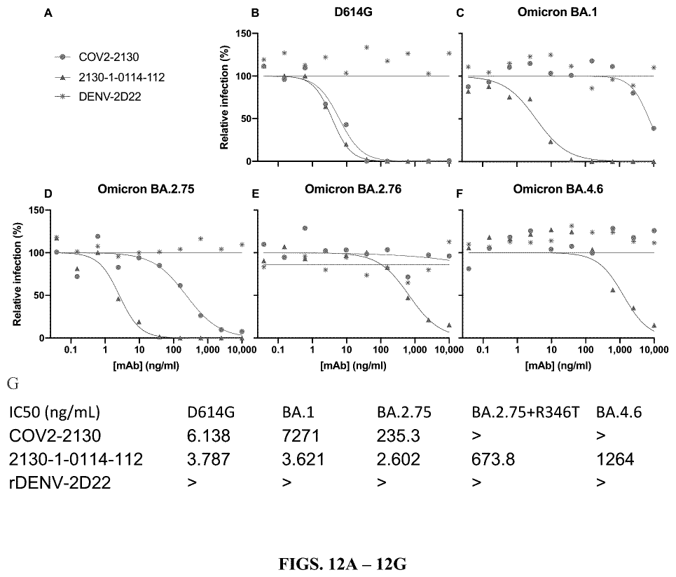

Pseudovirus neutralization assays was performed to characterize the functional performance of our antibody designs (FIGS. 6A-6E). The novel designs described herein maintained neutralization activity against pseudoviruses displaying historical spike proteins (D614G) and also achieved neutralization of those with Omicron BA.1 spikes. The single best candidate design, 2130-1-0114-112 (also referred to herein as LLNL199), restored potent neutralization in the context of BA.1.1 and showed a two-order-of-magnitude improvement in IC50 vs. parental COV2-2130 for BA.1 and BA.4. These pseudovirus neutralization test results showed that the designs neutralized BA.2 and BA.4 more potently than COV2-2130, despite the emergence of these VOCs after the conception of the designs described herein. 2130-1-0114-112's (also referred to herein as LLNL199) performance against BA.2.75, BA.4.6, which contains mutation R346T, among others, and an artificially-constructed BA.2.75+R346T, which matches the RBD sequence of BA.2.76 (FIGS. 12A-12G) were additionally tested. 2130-1-0114-112 (also referred to herein as LLNL199) outperforms COV2-2130, including maintaining potent neutralization of BA.2.75 (IC₅₀ 1.9 ng/ml), which reduces COV2-2130's potency by more than an order of magnitude, and measurable neutralization of BA.4.6 and BA.2.75+R346T, which are not measurably neutralized by COV2-2130.

### Disclosed Antibody, 2130-1-0114-112 (Also Referred to Herein as LLNL199), Restores Neutralization of Omicron Variants in an Authentic Virus Assay

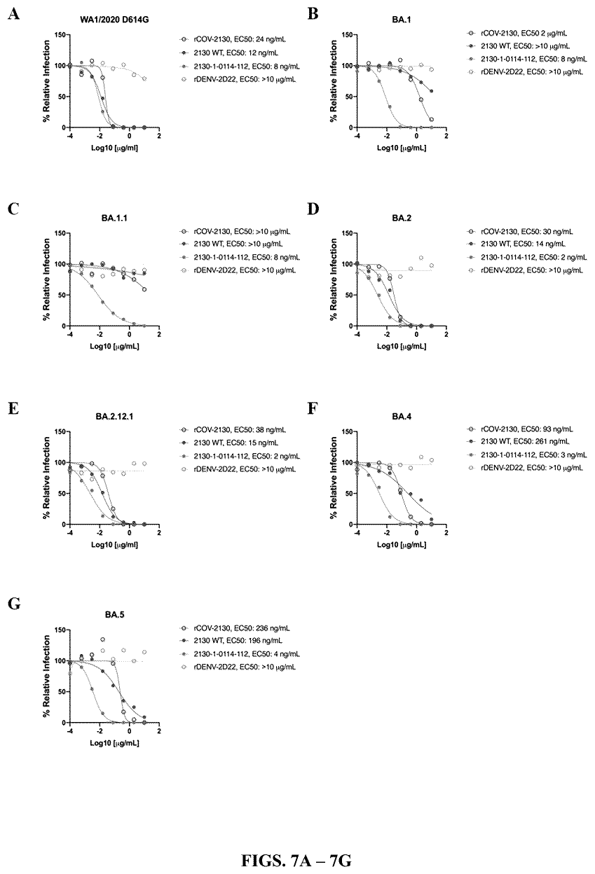

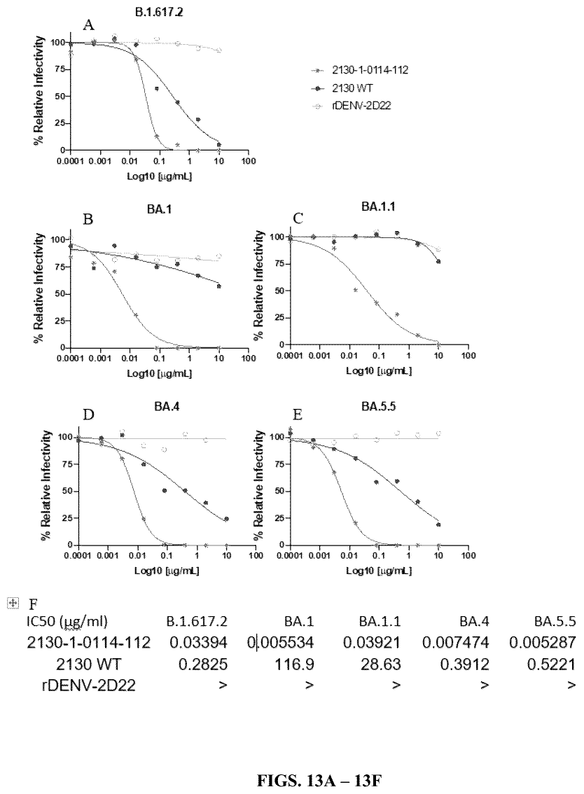

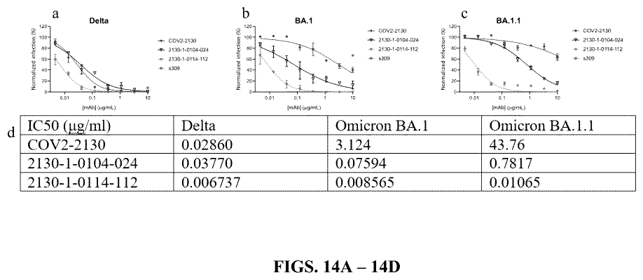

Antibody, 2130-1-0114-112 (also referred to herein as LLNL199) was evaluated, for authentic virus neutralization performance against WA1/2020 D614G, BA.1, BA.1.1, BA.2, BA.2.12.1, BA.4 and BA.5, by focus reduction neutralization test (FRNT) in Vero-TMPRSS2 cells (FIGS. 7A-7G). In all cases, 2130-1-0114-112 (also referred to herein as LLNL199) had an IC₅₀<10 ng/mL. Compared to the parental COV2-2130, 2130-1-0114-112 (also referred to herein as LLNL199) restored potent neutralization activity against BA.1 and BA.1.1, a more than 5-fold improvement in IC50 in against BA.2 and BA.2.12.1, and an approximately 50-fold improvement in IC50 against BA.4 and BA.5. Neutralization performance by FRNT for Delta (B.1.617.2), BA.1, BA.1.1, BA.4 and BA.5.5, (FIGS. 13A-13F) was separately evaluated. 2130-1-0114-112 (also referred to herein as LLNL199) strongly neutralizes Delta (IC₅₀=34 ng/ml) and BA.5.5 (IC₅₀=5.3 ng/ml); the other, overlapping strains show consistent results. 2130-1-0114-112 (also referred to herein as LLNL199) and a less-mutated alternative design, 2130-1-0104-024 (also referred to herein as LLNL193), in plaque assays with Vero E6-TMPRSS2-T2A-ACE2 cells were also evaluated As shown in FIGS. 14A-14D. IC₅₀ values for 2130-1-0104-024 (also referred to herein as LLNL193) were 0.0377 μg/ml, 0.07594 μg/ml and 0.7817 μg/ml for Delta, BA.1, and BA.1.1 viruses, respectively (FIG. 14D). Authentic virus neutralization was also evaluated for 2130-1-0121-354 (also referred to herein as B-133) and further evaluated for 2130-1-0114-112 (also referred to herein as LLNL199). 2130-1-0121-354 (also referred to herein as B-133) and 2130-1-0114-112 (also referred to herein as LLNL199) potently neutralized (FIG. 19A) B.1.617.2; (FIG. 19B) BA.1; (FIG. 19C) BA1.1; (FIG. 19D) BA.4; (FIG. 19E) BA.5.5 authentic viruses in focus reduction neutralization assays in Vero-TMPRSS2 cells. Interestingly, these data demonstrated that 2130-1-0121-354 (also referred to herein as B-133) is at least equivalent as compared to the closely related 2130-1-0114-112 (also referred to herein as LLNL199) in terms of authentic virus neutralization

Prophylaxis with 2130-1-0114-112 (Also Referred to Herein as LLNL199) Protects Against SARS-CoV-2 Variants.

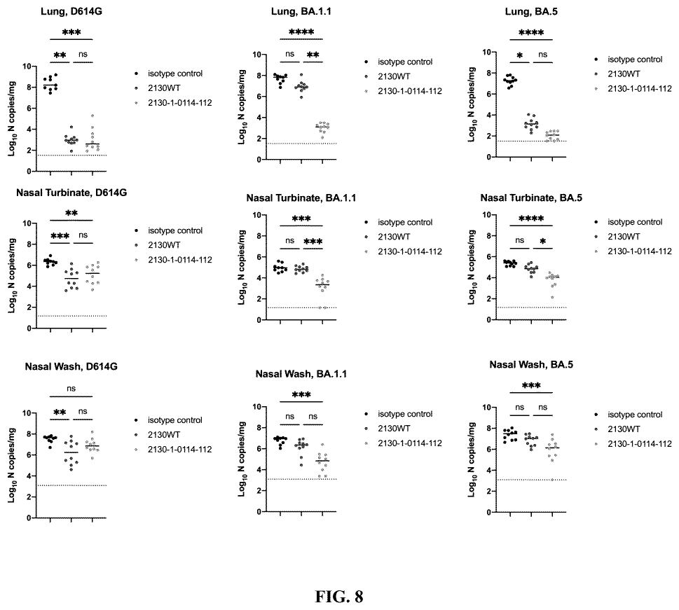

To assess the comparative efficacy of 2130-1-0114-112 (also referred to herein as LLNL199) and the parental COV2-2130 mAb in vivo, K18-hACE2 was administered to transgenic mice a single 100 μg (˜5 mg/kg total) dose one day prior to intranasal inoculation with WA1/2020 D614G, BA.1.1, or BA.5. Although Omicron lineage viruses are less pathogenic in mice, they replicated efficiently in the lungs of K18-hACE2 mice [Case2020][Halfmann2022][Uraki2022][Ying2022]. A total of 88 mice were used, 9-10 in each of the antibody and viral strain conditions. Viral RNA levels were measured at 4 days post-infection in the nasal washes, nasal turbinates, and lungs (FIG. 8). As expected, the parental COV2-2130 mAb effectively reduced WA1/2020 D614G infection in the lungs (180,930-fold), nasal turbinates (42-fold) and nasal washes (25-fold) compared to the isotype control mAb. However, the COV2-2130 mAb lost protective activity against BA.1.1 in all respiratory tract tissue, whereas against BA.5, protection was maintained in the lungs (13,622-fold) but not in the nasal turbinates or nasal washes. In comparison, 2130-1-0114-112 (also referred to herein as LLNL199) protected against lung infection by WA1/2020 D614G (399,945-fold reduction), BA.1.1 (53,468-fold reduction), and BA.5 (160,133-fold reduction) compared to the isotype control mAb (FIG. 8). Moreover, in the upper respiratory tract (nasal turbinates and washes), 2130-1-0114-112 (also referred to herein as LLNL199) also conferred protection against WA1/2020 D614G, BA.1.1, and BA.5. The differences in protection between the parental COV2-2130 and derivative 2130-1-0114-112 (also referred to herein as LLNL199) mAbs were most apparent in mice infected with BA.1.1, which directly parallels the neutralization data (FIGS. 7A-7G).

### Structural Basis for the Restored Potency of 2130-1-0114-112 (Also Referred to Herein as LLNL199)

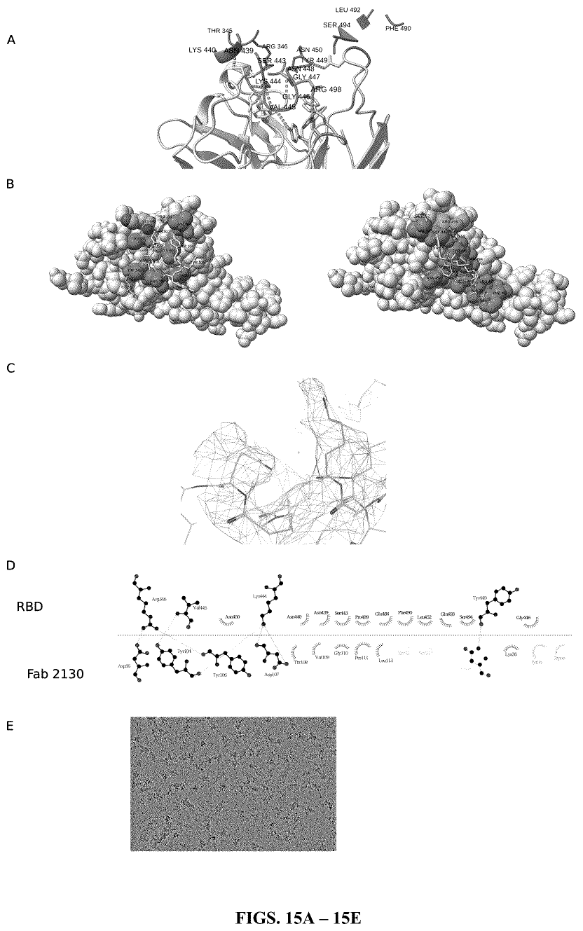

To understand the molecular mechanism and the atomic details of the recognition of Omicron RBD by 2130-1-0114-112 (also referred to herein as LLNL199), 3D reconstructions was performed by cryo-EM of 2130-1-0114-112 (also referred to herein as LLNL199) in complex with the SARS-CoV-2 Omicron BA.2 spike at 3.26 Å. Although the overall resolution was sufficient for model building, the interface region between the RBD and 2130-1-0114-112 (also referred to herein as LLNL199) was not well-resolved due to its flexibility. To address this, focused refinement of this portion of the structure to ˜3.6 Å (EMD-28198, EMD-28199, PDB 8EDK) was performed (FIGS. 9A-9E and FIG. 16, Table 7). This model showed the binding interface of 2130-1-0114-112 (also referred to herein as LLNL199)/RBD and can shed light about how 2130-1-0114-112 (also referred to herein as LLNL199) regained neutralization potency against Omicron VOCs and restored the potency of COV2-2130. COV2-2130 forms extensive interactions with the RBD through HCDR2 and HCDR3, as well as the LCDR1 and LCDR2 [Dong 2021]. The interaction of these loops includes hydrogen bond networks and hydrophobic interactions. SARS-CoV-2 Omicron BA.2 has two mutations in close proximity to (7 Å distance) (FIGS. 15A-15E) COV2-2130 (N440K and Q498R). To improve binding interactions with Omicron variants, 2130-1-0114-112 (also referred to herein as LLNL199) was modified three CDR loops (HCDR3, LCDR1 and LCDR2). The N440K mutation is on the edge of the RBD interface with the 2130-1-0114-112 (LLNL199) HCDR3 loop and does not make direct contact with G112E. However, this change introduced a positive charge to the local environment that has substantial hydrophobic contact. The negative charge introduced by the G112E substitution (FIG. 9C & FIG. 9D) on the HCDR3 loop can improve the electrostatic interactions in this region. Although the structure's resolution may not be sufficient for modeling water, it was possible that E112 and K440 could coordinate a water molecule. The local environment around the LCDR1 loop was mostly hydrophobic (comprised of RBD residues L452, F490 and L492, as well as the Omicron mutation E484A) with an N34 hydrogen bond (FIG. 9D). The hydrophilic-to-hydrophobic LCDR1 mutations introduced in 2130-1-0114-112 (also referred to herein as also referred to herein as LLNL199), S32A and S33A, may favor the local environment and strengthen hydrophobic interactions with the RBD (FIG. 9C & FIG. 9E). Lastly, the T59E mutation in the LCDR2 loop establishes a new salt bridge with the RBD residue R498 present in Omicron RBDs. This new salt bridge likely strengthens the interaction with the RBD (FIG. 9C & FIG. 9E). Altogether, the structural model of the 2130-1-0114-112 (also referred to herein as LLNL199) with the BA.2 RBD helps explain the observed restoration of potency against SARS-CoV-2 Omicron VOCs.

### DISCUSSION

Applicant describes herein novel derivatives of the COV2-2130 antibody that can restore potent in vitro neutralization against and/or bind BA.1 and BA.1.1 Omicron variants while maintaining binding and/or neutralization to previous VOCs. Additionally, the goal was to retain favorable thermostability properties and maintain the sequences' humanness, a data-driven measure of similarity to known human sequences. Despite the multiple mutations in the COV2-2130 epitope of Omicron BA.1 and BA.1.1, these simultaneous design objectives were achieved.

Several antibody candidates were successful in restoring pseudovirus neutralization potency to Omicron variants. In contrast, mutations at positions 32 and 33 in CDRL1 appear to be enriched, particularly to hydrophobic residues, consistent with Applicant's analysis of this part of the experimentally solved structure of 2130-1-0114-112 (also referred to herein as LLNL199) and BA.2 spike (FIG. 9C & FIG. 9D). As described above, the four designed mutations in this antibody appear to accommodate the mutations in Omicron and optimize both the electrostatic and the hydrophobic interactions with Omicron lineage RBD. Empirically, this design fully restores potent neutralization against BA.1 and BA.1.1 while maintaining D614G and Delta (B.1.617.2) neutralization. Notably, the novel antibody design disclosed herein neutralized BA.2 and BA.2.12.1 and was highly effective against BA.4, against which COV2-2130 suffered a small (<10-fold) reduction in potency, BA.5, and BA.5.5; and remained potently neutralizing against BA.2.75. 2130-1-0114-112 (LLNL199) was effective at neutralizing these VOCs

While COV2-2130 suffers total loss of pseudoviral neutralization against the BA.4.6 variant, which contains the mutation R346T, and a constructed BA.2.75 variant with the additional mutation R346T, matching the mutations in BA.2.76 RBD, 2130-1-0114-112 (also referred to herein as LLNL199) retains some pseudoviral neutralization against both (1264 and 674 ng/ml, respectively). These activities demonstrate that 2130-1-0114-112's (also referred to herein as LLNL199) can compensate for the loss of the salt bridge from RBD R346—to heavy chain D56, reducing this critical vulnerability as compared to COV2-2130.

The results described herein also show that improvements of more than one order of magnitude in neutralization IC₅₀ values with respect to Omicron BA.1 were possible with as few as two substitutions to the parental variable region sequence of COV2-2130, as in the similar 2130-1-0104-024 (also referred to herein as LLNL193), which has mutations SL32W, TL59E.

TABLE 1
Complementary Determining Regions
Table 1: Complementary Determining Regions

| Human ID | Mutations | CDRH1 | CDRH2 | CDRH3 | CDRL1 | CDRL2 | CDRL3 |
|---|---|---|---|---|---|---|---|
| mAb_RBD_ | IH55E, | gftfrdvw | ikskEdggtt | ttagsyyFdtvgpglpegkfdy | qsvlyWAnnkny | was | qqyystlt |
| 2130_FAB_ | YH106F, | | | | | | |
| 20211231_ | SL32W, | | | | | | |
| 00017 | SL33A | | | | | | |
| mAb_RBD_ | VH109R | gftfrdvw | ikskidggtt | ttagsyyydtRgpglpegkfdy | qsvlyssnnkny | was | qqyystlt |
| 2130_FAB_ | | | | | | | |
| 20211231_ | | | | | | | |
| 00174 | | | | | | | |
| mAb_RBD_ | IH55W | gftfrdvw | ikskWdggt | ttagsyyydtvgpglpegkfdy | qsvlyssnnkny | was | qqyystlt |
| 2130_FAB_ | | | t | | | | |
| 20211231_ | | | | | | | |
| 00194 | | | | | | | |
| mAb_RBD_ | SL33Y | gftfrdvw | ikskidggtt | ttagsyyydtvgpglpegkfdy | qsvlysYnnkny | was | qqyystlt |
| 2130_FAB_ | | | | | | | |
| 20211231_ | | | | | | | |
| 00199 | | | | | | | |
| mAb_RBD_ | SL33W | gftfrdvw | ikskidggtt | ttagsyyydtvgpglpegkfdy | qsvlysWnnkny | was | qqyystlt |
| 2130_FAB_ | | | | | | | |
| 20211231_ | | | | | | | |
| 00200 | | | | | | | |
| v2130_SFE_ | SL32Q, | gftfrdvw | ikskidggtt | ttagsyyydtvgpglpegkfdy | qsvlyQYnnkny | was | qqyystlt |
| 20220104_ | SL33Y, | | | | | | |
| 00015 | TL9H | | | | | | |
| v2130_SFE_ | SL32W, | gftfrdvw | ikskidggtt | ttagsyyydtvgpglpegkfdy | qsvlyWsnnkny | was | qqyystlt |
| 20220104_ | TL59E | | | | | | |
| 00024 | | | | | | | |
| mAb_v2130 | IH55D, | gftfrdvw | ikskDdggtt | ttagsyyydtvgpglpegkfdy | qsvlysFnnkny | was | qqyystlt |
| JustStableLines_ | SL33F, | | | | | | |
| 20220111_ | TL59E | | | | | | |
| 00002 | | | | | | | |
| mAb_v2130 | GH112E, | gftfrdvw | ikskidggtt | ttagsyyydtvgpElpegkfdy | qsvlyYVnnYny | was | qqyystlt |
| Vanderbilt_ | SL32Y, | | | | | | |
| 184_20220114_ | SL33V, | | | | | | |
| 00111 | KL36Y | | | | | | |
| mAb_v2130 | GH112E, | gftfrdvw | ikskidggtt | ttagsyyydtvgpElpegkfdy | qsvlyAAnnkny | was | qqyystlt |
| Vanderbilt_ | SL32A, | | | | | | |
| 184_20220114_ | SL33A, | | | | | | |
| 00112 | TL59E | | | | | | |
| v2130_upsample_ | YH106D, | gftfrdvw | ikskidggtt | ttagsyyDdtvgpglpegkldy | qsvlysVnnkny | was | qqyystlt |
| 20220121_ | FH1181, | | | | | | |
| 00009 | SL33V, | | | | | | |
| | YL55N | | | | | | |
| v2130_upsample_ | IH55E, | gftfrdvw | ikskEdggtt | ttagsyyHdtvgpglpegkfdy | qsvlyRsnnLny | was | qqyystlt |
| 20220121_ | YH106H, | | | | | | |
| 00298 | SL32R, | | | | | | |
| | KL36L | | | | | | |
| v2130_upsample_ | IH55H, | gftfrdvw | ikskHdggtt | ttagsyyydtvLpglpegkfdy | qsvlysYnnkny | was | qqyystlt |
| 20220121_ | GH110L, | | | | | | |
| 00314 | SL33Y, | | | | | | |
| | YL55E | | | | | | |
| v2130_upsample_ | VH109R, | gftfrdvw | ikskidggtt | ttagsyyydtRgpElpegkfdy | qsvlyAAnnkny | was | qqyystlt |
| 20220121_ | GH112E, | | | | | | |
| 00354 | SL32A, | | | | | | |
| | SL33A, | | | | | | |
| | TL59E | | | | | | |
| mAb_RBD_ | IH55E, | gftfrdvw | ikskEdggtt | ttagsyyFdtvgpglpegkfdy | qsvlyWAnnkny | ywastr | qqyystlt |
| 2130_FAB_ | YH106F, | | | | | | |
| 20211231_ | SL32W, | | | | | | |
| 00017 | SL33A | | | | | | |
| mAb_RBD_ | VH109R | gftfrdvw | ikskidggtt | ttagsyyydtRgpglpegkfdy | qsvlyssnnkny | ywastr | qqyystlt |
| 2130_FAB_ | | | | | | | |
| 20211231_ | | | | | | | |
| 00174 | | | | | | | |
| mAb_RBD_ | IH55W | gftfrdvw | ikskWdggt | ttagsyyydtvgpglpegkfdy | qsvlyssnnkny | ywastr | qqyystlt |
| 2130_FAB_ | | | t | | | | |
| 20211231_ | | | | | | | |
| 00194 | | | | | | | |
| mAb_RBD_ | SL33Y | gftfrdvw | ikskidggtt | ttagsyyydtvgpglpegkfdy | qsvlysYnnkny | ywastr | qqyystlt |
| 2130_FAB_ | | | | | | | |
| 20211231_ | | | | | | | |
| 00199 | | | | | | | |
| mAb_RBD_ | SL33W | gftfrdvw | ikskidggtt | ttagsyyydtvgpglpegkfdy | qsvlysWnnkny | ywastr | qqyystlt |
| 2130_FAB_ | | | | | | | |
| 20211231_ | | | | | | | |
| 00200 | | | | | | | |
| v2130_SFE_ | SL32Q, | gftfrdvw | ikskidggtt | ttagsyyydtvgpglpegkfdy | qsvlyQYnnkny | ywasHr | qqyystlt |
| 20220104_ | SL33Y, | | | | | | |
| 00015 | TL9H | | | | | | |
| v2130_SFE_ | SL32W, | gftfrdvw | ikskidggtt | ttagsyyydtvgpglpegkfdy | qsvlyWsnnkny | ywasEr | qqyystlt |
| 20220104_ | TL59E | | | | | | |
| 00024 | | | | | | | |
| mAb_v2130 | IH55D, | gftfrdvw | ikskDdggtt | ttagsyyydtvgpglpegkfdy | qsvlysFnnkny | ywasEr | qqyystlt |
| JustStableLines_ | SL33F, | | | | | | |
| 20220111_ | TL59E | | | | | | |
| 00002 | | | | | | | |
| mAb_v2130 | GH112E, | gftfrdvw | ikskidggtt | ttagsyyydtvgpElpegkfdy | qsvlyYVnnYny | ywastr | qqyystlt |
| Vanderbilt_ | SL32Y, | | | | | | |
| 184_20220114_ | SL33V, | | | | | | |
| 00111 | KL36Y | | | | | | |
| mAb_v2130 | GH112E, | gftfrdvw | ikskidggtt | ttagsyyydtvgpElpegkfdy | qsvlyAAnnkny | ywasEr | qqyystlt |
| Vanderbilt_ | SL32A, | | | | | | |
| 184_20220114_ | SL33A, | | | | | | |
| 00112 | TL59E | | | | | | |
| v2130_upsample_ | YH106D, | gftfrdvw | ikskidggtt | ttagsyyDdtvgpglpegkldy | qsvlysVnnkny | Nwastr | qqyystlt |
| 20220121_ | FH1181, | | | | | | |
| 00009 | SL33V, | | | | | | |
| | YL55N | | | | | | |
| v2130_upsample_ | IH55E, | gftfrdvw | ikskEdggtt | ttagsyyHdtvgpglpegkfdy | qsvlyRsnnLny | ywastr | qqyystlt |
| 20220121_ | YH106H, | | | | | | |
| 00298 | SL32R, | | | | | | |
| | KL36L | | | | | | |
| v2130_upsample_ | IH55H, | gftfrdvw | ikskHdggtt | ttagsyyydtvLpglpegkfdy | qsvlysYnnkny | Ewastr | qqyystlt |
| 20220121_ | GH110L, | | | | | | |
| 00314 | SL33Y, | | | | | | |
| | YL55E | | | | | | |
| v2130_upsample_ | VH109R, | gftfrdvw | ikskidggtt | ttagsyyydtRgpElpegkfdy | qsvlyAAnnkny | ywasEr | qqyystlt |
| 20220121_ | GH112E, | | | | | | |
| 00354 | SL32A, | | | | | | |
| | SL33A, | | | | | | |
| | TL59E | | | | | | |

TABLE 2
Amino acid sequences for the heavy chain (H) CDR1, CDR2, and CDR3 regions of the
anti-SARS_CoV_2 monoclonal antibodies
Table 2: Amino acid sequences for the heavy chain (H) CDRI, CDR2, and CDR3 regions
of the anti-SARS_CoV_2 monoclonal antibodies

| | | SEQ | | SEQ | | SEQ |
|---|---|---|---|---|---|---|
| | | ID | | ID | | ID |
| Human ID | CDRH1 | NO | CDRH2 | NO | CDRH3 | NO |
| FAB_00017 | gftfrdvw | 196 | ikskEdggtt | 197 | ttagsyyFdtvgpglpegkfdy | 202 |
| FAB_00174 | gftfrdvw | 196 | ikskidggtt | 198 | ttagsyyydtRgpglpegkfdy | 203 |
| FAB_00194 | gftfrdvw | 196 | ikskWdggtt | 199 | ttagsyyydtvgpglpegkfdy | 204 |
| FAB_00199 | gftfrdvw | 196 | ikskidggtt | 198 | ttagsyyydtvgpglpegkfdy | 204 |
| FAB_00200 | gftfrdvw | 196 | ikskidggtt | 198 | ttagsyyydtvgpglpegkfdy | 204 |
| SFE_00015 | gftfrdvw | 196 | ikskidggtt | 198 | ttagsyyydtvgpglpegkfdy | 204 |
| SFE_00024 | gftfrdvw | 196 | ikskidggtt | 198 | ttagsyyydtvgpglpegkfdy | 204 |
| JSL_00002 | gftfrdvw | 196 | ikskDdggtt | 200 | ttagsyyydtvgpglpegkfdy | 204 |
| Vander_00111 | gftfrdvw | 196 | ikskidggtt | 198 | ttagsyyydtvgpElpegkfdy | 205 |
| Vander_00112 | gftfrdvw | 196 | ikskidggtt | 198 | ttagsyyydtvgpElpegkfdy | 205 |
| Ups_00009 | gftfrdvw | 196 | ikskidggtt | 198 | ttagsyyDdtvgpglpegkIdy | 206 |
| Ups_00298 | gftfrdvw | 196 | ikskEdggtt | 197 | ttagsyyHdtvgpglpegkfdy | 207 |
| Ups_00314 | gftfrdvw | 196 | ikskHdggtt | 201 | ttagsyyydtvLpglpegkfdy | 208 |
| ups_00354 | gftfrdvw | 196 | ikskidggtt | 198 | ttagsyyydtRgpElpegkfdy | 209 |

TABLE 3
Amino acid sequences for the light chain (L) CDR1, CDR2, and CDR3 regions of the
novel anti-SARS_CoV_2 monoclonal antibodies
Table 3: Amino acid sequences for the light chain (L) CDR1, CDR2, and CDR3
regions of the novel anti-SARS_CoV_2 monoclonal antibodies

| | | SEQ | | SEQ | | |
|---|---|---|---|---|---|---|
| | | ID | | ID | | SEQ ID |
| Human ID | CDRL1 | NO | CDRL2 | NO | CDRL3 | NO |
| FAB_00017 | qsvlyWAnnkny | 226 | was | 73 | qqyystlt | 251 |
| FAB_00174 | qsvlyssnnkny | 227 | was | 73 | qqyystlt | 251 |
| FAB_00194 | qsvlyssnnkny | 227 | was | 73 | qqyystlt | 251 |
| FAB_00199 | qsvlysYnnkny | 228 | was | 73 | qqyystlt | 251 |
| FAB_00200 | qsvlysWnnkny | 229 | was | 73 | qqyystlt | 251 |
| SFE_00015 | qsvlyQYnnkny | 230 | was | 73 | qqyystlt | 251 |
| SFE_00024 | qsvlyWsnnkny | 231 | was | 73 | qqyystlt | 251 |
| JSL_00002 | qsvlysFnnkny | 232 | was | 73 | qqyystlt | 251 |
| Vandert_00111 | qsvlyYVnnYny | 233 | was | 73 | qqyystlt | 251 |
| Vander_00112 | qsvlyAAnnkny | 234 | was | 73 | qqyystlt | 251 |
| Ups_00009 | qsvlysVnnkny | 235 | was | 73 | qqyystlt | 251 |
| ups_00298 | qsvlyRsnnLny | 236 | was | 73 | qqyystlt | 251 |
| Ups_00314 | qsvlysYnnkny | 228 | was | 73 | qqyystlt | 251 |
| ups_00354 | qsvlyAAnnkny | 234 | was | 73 | qqyystlt | 251 |
| FAB_00017 | qsvlyWAnnkny | 226 | ywastr | 243 | qqyystlt | 251 |
| FAB_00174 | qsvlyssnnkny | 227 | ywastr | 243 | qqyystlt | 251 |
| FAB_00194 | qsvlyssnnkny | 227 | ywastr | 243 | qqyystlt | 251 |
| FAB_00199 | qsvlysYnnkny | 228 | ywastr | 243 | qqyystlt | 251 |
| FAB_00200 | qsvlysWnnkny | 229 | ywastr | 243 | qqyystlt | 251 |
| SFE_00015 | qsvlyQYnnkny | 230 | ywasHr | 244 | qqyystlt | 251 |
| SFE_00024 | qsvlyWsnnkny | 231 | ywasEr | 245 | qqyystlt | 251 |
| JSL_00002 | qsvlysFnnkny | 232 | ywasEr | 245 | qqyystlt | 251 |
| Vandert_00111 | qsvlyYVnnYny | 233 | ywastr | 243 | qqyystlt | 251 |
| Vander_00112 | qsvlyAAnnkny | 234 | ywasEr | 245 | qqyystlt | 251 |
| Ups_00009 | qsvlysVnnkny | 235 | Nwastr | 246 | qqyystlt | 251 |
| Ups_00298 | qsvlyRsnnLny | 236 | ywastr | 243 | qqyystlt | 251 |
| Ups_00314 | qsvlysYnnkny | 228 | Ewastr | 247 | qqyystlt | 251 |
| Ups_00354 | qsvlyAAnnkny | 234 | ywasEr | 245 | qqyystlt | 251 |

TABLE 4
Amino acid sequences of the novel anti-SARS_CoV_2 monoclonal antibodies
Table 4: Amino acid sequences of the novel anti-SARS_CoV_2 monoclonal antibodies

| SEQ | | |
|---|---|---|
| ID | | |
| NO: | DESCRIPTION | SEQUENCE |
| 1 | >md5_e3d1904966eaf73aed | EVQLVESGGGLVKPGGSLRLSCAASGFTFRDVW |
| | 30331f60b658e9 | MSWVRQAPGKGLEWVGRIKSKEDGGTTDYAAP |
| | Mutant_number: | VKGRFTISRDDSKNTLYLQMNSLKTEDTAVYYC |
| | md5_e3d1904966eaf73aed3 | TTAGSYYFDTVGPGLPEGKFDYWGQGTLVTVSS |
| | 0331f60b658e9 HumID: | DIVMTQSPDSLAVSLGERATINCKSSQSVLYWA |
| | mAb_RBD_2130_FAB_ | NNKNYLAWYQQKPGQPPKLLMYWASTRESGV |
| | 20211231_00017 Master: | PDRFSGSGSGAEFTLTISSLQAEDVAIYYCQQYY |
| | RBD_2130_FAB | STLTFGGGTKVEIKR |
| | Mutations: IH55E, | |
| | YH106F, SL32W, SL33A | |
| | scFv | |
| 100 | Heavy chain (V_{H}) | EVQLVESGGGLVKPGGSLRLSCAASGFTFRDVW |
| | | MSWVRQAPGKGLEWVGRIKSKEDGGTTDYAAP |
| | | VKGRFTISRDDSKNTLYLQMNSLKTEDTAVYYC |
| | | TTAGSYYFDTVGPGLPEGKFDYWGQGTLVTVSS |
| 196 | CDRH1 | GFTFRDVW |
| 197 | CDRH2 | IKSKEDGGTT |
| 202 | CDRH3 | TTAGSYYFDTVGPGLPEGKFDY |
| 155 | Light chain (V_{L}) | DIVMTQSPDSLAVSLGERATINCKSSQSVLYWA |
| | | NNKNYLAWYQQKPGQPPKLLMYWASTRESGV |
| | | PDRFSGSGSGAEFTLTISSLQAEDVAIYYCQQYY |
| | | STLTFGGGTKVEIKR |
| 226 | CDRL1 | QSVLYWANNKNY |
| 242 | CDRL2 | WAS |
| 243 | CDRL2 | YWASTR |
| 251 | CDRL3 | QQYYSTLT |
| 2 | >md5_d323d72bb6bc337ea | EVQLVESGGGLVKPGGSLRLSCAASGFTFRDVW |
| | 256bdd92ceba08e | MSWVRQAPGKGLEWVGRIKSKIDGGTTDYAAP |
| | Mutant_number: | VKGRFTISRDDSKNTLYLQMNSLKTEDTAVYYC |
| | md5_d323d72bb6bc337ea2 | TTAGSYYYDTRGPGLPEGKFDYWGQGTLVTVSS |
| | 56bdd92ceba08e HumID: | DIVMTQSPDSLAVSLGERATINCKSSQSVLYSSN |
| | mAb_RBD_2130_FAB_ | NKNYLAWYQQKPGQPPKLLMYWASTRESGVPD |
| | 20211231_00174 Master: | RFSGSGSGAEFTLTISSLQAEDVAIYYCQQYYSTL |
| | RBD_2130_FAB | TFGGGTKVEIKR |
| | Mutations: VH109R | |
| | scFv | |
| 101 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTRGPGLPEGKFDYWGQGTLVTVSS |
| 196 | CDRH1 | GFTFRDVW |
| 198 | CDRH2 | IKSKIDGGTT |
| 203 | CDRH3 | TTAGSYYYDTRGPGLPEGKFDY |
| 156 | Light chain (V_{L}) | DIVMTQSPDSLAVSLGERATINCKSSQSVLYSSN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 227 | CDRL1 | QSVLYSSNNKNY |
| 242 | CDRL2 | WAS |
| 243 | CDRL2 | YWASTR |
| 251 | CDRL3 | QQYYSTLT |
| 3 | >md5_9f6486808688a2b46 | EVQLVESGGGLVKPGGSLRLSCAASGFTFRDVW |
| | ec220af47cb95c5 | MSWVRQAPGKGLEWVGRIKSKWDGGTTDYAA |
| | Mutant_number: | PVKGRFTISRDDSKNTLYLQMNSLKTEDTAVYY |
| | md5_9f6486808688a2b46e | CTTAGSYYYDTVGPGLPEGKFDYWGQGTLVTV |
| | c220af47cb95c5 HumID: | SSDIVMTQSPDSLAVSLGERATINCKSSQSVLYS |
| | mAb_RBD_2130_FAB_ | SNNKNYLAWYQQKPGQPPKLLMYWASTRESG |
| | 20211231_00194 Master: | VPDRFSGSGSGAEFTLTISSLQAEDVAIYYCQQY |
| | RBD_2130_FAB | YSTLTFGGGTKVEIKR |
| | Mutations: IH55W | |
| | scFv | |
| 102 | Heavy chain (V_{H}) | EVQLVESGGGLVKPGGSLRLSCAASGFTFRDVW |
| | | MSWVRQAPGKGLEWVGRIKSKWDGGTTDYAA |
| | | PVKGRFTISRDDSKNTLYLQMNSLKTEDTAVYY |
| | | CTTAGSYYYDTVGPGLPEGKFDYWGQGTLVTVS |
| | | S |
| 196 | CDRH1 | GFTFRDVW |
| 199 | CDRH2 | IKSKWDGGTT |
| 204 | CDRH3 | TTAGSYYYDTVGPGLPEGKFDY |
| 156 | Light chain (V_{L}) | DIVMTQSPDSLAVSLGERATINCKSSQSVLYSSN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 227 | CDRL1 | QSVLYSSNNKNY |
| 242 | CDRL2 | WAS |
| 243 | CDRL2 | YWASTR |
| 251 | CDRL3 | QQYYSTLT |
| 4 | >md5_eebd2cc1786b72435 | EVQLVESGGGLVKPGGSLRLSCAASGFTFRDVW |
| | 8ced82fe314465c | MSWVRQAPGKGLEWVGRIKSKIDGGTTDYAAP |
| | Mutant_number: | VKGRFTISRDDSKNTLYLQMNSLKTEDTAVYYC |
| | md5_eebd2cc1786b724358 | TTAGSYYYDTVGPGLPEGKFDYWGQGTLVTVSS |
| | ced82fe314465c HumID: | DIVMTQSPDSLAVSLGERATINCKSSQSVLYSYN |
| | mAb_RBD_2130_FAB_ | NKNYLAWYQQKPGQPPKLLMYWASTRESGVPD |
| | 20211231_00199 Master: | RFSGSGSGAEFTLTISSLQAEDVAIYYCQQYYSTL |
| | RBD_2130_FAB | TFGGGTKVEIKR |
| | Mutations: SL33Y | |
| | scFv | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 157 | Light chain (V_{L}) | DIVMTQSPDSLAVSLGERATINCKSSQSVLYSYN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 228 | CDRL1 | QSVLYSYNNKNY |
| 242 | CDRL2 | WAS |
| 243 | CDRL2 | YWASTR |
| 251 | CDRL3 | QQYYSTLT |
| 5 | >md5_aaca5fa01a0f27e83e | EVQLVESGGGLVKPGGSLRLSCAASGFTFRDVW |
| | 39da01a4457347 | MSWVRQAPGKGLEWVGRIKSKIDGGTTDYAAP |
| | Mutant_number: | VKGRFTISRDDSKNTLYLQMNSLKTEDTAVYYC |
| | md5_aaca5fa01a0f27e83e3 | TTAGSYYYDTVGPGLPEGKFDYWGQGTLVTVSS |
| | 9da01a4457347 HumID: | DIVMTQSPDSLAVSLGERATINCKSSQSVLYSWN |
| | mAb_RBD_2130_FAB_ | NKNYLAWYQQKPGQPPKLLMYWASTRESGVPD |
| | 20211231_00200 Master: | RFSGSGSGAEFTLTISSLQAEDVAIYYCQQYYSTL |
| | RBD_2130_FAB | TFGGGTKVEIKR |
| | Mutations: SL33W | |
| | scFv | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 158 | Light chain (V_{L}) | DIVMTQSPDSLAVSLGERATINCKSSQSVLYSWN |
| | | NKNYLAWYQQKPGQPPKLLMYWASTRESGVPD |
| | | RFSGSGSGAEFTLTISSLQAEDVAIYYCQQYYSTL |
| | | TFGGGTKVEIKR |
| 229 | CDRL1 | QSVLYSWNNKNY |
| 242 | CDRL2 | WAS |
| 243 | CDRL2 | YWASTR |
| 251 | CDRL3 | QQYYSTLT |
| 6 | >md5_2041aa110908c8cde | EVQLVESGGGLVKPGGSLRLSCAASGFTFRDVW |
| | 47ed7aabc074be8 | MSWVRQAPGKGLEWVGRIKSKIDGGTTDYAAP |
| | Mutant_number: | VKGRFTISRDDSKNTLYLQMNSLKTEDTAVYYC |
| | md5_2041aa110908c8cde4 | TTAGSYYYDTVGPGLPEGKFDYWGQGTLVTVSS |
| | 7ed7aabc074be8 HumID: | DIVMTQSPDSLAVSLGERATINCKSSQSVLYQYN |
| | v2130_SFE_20220104_ | NKNYLAWYQQKPGQPPKLLMYWASHRESGVPD |
| | 00015 Master: | RFSGSGSGAEFTLTISSLQAEDVAIYYCQQYYSTL |
| | RBD_2130_FAB | TFGGGTKVEIKR |
| | Mutations: SL32Q, SL33Y, | |
| | TL59H | |
| | scFv | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 159 | Light chain (V_{L}) | DIVMTQSPDSLAVSLGERATINCKSSQSVLYQYN |
| | | NKNYLAWYQQKPGQPPKLLMYWASHRESGVPD |
| | | RFSGSGSGAEFTLTISSLQAEDVAIYYCQQYYSTL |
| | | TFGGGTKVEIKR |
| 230 | CDRL1 | QSVLYQYNNKNY |
| 242 | CDRL2 | WAS |
| 244 | CDRL2 | YWASHR |
| 251 | CDRL3 | QQYYSTLT |
| 7 | >md5_1089bd14be7c511aa | EVQLVESGGGLVKPGGSLRLSCAASGFTFRDVW |
| | 67dbef73eeb1e42 | MSWVRQAPGKGLEWVGRIKSKIDGGTTDYAAP |
| | Mutant_number: | VKGRFTISRDDSKNTLYLQMNSLKTEDTAVYYC |
| | md5_1089bd14be7c511aa6 | TTAGSYYYDTVGPGLPEGKFDYWGQGTLVTVSS |
| | 7dbef73eeb1e42 HumID: | DIVMTQSPDSLAVSLGERATINCKSSQSVLYWSN |
| | v2130_SFE_20220104_ | NKNYLAWYQQKPGQPPKLLMYWASERESGVPD |
| | 00024 Master: | RFSGSGSGAEFTLTISSLQAEDVAIYYCQQYYSTL |
| | RBD_2130_FAB | TFGGGTKVEIKR |
| | Mutations: SL32W, TL59E | |
| | scFv | |
| 103 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 198 | CDRH2 | IKSKIDGGTT |
| 204 | CDRH3 | TTAGSYYYDTVGPGLPEGKFDY |
| 160 | Light chain (V_{L}) | DIVMTQSPDSLAVSLGERATINCKSSQSVLYWSN |
| | | NKNYLAWYQQKPGQPPKLLMYWASERESGVPD |
| | | RFSGSGSGAEFTLTISSLQAEDVAIYYCQQYYSTL |
| | | TFGGGTKVEIKR |
| 231 | CDRL1 | QSVLYWSNNKNY |
| 242 | CDRL2 | WAS |
| 245 | CDRL2 | YWASER |
| 251 | CDRL3 | QQYYSTLT |
| 8 | >md5_689536fbc67d8ee33 | EVQLVESGGGLVKPGGSLRLSCAASGFTFRDVW |
| | e80ca5a6709d167 | MSWVRQAPGKGLEWVGRIKSKDDGGTTDYAAP |
| | Mutant_number: | VKGRFTISRDDSKNTLYLQMNSLKTEDTAVYYC |
| | md5_689536fbc67d8ee33e8 | TTAGSYYYDTVGPGLPEGKFDYWGQGTLVTVSS |
| | 0ca5a6709d167 HumID: | DIVMTQSPDSLAVSLGERATINCKSSQSVLYSFN |
| | mAb_v2130JustStableLines_ | NKNYLAWYQQKPGQPPKLLMYWASERESGVPD |
| | 20220111_00002 Master: | RFSGSGSGAEFTLTISSLQAEDVAIYYCQQYYSTL |
| | RBD_2130_FAB | TFGGGTKVEIKR |
| | Mutations: IH55D, SL33F, | |
| | TL59E | |
| | scFv | |
| 104 | Heavy chain (V_{H}) | EVQLVESGGGLVKPGGSLRLSCAASGFTFRDVW |
| | | MSWVRQAPGKGLEWVGRIKSKDDGGTTDYAAP |
| | | VKGRFTISRDDSKNTLYLQMNSLKTEDTAVYYC |
| | | TTAGSYYYDTVGPGLPEGKFDYWGQGTLVTVSS |
| 196 | CDRH1 | GFTFRDVW |
| 200 | CDRH2 | IKSKDDGGTT |
| 204 | CDRH3 | TTAGSYYYDTVGPGLPEGKFDY |
| 161 | Light chain (V_{L}) | DIVMTQSPDSLAVSLGERATINCKSSQSVLYSFN |
| | | NKNYLAWYQQKPGQPPKLLMYWASERESGVPD |
| | | RFSGSGSGAEFTLTISSLQAEDVAIYYCQQYYSTL |
| | | TFGGGTKVEIKR |
| 232 | CDRL1 | QSVLYSFNNKNY |
| 242 | CDRL2 | WAS |
| 245 | CDRL2 | YWASER |
| 251 | CDRL3 | QQYYSTLT |
| 9 | >md5_a314041f0f349a3dc6 | EVQLVESGGGLVKPGGSLRLSCAASGFTFRDVW |
| | b474c069163d9b | MSWVRQAPGKGLEWVGRIKSKIDGGTTDYAAP |
| | Mutant_number: | VKGRFTISRDDSKNTLYLQMNSLKTEDTAVYYC |
| | md5_a314041f0f349a3dc6b | TTAGSYYYDTVGPELPEGKFDYWGQGTLVTVSS |
| | 474c069163d9b HumID: | DIVMTQSPDSLAVSLGERATINCKSSQSVLYYVN |
| | mAb_v2130Vanderbilt_184_ | NYNYLAWYQQKPGQPPKLLMYWASTRESGVPD |
| | 20220114_00111 Master: | RFSGSGSGAEFTLTISSLQAEDVAIYYCQQYYSTL |
| | RBD_2130_FAB | TFGGGTKVEIKR |
| | Mutations: GH112E, | |
| | SL32Y, SL33V, KL36Y | |
| | scFv | |
| 105 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPELPEGKFDYWGQGTLVTVSS |
| 196 | CDRH1 | GFTFRDVW |
| 198 | CDRH2 | IKSKIDGGTT |
| 205 | CDRH3 | TTAGSYYYDTVGPELPEGKFDY |
| 162 | Light chain (V_{L}) | DIVMTQSPDSLAVSLGERATINCKSSQSVLYYVN |
| | | NYNYLAWYQQKPGQPPKLLMYWASTRESGVPD |
| | | RFSGSGSGAEFTLTISSLQAEDVAIYYCQQYYSTL |
| | | TFGGGTKVEIKR |
| 233 | CDRL1 | QSVLYYVNNYNY |
| 242 | CDRL2 | WAS |
| 243 | CDRL2 | YWASTR |
| 251 | CDRL3 | QQYYSTLT |
| 10 | >md5_3052c528d578179c0 | EVQLVESGGGLVKPGGSLRLSCAASGFTFRDVW |
| | 0211647380e5fb7 | MSWVRQAPGKGLEWVGRIKSKIDGGTTDYAAP |
| | Mutant_number: | VKGRFTISRDDSKNTLYLQMNSLKTEDTAVYYC |
| | md5_3052c528d578179c00 | TTAGSYYYDTVGPELPEGKFDYWGQGTLVTVSS |
| | 211647380e5fb7 HumID: | DIVMTQSPDSLAVSLGERATINCKSSQSVLYAAN |
| | mAb_v2130Vanderbilt_184_ | NKNYLAWYQQKPGQPPKLLMYWASERESGVPD |
| | 20220114_00112 Master: | RFSGSGSGAEFTLTISSLQAEDVAIYYCQQYYSTL |
| | RBD_2130_FAB | TFGGGTKVEIKR |
| | Mutations: GH112E, | |
| | SL32A, SL33A, TL59E | |
| | scFv | |
| 105 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTVGPELPEGKFDYWGQGTLVTVS |
| | | S |
| 196 | CDRH1 | GFTFRDVW |
| 198 | CDRH2 | IKSKIDGGTT |
| 205 | CDRH3 | TTAGSYYYDTVGPELPEGKFDY |
| 163 | Light chain (V_{L}) | DIVMTQSPDSLAVSLGERATINCKSSQSVLYAAN |
| | | NKNYLAWYQQKPGQPPKLLMYWASERESGVP |
| | | DRFSGSGSGAEFTLTISSLQAEDVAIYYCQQYYS |
| | | TLTFGGGTKVEIKR |
| 234 | CDRL1 | QSVLYAANNKNY |
| 242 | CDRL2 | WAS |
| 245 | CDRL2 | YWASER |
| 251 | CDRL3 | QQYYSTLT |
| 11 | >md5_2cdc3bd28ff984b7c5 | EVQLVESGGGLVKPGGSLRLSCAASGFTFRDVW |
| | b8d2a86ae9553f | MSWVRQAPGKGLEWVGRIKSKIDGGTTDYAAP |
| | Mutant_number: | VKGRFTISRDDSKNTLYLQMNSLKTEDTAVYYC |
| | md5_2cdc3bd28ff984b7c5b | TTAGSYYDDTVGPGLPEGKIDYWGQGTLVTVSS |
| | 8d2a86ae9553f HumID: | DIVMTQSPDSLAVSLGERATINCKSSQSVLYSVN |
| | v2130_upsample_20220121_ | NKNYLAWYQQKPGQPPKLLMNWASTRESGVPD |
| | 00009 Master: | RFSGSGSGAEFTLTISSLQAEDVAIYYCQQYYSTL |
| | RBD_2130_FAB | TFGGGTKVEIKR |
| | Mutations: YH106D, | |
| | FH118I, SL33V, YL55N | |
| | scFv | |
| 106 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYDDTVGPGLPEGKIDYWGQGTLVTVSS |
| 196 | CDRH1 | GFTFRDVW |
| 198 | CDRH2 | IKSKIDGGTT |
| 206 | CDRH3 | TTAGSYYDDTVGPGLPEGKIDY |
| 164 | Light chain (V_{L}) | DIVMTQSPDSLAVSLGERATINCKSSQSVLYSVN |
| | | NKNYLAWYQQKPGQPPKLLMNWASTRESGVP |
| | | DRFSGSGSGAEFTLTISSLQAEDVAIYYCQQYYS |
| | | TLTFGGGTKVEIKR |
| 235 | CDRL1 | QSVLYSVNNKNY |
| 242 | CDRL2 | WAS |
| 246 | CDRL2 | NWASTR |
| 251 | CDRL3 | QQYYSTLT |
| 12 | >md5_6e9c26854d3e83e74 | EVQLVESGGGLVKPGGSLRLSCAASGFTFRDVW |
| | f379947355e3c6a | MSWVRQAPGKGLEWVGRIKSKEDGGTTDYAAP |
| | Mutant_number: | VKGRFTISRDDSKNTLYLQMNSLKTEDTAVYYC |
| | md5_6e9c26854d3e83e74f3 | TTAGSYYHDTVGPGLPEGKFDYWGQGTLVTVSS |
| | 79947355e3c6a HumID: | DIVMTQSPDSLAVSLGERATINCKSSQSVLYRSN |
| | v2130_upsample_20220121 | NLNYLAWYQQKPGQPPKLLMYWASTRESGVPD |
| | 00298 Master: | RFSGSGSGAEFTLTISSLQAEDVAIYYCQQYYSTL |
| | RBD_2130_FAB_ | TFGGGTKVEIKR |
| | Mutations: IH55E, | |
| | YH106H, SL32R, KL36L | |
| | scFv | |
| 107 | Heavy chain (V_{H}) | EVQLVESGGGLVKPGGSLRLSCAASGFTFRDVW |
| | | MSWVRQAPGKGLEWVGRIKSKEDGGTTDYAAP |
| | | VKGRFTISRDDSKNTLYLQMNSLKTEDTAVYYC |
| | | TTAGSYYHDTVGPGLPEGKFDYWGQGTLVTVS |
| | | S |
| 196 | CDRH1 | GFTFRDVW |
| 197 | CDRH2 | IKSKEDGGTT |
| 207 | CDRH3 | TTAGSYYHDTVGPGLPEGKFDY |
| 165 | Light chain (V_{L}) | DIVMTQSPDSLAVSLGERATINCKSSQSVLYRSN |
| | | NLNYLAWYQQKPGQPPKLLMYWASTRESGVPD |
| | | RFSGSGSGAEFTLTISSLQAEDVAIYYCQQYYST |
| | | LTFGGGTKVEIKR |
| 236 | CDRL1 | QSVLYRSNNLNY |
| 242 | CDRL2 | WAS |
| 243 | CDRL2 | YWASTR |
| 251 | CDRL3 | QQYYSTLT |
| 13 | >md5_8813a032f188710d1 | EVQLVESGGGLVKPGGSLRLSCAASGFTFRDVW |
| | acf700cb2d61380 | MSWVRQAPGKGLEWVGRIKSKHDGGTTDYAAP |
| | Mutant_number: | VKGRFTISRDDSKNTLYLQMNSLKTEDTAVYYC |
| | md5_8813a032f188710d1a | TTAGSYYYDTVLPGLPEGKFDYWGQGTLVTVSS |
| | cf700cb2d61380 HumID: | DIVMTQSPDSLAVSLGERATINCKSSQSVLYSYN |
| | v2130_upsample_20220121_ | NKNYLAWYQQKPGQPPKLLMEWASTRESGVPD |
| | 00314 Master: | RFSGSGSGAEFTLTISSLQAEDVAIYYCQQYYSTL |
| | RBD_2130_FAB | TFGGGTKVEIKR |
| | Mutations: IH55H, | |
| | GH110L, SL33Y, YL55E | |
| | scFv | |
| 108 | Heavy chain (V_{H}) | EVQLVESGGGLVKPGGSLRLSCAASGFTFRDVW |
| | | MSWVRQAPGKGLEWVGRIKSKHDGGTTDYAA |
| | | PVKGRFTISRDDSKNTLYLQMNSLKTEDTAVYY |
| | | CTTAGSYYYDTVLPGLPEGKFDYWGQGTLVTV |
| | | SS |
| 196 | CDRH1 | GFTFRDVW |
| 201 | CDRH2 | IKSKHDGGTT |
| 208 | CDRH3 | TTAGSYYYDTVLPGLPEGKFDY |
| 166 | Light chain (V_{L}) | DIVMTQSPDSLAVSLGERATINCKSSQSVLYSYN |
| | | NKNYLAWYQQKPGQPPKLLMEWASTRESGVPD |
| | | RFSGSGSGAEFTLTISSLQAEDVAIYYCQQYYST |
| | | LTFGGGTKVEIKR |
| 228 | CDRL1 | QSVLYSYNNKNY |
| 242 | CDRL2 | WAS |
| 247 | CDRL2 | EWASTR |
| 251 | CDRL3 | QQYYSTLT |
| 14 | >md5_6362c40ele8234bda | EVQLVESGGGLVKPGGSLRLSCAASGFTFRDVW |
| | 1f78cd76d72ca17 | MSWVRQAPGKGLEWVGRIKSKIDGGTTDYAAP |
| | Mutant_number: | VKGRFTISRDDSKNTLYLQMNSLKTEDTAVYYC |
| | md5_6362c40ele8234bda1f | TTAGSYYYDTRGPELPEGKFDYWGQGTLVTVSS |
| | 78cd76d72ca17 HumID: | DIVMTQSPDSLAVSLGERATINCKSSQSVLYAAN |
| | v2130_upsample_20220121_ | NKNYLAWYQQKPGQPPKLLMYWASERESGVPD |
| | 00354 Master: | RFSGSGSGAEFTLTISSLQAEDVAIYYCQQYYSTL |
| | RBD_2130_FAB | TFGGGTKVEIKR |
| | Mutations: VH109R, | |
| | GH112E, SL32A, SL33A, | |
| | TL59E | |
| | scFv | |
| 109 | Heavy chain (V_{H}) | EVQLVESGGGLVKPGGSLRLSCAASGFTFRDVW |
| | | MSWVRQAPGKGLEWVGRIKSKIDGGTTDYAAP |
| | | VKGRFTISRDDSKNTLYLQMNSLKTEDTAVYYC |
| | | TTAGSYYYDTRGPELPEGKFDYWGQGTLVTVSS |
| 196 | CDRH1 | GFTFRDVW |
| 198 | CDRH2 | IKSKIDGGTT |
| 209 | CDRH3 | TTAGSYYYDTRGPELPEGKFDY |
| 163 | Light chain (V_{L}) | DIVMTQSPDSLAVSLGERATINCKSSQSVLYAAN |
| | | NKNYLAWYQQKPGQPPKLLMYWASERESGVP |
| | | DRFSGSGSGAEFTLTISSLQAEDVAIYYCQQYYS |
| | | TLTFGGGTKVEIKR |
| 234 | CDRL1 | QSVLYAANNKNY |
| 242 | CDRL2 | WAS |
| 245 | CDRL2 | YWASER |
| 251 | CDRL3 | QQYYSTLT |

TABLE 6
Thermal stability and expression yield of selected IgGs. Melting
temperature (Tm) was determined using a fluorescence-based protein
thermal shift assay (GloMeltTM, Biotium). Yield was determined
by measuring optical density at 280 nm and deriving antibody
quantity using the calculated extinction coefficient.

| | Tm-Boltzmann (C.; | Yield (mg) per |
|---|---|---|
| mAb | mean +/− std) | 100 mL culture |
| 2130- wt | 68.54 ± 0.27 | 28.31 |
| 2130-1-0111-002 | 68.41 ± 0.06 | 31.64 |
| 2130-1-0121-009 #2 | 66.29 ± 0.20 | 0.17 (yield when |
| | | repeated.: 26.07) |
| 2130-1-0104-015 | 68.35 ± 0.22 | 27.77 |
| 2130-1-1231-017 | 68.52 ± 0.42 | 19.34 |
| 2130-1-0104-024 | 67.81 ± 0.35 | 26.93 |
| 2130-1-0114-111 | 65.53 ± 0.25 | 26.54 |
| 2130-1-0114-112 | 68.23 ± 0.26 | 48.12 |
| 2130-1-1231-174 | 69.02 ± 0.33 | 32.63 |
| 2130-1-1231-200 | 68.07 ± 0.27 | 47.72 |
| 2130-1-0121-354 | 69.82 ± 0.24 | 29.46 |

TABLE 7
Parameters of CryoEM procedures.

| | | Cov2 BA.2 spike | Cov2 BA.2 RBD |
|---|---|---|---|
| | | 2130-1-0114-112 | 2130-1-0114-112* |
| Data | EMDB | EMD-28198 | EMD-28199 |
| Deposition | PDB | — | 8EDK |
| Microscope | Microscope | Krios | Krios |
| setting | Voltage (kV) | 300 | 300 |
| | Detector | K3 | K3 |
| | Mag | X130,000 | X130,000 |
| | Pixel size | 0.647 | 0.647 |
| | Exposure (e−/Å2) | 52.2 | 52.2 |
| | Defocus range | 0.8-1.8 | 0.8-1.8 |
| | (μm) | | |
| Data | # Micrographs | 18789 | 18789 |
| | # particles | 1290705 | |
| | # particle after | 844554 | |
| | 2D | | |
| | Final particles # | 291461 | 386950 |
| | Symmetry | C3 | C1 |
| | Resolution | 3.26 | 3.6 |
| | FSC = 0.143 | | |
| Model | Protein residues | | 441 |
| refinement | Map CC | | 0.63 |
| and | RMSD | | |
| validation | Bond lengths (Å) | | 0.004 |
| | Bond angles | | 0.738 |
| | Ramachandran | | |
| | Outliers (%) | | |
| | Allowed (%) | | 6.02 |
| | Favored (%) | | 93.98 |
| | Poor rotamers | | 0.88 |
| | (%) | | |
| | MolProbity score | | 2.17 |
| | Clash score | | 18.79 |

*Focus refinement

TABLE 8
Thermal Shift (Performed at LLNL)(Empty cells not tested)

| | Tm-Boltzmann (C.; | Yield (mg) per |
|---|---|---|
| mAb | mean +/− std) | 100 mL culture |
| S309 | | |
| 2130- wt | 68.54 ± 0.27 | 28.31 |
| 2130-1-0111-002 | 68.41 ± 0.06 | 31.64 |
| 2130-1-0121-009 #2 | 66.29 ± 0.20 | 0.17 (rep.: 26.07) |
| 2130-1-0104-015 | 68.35 ± 0.22 | 27.77 |
| 2130-1-1231-017 | 68.52 ± 0.42 | 19.34 |
| 2130-1-0104-024 | 67.81 ± 0.35 | 26.93 |
| 2130-1-0114-111 | 65.53 ± 0.25 | 26.54 |
| 2130-1-0114-112 | 68.23 ± 0.26 | 48.12 |
| 2130-1-1231-174 | 69.02 ± 0.33 | 32.63 |
| 2130-1-1231-200 | 68.07 ± 0.27 | 47.72 |
| 2130-1-0121-354 | 69.82 ± 0.24 | 29.46 |
| 2130-1-0121_314 | | |
| 2130-1-0121_298 | | |
| 2130-1-1231-199 | | |
| 2130-1-1231_194 | | |

TABLE 9
Pseudovirus neutralization (IC₅₀)

| | | NewStyleID | | | | | | |
|---|---|---|---|---|---|---|---|---|
| | | 2130-1- | 2130-1- | 2130-1- | 2130-1- | 2130-1- | 2130-1- | 2130-1- |
| | | 1231- | 1231- | 1231- | 1231- | 1231- | 0104- | 1231- |
| | | 174 | 185 | 194 | 199 | 200 | 015 | 017 |
| | | OldStyleID | | | | | | |
| | | mAb_RBD_ | mAb_RBD_ | mAb_RBD_ | | | v2130_ | |
| | | 2130_FAB_ | 2130_FAB_ | 2130_FAB_ | | | SFE_ | |
| | | 20211231_ | 20211231_ | 20211231_ | | | 20220104_ | |
| | | 00174 | 00185 | 00194 | LLNL175 | LLNL174 | 00015 | LLNL171 |
| IC50 (ng/ml) | BA.1 | 9.406E+53 | Unstable | 909 | 555.9 | 489.8 | 530.2 | 136.1 |
| | BA.1.1 | Unstable | 12687 | Unstable | NC | Unstable | NC | 1.858E+24 |
| | BA.2 | Unstable | Malformed- | 2.612 | 1.107 | 1.303 | 0.5927 | 0.4236 |
| | | | OrS [symbol] | | | | | |
| | | NewStyleID | | | | | | |
| | | 2130-1- | 2130-1- | | 2130-1- | 2130-1- | 2130-1- | 2130- |
| | | 0104- | 0104- | | 0121- | 0121- | 0121- | 1-0121- |
| | | 015 | 024 | | 354 | 009 | 314 | 298 |
| | | OldStyleID | | | | | | |
| | | V2130_ | | | | | | |
| | | SFE_ | | | | | | |
| | | 20220104_ | | | | | | |
| | | 00015 | LLNL193 | 2130_wt | B-133 | B-135 | B-178 | B-182 |
| IC50 (ng/ml) | BA.1 | 429 | 10.34 | 4.743 | 0.1778 | NC | Malformed- | 625.6 |
| | | | | | | | OrS [symbol] | |
| | BA.1.1 | 41.79 | 10293 | 8.074 | 0.4753 | Unstable | Unstable | Unstable |
| | BA.2 | 1.299 | 0.7141 | 0.245 | 0.5151 | Malformed- | Malformed- | 566 |
| | | | | | | OrS [symbol] | OrS [symbol] | |
| | | NewStyleID | | | | | | |
| | | | | 2130- | 2130- | 2130- | | |
| | | | | 1-0111- | 1-0114- | 1-0114- | | |
| | | | | 002 | 111 | 112 | | |
| | | OldStyleID | | | | | | |
| | | | | LLNL10 | LLNL198 | LLNL199 | S309 | 2D22 |
| | | IC50 (ng/ml) | BA.1 | 339.1 | 117.7 | 1.142 | 63.22 | Unstable |
| | | | BA.1.1 | NC | 602.8 | 3.208 | 33.16 | Unstable |
| | | | BA.2 | 1.147 | 0.03916 | 0.8522 | 109 | Malformed- |
| | | | | | | | | OrSuspect |

[symbol]

indicates data missing or illegible when filed

TABLE 10
Authentic Virus Neutralization

| IC50 (μg/ml) | B.1.617.2 | BA.1 | BA.1.1 | BA.4 | BA.5.5 |
|---|---|---|---|---|---|
| 2130-1- | 0.006232 | 0.00423 | 0.009648 | 0.003045 | 0.002816 |
| 0121-354 | | | | | |
| 2130-1- | 0.03394 | 0.005534 | 0.03921 | 0.007474 | 0.005287 |
| 0114-112 | | | | | |
| 2130 WT | 0.2825 | > | > | 0.3912 | 0.5221 |
| rDENV- | > | > | > | > | > |
| 2D22 | | | | | |

> indicates greater than 10 μg/ml

TABLE 11
Normalized pseudoneutralization of five antibodies vs. five
SARS-COV-2 strains (“>” indicates a value >10,000
ng/mL; NC indicates positive hill slope or failure to converge):

| IC50 (ng/mL) | D614G | BA.1 | BA.1.1 | BA.2 | BA.4 |
|---|---|---|---|---|---|
| rDENV-2D22 | NC | NC | > | NC | > |
| rS2K146 | 6.562 | 4.375 | 2.092 | 3.358 | 36.01 |
| rCOV2-2130 | 3.595 | 157.5 | NC | 2.525 | 64.31 |
| 2130-1-0111-002 | 4.92 | 34.8 | > | 1.47 | 4.689 |
| 2130-1-1231-017 | 2.43 | 61.26 | > | 0.5686 | 17.06 |
| 2130-1-0104-024 | 2.986 | 13.25 | 78.46 | 0.2201 | 2.267 |
| 2130-1-0114-111 | 3.023 | 32 | 4027 | 0.1616 | 34.27 |
| 2130-1-0114-112 | 4.081 | 2.56 | 6.249 | 0.9845 | 0.7787 |

TABLE 12
Antibody Nomenclature Legend

| | | | | MMDD_ | | | NewFormat |
|---|---|---|---|---|---|---|---|
| ShortID | Mutations | Antibody Hash ID | AntibodyHumID | nnNNN | MMDD | NNN | ID |
| LLNL010 | IH55D, | md5_689536fbc67d | mAb_ | 0111_ | 0111 | 002 | 2130-1- |
| | SL33F, | 8ce33e80ca5a6709d | v2130JustStableLines_ | 00002 | | | 0111-002 |
| | TL59E | 167 | 20220111_00002 | | | | |
| LLNL108 | SL32Q, | md5_2041aa110908 | v2130_SFE_20220104_ | 0104_ | 0104 | 015 | 2130-1- |
| | SL33Y, | c8cde47ed7aabc074 | 00015 | 00015 | | | 0104-015 |
| | TL59H | be8 | | | | | |
| LLNL171 | IH55E, | md5_e3d1904966ea | mAb_RBD_2130_ | 1231_ | 1231 | 017 | 2130-1- |
| | YH106F, | f73aed30331f60b65 | FAB_20211231_00017 | 00017 | | | 1231-017 |
| | SL32W, | 8e9 | | | | | |
| | SL33A | | | | | | |
| LLNL174 | SL33W | md5_aaca5fa01a0f2 | mAb_RBD_2130_ | 1231_ | 1231 | 200 | 2130-1- |
| | | 7e83e39da01a44573 | FAB_20211231_00200 | 00200 | | | 1231-200 |
| | | 47 | | | | | |
| LLNL175 | SL33Y | md5_eebd2cc1786b | mAb_RBD__2130_ | 1231_ | 1231 | 199 | 2130-1- |
| | | 724358ced82fe3144 | FAB_20211231_00199 | 00199 | | | 1231-199 |
| | | 65c | | | | | |
| LLNL193 | SL32W, | md5_1089bd14be7c | v2130_SFE_20220104_ | 0104_ | 0104 | 024 | 2130-1- |
| | TL59E | 511aa67dbef73eeb1 | 00024 | 00024 | | | 0104-024 |
| | | e42 | | | | | |
| LLNL198 | GH112E, | md5_a314041f0f34 | mAb_v2130Vanderbilt_ | 0114_ | 0114 | 111 | 2130-1- |
| | SL32Y, | 9a3dc6b474c069163 | 184_20220114_00111 | 00111 | | | 0114-111 |
| | SL33V, | d9b | | | | | |
| | KL36Y | | | | | | |
| LLNL199 | GH112E, | md5_3052c528d578 | mAb_v2130Vanderbilt_ | 0114_ | 0114 | 112 | 2130-1- |
| | SL32A, | 179c00211647380e | 184_20220114_00112 | 00112 | | | 0114-112 |
| | SL33A, | 5fb7 | | | | | |
| | TL59E | | | | | | |
| B-133 | VH109R, | md5_6362c40e1e82 | v2130_upsample_ | 0121_ | 0121 | 354 | 2130-1- |
| | GH112E, | 34bda1f78cd76d72c | 20220121_00354 | 00354 | | | 0121-354 |
| | SL32A, | a17 | | | | | |
| | SL33A, | | | | | | |
| | TL59E | | | | | | |
| B-135 | YH106D, | md5_2cdc3bd28ff98 | v2130_upsample_ | 0121_ | 0121 | 009 | 2130-1- |
| | FH118I, | 4b7c5b8d2a86ae955 | 20220121_00009 | 00009 | | | 0121-009 |
| | SL33V, | 3f | | | | | |
| | YL55N | | | | | | |
| B-178 | IH55H, | md5_8813a032f188 | v2130_upsample_ | 0121_ | 0121 | 314 | 2130-1- |
| | GH110L, | 710d1acf700cb2d61 | 20220121_00314 | 00314 | | | 0121-314 |
| | SL33Y, | 380 | | | | | |
| | YL55E | | | | | | |
| B-182 | IH55E, | md5_6e9c26854d3e | v2130_upsample_ | 0121_ | 0121 | 298 | 2130-1- |
| | YH106H, | 83e74f379947355e3 | 20220121_00298 | 00298 | | | 0121-298 |
| | SL32R, | c6a | | | | | |
| | KL36L | | | | | | |
| | VH109R | md5_d323d72bb6bc | mAb_RBD_2130_ | 1231_ | 1231 | 174 | 2130-1- |
| | | 337ea256bdd92ceba | FAB_20211231_00174 | 00174 | | | 1231-174 |
| | | 08e | | | | | |
| | IH55W | md5_9f6486808688 | mAb_RBD_2130_ | 1231_ | 1231 | 194 | 2130-1- |
| | | a2b46ec220af47cb9 | FAB_20211231_00194 | 00194 | | | 1231-194 |
| | | 5c5 | | | | | |

Table 13: SEQ ID NOs for Full Abs and Corresponding CDRS

| | | | SEQ ID NO | | | | | | | |
|---|---|---|---|---|---|---|---|---|---|---|
| | | | VH + VL | | | | | | | |
| HumID | HashID | Mutations | Seq | H1 | H2 | H3 | L1 | L2 | L2ext | L3 |
| mAb_RBD_2130_ | md5_e3d1904966 | IH55E, | 1 | 196 | 197 | 202 | 226 | 242 | 243 | 251 |
| FAB_20211231_ | eaf73aed30331f | YH106F, | | | | | | | | |
| 00017 | 60b658e9 | SL32W, | | | | | | | | |
| | | SL33A | | | | | | | | |
| mAb_RBD_ | md5_d323d72bb6 | VH109R | 2 | 196 | 198 | 203 | 227 | 242 | 243 | 251 |
| 2130_FAB_ | bc337ea256bdd9 | | | | | | | | | |
| 20211231_00174 | 2ceba08e | | | | | | | | | |
| mAb_RBD_ | md5_9f64868086 | IH55W | 3 | 196 | 199 | 204 | 227 | 242 | 243 | 251 |
| 2130_FAB_ | 88a2b46ec220af | | | | | | | | | |
| 20211231_00194 | 47cb95c5 | | | | | | | | | |
| mAb_RBD_ | md5_eebd2cc178 | SL33Y | 4 | 196 | 198 | 204 | 228 | 242 | 243 | 251 |
| 2130_FAB_ | 6b724358ced82f | | | | | | | | | |
| 20211231_00199 | e314465c | | | | | | | | | |
| mAb_RBD_ | md5_aaca5fa01a | SL33W | 5 | 196 | 198 | 204 | 229 | 242 | 243 | 251 |
| 2130_FAB_ | 0f27e83e39da01 | | | | | | | | | |
| 20211231_00200 | a4457347 | | | | | | | | | |
| v2130_SFE_ | md5_2041aa1109 | SL32Q, | 6 | 196 | 198 | 204 | 230 | 242 | 244 | 251 |
| 20220104_ | 08c8cde47ed7aa | SL33Y, | | | | | | | | |
| 00015 | bc074be8 | TL59H | | | | | | | | |
| v2130_SFE_ | md5_1089bd14be | SL32W, | 7 | 196 | 198 | 204 | 231 | 242 | 245 | 251 |
| 20220104_ | 7c511aa67dbef7 | TL59E | | | | | | | | |
| 00024 | 3eeb1e42 | | | | | | | | | |
| mAb_v2130 | md5_689536fbc6 | IH55D, | 8 | 196 | 200 | 204 | 232 | 242 | 245 | 251 |
| JustStableLines_ | 7d8ee33e80ca5a | SL33F, | | | | | | | | |
| 20220111_ | 6709d167 | TL59E | | | | | | | | |
| 00002 | | | | | | | | | | |
| mAb_ | md5_a314041f0f | GH112E, | 9 | 196 | 198 | 205 | 233 | 242 | 243 | 251 |
| v2130Vanderbilt_ | 349a3dc6b474c0 | SL32Y, | | | | | | | | |
| 184_20220114_ | 69163d9b | SL33V, | | | | | | | | |
| 00111 | | KL36Y | | | | | | | | |
| mAb_ | md5_3052c528d5 | GH112E, | 10 | 196 | 198 | 205 | 234 | 242 | 245 | 251 |
| v2130Vanderbilt_ | 78179c00211647 | SL32A, | | | | | | | | |
| 184_20220114_ | 380e5fb7 | SL33A, | | | | | | | | |
| 00112 | | TL59E | | | | | | | | |
| v2130_ | md5_2cdc3bd28f | YH106D, | 11 | 196 | 198 | 206 | 235 | 242 | 246 | 251 |
| upsample_ | f984b7c5b8d2a8 | FH118I, | | | | | | | | |
| 20220121_00009 | 6ae9553f | SL33V, | | | | | | | | |
| | | YL55N | | | | | | | | |
| v2130_ | md5_6e9c26854d | IH55E, | 12 | 196 | 197 | 207 | 236 | 242 | 243 | 251 |
| upsample_ | 3e83e74f379947 | YH106H, | | | | | | | | |
| 20220121_00298 | 355e3c6a | SL32R, | | | | | | | | |
| | | KL36L | | | | | | | | |
| v2130_ | md5_8813a032f1 | IH55H, | 13 | 196 | 201 | 208 | 228 | 242 | 247 | 251 |
| upsample_ | 88710d1acf700c | GH110L, | | | | | | | | |
| 20220121_00314 | b2d61380 | SL33Y, | | | | | | | | |
| | | YL55E | | | | | | | | |
| v2130_ | md5_6362c40e1e | VH109R, | 14 | 196 | 198 | 209 | 234 | 242 | 245 | 251 |
| upsample_ | 8234bda1f78cd7 | GH112E, | | | | | | | | |
| 20220121_00354 | 6d72ca17 | SL32A, | | | | | | | | |
| | | SL33A, | | | | | | | | |
| | | TL59E | | | | | | | | |
| 2130_ | md5_ac4828a297 | 112E | 15 | 196 | 198 | 205 | 227 | 242 | 243 | 251 |
| Iteration1.5_ | 03823447738469 | | | | | | | | | |
| 00011 | ab260f6a | | | | | | | | | |
| 2130_ | md5_7b02dbd189 | 162A | 16 | 196 | 198 | 204 | 237 | 242 | 243 | 251 |
| Iteration1.5_ | 9b0826cc7d6fea | | | | | | | | | |
| 00012 | 6f5d88df | | | | | | | | | |
| 2130_ | md5_844594c4fc | 163A | 17 | 196 | 198 | 204 | 238 | 242 | 243 | 251 |
| Iteration1.5_ | fe975063269346 | | | | | | | | | |
| 00013 | 5ec9abb3 | | | | | | | | | |
| 2130_ | md5_b33188496f | 189E | 18 | 196 | 198 | 204 | 227 | 242 | 245 | 251 |
| Iteration1.5_ | de3cf4558f2c57 | | | | | | | | | |
| 00014 | 9cd5de40 | | | | | | | | | |
| 2130_ | md5_f3b8e8673d | 189H | 19 | 196 | 198 | 204 | 227 | 242 | 244 | 251 |
| Iteration1.5_ | 31b5b9fcffcc30 | | | | | | | | | |
| 00015 | 87f21318 | | | | | | | | | |
| 2130_ | md5_c66c7b0b82 | 55E | 20 | 196 | 197 | 204 | 227 | 242 | 243 | 251 |
| Iteration1.5_ | bfaf9bbb460ec3 | | | | | | | | | |
| 00016 | 772fcd18 | | | | | | | | | |
| 2130_ | md5_e3e94e79c2 | 55W, | 21 | 196 | 199 | 209 | 234 | 242 | 245 | 251 |
| Iteration1.5_ | 8dfaed07e6df24 | 109R, 112E, | | | | | | | | |
| 00017 | 598b4e1d | 162A, | | | | | | | | |
| | | 163A, 189E | | | | | | | | |
| 2130_ | md5_62ee8bbf44 | 55E, | 22 | 196 | 197 | 209 | 234 | 242 | 245 | 251 |
| Iteration1.5_ | 2fcabb49570777 | 109R, 112E, | | | | | | | | |
| 00018 | fcfead32 | 162A, | | | | | | | | |
| | | 163A, 189E | | | | | | | | |
| 2130_ | md5_99a2910274 | 109R, | 23 | 196 | 198 | 210 | 234 | 242 | 245 | 251 |
| Iteration1.5_ | 7e691465b2e14d | 112E, 113V, | | | | | | | | |
| 00019 | 9a903548 | 162A, | | | | | | | | |
| | | 163A, | | | | | | | | |
| | | 189E | | | | | | | | |
| 2130_ | md5_e4daa4c377 | 109R, | 24 | 196 | 198 | 209 | 239 | 242 | 245 | 251 |
| Iteration1.5_ | 6ff69b0bd45f85 | 112E, 162A, | | | | | | | | |
| 00020 | b4c8da3d | 163Y, | | | | | | | | |
| | | 189E | | | | | | | | |
| 2130_ | md5_ed7e966157 | 55W, | 25 | 196 | 199 | 210 | 234 | 242 | 245 | 251 |
| Iteration1.5_ | c0b81cb332148f | 109R, 112E, | | | | | | | | |
| 00021 | e52d35d4 | 113V, | | | | | | | | |
| | | 162A, 163A, | | | | | | | | |
| | | 189E | | | | | | | | |
| 2130_ | md5_698c4a4679 | 55E, 109R, | 26 | 196 | 197 | 210 | 234 | 242 | 245 | 251 |
| Iteration1.5_ | aedfb0cae31cd7 | 112E, | | | | | | | | |
| 00022 | 4e473906 | 113V, | | | | | | | | |
| | | 162A, | | | | | | | | |
| | | 163A, 189E | | | | | | | | |
| 2130_ | md5_f2345e1267 | 55W, | 27 | 196 | 199 | 205 | 234 | 242 | 245 | 251 |
| Iteration1.5_ | 9b385c2e3aa594 | 112E, 162A, | | | | | | | | |
| 00023 | f10c6dc0 | 163A, | | | | | | | | |
| | | 189E | | | | | | | | |
| 2130_ | md5_778c2db623 | 55E, | 28 | 196 | 197 | 205 | 234 | 242 | 245 | 251 |
| Iteration1.5_ | a1f1fb15a9aa3d | 112E, 162A, | | | | | | | | |
| 00024 | 68c34812 | 163A, | | | | | | | | |
| | | 189E | | | | | | | | |
| 2130_ | md5_685d04f38f | 112E, | 29 | 196 | 198 | 211 | 234 | 242 | 245 | 251 |
| Iteration1.5_ | a28de4ba7d9db1 | 113V, 162A, | | | | | | | | |
| 00025 | 4a3453bc | 163A, | | | | | | | | |
| | | 189E | | | | | | | | |
| 2130_ | md5_c9601b5954 | 112E, | 30 | 196 | 198 | 205 | 239 | 242 | 245 | 251 |
| Iteration1.5_ | 7deb9e0841d3d9 | 162A, 163Y, | | | | | | | | |
| 00026 | f45a1afc | 189E | | | | | | | | |
| 2130_ | md5_f0562398a3 | 55W, | 31 | 196 | 199 | 206 | 235 | 242 | 246 | 251 |
| Iteration1.5_ | 3d00e711906550 | 106D, 118I, | | | | | | | | |
| 00027 | f19b293e | 163V, | | | | | | | | |
| | | 185N | | | | | | | | |
| 2130_ | md5_8c6a6c711a | 55E, 106D, | 32 | 196 | 197 | 206 | 235 | 242 | 246 | 251 |
| Iteration1.5_ | a8191a68d8d423 | 118I, | | | | | | | | |
| 00028 | aefa3cf0 | 163V, | | | | | | | | |
| | | 185N | | | | | | | | |
| 2130_ | md5_56747677dc | 106D, | 33 | 196 | 198 | 212 | 235 | 242 | 246 | 251 |
| Iteration1.5_ | dee3db6bf31dc9 | 109R, 118I, | | | | | | | | |
| 00029 | 58bd4ec9 | 163V, | | | | | | | | |
| | | 185N | | | | | | | | |
| 2130_ | md5_88ff4f3db5 | 106D, | 34 | 196 | 198 | 213 | 235 | 242 | 246 | 251 |
| Iteration1.5_ | 4437166d0ca5bf | 112E, 118I, | | | | | | | | |
| 00030 | 0542264a | 163V, | | | | | | | | |
| | | 185N | | | | | | | | |
| 2130_ | md5_768ff12c1a | 106D, | 35 | 196 | 198 | 214 | 235 | 242 | 246 | 251 |
| Iteration1.5_ | 4b8beaf0ab30ca | 113V, 118I, | | | | | | | | |
| 00031 | d7407dac | 163V, | | | | | | | | |
| | | 185N | | | | | | | | |
| 2130_ | md5_71da11c6a5 | 106D, 118I, | 36 | 196 | 198 | 206 | 235 | 242 | 248 | 251 |
| Iteration1.5_ | f4fca7713a7976 | 163V, | | | | | | | | |
| 00032 | c1429c39 | 185N, | | | | | | | | |
| | | 189E | | | | | | | | |
| 2130_ | md5_d70e5cafa3 | 106D, 118I, | 37 | 196 | 198 | 206 | 235 | 242 | 249 | 251 |
| Iteration1.5_ | b8348a029dc50a | 163V, | | | | | | | | |
| 00033 | f54e2b00 | 185N, | | | | | | | | |
| | | 189H | | | | | | | | |
| 2130_ | md5_61311844b4 | 55E, 110L, | 38 | 196 | 197 | 208 | 228 | 242 | 247 | 251 |
| Iteration1.5_ | 35d59f5cb57beb | 110L, | | | | | | | | |
| 00034 | ba4c5f70 | 163Y, | | | | | | | | |
| | | 185E | | | | | | | | |
| 2130_ | md5_d62aa0dc67 | 55W, 110L, | 39 | 196 | 199 | 208 | 228 | 242 | 247 | 251 |
| Iteration1.5_ | f56e1baefc5da9 | 110L, | | | | | | | | |
| 00035 | acd1237c | 163Y, | | | | | | | | |
| | | 185E | | | | | | | | |
| 2130_ | md5_f3c22c1c68 | 55H, | 40 | 196 | 201 | 215 | 228 | 242 | 247 | 251 |
| Iteration1.5_ | 763fa02ca4f48a | 109R, 110L, | | | | | | | | |
| 00036 | 93cb8eeb | 110L, | | | | | | | | |
| | | 163Y, 185E | | | | | | | | |
| 2130_ | md5_bdf4629c9c | 55H, 110L, | 41 | 196 | 201 | 216 | 228 | 242 | 247 | 251 |
| Iteration1.5_ | 1a56f229956cf9 | 110L, | | | | | | | | |
| 00037 | 299ca568 | 112E, | | | | | | | | |
| | | 163Y, 185E | | | | | | | | |
| 2130_ | md5_b0c846f01f | 55H, | 42 | 196 | 201 | 217 | 228 | 242 | 247 | 251 |
| Iteration1.5_ | d4ee8eeac158d0 | 110L, 110L, | | | | | | | | |
| 00038 | 81bfb2be | 113V, | | | | | | | | |
| | | 163Y, 185E | | | | | | | | |
| 2130_ | md5_d99535affb | 55H, 110L, | 43 | 196 | 201 | 208 | 228 | 242 | 250 | 251 |
| Iteration1.5_ | 9946f40586d7a1 | 110L, | | | | | | | | |
| 00039 | 573072de | 163Y, | | | | | | | | |
| | | 185E, 189H | | | | | | | | |
| 2130_ | md5_052e2bc57e | 55E, 106H, | 44 | 196 | 197 | 218 | 236 | 242 | 243 | 251 |
| Iteration1.5_ | 68e1b4530dc038 | 109R, | | | | | | | | |
| 00040 | 246eee29 | 162R, | | | | | | | | |
| | | 166L | | | | | | | | |
| 2130_ | md5_8cd253058f | 55E, 106H, | 45 | 196 | 197 | 219 | 236 | 242 | 243 | 251 |
| Iteration1.5_ | dec72fbaf58cf4 | 112E, | | | | | | | | |
| 00041 | 1393724f | 162R, | | | | | | | | |
| | | 166L | | | | | | | | |
| 2130_ | md5_702355da28 | 55E, 106H, | 46 | 196 | 197 | 220 | 236 | 242 | 243 | 251 |
| Iteration1.5_ | ed59666ce6cf3a | 109R, | | | | | | | | |
| 00042 | 65d9e59f | 112E, 162R, | | | | | | | | |
| | | 166L | | | | | | | | |
| 2130_ | md5_bf465b80df | 55E, 106H, | 47 | 196 | 197 | 218 | 236 | 242 | 245 | 251 |
| Iteration1.5_ | bcfb6dd36bcd17 | 109R, | | | | | | | | |
| 00043 | 17737f7d | 162R, 166L, | | | | | | | | |
| | | 189E | | | | | | | | |
| 2130_ | md5_c209ad3a42 | 55E, 106H, | 48 | 196 | 197 | 207 | 236 | 242 | 245 | 251 |
| Iteration1.5_ | be1fb8fda0518f | 162R, | | | | | | | | |
| 00044 | 8cc21c2c | 166L, | | | | | | | | |
| | | 189E | | | | | | | | |
| 2130_ | md5_1e9c952de6 | 55W, 106H, | 49 | 196 | 199 | 207 | 236 | 242 | 243 | 251 |
| Iteration1.5_ | 3aa8327acff1cc | 162R, | | | | | | | | |
| 00045 | 2d2abbc7 | 166L | | | | | | | | |
| 2130_ | md5_2af950cc1c | 55W, 163F, | 50 | 196 | 199 | 204 | 232 | 242 | 245 | 251 |
| Iteration1.5_ | 1898e0a49b94ea | 189E | | | | | | | | |
| 00046 | b292aa66 | | | | | | | | | |
| 2130_ | md5_aab1483540 | 55E, 163F, | 51 | 196 | 197 | 204 | 232 | 242 | 245 | 251 |
| Iteration1.5_ | e1ae1278dc268a | 189E | | | | | | | | |
| 00047 | 9ab2565d | | | | | | | | | |
| 2130_ | md5_460d3280c2 | 55D, 109R, | 52 | 196 | 200 | 203 | 232 | 242 | 245 | 251 |
| Iteration1.5_ | 7f11b4fc0946ba | 163F, | | | | | | | | |
| 00048 | 8df54759 | 189E | | | | | | | | |
| 2130_ | md5_19b8e1f3e1 | 55D, 112E, | 53 | 196 | 200 | 205 | 232 | 242 | 245 | 251 |
| Iteration1.5_ | 6d175d2f1f038e | 163F, | | | | | | | | |
| 00049 | b9e3ad3f | 189E | | | | | | | | |
| 2130_ | md5_259e5b1dfa | 55D, 113V, | 54 | 196 | 200 | 221 | 232 | 242 | 245 | 251 |
| Iteration1.5_ | 4215ab7683e6bf | 163F, | | | | | | | | |
| 00050 | 24b7b939 | 189E | | | | | | | | |
| 2130_ | md5_a2492428ec | 55D, 109R, | 55 | 196 | 200 | 209 | 232 | 242 | 245 | 251 |
| Iteration1.5_ | b5126bc8c8c03e | 112E, | | | | | | | | |
| 00051 | 97de7eb0 | 163F, | | | | | | | | |
| | | 189E | | | | | | | | |
| 2130_ | md5_98948967f2 | 55D, 163A, | 56 | 196 | 200 | 204 | 238 | 242 | 245 | 251 |
| Iteration1.5_ | e5d0166316678e | 189E | | | | | | | | |
| 00052 | 80ae2446 | | | | | | | | | |
| 2130_ | md5_423d9b5486 | 55D, 163W, | 57 | 196 | 200 | 204 | 229 | 242 | 245 | 251 |
| Iteration1.5_ | 5843a72c7888f5 | 189E | | | | | | | | |
| 00053 | 802171a6 | | | | | | | | | |
| 2130_ | md5_cb395904fd | 55D, 163Y, | 58 | 196 | 200 | 204 | 228 | 242 | 245 | 251 |
| Iteration1.5_ | ef0858edf0cdb6 | 189E | | | | | | | | |
| 00054 | 0c59f338 | | | | | | | | | |
| 2130_ | md5_3d12c9e26e | 55W, 106F, | 59 | 196 | 199 | 202 | 226 | 242 | 243 | 251 |
| Iteration1.5_ | 1bef1a735ac575 | 162W, | | | | | | | | |
| 00055 | 6a577448 | 163A | | | | | | | | |
| 2130_ | md5_7c235b7cd7 | 55E, 106F, | 60 | 196 | 197 | 222 | 226 | 242 | 243 | 251 |
| Iteration1.5_ | 68b595619b8e30 | 109R, | | | | | | | | |
| 00056 | 06608a13 | 162W, | | | | | | | | |
| | | 163A | | | | | | | | |
| 2130_ | md5_c2933cc413 | 55E, 106F, | 61 | 196 | 197 | 223 | 226 | 242 | 243 | 251 |
| Iteration1.5_ | d4a1987d1f39c0 | 112E, | | | | | | | | |
| 00057 | 47717b51 | 162W, | | | | | | | | |
| | | 163A | | | | | | | | |
| 2130_ | md5_1dae53d51c | 55E, 106F, | 62 | 196 | 197 | 224 | 226 | 242 | 243 | 251 |
| Iteration1.5_ | edc8e8ea0f1ebc | 113V, | | | | | | | | |
| 00058 | 93170ea2 | 162W, | | | | | | | | |
| | | 163A | | | | | | | | |
| 2130_ | md5_6a5d7f58da | 55E, 106F, | 63 | 196 | 197 | 225 | 226 | 242 | 243 | 251 |
| Iteration1.5_ | 6a6e0d73f4198e | 109R, | | | | | | | | |
| 00059 | 1bd0d263 | 112E, | | | | | | | | |
| | | 162W, 163A | | | | | | | | |
| 2130_ | md5_7b91828641 | 55E, 106F, | 64 | 196 | 197 | 202 | 226 | 242 | 245 | 251 |
| Iteration1.5_ | 9ed897274892be | 162W, | | | | | | | | |
| 00060 | 92a5a1da | 163A, | | | | | | | | |
| | | 189E | | | | | | | | |
| 2130_ | md5_1f3ad4f915 | 55W, 162W, | 65 | 196 | 199 | 204 | 231 | 242 | 245 | 251 |
| Iteration1._5_ | 219eabdb9d95ef | 189E | | | | | | | | |
| 00061 | 4455cb6f | | | | | | | | | |
| 2130_ | md5_96f6cf6438 | 55E, 162W, | 66 | 196 | 197 | 204 | 231 | 242 | 245 | 251 |
| Iteration1.5_ | 0146cd84af6a7c | 189E | | | | | | | | |
| 00062 | e3fd7fb9 | | | | | | | | | |
| 2130_ | md5_14d2e59a93 | 109R, 162W, | 67 | 196 | 198 | 203 | 231 | 242 | 245 | 251 |
| Iteration1.5_ | 698d095fd22c7b | 189E | | | | | | | | |
| 00063 | a01fcbce | | | | | | | | | |
| 2130_ | md5_d320606976 | 112E, 162W, | 68 | 196 | 198 | 205 | 231 | 242 | 245 | 251 |
| Iteration1.5_ | fb803b69f33404 | 189E | | | | | | | | |
| 00064 | 6fc84543 | | | | | | | | | |
| 2130_ | md5_86730c5731 | 113V, 162W, | 69 | 196 | 198 | 221 | 231 | 242 | 245 | 251 |
| Iteration1.5_ | 9dd14c86fe94bc | 189E | | | | | | | | |
| 00065 | 812ad5fa | | | | | | | | | |
| 2130_ | md5_7da0538212 | 55W, 109R, | 70 | 196 | 199 | 203 | 231 | 242 | 245 | 251 |
| Iteration1.5_ | 84a0d91161e549 | 162W, | | | | | | | | |
| 00066 | a39b0c8e | 189E | | | | | | | | |
| 2130_ | md5_d3ec50e2db | 55W, 112E, | 71 | 196 | 199 | 205 | 231 | 242 | 245 | 251 |
| Iteration1.5_ | b15669959be94d | 162W, | | | | | | | | |
| 00067 | c006a449 | 189E | | | | | | | | |
| 2130_ | md5_cc3ee0fe85 | 55E, 109R, | 72 | 196 | 197 | 203 | 231 | 242 | 245 | 251 |
| Iteration1.5_ | b16e315e8daee6 | 162W, | | | | | | | | |
| 00068 | 04f29840 | 189E | | | | | | | | |
| 2130_ | md5_9315a7cc3d | 55E, 112E, | 73 | 196 | 197 | 205 | 231 | 242 | 245 | 251 |
| Iteration1.5_ | ae1e80c6cefa1a | 162W, | | | | | | | | |
| 00069 | 7840e9d1 | 189E | | | | | | | | |
| 2130_ | md5_ee41994614 | 162W, 163A, | 74 | 196 | 198 | 204 | 226 | 242 | 245 | 251 |
| Iteration1.5_ | eee7e3131741e9 | 189E | | | | | | | | |
| 00070 | 86153676 | | | | | | | | | |
| 2130_ | md5_b2200f7396 | 109R, 112E, | 75 | 196 | 198 | 209 | 231 | 242 | 245 | 251 |
| Iteration1.5_ | e3968080599cd4 | 162W, | | | | | | | | |
| 00071 | e6f8f81b | 189E | | | | | | | | |
| 2130_ | md5_0f4532e0ae | 55W, 109R, | 76 | 196 | 199 | 209 | 231 | 242 | 245 | 251 |
| Iteration1.5_ | 4e7755d3d48072 | 112E, | | | | | | | | |
| 00072 | aea1f18c | 162W, | | | | | | | | |
| | | 189E | | | | | | | | |
| 2130_ | md5_d9e89da7b0 | 55E, | 77 | 196 | 197 | 209 | 231 | 242 | 245 | 251 |
| Iteration1.5_ | 1686bda150c990 | 109R, 112E, | | | | | | | | |
| 00073 | ddbf903c | 162W, | | | | | | | | |
| | | 189E | | | | | | | | |
| 2130_ | md5_937dab20e7 | 55W, | 78 | 196 | 199 | 205 | 233 | 242 | 243 | 251 |
| Iteration1.5_ | 5d4d8a1838dc38 | 112E, 162Y, | | | | | | | | |
| 00074 | 2cb48b5b | 163V, | | | | | | | | |
| | | 166Y | | | | | | | | |
| 2130_ | md5_f5378fc519 | 55E, 112E, | 79 | 196 | 197 | 205 | 233 | 242 | 243 | 251 |
| Iteration1.5_ | 27d65e51c662b5 | 162Y, | | | | | | | | |
| 00075 | ea017638 | 163V, | | | | | | | | |
| | | 166Y | | | | | | | | |
| 2130_ | md5_2753914b7c | 109R, | 80 | 196 | 198 | 209 | 233 | 242 | 243 | 251 |
| Iteration1.5_ | 94c5cfe1024c3c | 112E, 162Y, | | | | | | | | |
| 00076 | 43176c53 | 163V, | | | | | | | | |
| | | 166Y | | | | | | | | |
| 2130_ | md5_2093fe026e | 112E, | 81 | 196 | 198 | 211 | 233 | 242 | 243 | 251 |
| Iteration1.5_ | dc51c3017ac5da | 113V, 162Y, | | | | | | | | |
| 00077 | 316ed0d4 | 163V, | | | | | | | | |
| | | 166Y | | | | | | | | |
| 2130_ | md5_a3a3295e22 | 112E, 162Y, | 82 | 196 | 198 | 205 | 240 | 242 | 243 | 251 |
| Iteration1.5_ | bbd7a4f09f451a | 166Y | | | | | | | | |
| 00078 | cd976ee4 | | | | | | | | | |
| 2130_ | md5_b4f5565718 | 112E, 162Y, | 83 | 196 | 198 | 205 | 241 | 242 | 243 | 251 |
| Iteration1.5_ | de43287bb9ef3f | 163A, | | | | | | | | |
| 00079 | 80db822b | 166Y | | | | | | | | |
| 2130_ | md5_e40fd800f7 | 55W, 109R, | 84 | 196 | 199 | 209 | 233 | 242 | 243 | 251 |
| Iteration1.5_ | 1a4cf8cd84f490 | 112E, | | | | | | | | |
| 00080 | f2d916f2 | 162Y, | | | | | | | | |
| | | 163V, 166Y | | | | | | | | |
| 2130_ | md5_442d78ecdb | 55W, 109R, | 85 | 196 | 199 | 209 | 233 | 242 | 245 | 251 |
| Iteration1.5_ | 922e86c6fa9fdf | 112E, | | | | | | | | |
| 00081 | 25560d62 | 162Y, | | | | | | | | |
| | | 163V, 166Y, | | | | | | | | |
| | | 189E | | | | | | | | |
| 2130_ | md5_54cbd3b086 | 55W, 162Q, | 86 | 196 | 199 | 204 | 230 | 242 | 244 | 251 |
| Iteration1.5_ | 9bf439fdb44b21 | 163Y, | | | | | | | | |
| 00082 | 34d831a7 | 189H | | | | | | | | |
| 2130_ | md5_4ef9d06c52 | 109R, 162Q, | 87 | 196 | 198 | 203 | 230 | 242 | 244 | 251 |
| Iteration1.5_ | ca21a3c72edb3d | 163Y, | | | | | | | | |
| 00083 | 1e34d887 | 189H | | | | | | | | |
| 2130_ | md5_6989c5e6f7 | 112E, | 88 | 196 | 198 | 205 | 230 | 242 | 244 | 251 |
| Iteration1.5_ | 6eb49570038d3d | 162Q, 163Y, | | | | | | | | |
| 00084 | 5776410b | 189H | | | | | | | | |
| 2130_ | md5_98860fd1b4 | 113V, | 89 | 196 | 198 | 221 | 230 | 242 | 244 | 251 |
| Iteration1.5_ | 37cce384562f2d | 162Q, 163Y, | | | | | | | | |
| 00085 | c1cf9d28 | 189H | | | | | | | | |
| 2130_ | md5_975f7cd499 | 55E, 109R, | 90 | 196 | 197 | 203 | 230 | 242 | 244 | 251 |
| Iteration1.5_ | b5467063d15822 | 162Q, | | | | | | | | |
| 00086 | 57633fd1 | 163Y, | | | | | | | | |
| | | 189H | | | | | | | | |
| 2130_ | md5_23639310c6 | 55W, 109R, | 91 | 196 | 199 | 203 | 230 | 242 | 244 | 251 |
| Iteration1.5_ | 86f78739fd6a99 | 162Q, | | | | | | | | |
| 00087 | 80e5f424 | 163Y, | | | | | | | | |
| | | 189H | | | | | | | | |
| 2130_ | md5_67638acfdb | 55E, 112E, | 92 | 196 | 197 | 205 | 230 | 242 | 244 | 251 |
| Iteration1.5_ | 3bf174f9dc45d5 | 162Q, | | | | | | | | |
| 00088 | d551d07d | 163Y, | | | | | | | | |
| | | 189H | | | | | | | | |
| 2130_ | md5_4c674020ad | 55W, 112E, | 93 | 196 | 199 | 205 | 230 | 242 | 244 | 251 |
| Iteration1.5_ | 4709db9d5874af | 162Q, | | | | | | | | |
| 00089 | 1b2f0c9b | 163Y, | | | | | | | | |
| | | 189H | | | | | | | | |
| 2130_ | md5_ddfa55c0c4 | 55E, 109R, | 94 | 196 | 197 | 209 | 230 | 242 | 244 | 251 |
| Iteration1.5_ | afa535fdefbe02 | 112E, | | | | | | | | |
| 00090 | 93a7a027 | 162Q, | | | | | | | | |
| | | 163Y, 189H | | | | | | | | |
| 2130_ | md5_5a3100e97e | 55W, 109R, | 95 | 196 | 199 | 209 | 230 | 242 | 244 | 251 |
| Iteration1.5_ | a251ab9b1dceb4 | 112E, | | | | | | | | |
| 00091 | 7049173f | 162Q, 163Y, | | | | | | | | |
| | | 189H | | | | | | | | |
| 2130_ | md5_2b3d375d70 | 55W, 109R, | 96 | 196 | 199 | 209 | 230 | 242 | 245 | 251 |
| Iteration1.5_ | f4dfc682a73c79 | 112E, | | | | | | | | |
| 00092 | d6192c36 | 162Q, 163Y, | | | | | | | | |
| | | 189E | | | | | | | | |
| 2130_ | md5_cb1732065c | 55W, 109R, | 97 | 196 | 199 | 209 | 229 | 242 | 243 | 251 |
| Iteration1.5_ | 938f7bdf6a12d3 | 112E, | | | | | | | | |
| 00093 | 9a72f90c | 163W | | | | | | | | |
| 2130_ | md5_262e96c1bd | 55W, 109R, | 98 | 196 | 199 | 209 | 228 | 242 | 243 | 251 |
| Iteration1.5_ | c514d4b4609a39 | 112E, | | | | | | | | |
| 00094 | c613a3f4 | 163Y | | | | | | | | |
| 2130_ | md5_ef201091f7 | 55W, 109R, | 99 | 196 | 199 | 209 | 238 | 242 | 243 | 251 |
| Iteration1.5_ | 97b7f61249f3c6 | 112E, | | | | | | | | |
| 00095 | 85e6a321 | 163A | | | | | | | | |

TABLE 14
VH and VL sequences of the disclosed antibodies

| HumID | HashID | VH SEQ ID NO | VL SEQ ID NO |
|---|---|---|---|
| mAb_RBD_2130_FAB_20211231_00017 | md5_e3d1904966eaf73aed30331f60b658e9 | 100 | 155 |
| mAb_RBD_2130_FAB_20211231_00174 | md5_d323d72bb6bc337ea256bdd92ceba08e | 101 | 156 |
| mAb_RBD_2130_FAB_20211231_00194 | md5_9f6486808688a2b46ec220af47cb95c5 | 102 | 156 |
| mAb_RBD_2130_FAB_20211231_00199 | md5_eebd2cc1786b724358ced82fe314465c | 103 | 157 |
| mAb_RBD_2130_FAB_20211231_00200 | md5_aaca5fa01a0f27e83e39da01a4457347 | 103 | 158 |
| v2130_SFE_20220104_00015 | md5_2041aa110908c8cde47ed7aabc074be8 | 103 | 159 |
| v2130_SFE_20220104_00024 | md5_1089bd14be7c511aa67dbef73eeb1e42 | 103 | 160 |
| mAb_v2130JustStableLines_20220111_00002 | md5_689536fbc67d8ee33e80ca5a6709d167 | 104 | 161 |
| mAb_v2130Vanderbilt_184_20220114_00111 | md5_a314041f0f349a3dc6b474c069163d9b | 105 | 162 |
| mAb_v2130Vanderbilt_184_20220114_00112 | md5_3052c528d578179c00211647380e5fb7 | 105 | 163 |
| v2130_upsample_20220121_00009 | md5_2cdc3bd28ff984b7c5b8d2a86ae9553f | 106 | 164 |
| v2130_upsample_20220121_00298 | md5_6e9c26854d3e83e74f379947355e3c6a | 107 | 165 |
| v2130_upsample_20220121_00314 | md5_8813a032f188710d1acf700cb2d61380 | 108 | 166 |
| v2130_upsample_20220121_00354 | md5_6362c40e1e8234bda1f78cd76d72ca17 | 109 | 163 |
| 2130_Iteration1.5_00011 | md5_ac4828a29703823447738469ab260f6a | 110 | 167 |
| 2130_Iteration1.5_00012 | md5_7b02dbd1899b0826cc7d6fea6f5d88df | 111 | 168 |
| 2130_Iteration1.5_00013 | md5_844594c4fcfe9750632693465ec9abb3 | 111 | 169 |
| 2130_Iteration1.5_00014 | md5_b33188496fde3cf4558f2c579cd5de40 | 111 | 170 |
| 2130_Iteration1.5_00015 | md5_f3b8e8673d31b5b9fcffcc3087f21318 | 111 | 171 |
| 2130_Iteration1.5_00016 | md5_c66c7b0b82bfaf9bbb460ec3772fcd18 | 112 | 167 |
| 2130_Iteration1.5_00017 | md5_e3e94e79c28dfaed07e6df24598b4e1d | 113 | 172 |
| 2130_Iteration1.5_00018 | md5_62ee8bbf442fcabb49570777fcfead32 | 114 | 172 |
| 2130_Iteration1.5_00019 | md5_99a29102747e691465b2e14d9a903548 | 115 | 172 |
| 2130_Iteration1.5_00020 | md5_e4daa4c3776ff69b0bd45f85b4c8da3d | 116 | 173 |
| 2130_Iteration1.5_00021 | md5_ed7e966157c0b81cb332148fe52d35d4 | 117 | 172 |
| 2130_Iteration1.5_00022 | md5_698c4a4679aedfb0cae31cd74e473906 | 118 | 172 |
| 2130_Iteration1.5_00023 | md5_f2345e12679b385c2e3aa594f10c6dc0 | 119 | 172 |
| 2130_Iteration1.5_00024 | md5_778c2db623a1f1fb15a9aa3d68c34812 | 120 | 172 |
| 2130_Iteration1.5_00025 | md5_685d04f38fa28de4ba7d9db14a3453bc | 121 | 172 |
| 2130_Iteration1.5_00026 | md5_c9601b59547deb9e0841d3d9f45a1afc | 110 | 173 |
| 2130_Iteration1.5_00027 | md5_f0562398a33d00e711906550f19b293e | 122 | 174 |
| 2130_Iteration1.5_00028 | md5_8c6a6c711aa8191a68d8d423aefa3cf0 | 123 | 174 |
| 2130_Iteration1.5_00029 | md5_56747677dcdee3db6bf31dc958bd4ec9 | 124 | 174 |
| 2130_Iteration1.5_00030 | md5_88ff4f3db54437166d0ca5bf0542264a | 125 | 174 |
| 2130_Iteration1.5_00031 | md5_768ff12c1a4b8beaf0ab30cad7407dac | 126 | 174 |
| 2130_Iteration1.5_00032 | md5_71da11c6a5f4fca7713a7976c1429c39 | 127 | 175 |
| 2130_Iteration1.5_00033 | md5_d70e5cafa3b8348a029dc50af54e2b00 | 127 | 176 |
| 2130_Iteration1.5_00034 | md5_61311844b435d59f5cb57bebba4c5f70 | 128 | 177 |
| 2130_Iteration1.5_00035 | md5_d62aa0dc67f56e1baefc5da9acd1237c | 129 | 177 |
| 2130_Iteration1.5_00036 | md5_f3c22c1c68763fa02ca4f48a93cb8eeb | 130 | 177 |
| 2130_Iteration1.5_00037 | md5_bdf4629c9c1a56f229956cf9299ca568 | 131 | 177 |
| 2130_Iteration1.5_00038 | md5_b0c846f01fd4ee8eeac158d081bfb2be | 132 | 177 |
| 2130_Iteration1.5_00039 | md5_d99535affb9946f40586d7a1573072de | 133 | 178 |
| 2130_Iteration1.5_00040 | md5_052e2bc57e68e1b4530dc038246eee29 | 134 | 179 |
| 2130_Iteration1.5_00041 | md5_8cd253058fdec72fbaf58cf41393724f | 135 | 179 |
| 2130_Iteration1.5_00042 | md5_702355da28ed59666ce6cf3a65d9e59f | 136 | 179 |
| 2130_Iteration1.5_00043 | md5_bf465b80dfbcfb6dd36bcd1717737f7d | 134 | 180 |
| 2130_Iteration1.5_00044 | md5_c209ad3a42be1fb8fda0518f8cc21c2c | 137 | 180 |
| 2130_Iteration1.5_00045 | md5_1e9c952de63aa8327acff1cc2d2abbc7 | 138 | 179 |
| 2130_Iteration1.5_00046 | md5_2af950cc1c1898e0a49b94eab292aa66 | 139 | 181 |
| 2130_Iteration1.5_00047 | md5_aab1483540e1ae1278dc268a9ab2565d | 112 | 181 |
| 2130_Iteration1.5_00048 | md5_460d3280c27f11b4fc0946ba8df54759 | 140 | 181 |
| 2130_Iteration1.5_00049 | md5_19b8e1f3e16d175d2f1f038eb9e3ad3f | 141 | 181 |
| 2130_Iteration1.5_00050 | md5_259e5b1dfa4215ab7683e6bf24b7b939 | 142 | 181 |
| 2130_Iteration1.5_00051 | md5_a2492428ecb5126bc8c8c03e97de7eb0 | 143 | 181 |
| 2130_Iteration1.5_00052 | md5_98948967f2e5d0166316678e80ae2446 | 144 | 182 |
| 2130_Iteration1.5_00053 | md5_423d9b54865843a72c7888f5802171a6 | 144 | 183 |
| 2130_Iteration1.5_00054 | md5_cb395904fdef0858edf0cdb60c59f338 | 144 | 184 |
| 2130_Iteration1.5_00055 | md5_3d12c9e26e1bef1a735ac5756a577448 | 145 | 185 |
| 2130_Iteration1.5_00056 | md5_7c235b7cd768b595619b8e3006608a13 | 146 | 185 |
| 2130_Iteration1.5_00057 | md5_c2933cc413d4a1987d1f39c047717b51 | 147 | 185 |
| 2130_Iteration1.5_00058 | md5_1dae53d51cedc8e8ea0f1ebc93170ea2 | 148 | 185 |
| 2130_Iteration1.5_00059 | md5_6a5d7f58da6a6e0d73f4198e1bd0d263 | 149 | 185 |
| 2130_Iteration1.5_00060 | md5_7b918286419ed897274892be92a5a1da | 150 | 186 |
| 2130_Iteration1.5_00061 | md5_1f3ad4f915219eabdb9d95ef4455cb6f | 139 | 187 |
| 2130_Iteration1.5_00062 | md5_96f6cf64380146cd84af6a7ce3fd7fb9 | 112 | 187 |
| 2130_Iteration1.5_00063 | md5_14d2e59a93698d095fd22c7ba01fcbce | 151 | 187 |
| 2130_Iteration1.5_00064 | md5_d320606976fb803b69f334046fc84543 | 110 | 187 |
| 2130_Iteration1.5_00065 | md5_86730c57319dd14c86fe94bc812ad5fa | 152 | 187 |
| 2130_Iteration1.5_00066 | md5_7da053821284a0d91161e549a39b0c8e | 153 | 187 |
| 2130_Iteration1.5_00067 | md5_d3ec50e2dbb15669959be94dc006a449 | 119 | 187 |
| 2130_Iteration1.5_00068 | md5_cc3ee0fe85b16e315e8daee604f29840 | 154 | 187 |
| 2130_Iteration1.5_00069 | md5_9315a7cc3dae1e80c6cefa1a7840e9d1 | 120 | 187 |
| 2130_Iteration1.5_00070 | md5_ee41994614eee7e3131741e986153676 | 111 | 186 |
| 2130_Iteration1.5_00071 | md5_b2200f7396e3968080599cd4e6f8f81b | 116 | 187 |
| 2130_Iteration1.5_00072 | md5_0f4532e0ae4e7755d3d48072aea1f18c | 113 | 187 |
| 2130_Iteration1.5_00073 | md5_d9e89da7b01686bda150c990ddbf903c | 114 | 187 |
| 2130_Iteration1.5_00074 | md5_937dab20e75d4d8a1838dc382cb48b5b | 119 | 188 |
| 2130_Iteration1.5_00075 | md5_f5378fc51927d65e51c662b5ea017638 | 120 | 188 |
| 2130_Iteration1.5_00076 | md5_2753914b7c94c5cfe1024c3c43176c53 | 116 | 188 |
| 2130_Iteration1.5_00077 | md5_2093fe026edc51c3017ac5da316ed0d4 | 121 | 188 |
| 2130_Iteration1.5_00078 | md5_a3a3295e22bbd7a4f09f451acd976ee4 | 110 | 189 |
| 2130_Iteration1.5_00079 | md5_b4f5565718de43287bb9ef3f80db822b | 110 | 190 |
| 2130_Iteration1.5_00080 | md5_e40fd800f71a4cf8cd84f490f2d916f2 | 113 | 188 |
| 2130_Iteration1.5_00081 | md5_442d78ecdb922e86c6fa9fdf25560d62 | 113 | 191 |
| 2130_Iteration1.5_00082 | md5_54cbd3b0869bf439fdb44b2134d831a7 | 139 | 192 |
| 2130_Iteration1.5_00083 | md5_4ef9d06c52ca21a3c72edb3d1e34d887 | 151 | 192 |
| 2130_Iteration1.5_00084 | md5_6989c5e6f76eb49570038d3d5776410b | 110 | 192 |
| 2130_Iteration1.5_00085 | md5_98860fd1b437cce384562f2dc1cf9d28 | 152 | 192 |
| 2130_Iteration1.5_00086 | md5_975f7cd499b5467063d1582257633fd1 | 154 | 192 |
| 2130_Iteration1.5_00087 | md5_23639310c686f78739fd6a9980e5f424 | 153 | 192 |
| 2130_Iteration1.5_00088 | md5_67638acfdb3bf174f9dc45d5d551d07d | 120 | 192 |
| 2130_Iteration1.5_00089 | md5_4c674020ad4709db9d5874af1b2f0c9b | 119 | 192 |
| 2130_Iteration1.5_00090 | md5_ddfa55c0c4afa535fdefbe0293a7a027 | 114 | 192 |
| 2130_Iteration1.5_00091 | md5_5a3100e97ea251ab9b1dceb47049173f | 113 | 192 |
| 2130_Iteration1.5_00092 | md5_2b3d375d70f4dfc682a73c79d6192c36 | 113 | 193 |
| 2130_Iteration1.5_00093 | md5_cb1732065c938f7bdf6a12d39a72f90c | 113 | 194 |
| 2130_Iteration1.5_00094 | md5_262e96c1bdc514d4b4609a39c613a3f4 | 113 | 195 |
| 2130_Iteration1.5_00095 | md5_ef201091f797b7f61249f3c685e6a321 | 113 | 169 |

TABLE 15
Antibody Concentration and Binding Data

| | | Conc | LLNL Gyros Intensity | | |
|---|---|---|---|---|---|
| HumID | AntibodyHashID | (mg/mL) | BA1.1 | BA1 | Wuhan |
| 2130_Iteration1.5_00011 | md5_ac4828a29703823447738469ab260f6a | 1.8 | 111.321 | 32.2256 | 35.0927 |
| 2130_Iteration1.5_00012 | md5_7b02dbd1899b0826cc7d6fea6f5d88df | 2.01 | 75.6713 | 21.7229 | 28.6375 |
| 2130_Iteration1.5_00013 | md5_844594c4fcfe9750632693465ec9abb3 | 2.11 | 88.0533 | 29.2824 | 33.1889 |
| 2130_Iteration1.5_00014 | md5_b33188496fde3cf4558f2c579cd5de40 | 2.07 | 98.1288 | 16.342 | 25.6569 |
| 2130_Iteration1.5_00015 | md5_f3b8e8673d31b5b9fcffcc3087f21318 | 1.47 | 12.113 | 12.3639 | 26.8665 |
| 2130_Iteration1.5_00016 | md5_c66c7b0b82bfaf9bbb460ec3772fcd18 | 1.78 | 4.2282 | 11.982 | 25.4348 |
| 2130_Iteration1.5_00017 | md5_e3e94e79c28dfaed07e6df24598b4e1d | 3.43 | 148.093 | 53.8506 | 40.8123 |
| 2130_Iteration1.5_00018 | md5_62ee8bbf442fcabb49570777fcfead32 | 3.39 | 142.141 | 49.1111 | 39.7677 |
| 2130_Iteration1.5_00019 | md5_99a29102747e691465b2e14d9a903548 | 2.95 | 151.741 | 34.0645 | 40.5927 |
| 2130_Iteration1.5_00020 | md5_e4daa4c3776ff69b0bd45f85b4c8da3d | 2.16 | 139.534 | 18.2444 | 38.0149 |
| 2130_Iteration1.5_00021 | md5_ed7e966157c0b81cb332148fe52d35d4 | 3.28 | 148.22 | 49.3664 | 38.2721 |
| 2130_Iteration1.5_00022 | md5_698c4a4679aedfb0cae31cd74e473906 | 3.13 | 148.402 | 36.3858 | 38.6501 |
| 2130_Iteration1.5_00023 | md5_f2345e12679b385c2e3aa594f10c6dc0 | 3.11 | 144.946 | 49.2889 | 37.279 |
| 2130_Iteration1.5_00024 | md5_778c2db623a1f1fb15a9aa3d68c34812 | 3.04 | 148.268 | 44.0553 | 37.1894 |
| 2130_Iteration1.5_00025 | md5_685d04f38fa28de4ba7d9db14a3453bc | 2.63 | 143.255 | 41.4493 | 36.8551 |
| 2130_Iteration1.5_00026 | md5_c9601b59547deb9e0841d3d9f45a1afc | 1.96 | 140.973 | 40.9795 | 37.7057 |
| 2130_Iteration1.5_00027 | md5_f0562398a33d00e711906550f19b293e | 0.74 | 1.73206 | 0.517428 | 0.760431 |
| 2130_Iteration1.5_00028 | md5_8c6a6c711aa8191a68d8d423aefa3cf0 | 0.87 | 0.97429 | 0.320077 | 1.33263 |
| 2130_Iteration1.5_00029 | md5_56747677dcdee3db6bf31dc958bd4ec9 | 0.92 | 1.49001 | 0.315946 | 0.533818 |
| 2130_Iteration1.5_00030 | md5_88ff4f3db54437166d0ca5bf0542264a | 1.02 | 0.630642 | 0.174437 | 0.736928 |
| 2130_Iteration1.5_00031 | md5_768ff12c1a4b8beaf0ab30cad7407dac | 0.99 | 0.762007 | 0.24662 | 0.64053 |
| 2130_Iteration1.5_00032 | md5_71da11c6a5f4fca7713a7976c1429c39 | 2.71 | 0.695505 | 0.224465 | 1.02104 |
| 2130_Iteration1.5_00033 | md5_d70e5cafa3b8348a029dc50af54e2b00 | 1.74 | 1.02296 | 0.327917 | 1.33729 |
| 2130_Iteration1.5_00034 | md5_61311844b435d59f5cb57bebba4c5f70 | 0.77 | 0.643359 | 0.130493 | 0.450442 |
| 2130_Iteration1.5_00035 | md5_d62aa0dc67f56e1baefc5da9acd1237c | 0.72 | 0.540889 | 0.12567 | 0.364432 |
| 2130_Iteration1.5_00036 | md5_f3c22c1c68763fa02ca4f48a93cb8eeb | 0.72 | 0.5964 | 0.196908 | 0.528834 |
| 2130_Iteration1.5_00037 | md5_bdf4629c9c1a56f229956cf9299ca568 | 0.71 | 0.400039 | 0.110237 | 0.40851 |
| 2130_Iteration1.5_00038 | md5_b0c846f01fd4ee8eeac158d081bfb2be | 0.58 | 0.368304 | 0.13844 | 0.509433 |
| 2130_Iteration1.5_00039 | md5_d99535affb9946f40586d7a1573072de | 0.84 | 0.508622 | 0.183172 | 0.181043 |
| 2130_Iteration1.5_00040 | md5_052e2bc57e68e1b4530dc038246eee29 | 1.63 | 6.77768 | 3.50609 | 16.9537 |
| 2130_Iteration1.5_00041 | md5_8cd253058fdec72fbaf58cf41393724f | 1.41 | 6.45993 | 8.53591 | 12.6453 |
| 2130_Iteration1.5_00042 | md5_702355da28ed59666ce6cf3a65d9e59f | 1.42 | 87.2083 | 21.7326 | 20.5933 |
| 2130_Iteration1.5_00043 | md5_bf465b80dfbcfb6dd36bcd1717737f7d | 1.52 | 62.3827 | 15.0215 | 15.4485 |
| 2130_Iteration1.5_00044 | md5_c209ad3a42be1fb8fda0518f8cc21c2c | 1.39 | 2.41816 | 2.82817 | 2.22193 |
| 2130_Iteration1.5_00045 | md5_1e9c952de63aa8327acff1cc2d2abbc7 | 1.11 | 3.93661 | 1.25403 | 7.86726 |
| 2130_Iteration1.5_00046 | md5_2af950cc1c1898e0a49b94eab292aa66 | 2.2 | 91.0252 | 33.4307 | 29.3824 |
| 2130_Iteration1.5_00047 | md5_aab1483540e1ae1278dc268a9ab2565d | 1.68 | 80.1937 | 21.0463 | 27.945 |
| 2130_Iteration1.5_00048 | md5_460d3280c27f11b4fc0946ba8df54759 | 1.31 | 136.01 | 30.3281 | 34.9232 |
| 2130_Iteration1.5_00049 | md5_19b8e1f3e16d175d2f1f038eb9e3ad3f | 1.69 | 134.908 | 31.656 | 32.29 |
| 2130_Iteration1.5_00050 | md5_259e5b1dfa4215ab7683e6bf24b7b939 | 1.09 | 108.241 | 22.9298 | 29.1855 |
| 2130_Iteration1.5_00051 | md5_a2492428ecb5126bc8c8c03e97de7eb0 | 1.64 | 149.307 | 37.4582 | 36.8777 |
| 2130_Iteration1.5_00052 | md5_98948967f2e5d0166316678e80ae2446 | 2.31 | 116.733 | 23.1536 | 28.5454 |
| 2130_Iteration1.5_00053 | md5_423d9b54865843a72c7888f5802171a6 | 2 | 100.15 | 20.8944 | 26.1733 |
| 2130_Iteration1.5_00054 | md5_cb395904fdef0858edf0cdb60c59f338 | 1.88 | 89.0101 | 17.2685 | 25.6256 |
| 2130_Iteration1.5_00055 | md5_3d12c9e26e1bef1a735ac5756a577448 | 2.25 | 143.949 | 24.9477 | 28.492 |
| 2130_Iteration1.5_00056 | md5_7c235b7cd768b595619b8e3006608a13 | 2.32 | 144.117 | 27.7106 | 36.7206 |
| 2130_Iteration1.5_00057 | md5_c2933cc413d4a1987d1f39c047717b51 | 2.28 | 141.909 | 29.4421 | 35.0351 |
| 2130_Iteration1.5_00058 | md5_1dae53d51cedc8e8ea0f1ebc93170ea2 | 2.08 | 128.513 | 21.9091 | 33.0777 |
| 2130_Iteration1.5_00059 | md5_6a5d7f58da6a6e0d73f4198e1bd0d263 | 2.35 | 155.096 | 35.6445 | 40.4948 |
| 2130_Iteration1.5_00060 | md5_7b918286419ed897274892be92a5a1da | 2.53 | 139.199 | 25.501 | 27.5819 |
| 2130_Iteration1.5_00061 | md5_1f3ad4f915219eabdb9d95ef4455cb6f | 1.79 | 130.462 | 38.7799 | 25.8578 |
| 2130_Iteration1.5_00062 | md5_96f6cf64380146cd84af6a7ce3fd7fb9 | 1.14 | 106.457 | 16.1998 | 20.9005 |
| 2130_Iteration1.5_00063 | md5_14d2e59a93698d095fd22c7ba01fcbce | 2.39 | 127.716 | 43.2218 | 32.6675 |
| 2130_Iteration1.5_00064 | md5_d320606976fb803b69f334046fc84543 | 1.38 | 143.638 | 34.175 | 27.3982 |
| 2130_Iteration1.5_00065 | md5_86730c57319dd14c86fe94bc812ad5fa | 2.09 | 121.378 | 35.7953 | 26.2641 |
| 2130_Iteration1.5_00066 | md5_7da053821284a0d91161e549a39b0c8e | 1.35 | 153.902 | 30.8952 | 29.0611 |
| 2130_Iteration1.5_00067 | md5_d3ec50e2dbb15669959be94dc006a449 | 1.54 | 147.291 | 36.0591 | 28.0187 |
| 2130_Iteration1.5_00068 | md5_cc3ee0fe85b16e315e8daee604f29840 | 1.97 | 147.163 | 35.4038 | 31.0022 |
| 2130_Iteration1.5_00069 | md5_9315a7cc3dae1e80c6cefa1a7840e9d1 | 1.86 | 140.073 | 35.369 | 26.0679 |
| 2130_Iteration1.5_00070 | md5_ee41994614eee7e3131741e986153676 | 2.85 | 148.379 | 29.1134 | 28.2514 |
| 2130_Iteration1.5_00071 | md5_b2200f7396e3968080599cd4e6f8f81b | 2.1 | 154.55 | 35.8248 | 35.5988 |
| 2130_Iteration1.5_00072 | md5_0f4532e0ae4e7755d3d48072aea1f18c | 1.93 | 125.42 | 41.2228 | 33.1797 |
| 2130_Iteration1.5_00073 | md5_d9e89da7b01686bda150c990ddbf903c | 2.09 | 151.223 | 43.7334 | 34.6358 |
| 2130_Iteration1.5_00074 | md5_937dab20e75d4d8a1838dc382cb48b5b | 0.83 | 109.145 | 15.4655 | 15.4915 |
| 2130_Iteration1.5_00075 | md5_f5378fc51927d65e51c662b5ea017638 | 1.05 | 11.6037 | 7.55911 | 13.7385 |
| 2130_Iteration1.5_00076 | md5_2753914b7c94c5cfe1024c3c43176c53 | 1 | 138.938 | 22.2061 | 23.6115 |
| 2130_Iteration1.5_00077 | md5_2093fe026edc51c3017ac5da316ed0d4 | 0.73 | 11.9094 | 4.64887 | 14.8819 |
| 2130_Iteration1.5_00078 | md5_a3a3295e22bbd7a4f09f451acd976ee4 | 0.84 | 71.3569 | 11.9855 | 16.4658 |
| 2130_Iteration1.5_00079 | md5_b4f5565718de43287bb9ef3f80db822b | 0.89 | 121.734 | 20.0474 | 17.503 |
| 2130_Iteration1.5_00080 | md5_e40fd800f71a4cf8cd84f490f2d916f2 | 0.93 | 133.598 | 24.9923 | 22.3217 |
| 2130_Iteration1.5_00081 | md5_442d78ecdb922e86c6fa9fdf25560d62 | 0.7 | 140.355 | 30.3194 | 20.3884 |
| 2130_Iteration1.5_00082 | md5_54cbd3b0869bf439fdb44b2134d831a7 | 1.67 | 114.519 | 28.3891 | 33.3305 |
| 2130_Iteration1.5_00083 | md5_4ef9d06c52ca21a3c72edb3d1e34d887 | 2.16 | 126.773 | 35.8146 | 40.2452 |
| 2130_Iteration1.5_00084 | md5_6989c5e6f76eb49570038d3d5776410b | 1.68 | 126.497 | 25.9446 | 37.7035 |
| 2130_Iteration1.5_00085 | md5_98860fd1b437cce384562f2dc1cf9d28 | 2.01 | 106.522 | 29.5015 | 36.8901 |
| 2130_Iteration1.5_00086 | md5_975f7cd499b5467063d1582257633fd1 | 1.7 | 138.72 | 24.902 | 39.6329 |
| 2130_Iteration1.5_00087 | md5_23639310c686f78739fd6a9980e5f424 | 0.92 | 96.2368 | 19.2254 | 34.7445 |
| 2130_Iteration1.5_00088 | md5_67638acfdb3bf174f9dc45d5d551d07d | 1.76 | 131.793 | 21.4707 | 35.7137 |
| 2130_Iteration1.5_00089 | md5_4c674020ad4709db9d5874af1b2f0c9b | 1.24 | 137.681 | 40.2764 | 46.7019 |
| 2130_Iteration1.5_00090 | md5_ddfa55c0c4afa535fdefbe0293a7a027 | 1.53 | 149.735 | 34.1782 | 41.4905 |
| 2130_Iteration1.5_00091 | md5_5a3100e97ea251ab9b1dceb47049173f | 1.19 | 145.929 | 36.1103 | 38.4863 |
| 2130_Iteration1.5_00092 | md5_2b3d375d70f4dfc682a73c79d6192c36 | 1.3 | 148.869 | 46.1453 | 37.7934 |
| 2130_Iteration1.5_00093 | md5_cb1732065c938f7bdf6a12d39a72f90c | 1.54 | 154.352 | 36.1243 | 41.5974 |
| 2130_Iteration1.5_00094 | md5_262e96c1bdc514d4b4609a39c613a3f4 | 1.43 | 145.217 | 32.63 | 38.9733 |
| 2130_Iteration1.5_00095 | md5_ef201091f797b7f61249f3c685e6a321 | 1.76 | 147.913 | 32.4579 | 38.6373 |

List of Sequences

SEQ ID NO: 1, md5_e3d1904966eaf73aed30331f60b658e9 - mAb_RBD_2130_FAB_20211231_00017
amino acid sequence
EVQLVESGGGLVKPGGSLRLSCAASGFTFRDVWMSWVRQAPGKGLEWVGRIKSKEDGGTTDYAA
PVKGRFTISRDDSKNTLYLQMNSLKTEDTAVYYCTTAGSYYFDTVGPGLPEGKFDYWGQGTLVTV
SSDIVMTQSPDSLAVSLGERATINCKSSQSVLYWANNKNYLAWYQQKPGQPPKLLMYWASTRESG
VPDRFSGSGSGAEFTLTISSLQAEDVAIYYCQQYYSTLTFGGGTKVEIKR
SEQ ID NO: 2, md5_d323d72bb6bc337ea256bdd92ceba08e - mAb_RBD_2130_FAB_20211231_00174
amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTRGPGLPEGKFDYWGQGTLVTV
SSDIVMTQSPDSLAVSLGERATINCKSSQSVLYSSNNKNYLAWYQQKPGQPPKLLMYWASTRESGV
PDRFSGSGSGAEFTLTISSLQAEDVAIYYCQQYYSTLTFGGGTKVEIKR
SEQ ID NO: 3, md5_9f6486808688a2b46ec220af47cb95c5 - mAb_RBD_2130_FAB_20211231_00194
amino acid sequence
EVQLVESGGGLVKPGGSLRLSCAASGFTFRDVWMSWVRQAPGKGLEWVGRIKSKWDGGTTDYA
APVKGRFTISRDDSKNTLYLQMNSLKTEDTAVYYCTTAGSYYYDTVGPGLPEGKFDYWGQGTLVT
VSSDIVMTQSPDSLAVSLGERATINCKSSQSVLYSSNNKNYLAWYQQKPGQPPKLLMYWASTRESG
VPDRFSGSGSGAEFTLTISSLQAEDVAIYYCQQYYSTLTFGGGTKVEIKR
SEQ ID NO: 4, md5_eebd2cc1786b724358ced82fe314465c - mAb_RBD_2130_FAB_20211231_00199
amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SSDIVMTQSPDSLAVSLGERATINCKSSQSVLYSYNNKNYLAWYQQKPGQPPKLLMYWASTRESG
VPDRFSGSGSGAEFTLTISSLQAEDVAIYYCQQYYSTLTFGGGTKVEIKR
SEQ ID NO: 5, md5_aaca5fa01a0f27e83e39da01a4457347 - mAb_RBD_2130_FAB_20211231_00200
amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SSDIVMTQSPDSLAVSLGERATINCKSSQSVLYSWNNKNYLAWYQQKPGQPPKLLMYWASTRESG
VPDRFSGSGSGAEFTLTISSLQAEDVAIYYCQQYYSTLTFGGGTKVEIKR
SEQ ID NO: 6, md5_2041aa110908c8cde47ed7aabc074be8 - v2130_SFE_20220104_00015 amino acid
sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SSDIVMTQSPDSLAVSLGERATINCKSSQSVLYQYNNKNYLAWYQQKPGQPPKLLMYWASHRESG
VPDRFSGSGSGAEFTLTISSLQAEDVAIYYCQQYYSTLTFGGGTKVEIKR
SEQ ID NO: 7, md5_1089bd14be7c511aa67dbef73eeb1e42 - v2130_SFE_20220104_00024 amino acid
sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SSDIVMTQSPDSLAVSLGERATINCKSSQSVLYWSNNKNYLAWYQQKPGQPPKLLMYWASERESG
VPDRFSGSGSGAEFTLTISSLQAEDVAIYYCQQYYSTLTFGGGTKVEIKR
SEQ ID NO: 8, md5_689536fbc67d8ee33e80ca5a6709d167 -
mAb_v2130JustStableLines_20220111_00002 amino acid sequence
EVQLVESGGGLVKPGGSLRLSCAASGFTFRDVWMSWVRQAPGKGLEWVGRIKSKDDGGTTDYAA
PVKGRFTISRDDSKNTLYLQMNSLKTEDTAVYYCTTAGSYYYDTVGPGLPEGKFDYWGQGTLVTV
SSDIVMTQSPDSLAVSLGERATINCKSSQSVLYSFNNKNYLAWYQQKPGQPPKLLMYWASERESGV
PDRFSGSGSGAEFTLTISSLQAEDVAIYYCQQYYSTLTFGGGTKVEIKR
SEQ ID NO: 9, md5_a314041f0f349a3dc6b474c069163d9b -
mAb_v2130Vanderbilt_184_20220114_00111 amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPELPEGKFDYWGQGTLVTV
SSDIVMTQSPDSLAVSLGERATINCKSSQSVLYYVNNYNYLAWYQQKPGQPPKLLMYWASTRESG
VPDRFSGSGSGAEFTLTISSLQAEDVAIYYCQQYYSTLTFGGGTKVEIKR
SEQ ID NO: 10, md5_3052c528d578179c00211647380e5fb7 -
mAb_v2130Vanderbilt_184 20220114_00112 amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPELPEGKFDYWGQGTLVTV
SSDIVMTQSPDSLAVSLGERATINCKSSQSVLYAANNKNYLAWYQQKPGQPPKLLMYWASERESG
VPDRFSGSGSGAEFTLTISSLQAEDVAIYYCQQYYSTLTFGGGTKVEIKR
SEQ ID NO: 11, md5_2cdc3bd28ff984b7c5b8d2a86ae9553f - v2130_upsample_20220121_00009 amino
acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYDDTVGPGLPEGKIDYWGQGTLVTV
SSDIVMTQSPDSLAVSLGERATINCKSSQSVLYSVNNKNYLAWYQQKPGQPPKLLMNWASTRESG
VPDRFSGSGSGAEFTLTISSLQAEDVAIYYCQQYYSTLTFGGGTKVEIKR
SEQ ID NO: 12, md5_6e9c26854d3e83e74f379947355e3c6a - v2130_upsample_20220121_00298 amino
acid sequence
EVQLVESGGGLVKPGGSLRLSCAASGFTFRDVWMSWVRQAPGKGLEWVGRIKSKEDGGTTDYAA
PVKGRFTISRDDSKNTLYLQMNSLKTEDTAVYYCTTAGSYYHDTVGPGLPEGKFDYWGQGTLVTV
SSDIVMTQSPDSLAVSLGERATINCKSSQSVLYRSNNLNYLAWYQQKPGQPPKLLMYWASTRESGV
PDRFSGSGSGAEFTLTISSLQAEDVAIYYCQQYYSTLTFGGGTKVEIKR
SEQ ID NO: 13, md5_8813a032f188710d1acf700cb2d61380 - v2130_upsample_20220121_00314 amino
acid sequence
EVQLVESGGGLVKPGGSLRLSCAASGFTFRDVWMSWVRQAPGKGLEWVGRIKSKHDGGTTDYAA
PVKGRFTISRDDSKNTLYLQMNSLKTEDTAVYYCTTAGSYYYDTVLPGLPEGKFDYWGQGTLVTV
SSDIVMTQSPDSLAVSLGERATINCKSSQSVLYSYNNKNYLAWYQQKPGQPPKLLMEWASTRESG
VPDRFSGSGSGAEFTLTISSLQAEDVAIYYCQQYYSTLTFGGGTKVEIKR
SEQ ID NO: 14, md5_6362c40ele8234bdalf78cd76d72ca17 - v2130_upsample_20220121_00354 amino
acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTRGPELPEGKFDYWGQGTLVTV
SSDIVMTQSPDSLAVSLGERATINCKSSQSVLYAANNKNYLAWYQQKPGQPPKLLMYWASERESG
VPDRFSGSGSGAEFTLTISSLQAEDVAIYYCQQYYSTLTFGGGTKVEIKR
SEQ ID NO: 15, md5_ac4828a29703823447738469ab260f6 - Hum ID = 2130_Iteration1.5_00011amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlyssnnknylawyqqkpgqppkllmywastresgvpdr
fsgsgsgaeftltisslqaedvaiyycqqyystltfgggtkveik
SEQ ID NO: 16, md5_7b02dbd1899b0826cc7d6fea6f5d88df - Hum ID = 2130_Iteration1.5_00012 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvsdivmtqspdslavslgeratinckssqsvlyasnnknylawyqqkpgqppkllmywastresgvpd
rfsgsgsgaeftltisslqaedvaiyycqqyystltfgggtkveik
SEQ ID NO: 17, md5_844594c4fcfe9750632693465ec9abb3 - Hum ID = 2130_Iteration1.5_00013 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvsdivmtqspdslavslgeratinckssqsvlysannknylawyqqkpgqppkllmywastresgvpd
rfsgsgsgaeftltisslqaedvaiyycqqyystltfgggtkveik
SEQ ID NO: 18, md5_b33188496fde3cf4558f2c579cd5de40 - Hum ID = 2130_Iteration1.5_00014 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvsdivmtqspdslavslgeratinckssqsvlyssnnknylawyqqkpgqppkllmywaseresgvpd
rfsgsgsgaeftltisslqaedvaiyycqqyystltfgggtkveik
SEQ ID NO: 19, md5_f3b8e8673d31b5b9fcffcc3087f21318-Hum_ID = 2130_Iteration1.5_00015 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvsdivmtqspdslavslgeratinckssqsvlyssnnknylawyqqkpgqppkllmywashresgvpd
rfsgsgsgaeftltisslqaedvaiyycqqyystltfgggtkveik
SEQ ID NO: 20, md5_c66c7b0b82bfaf9bbb460ec3772fcd18- Hum_ID = 2130_Iteration1.5_00016 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpglpegkfdywgqgtlvtvsdivmtqspdslavslgeratinckssqsvlyssnnknylawyqqkpgqppkllmywastresgvpd
rfsgsgsgaeftltisslqaedvaiyycqqyystltfgggtkveik
SEQ ID NO: 21, md5_e3e94e79c28dfaed07e6df24598b4e1d - Hum_ID = 2130_Iteration1.5_00017 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 22, md5_62ee8bbf442fcabb49570777fcfead32 - Hum_ID = 2130_Iteration1.5_00018 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpelpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 23, md5_99a29102747e691465b2e14d9a903548 - Hum_ID = 2130_Iteration1.5_00019 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpevpegkfdywgqgtlvtvsdivmtqspdslavslgeratinckssqsvlyaannknylawyqqkpgqppkllmywaseresgvpd
rfsgsgsgaeftltisslqaedvaiyycqqyystltfgggtkveik
SEQ ID NO: 24, md5_e4daa4c3776ff69b0bd45f85b4c8da3d - Hum_ID = 2130_Iteration1.5_00020 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpelpegkfdywgqgtlvtvsdivmtqspdslavslgeratinckssqsvlyaynnknylawyqqkpgqppkllmywaseresgvpd
rfsgsgsgaeftltisslqaedvaiyycqqyystltfgggtkveik
SEQ ID NO: 25, md5_ed7e966157c0b81cb332148fe52d35d4- Hum_ID = 2130_Iteration1.5_00021 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpevpegkfdywgqgtlvtvsdivmtqspdslavslgeratinckssqsvlyaannknylawyqqkpgqppkllmywaseresgv
pdrfsgsgsgaeftltisslqaedvaiyycqqyystltfgggtkveik
SEQ ID NO: 26, md5_698c4a4679aedfb0cae31cd74e473906 - Hum_ID = 2130_Iteration1.5_00022 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpevpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 27, md5_f2345e12679b385c2e3aa594f10c6dc0 - Hum_ID = 2130_Iteration1.5_00023 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpelpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 28, md5_778c2db623alf1fb15a9aa3d68c34812 -Hum_ID = 2130_Iteration1.5_00024 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpelpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 29, md5_685d04f38fa28de4ba7d9db14a3453bc - Hum_ID = 2130_Iteration1.5_00025 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpevpegkfdywgqgtlvtvsdivmtqspdslavslgeratinckssqsvlyaannknylawyqqkpgqppkllmywaseresgvp
drfsgsgsgaeftltisslqaedvaiyycqqyystltfgggtkveik
SEQ ID NO: 30, md5_c9601b59547deb9e0841d3d9f45a1afc - Hum_ID = 2130_Iteration1.5_00026 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlyaynnknylawyqqkpgqppkllmywaseresgvpd
rfsgsgsgaeftltisslqaedvaiyycqqyystltfgggtkveik
SEQ ID NO: 31, md5_f0562398a33d00e711906550f196293e -Hum_ID = 2130_Iteration1.5_00027 amino
acid sequence
evqlvesggglvkpggslrlscaasgftfrdvwmswvrqapgkglewvgrikskwdggttdyaapvkgrftisrddskntlylqmnslktedtavyyc
ttagsyyddtvgpglpegkidywgqgtlvtvsdivmtqspdslavslgeratinckssqsvlysvnnknylawyqqkpgqppkllmnwastresgvp
drfsgsgsgaeftltisslqaedvaiyycqqyystltfgggtkveik
SEQ ID NO: 32, md5_8c6a6c711aa8191a68d8d423aefa3cf0 - Hum_ID = 2130_Iteration1.5_00028 amino
acid sequence
evqlvesggglvkpggslrlscaasgftfrdvwmswvrqapgkglewvgrikskedggttdyaapvkgrftisrddskntlylqmnslktedtavyyct
tagsyyddtvgpglpegkidywgqgtlvtvsdivmtqspdslavslgeratinckssqsvlysvnnknylawyqqkpgqppkllmnwastresgvp
drfsgsgsgaeftltisslqaedvaiyycqqyystltfgggtkveik
SEQ ID NO: 33, md5_56747677dcdee3db6bf31dc958bd4ec9 - Hum_ID = 2130_Iteration1.5_00029 amino
acid sequence
evqlvesggglvkpggslrlscaasgftfrdvwmswvrqapgkglewvgrikskidggttdyaapvkgrftisrddskntlylqmnslktedtavyyctt
agsyyddtrgpglpegkidywgqgtlvtvsdivmtqspdslavslgeratinckssqsvlysvnnknylawyqqkpgqppkllmnwastresgvpdr
fsgsgsgaeftltisslqaedvaiyycqqyystltfgggtkveik
SEQ ID NO: 34, md5_88ff4f3db54437166d0ca5bf0542264a - Hum_ID = 2130_Iteration1.5_00030 amino
acid sequence
evqlvesggglvkpggslrlscaasgftfrdvwmswvrqapgkglewvgrikskidggttdyaapvkgrftisrddskntlylqmnslktedtavyyctt
agsyyddtvgpelpegkidywgqgtlvtvsdivmtqspdslavslgeratinckssqsvlysvnnknylawyqqkpgqppkllmnwastresgvpd
rfsgsgsgaeftltisslqaedvaiyycqqyystltfgggtkveik
SEQ ID NO: 35, md5_768ff12cla4b8beaf0ab30cad7407dac - Hum_ID = 2130_Iteration1.5_00031 amino
acid sequence
evqlvesggglvkpggslrlscaasgftfrdvwmswvrqapgkglewvgrikskidggttdyaapvkgrftisrddskntlylqmnslktedtavyyctt
agsyyddtvgpgvpegkidywgqgtlvtvsdivmtqspdslavslgeratinckssqsvlysvnnknylawyqqkpgqppkllmnwastresgvpd
rfsgsgsgaeftltisslqaedvaiyycqqyystltfgggtkveik
SEQ ID NO: 36, md5_71dal1c6a5f4fca7713a7976c1429c39 - Hum_ID = 2130_Iteration1.5_00032 amino
acid sequence
evqlvesggglvkpggslrlscaasgftfrdvwmswvrqapgkglewvgrikskidggttdyaapvkgrftisrddskntlylqmnslktedtavyyctt
agsyyddtvgpglpegkidywgqgtlvtvsdivmtqspdslavslgeratinckssqsvlysvnnknylawyqqkpgqppkllmnwaseresgvpd
rfsgsgsgaeftltisslqaedvaiyycqqyystltfgggtkveik
SEQ ID NO: 37, md5_d70e5cafa3b8348a029dc50af54e2b00 - Hum_ID = 2130_Iteration1.5_00033 amino
acid sequence
evqlvesggglvkpggslrlscaasgftfrdvwmswvrqapgkglewvgrikskidggttdyaapvkgrftisrddskntlylqmnslktedtavyyctt
agsyyddtvgpglpegkidywgqgtlvtvsdivmtqspdslavslgeratinckssqsvlysvnnknylawyqqkpgqppkllmnwashresgvpd
rfsgsgsgaeftltisslqaedvaiyycqqyystltfgggtkveik
SEQ ID NO: 38, md5_61311844b435d59f5cb57bebba4c5f70 - Hum_ID = 2130_Iteration1.5_00034 amino
acid sequence
evqlvesggglvkpggslrlscaasgftfrdvwmswvrqapgkglewvgrikskedggttdyaapvkgrftisrddskntlylqmnslktedtavyyct
tagsyyydtvlpglpegkfdywgqgtlvtvsdivmtqspdslavslgeratinckssqsvlysynnknylawyqqkpgqppkllmewastresgvpd
rfsgsgsgaeftltisslqaedvaiyycqqyystltfgggtkveik
SEQ ID NO: 39, md5_d62aa0dc67f56elbaefc5da9acd1237c - Hum_ID = 2130_Iteration1.5_00035 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvlpglpegkfdywgqgtlvtvsdivmtqspdslavslgeratinckssqsvlysynnknylawyqqkpgqppkllmewastresgvp
drfsgsgsgaeftltisslqaedvaiyycqqyystltfgggtkveik
SEQ ID NO: 40, md5_f3c22c1c68763fa02ca4f48a93cb8eeb - Hum_ID = 2130_Iteration1.5_00036 amino
acid sequence
evqlvesggglvkpggslrlscaasgftfrdvwmswvrqapgkglewvgrikskhdggttdyaapvkgrftisrddskntlylqmnslktedtavyyct
tagsyyydtrlpglpegkfdywgqgtlvtvsdivmtqspdslavslgeratinckssqsvlysynnknylawyqqkpgqppkllmewastresgvpdr
fsgsgsgaeftltisslqaedvaiyycqqyystltfgggtkveik
SEQ ID NO: 41, md5_bdf4629c9cla56f229956cf9299ca568 - Hum_ID = 2130_Iteration1.5_00037 amino
acid sequence
evqlvesggglvkpggslrlscaasgftfrdvwmswvrqapgkglewvgrikskhdggttdyaapvkgrftisrddskntlylqmnslktedtavyyct
tagsyyydtvlpelpegkfdywgqgtlvtvsdivmtqspdslavslgeratinckssqsvlysynnknylawyqqkpgqppkllmewastresgvpd
rfsgsgsgaeftltisslqaedvaiyycqqyystltfgggtkveik
SEQ ID NO: 42, md5_b0c846f01fd4ee8eeac158d081bfb2be - Hum_ID = 2130_Iteration1.5_00038 amino
acid sequence
evqlvesggglvkpggslrlscaasgftfrdvwmswvrqapgkglewvgrikskhdggttdyaapvkgrftisrddskntlylqmnslktedtavyyct
tagsyyydtvlpgvpegkfdywgqgtlvtvsdivmtqspdslavslgeratinckssqsvlysynnknylawyqqkpgqppkllmewastresgvp
drfsgsgsgaeftltisslqaedvaiyycqqyystltfgggtkveik
SEQ ID NO: 43, md5_d99535affb9946f40586d7a1573072de - Hum_ID = 2130_Iteration1.5_00039 amino
acid sequence
evqlvesggglvkpggslrlscaasgftfrdvwmswvrqapgkglewvgrikskhdggttdyaapvkgrftisrddskntlylqmnslktedtavyyct
tagsyyydtvlpglpegkfdywgqgtlvtvsdivmtqspdslavslgeratinckssqsvlysynnknylawyqqkpgqppkllmewashresgvp
drfsgsgsgaeftltisslqaedvaiyycqqyystltfgggtkveik
SEQ ID NO: 44, md5_052e2bc57e68e1b4530dc038246eee29 - Hum_ID = 2130_Iteration1.5_00040 amino
acid sequence
evqlvesggglvkpggslrlscaasgftfrdvwmswvrqapgkglewvgrikskedggttdyaapvkgrftisrddskntlylqmnslktedtavyyct
tagsyyhdtrgpglpegkfdywgqgtlvtvsdivmtqspdslavslgeratinckssqsvlyrsnnlnylawyqqkpgqppkllmywastresgvpdr
fsgsgsgaeftltisslqaedvaiyycqqyystltfgggtkveik
SEQ ID NO: 45, md5_8cd253058fdec72fbaf58cf41393724f - Hum_ID = 2130_Iteration1.5_00041 amino
acid sequence
evqlvesggglvkpggslrlscaasgftfrdvwmswvrqapgkglewvgrikskedggttdyaapvkgrftisrddskntlylqmnslktedtavyyct
tagsyyhdtvgpelpegkfdywgqgtlvtvsdivmtqspdslavslgeratinckssqsvlyrsnnlnylawyqqkpgqppkllmywastresgvpdr
fsgsgsgaeftltisslqaedvaiyycqqyystltfgggtkveik
SEQ ID NO: 46, md5_702355da28ed59666ce6cf3a65d9e59f - Hum_ID = 2130_Iteration1.5_00042 amino
acid sequence
evqlvesggglvkpggslrlscaasgftfrdvwmswvrqapgkglewvgrikskedggttdyaapvkgrftisrddskntlylqmnslktedtavyyct
tagsyyhdtrgpelpegkfdywgqgtlvtvsdivmtqspdslavslgeratinckssqsvlyrsnnlnylawyqqkpgqppkllmywastresgvpdr
fsgsgsgaeftltisslqaedvaiyycqqyystltfgggtkveik
SEQ ID NO: 47, md5_bf465b80dfbcfb6dd36bcd1717737f7d - Hum_ID = 2130_Iteration1.5_00043 amino
acid sequence
evqlvesggglvkpggslrlscaasgftfrdvwmswvrqapgkglewvgrikskedggttdyaapvkgrftisrddskntlylqmnslktedtavyyct
tagsyyhdtrgpglpegkfdywgqgtlvtvsdivmtqspdslavslgeratinckssqsvlyrsnnlnylawyqqkpgqppkllmywaseresgvpd
rfsgsgsgaeftltisslqaedvaiyycqqyystltfgggtkveik
SEQ ID NO: 48, md5_c209ad3a42be1fb8fda0518f8cc21c2c -Hum ID = 2130_Iteration1.5_00044 amino
acid sequence
evqlvesggglvkpggslrlscaasgftfrdvwmswvrqapgkglewvgrikskedggttdyaapvkgrftisrddskntlylqmnslktedtavyyct
tagsyyhdtvgpglpegkfdywgqgtlvtvsdivmtqspdslavslgeratinckssqsvlyrsnnlnylawyqqkpgqppkllmywaseresgvpd
rfsgsgsgaeftltisslqaedvaiyycqqyystltfgggtkveik
SEQ ID NO: 49, md5_1e9c952de63aa8327acff1cc2d2abbc7 - Hum ID = 2130_Iteration1.5_00045 amino
acid sequence
evqlvesggglvkpggslrlscaasgftfrdvwmswvrqapgkglewvgrikskwdggttdyaapvkgrftisrddskntlylqmnslktedtavyyc
ttagsyyhdtvgpglpegkfdywgqgtlvtvsdivmtqspdslavslgeratinckssqsvlyrsnnlnylawyqqkpgqppkllmywastresgvpd
rfsgsgsgaeftltisslqaedvaiyycqqyystltfgggtkveik
SEQ ID NO: 50, md5_2af950cc1c1898e0a49b94eab292aa66 - Hum ID = 2130_Iteration1.5_00046 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpglpegkfdywgqgtlvtvsdivmtqspdslavslgeratinckssqsvlysfnnknylawyqqkpgqppkllmywaseresgvp
drfsgsgsgaeftltisslqaedvaiyycqqyystltfgggtkveik
SEQ ID NO: 51, md5_aab1483540e1ae1278dc268a9ab2565d - Hum ID = 2130_Iteration1.5_00047 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpglpegkfdywgqgtlvtvsdivmtqspdslavslgeratinckssqsvlysfnnknylawyqqkpgqppkllmywaseresgvp
drfsgsgsgaeftltisslqaedvaiyycqqyystltfgggtkveik
SEQ ID NO: 52, md5_460d3280c27f11b4fc0946ba8df54759 - Hum ID = 2130_Iteration1.5_00048 amino
acid sequence
evqlvesggglvkpggslrlscaasgftfrdvwmswvrqapgkglewvgrikskddggttdyaapvkgrftisrddskntlylqmnslktedtavyyct
tagsyyydtrgpglpegkfdywgqgtlvtvsdivmtqspdslavslgeratinckssqsvlysfnnknylawyqqkpgqppkllmywaseresgvpd
rfsgsgsgaeftltisslqaedvaiyycqqyystltfgggtkveik
SEQ ID NO: 53, md5_19b8elf3e16d175d2f1f038eb9e3ad3f - Hum_ID = 2130_Iteration1.5_00049 amino
acid sequence
evqlvesggglvkpggslrlscaasgftfrdvwmswvrqapgkglewvgrikskddggttdyaapvkgrftisrddskntlylqmnslktedtavyyct
tagsyyydtvgpelpegkfdywgqgtlvtvsdivmtqspdslavslgeratinckssqsvlysfnnknylawyqqkpgqppkllmywaseresgvp
drfsgsgsgaeftltisslqaedvaiyycqqyystltfgggtkveik
SEQ ID NO: 54, md5_259e5b1dfa4215ab7683e6bf24b7b939 - Hum_ID = 2130_Iteration1.5_00050 amino
acid sequence
evqlvesggglvkpggslrlscaasgftfrdvwmswvrqapgkglewvgrikskddggttdyaapvkgrftisrddskntlylqmnslktedtavyyct
tagsyyydtvgpgvpegkfdywgqgtlvtvsdivmtqspdslavslgeratinckssqsvlysfnnknylawyqqkpgqppkllmywaseresgvp
drfsgsgsgaeftltisslqaedvaiyycqqyystltfgggtkveik
SEQ ID NO: 55, md5_a2492428ecb5126bc8c8c03e97de7eb0 - Hum_ID = 2130_Iteration1.5_00051 amino
acid sequence
evqlvesggglvkpggslrlscaasgftfrdvwmswvrqapgkglewvgrikskddggttdyaapvkgrftisrddskntlylqmnslktedtavyyct
tagsyyydtrgpelpegkfdywgqgtlvtvsdivmtqspdslavslgeratinckssqsvlysfnnknylawyqqkpgqppkllmywaseresgvpd
rfsgsgsgaeftltisslqaedvaiyycqqyystltfgggtkveik
SEQ ID NO: 56, md5_98948967f2e5d0166316678e80ae2446 - Hum_ID = 2130_Iteration1.5_00052 amino
acid sequence
evqlvesggglvkpggslrlscaasgftfrdvwmswvrqapgkglewvgrikskddggttdyaapvkgrftisrddskntlylqmnslktedtavyyct
tagsyyydtvgpglpegkfdywgqgtlvtvsdivmtqspdslavslgeratinckssqsvlysannknylawyqqkpgqppkllmywaseresgvp
drfsgsgsgaeftltisslqaedvaiyycqqyystltfgggtkveik
SEQ ID NO: 57, md5_423d9654865843a72c7888f5802171a6 - Hum_ID = 2130_Iteration1.5_00053 amino
acid sequence
evqlvesggglvkpggslrlscaasgftfrdvwmswvrqapgkglewvgrikskddggttdyaapvkgrftisrddskntlylqmnslktedtavyyct
tagsyyydtvgpglpegkfdywgqgtlvtvsdivmtqspdslavslgeratinckssqsvlyswnnknylawyqqkpgqppkllmywaseresgvp
drfsgsgsgaeftltisslqaedvaiyycqqyystltfgggtkveik
SEQ ID NO: 58, md5_cb395904fdef0858edf0cdb60c59f338 - Hum_ID = 2130_Iteration1.5_00054 amino
acid sequence
evqlvesggglvkpggslrlscaasgftfrdvwmswvrqapgkglewvgrikskddggttdyaapvkgrftisrddskntlylqmnslktedtavyyct
tagsyyydtvgpglpegkfdywgqgtlvtvsdivmtqspdslavslgeratinckssqsvlysynnknylawyqqkpgqppkllmywaseresgvp
drfsgsgsgaeftltisslqaedvaiyycqqyystltfgggtkveik
SEQ ID NO: 59, md5_3d12c9e26e1bef1a735ac5756a577448 - Hum ID = 2130_Iteration1.5_00055 amino
acid sequence
evqlvesggglvkpggslrlscaasgftfrdvwmswvrqapgkglewvgrikskwdggttdyaapvkgrftisrddskntlylqmnslktedtavyyc
ttagsyyfdtvgpglpegkfdywgqgtlvtvsdivmtqspdslavslgeratinckssqsvlywannknylawyqqkpgqppkllmywastresgvp
drfsgsgsgaeftltisslqaedvaiyycqqyystltfgggtkveik
SEQ ID NO: 60, md5_7c235b7cd768b595619b8e3006608a13 - Hum_ID = 2130_Iteration1.5_00056 amino
acid sequence
evqlvesggglvkpggslrlscaasgftfrdvwmswvrqapgkglewvgrikskedggttdyaapvkgrftisrddskntlylqmnslktedtavyyct
tagsyyfdtrgpglpegkfdywgqgtlvtvsdivmtqspdslavslgeratinckssqsvlywannknylawyqqkpgqppkllmywastresgvp
drfsgsgsgaeftltisslqaedvaiyycqqyystltfgggtkveik
SEQ ID NO: 61, md5_c2933cc413d4a1987d1f39c047717b51 - Hum ID = 2130_Iteration1.5_00057 amino
acid sequence
evqlvesggglvkpggslrlscaasgftfrdvwmswvrqapgkglewvgrikskedggttdyaapvkgrftisrddskntlylqmnslktedtavyyct
tagsyyfdtvgpelpegkfdywgqgtlvtvsdivmtqspdslavslgeratinckssqsvlywannknylawyqqkpgqppkllmywastresgvp
drfsgsgsgaeftltisslqaedvaiyycqqyystltfgggtkveik
SEQ ID NO: 62, md5_1dae53d51cedc8e8ea0f1ebc93170ea2 - Hum_ID = 2130_Iteration1.5_00058 amino
acid sequence
evqlvesggglvkpggslrlscaasgftfrdvwmswvrqapgkglewvgrikskedggttdyaapvkgrftisrddskntlylqmnslktedtavyyct
tagsyyfdtvgpgvpegkfdywgqgtlvtvsdivmtqspdslavslgeratinckssqsvlywannknylawyqqkpgqppkllmywastresgvp
drfsgsgsgaeftltisslqaedvaiyycqqyystltfgggtkveik
SEQ ID NO: 63, md5_6a5d7f58da6a6e0d73f4198e1bd0d263 - Hum_ID = 2130_Iteration1.5_00059 amino
acid sequence
evqlvesggglvkpggslrlscaasgftfrdvwmswvrqapgkglewvgrikskedggttdyaapvkgrftisrddskntlylqmnslktedtavyyct
tagsyyfdtrgpelpegkfdywgqgtlvtvsdivmtqspdslavslgeratinckssqsvlywannknylawyqqkpgqppkllmywastresgvpd
rfsgsgsgaeftltisslqaedvaiyycqqyystltfgggtkveik
SEQ ID NO: 64, md5_7b918286419ed897274892be92a5a1da - Hum_ID = 2130_Iteration1.5_00060 amino
acid sequence
evqlvesggglvkpggslrlscaasgftfrdvwmswvrqapgkglewvgrikskedggttdyaapvkgrftisrddskntlylqmnslktedtavyyct
tagsyyfdtvgpglpegkfdywgqgtlvtvsdivmtqspdslavslgeratinckssqsvlywannknylawyqqkpgqppkllmywaseresgvp
drfsgsgsgaeftltisslqaedvaiyycqqyystltfgggtkveik
SEQ ID NO: 65, md5_1f3ad4f915219eabdb9d95ef4455cb6f - Hum_ID = 2130_Iteration1.5_00061 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpglpegkfdywgqgtlvtvsdivmtqspdslavslgeratinckssqsvlywsnnknylawyqqkpgqppkllmywaseresgv
pdrfsgsgsgaeftltisslqaedvaiyycqqyystltfgggtkveik
SEQ ID NO: 66, md5_96f6cf64380146cd84af6a7ce3fd7fb9 - Hum_ID = 2130_Iteration1.5_00062 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpglpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 67, md5_14d2e59a93698d095fd22c7ba01fcbce -Hum ID = 2130_Iteration1.5_00063 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpglpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 68, md5_d320606976fb803b69f334046fc84543 - Hum ID = 2130_Iteration1.5_00064 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 69, md5_86730c57319dd14c86fe94bc812ad5fa - Hum_ID = 2130_Iteration1.5_00065 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpgvpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 70, md5_7da053821284a0d91161e549a39b0c8e - Hum ID = 2130_Iteration1.5_00066 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpglpegkfdywgqgtlvtvsdivmtqspdslavslgeratinckssqsvlywsnnknylawyqqkpgqppkllmywaseresgv
pdrfsgsgsgaeftltisslqaedvaiyycqqyystltfgggtkveik
SEQ ID NO: 71, md5_d3ec50e2dbb15669959be94dc006a449 - Hum_ID = 2130_Iteration1.5_00067 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpelpegkfdywgqgtlvtvsdivmtqspdslavslgeratinckssqsvlywsnnknylawyqqkpgqppkllmywaseresgv
pdrfsgsgsgaeftltisslqaedvaiyycqqyystltfgggtkveik
SEQ ID NO: 72, md5_cc3ee0fe85b16e315e8daee604f29840 - Hum_ID = 2130_Iteration1.5_00068 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpglpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 73, md5_9315a7cc3daele80c6cefala7840e9d1 - Hum_ID = 2130_Iteration1.5_00069 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpelpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 74, md5_ee41994614eee7e3131741e986153676 - Hum ID = 2130_Iteration1.5_00070 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvsdivmtqspdslavslgeratinckssqsvlywannknylawyqqkpgqppkllmywaseresgvp
drfsgsgsgaeftltisslqaedvaiyycqqyystltfgggtkveik
SEQ ID NO: 75, md5_b2200f7396e3968080599cd4e6f8f81b - Hum_ID = 2130_Iteration1.5_00071 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpelpegkfdywgqgtlvtvsdivmtqspdslavslgeratinckssqsvlywsnnknylawyqqkpgqppkllmywaseresgvpd
rfsgsgsgaeftltisslqaedvaiyycqqyystltfgggtkveik
SEQ ID NO: 76, md5_0f4532e0ae4e7755d3d48072aea1f18c - Hum_ID = 2130_Iteration1.5_00072 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlywsnnknylawyqqkpgqppkllmywaseresgv
pdrfsgsgsgaeftltisslqaedvaiyycqqyystltfgggtkveik
SEQ ID NO: 77, md5_d9e89da7b01686bda150c990ddbf903c - Hum_ID = 2130_Iteration1.5_00073 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpelpegkfdywgqgtlvtvsdivmtqspdslavslgeratinckssqsvlywsnnknylawyqqkpgqppkllmywaseresgvp
drfsgsgsgaeftltisslqaedvaiyycqqyystltfgggtkveik
SEQ ID NO: 78, md5_937dab20e75d4d8a1838dc382cb48b5b - Hum_ID = 2130_Iteration1.5_00074 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpelpegkfdywgqgtlvtvsdivmtqspdslavslgeratinckssqsvlyyvnnynylawyqqkpgqppkllmywastresgvp
drfsgsgsgaeftltisslqaedvaiyycqqyystltfgggtkveik
SEQ ID NO: 79, md5_f5378fc51927d65e51c662b5ea017638 - Hum_ID = 2130_Iteration1.5_00075 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpelpegkfdywgqgtlvtvsdivmtqspdslavslgeratinckssqsvlyyvnnynylawyqqkpgqppkllmywastresgvp
drfsgsgsgaeftltisslqaedvaiyycqqyystltfgggtkveik
SEQ ID NO: 80, md5_2753914b7c94c5cfe1024c3c43176c53 - Hum_ID = 2130_Iteration1.5_00076 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpelpegkfdywgqgtlvtvsdivmtqspdslavslgeratinckssqsvlyyvnnynylawyqqkpgqppkllmywastresgvpd
rfsgsgsgaeftltisslqaedvaiyycqqyystltfgggtkveik
SEQ ID NO: 81, md5_2093fe026edc51c3017ac5da316ed0d4 - Hum_ID = 2130_Iteration1.5_00077 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpevpegkfdywgqgtlvtvsdivmtqspdslavslgeratinckssqsvlyyvnnynylawyqqkpgqppkllmywastresgvp
drfsgsgsgaeftltisslqaedvaiyycqqyystltfgggtkveik
SEQ ID NO: 82, md5_a3a3295e22bbd7a4f09f451acd976ee4 - Hum ID = 2130_Iteration1.5_00078 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlyysnnynylawyqqkpgqppkllmywastresgvpd
rfsgsgsgaeftltisslqaedvaiyycqqyystltfgggtkveik
SEQ ID NO: 83, md5_b4f5565718de43287bb9ef3f80db822b - Hum_ID = 2130_Iteration1.5_00079 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlyyannynylawyqqkpgqppkllmywastresgvpd
rfsgsgsgaeftltisslqaedvaiyycqqyystltfgggtkveik
SEQ ID NO: 84, md5_e40fd800f71a4cf8cd84f490f2d916f2 - Hum_ID = 2130_Iteration1.5_00080 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyyvnnynylawyqqkpgqppkllmywastresgvp
drfsgsgsgaeftltisslqaedvaiyycqqyystltfgggtkveik
SEQ ID NO: 85, md5_442d78ecdb922e86c6fa9fdf25560d62 - Hum ID = 2130_Iteration1.5_00081 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyyvnnynylawyqqkpgqppkllmywaseresgvp
drfsgsgsgaeftltisslqaedvaiyycqqyystltfgggtkveik
SEQ ID NO: 86, md5_54cbd3b0869bf439fdb44b2134d831a7 - Hum ID = 2130_Iteration1.5_00082 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpglpegkfdywgqgtlvtvsdivmtqspdslavslgeratinckssqsvlyqynnknylawyqqkpgqppkllmywashresgv
pdrfsgsgsgaeftltisslqaedvaiyycqqyystltfgggtkveik
SEQ ID NO: 87, md5_4ef9d06c52ca21a3c72edb3d1e34d887 - Hum_ID = 2130_Iteration1.5_00083 amino
acid sequence
evqlvesggglvkpggslrlscaasgftfrdvwmswvrqapgkglewvgrikskidggttdyaapvkgrftisrddskntlylqmnslktedtavyyctt
agsyyydtrgpglpegkfdywgqgtlvtvsdivmtqspdslavslgeratinckssqsvlyqynnknylawyqqkpgqppkllmywashresgvpd
rfsgsgsgaeftltisslqaedvaiyycqqyystltfgggtkveik
SEQ ID NO: 88, md5_6989c5e6f76eb49570038d3d5776410b - Hum_ID = 2130_Iteration1.5_00084 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 89, md5_98860fd1b437cce384562f2dc1cf9d28 - Hum_ID = 2130_Iteration1.5_00085 amino
acid sequence
evqlvesggglvkpggslrlscaasgftfrdvwmswvrqapgkglewvgrikskidggttdyaapvkgrftisrddskntlylqmnslktedtavyyctt
agsyyydtvgpgvpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 90, md5_975f7cd49965467063d1582257633fd1 - Hum ID = 2130_Iteration1.5_00086 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpglpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 91, md5_23639310c686f78739fd6a9980e5f424 - Hum_ID = 2130_Iteration1.5_00087 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpglpegkfdywgqgtlvtvsdivmtqspdslavslgeratinckssqsvlyqynnknylawyqqkpgqppkllmywashresgv
pdrfsgsgsgaeftltisslqaedvaiyycqqyystltfgggtkveik
SEQ ID NO: 92, md5_67638acfdb3bf174f9dc45d5d551d07d-Hum_ID = 2130_Iteration1.5_00088 amino
acid sequence
evqlvesggglvkpggslrlscaasgftfrdvwmswvrqapgkglewvgrikskedggttdyaapvkgrftisrddskntlylqmnslktedtavyyct
tagsyyydtvgpelpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 93, md5_4c674020ad4709db9d5874af1b2f0c96 - Hum_ID = 2130_Iteration1.5_00089 amino
acid sequence
evqlvesggglvkpggslrlscaasgftfrdvwmswvrqapgkglewvgrikskwdggttdyaapvkgrftisrddskntlylqmnslktedtavyyc
ttagsyyydtvgpelpegkfdywgqgtlvtvsdivmtqspdslavslgeratinckssqsvlyqynnknylawyqqkpgqppkllmywashresgv
pdrfsgsgsgaeftltisslqaedvaiyycqqyystltfgggtkveik
SEQ ID NO: 94, md5_ddfa55c0c4afa535fdefbe0293a7a027 - Hum ID = 2130_Iteration1.5_00090 amino
acid sequence
evqlvesggglvkpggslrlscaasgftfrdvwmswvrqapgkglewvgrikskedggttdyaapvkgrftisrddskntlylqmnslktedtavyyct
tagsyyydtrgpelpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 95, md5_5a3100e97ea251ab9b1dceb47049173f - Hum_ID = 2130_Iteration1.5_00091 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyqynnknylawyqqkpgqppkllmywashresgvp
drfsgsgsgaeftltisslqaedvaiyycqqyystltfgggtkveik
SEQ ID NO: 96, md5_2b3d375d70f4dfc682a73c79d6192c36 - Hum_ID = 2130_Iteration1.5_00092 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyqynnknylawyqqkpgqppkllmywaseresgvp
drfsgsgsgaeftltisslqaedvaiyycqqyystltfgggtkveik
SEQ ID NO: 97, md5_cb1732065c938f7bdf6a12d39a72f90c - Hum_ID = 2130_Iteration1.5_00093 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlyswnnknylawyqqkpgqppkllmywastresgvp
drfsgsgsgaeftltisslqaedvaiyycqqyystltfgggtkveik
SEQ ID NO: 98, md5_262e96c1bdc514d4b4609a39c613a3f4 - Hum_ID = 2130_Iteration1.5_00094 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlysynnknylawyqqkpgqppkllmywastresgvp
drfsgsgsgaeftltisslqaedvaiyycqqyystltfgggtkveik
SEQ ID NO: 99, md5_ef201091f797b7f61249f3c685e6a321 -Hum ID = 2130_Iteration1.5_00095 amino
acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvsdivmtqspdslavslgeratinckssqsvlysannknylawyqqkpgqppkllmywastresgvp
drfsgsgsgaeftltisslqaedvaiyycqqyystltfgggtkveik
SEQ ID NO: 100, VH amino acid sequence
EVQLVESGGGLVKPGGSLRLSCAASGFTFRDVWMSWVRQAPGKGLEWVGRIKSKEDGGTTDYAA
PVKGRFTISRDDSKNTLYLQMNSLKTEDTAVYYCTTAGSYYFDTVGPGLPEGKFDYWGQGTLVTV
SS
SEQ ID NO: 101, VH amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTRGPGLPEGKFDYWGQGTLVTV
SS
SEQ ID NO: 102, VH amino acid sequence
EVQLVESGGGLVKPGGSLRLSCAASGFTFRDVWMSWVRQAPGKGLEWVGRIKSKWDGGTTDYA
APVKGRFTISRDDSKNTLYLQMNSLKTEDTAVYYCTTAGSYYYDTVGPGLPEGKFDYWGQGTLVT
VSS
SEQ ID NO: 103, VH amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SS
SEQ ID NO: 104, VH amino acid sequence
EVQLVESGGGLVKPGGSLRLSCAASGFTFRDVWMSWVRQAPGKGLEWVGRIKSKDDGGTTDYAA
PVKGRFTISRDDSKNTLYLQMNSLKTEDTAVYYCTTAGSYYYDTVGPGLPEGKFDYWGQGTLVTV
SS
SEQ ID NO: 105, VH amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPELPEGKFDYWGQGTLVTV
SS
SEQ ID NO: 106, VH amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYDDTVGPGLPEGKIDYWGQGTLVTV
SS
SEQ ID NO: 107, VH amino acid sequence
EVQLVESGGGLVKPGGSLRLSCAASGFTFRDVWMSWVRQAPGKGLEWVGRIKSKEDGGTTDYAA
PVKGRFTISRDDSKNTLYLQMNSLKTEDTAVYYCTTAGSYYHDTVGPGLPEGKFDYWGQGTLVTV
SS
SEQ ID NO: 108, VH amino acid sequence
EVQLVESGGGLVKPGGSLRLSCAASGFTFRDVWMSWVRQAPGKGLEWVGRIKSKHDGGTTDYAA
PVKGRFTISRDDSKNTLYLQMNSLKTEDTAVYYCTTAGSYYYDTVLPGLPEGKFDYWGQGTLVTV
SS
SEQ ID NO: 109, VH amino acid sequence
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTRGPELPEGKFDYWGQGTLVTV
SS
SEQ ID NO: 110, VH amino acid sequence
evqlvesggglvkpggslrlscaasgftfrdvwmswvrqapgkglewvgrikskidggttdyaapvkgrftisrddskntlylqmnslktedtavyyctt
agsyyydtvgpelpegkfdywgqgtlvtvs
SEQ ID NO: 111, VH amino acid sequence
evqlvesggglvkpggslrlscaasgftfrdvwmswvrqapgkglewvgrikskidggttdyaapvkgrftisrddskntlylqmnslktedtavyyctt
agsyyydtvgpglpegkfdywgqgtlvtvs
SEQ ID NO: 112, VH amino acid sequence
Evqlvesggglvkpggslrlscaasgftfrdvwmswvrqapgkglewvgrikskedggttdyaapvkgrftisrddskntlylqmnslktedtavyyc
ttagsyyydtvgpglpegkfdywgqgtlvtvs
SEQ ID NO: 113, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtrgpelpegkfdywgqgtlvtvs
SEQ ID NO: 114, VH amino acid sequence
evqlvesggglvkpggslrlscaasgftfrdvwmswvrqapgkglewvgrikskedggttdyaapvkgrftisrddskntlylqmnslktedtavyyct
tagsyyydtrgpelpegkfdywgqgtlvtvs
SEQ ID NO: 115, VH amino acid sequence
evqlvesggglvkpggslrlscaasgftfrdvwmswvrqapgkglewvgrikskidggttdyaapvkgrftisrddskntlylqmnslktedtavyyctt
agsyyydtrgpevpegkfdywgqgtlvtvs
SEQ ID NO: 116, VH amino acid sequence
evqlvesggglvkpggslrlscaasgftfrdvwmswvrqapgkglewvgrikskidggttdyaapvkgrftisrddskntlylqmnslktedtavyyctt
agsyyydtrgpelpegkfdywgqgtlvtvs
SEQ ID NO: 117, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtrgpevpegkfdywgqgtlvtvs
SEQ ID NO: 118, VH amino acid sequence
evqlvesggglvkpggslrlscaasgftfrdvwmswvrqapgkglewvgrikskedggttdyaapvkgrftisrddskntlylqmnslktedtavyyct
tagsyyydtrgpevpegkfdywgqgtlvtvs
SEQ ID NO: 119, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtvgpelpegkfdywgqgtlvtvs
SEQ ID NO: 120, VH amino acid sequence
evqlvesggglvkpggslrlscaasgftfrdvwmswvrqapgkglewvgrikskedggttdyaapvkgrftisrddskntlylqmnslktedtavyyct
tagsyyydtvgpelpegkfdywgqgtlvtvs
SEQ ID NO: 121, VH amino acid sequence
evqlvesggglvkpggslrlscaasgftfrdvwmswvrqapgkglewvgrikskidggttdyaapvkgrftisrddskntlylqmnslktedtavyyctt
agsyyydtvgpevpegkfdywgqgtlvtvs
SEQ ID NO: 122, VH amino acid sequence
evqlvesggglvkpggslrlscaasgftfrdvwmswvrqapgkglewvgrikskwdggttdyaapvkgrftisrddskntlylqmnslktedtavyyc
ttagsyyddtvgpglpegkidywgqgtlvtvs
SEQ ID NO: 123, VH amino acid sequence
evqlvesggglvkpggslrlscaasgftfrdvwmswvrqapgkglewvgrikskedggttdyaapvkgrftisrddskntlylqmnslktedtavyyct
tagsyyddtvgpglpegkidywgqgtlvtvs
SEQ ID NO: 124, VH amino acid sequence
evqlvesggglvkpggslrlscaasgftfrdvwmswvrqapgkglewvgrikskidggttdyaapvkgrftisrddskntlylqmnslktedtavyyctt
agsyyddtrgpglpegkidywgqgtlvtvs
SEQ ID NO: 125, VH amino acid sequence
evqlvesggglvkpggslrlscaasgftfrdvwmswvrqapgkglewvgrikskidggttdyaapvkgrftisrddskntlylqmnslktedtavyyctt
agsyyddtvgpelpegkidywgqgtlvtvs
SEQ ID NO: 126, VH amino acid sequence
evqlvesggglvkpggslrlscaasgftfrdvwmswvrqapgkglewvgrikskidggttdyaapvkgrftisrddskntlylqmnslktedtavyyctt
agsyyddtvgpgvpegkidywgqgtlvtvs
SEQ ID NO: 127, VH amino acid sequence
evqlvesggglvkpggslrlscaasgftfrdvwmswvrqapgkglewvgrikskidggttdyaapvkgrftisrddskntlylqmnslktedtavyyctt
agsyyddtvgpglpegkidywgqgtlvtvs
SEQ ID NO: 128, VH amino acid sequence
evqlvesggglvkpggslrlscaasgftfrdvwmswvrqapgkglewvgrikskedggttdyaapvkgrftisrddskntlylqmnslktedtavyyct
tagsyyydtvlpglpegkfdywgqgtlvtvs
SEQ ID NO: 129, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtvlpglpegkfdywgqgtlvtvs
SEQ ID NO: 130, VH amino acid sequence
evqlvesggglvkpggslrlscaasgftfrdvwmswvrqapgkglewvgrikskhdggttdyaapvkgrftisrddskntlylqmnslktedtavyyct
tagsyyydtrlpglpegkfdywgqgtlvtvs
SEQ ID NO: 131, VH amino acid sequence
evqlvesggglvkpggslrlscaasgftfrdvwmswvrqapgkglewvgrikskhdggttdyaapvkgrftisrddskntlylqmnslktedtavyyct
tagsyyydtvlpelpegkfdywgqgtlvtvs
SEQ ID NO: 132, VH amino acid sequence
evqlvesggglvkpggslrlscaasgftfrdvwmswvrqapgkglewvgrikskhdggttdyaapvkgrftisrddskntlylqmnslktedtavyyct
tagsyyydtvlpgvpegkfdywgqgtlvtvs
SEQ ID NO: 133, VH amino acid sequence
evqlvesggglvkpggslrlscaasgftfrdvwmswvrqapgkglewvgrikskhdggttdyaapvkgrftisrddskntlylqmnslktedtavyyct
tagsyyydtvlpglpegkfdywgqgtlvtvs
SEQ ID NO: 134, VH amino acid sequence
evqlvesggglvkpggslrlscaasgftfrdvwmswvrqapgkglewvgrikskedggttdyaapvkgrftisrddskntlylqmnslktedtavyyct
tagsyyhdtrgpglpegkfdywgqgtlvtvs
SEQ ID NO: 135, VH amino acid sequence
evqlvesggglvkpggslrlscaasgftfrdvwmswvrqapgkglewvgrikskedggttdyaapvkgrftisrddskntlylqmnslktedtavyyct
tagsyyhdtvgpelpegkfdywgqgtlvtvs
SEQ ID NO: 136, VH amino acid sequence
evqlvesggglvkpggslrlscaasgftfrdvwmswvrqapgkglewvgrikskedggttdyaapvkgrftisrddskntlylqmnslktedtavyyct
tagsyyhdtrgpelpegkfdywgqgtlvtvs
SEQ ID NO: 137, VH amino acid sequence
evqlvesggglvkpggslrlscaasgftfrdvwmswvrqapgkglewvgrikskedggttdyaapvkgrftisrddskntlylqmnslktedtavyyct
tagsyyhdtvgpglpegkfdywgqgtlvtvs
SEQ ID NO: 138, VH amino acid sequence
evqlvesggglvkpggslrlscaasgftfrdvwmswvrqapgkglewvgrikskwdggttdyaapvkgrftisrddskntlylqmnslktedtavyyc
ttagsyyhdtvgpglpegkfdywgqgtlvtvs
SEQ ID NO: 139, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtvgpglpegkfdywgqgtlvtvs
SEQ ID NO: 140, VH amino acid sequence
evqlvesggglvkpggslrlscaasgftfrdvwmswvrqapgkglewvgrikskddggttdyaapvkgrftisrddskntlylqmnslktedtavyyct
tagsyyydtrgpglpegkfdywgqgtlvtvs
SEQ ID NO: 141, VH amino acid sequence
evqlvesggglvkpggslrlscaasgftfrdvwmswvrqapgkglewvgrikskddggttdyaapvkgrftisrddskntlylqmnslktedtavyyct
tagsyyydtvgpelpegkfdywgqgtlvtvs
SEQ ID NO: 142, VH amino acid sequence
evqlvesggglvkpggslrlscaasgftfrdvwmswvrqapgkglewvgrikskddggttdyaapvkgrftisrddskntlylqmnslktedtavyyct
tagsyyydtvgpgvpegkfdywgqgtlvtvs
SEQ ID NO: 143, VH amino acid sequence
evqlvesggglvkpggslrlscaasgftfrdvwmswvrqapgkglewvgrikskddggttdyaapvkgrftisrddskntlylqmnslktedtavyyct
tagsyyydtrgpelpegkfdywgqgtlvtvs
SEQ ID NO: 144, VH amino acid sequence
evqlvesggglvkpggslrlscaasgftfrdvwmswvrqapgkglewvgrikskddggttdyaapvkgrftisrddskntlylqmnslktedtavyyct
tagsyyydtvgpglpegkfdywgqgtlvtvs
SEQ ID NO: 145, VH amino acid sequence
evqlvesggglvkpggslrlscaasgftfrdvwmswvrqapgkglewvgrikskwdggttdyaapvkgrftisrddskntlylqmnslktedtavyyc
ttagsyyfdtvgpglpegkfdywgqgtlvtvs
SEQ ID NO: 146, VH amino acid sequence
evqlvesggglvkpggslrlscaasgftfrdvwmswvrqapgkglewvgrikskedggttdyaapvkgrftisrddskntlylqmnslktedtavyyct
tagsyyfdtrgpglpegkfdywgqgtlvtvs
SEQ ID NO: 147, VH amino acid sequence
evqlvesggglvkpggslrlscaasgftfrdvwmswvrqapgkglewvgrikskedggttdyaapvkgrftisrddskntlylqmnslktedtavyyct
tagsyyfdtvgpelpegkfdywgqgtlvtvs
SEQ ID NO: 148, VH amino acid sequence
evqlvesggglvkpggslrlscaasgftfrdvwmswvrqapgkglewvgrikskedggttdyaapvkgrftisrddskntlylqmnslktedtavyyct
tagsyyfdtvgpgvpegkfdywgqgtlvtvs
SEQ ID NO: 149, VH amino acid sequence
evqlvesggglvkpggslrlscaasgftfrdvwmswvrqapgkglewvgrikskedggttdyaapvkgrftisrddskntlylqmnslktedtavyyct
tagsyyfdtrgpelpegkfdywgqgtlvtvs
SEQ ID NO: 150, VH amino acid sequence
evqlvesggglvkpggslrlscaasgftfrdvwmswvrqapgkglewvgrikskedggttdyaapvkgrftisrddskntlylqmnslktedtavyyct
tagsyyfdtvgpglpegkfdywgqgtlvtvs
SEQ ID NO: 151, VH amino acid sequence
evqlvesggglvkpggslrlscaasgftfrdvwmswvrqapgkglewvgrikskidggttdyaapvkgrftisrddskntlylqmnslktedtavyyctt
agsyyydtrgpglpegkfdywgqgtlvtvs
SEQ ID NO: 152, VH amino acid sequence
evqlvesggglvkpggslrlscaasgftfrdvwmswvrqapgkglewvgrikskidggttdyaapvkgrftisrddskntlylqmnslktedtavyyctt
agsyyydtvgpgvpegkfdywgqgtlvtvs
SEQ ID NO: 153, VH amino acid sequence
evqlvesggglvkpggslrlscaasgftfrdvwmswvrqapgkglewvgrikskwdggttdyaapvkgrftisrddskntlylqmnslktedtavyyc
ttagsyyydtrgpglpegkfdywgqgtlvtvs
SEQ ID NO: 154, VH amino acid sequence
evqlvesggglvkpggslrlscaasgftfrdvwmswvrqapgkglewvgrikskedggttdyaapvkgrftisrddskntlylqmnslktedtavyyct
tagsyyydtrgpglpegkfdywgqgtlvtvs
SEQ ID NO: 155, VL amino acid sequence
DIVMTQSPDSLAVSLGERATINCKSSQSVLYWANNKNYLAWYQQKPGQPPKLLMYWASTRESGVP
DRFSGSGSGAEFTLTISSLQAEDVAIYYCQQYYSTLTFGGGTKVEIKR
SEQ ID NO: 156, VL amino acid sequence
DIVMTQSPDSLAVSLGERATINCKSSQSVLYSSNNKNYLAWYQQKPGQPPKLLMYWASTRESGVP
DRFSGSGSGAEFTLTISSLQAEDVAIYYCQQYYSTLTFGGGTKVEIKR
SEQ ID NO: 157, VL amino acid sequence
DIVMTQSPDSLAVSLGERATINCKSSQSVLYSYNNKNYLAWYQQKPGQPPKLLMYWASTRESGVP
DRFSGSGSGAEFTLTISSLQAEDVAIYYCQQYYSTLTFGGGTKVEIKR
SEQ ID NO: 158, VL amino acid sequence
DIVMTQSPDSLAVSLGERATINCKSSQSVLYSWNNKNYLAWYQQKPGQPPKLLMYWASTRESGVP
DRFSGSGSGAEFTLTISSLQAEDVAIYYCQQYYSTLTFGGGTKVEIKR
SEQ ID NO: 159, VL amino acid sequence
DIVMTQSPDSLAVSLGERATINCKSSQSVLYQYNNKNYLAWYQQKPGQPPKLLMYWASHRESGVP
DRFSGSGSGAEFTLTISSLQAEDVAIYYCQQYYSTLTFGGGTKVEIKR
SEQ ID NO: 160, VL amino acid sequence
DIVMTQSPDSLAVSLGERATINCKSSQSVLYWSNNKNYLAWYQQKPGQPPKLLMYWASERESGVP
DRFSGSGSGAEFTLTISSLQAEDVAIYYCQQYYSTLTFGGGTKVEIKR
SEQ ID NO: 161, VL amino acid sequence
DIVMTQSPDSLAVSLGERATINCKSSQSVLYSFNNKNYLAWYQQKPGQPPKLLMYWASERESGVP
DRFSGSGSGAEFTLTISSLQAEDVAIYYCQQYYSTLTFGGGTKVEIKR
SEQ ID NO: 162, VL amino acid sequence
DIVMTQSPDSLAVSLGERATINCKSSQSVLYYVNNYNYLAWYQQKPGQPPKLLMYWASTRESGVP
DRFSGSGSGAEFTLTISSLQAEDVAIYYCQQYYSTLTFGGGTKVEIKR
SEQ ID NO: 163, VL amino acid sequence
DIVMTQSPDSLAVSLGERATINCKSSQSVLYAANNKNYLAWYQQKPGQPPKLLMYWASERESGVP
DRFSGSGSGAEFTLTISSLQAEDVAIYYCQQYYSTLTFGGGTKVEIKR
SEQ ID NO: 164, VL amino acid sequence
DIVMTQSPDSLAVSLGERATINCKSSQSVLYSVNNKNYLAWYQQKPGQPPKLLMNWASTRESGVP
DRFSGSGSGAEFTLTISSLQAEDVAIYYCQQYYSTLTFGGGTKVEIKR
SEQ ID NO: 165, VL amino acid sequence
DIVMTQSPDSLAVSLGERATINCKSSQSVLYRSNNLNYLAWYQQKPGQPPKLLMYWASTRESGVP
DRFSGSGSGAEFTLTISSLQAEDVAIYYCQQYYSTLTFGGGTKVEIKR
SEQ ID NO: 166, VL amino acid sequence
DIVMTQSPDSLAVSLGERATINCKSSQSVLYSYNNKNYLAWYQQKPGQPPKLLMEWASTRESGVP
DRFSGSGSGAEFTLTISSLQAEDVAIYYCQQYYSTLTFGGGTKVEIKR
SEQ ID NO: 167, VL amino acid sequence
divmtqspdslavslgeratinckssqsvlyssnnknylawyqqkpgqppkllmywastresgvpdrfsgsgsgaeftltisslqaedvaiyycqqyys
tltfgggtkveik
SEQ ID NO: 168, VL amino acid sequence
divmtqspdslavslgeratinckssqsvlyasnnknylawyqqkpgqppkllmywastresgvpdrfsgsgsgaeftltisslqaedvaiyycqqyys
tltfgggtkveik
SEQ ID NO: 169, VL amino acid sequence
divmtqspdslavslgeratinckssqsvlysannknylawyqqkpgqppkllmywastresgvpdrfsgsgsgaeftltisslqaedvaiyycqqyys
tltfgggtkveik
SEQ ID NO: 170, VL amino acid sequence
divmtqspdslavslgeratinckssqsvlyssnnknylawyqqkpgqppkllmywaseresgvpdrfsgsgsgaeftltisslqaedvaiyycqqyys
tltfgggtkveik
SEQ ID NO: 171, VL amino acid sequence
divmtqspdslavslgeratinckssqsvlyssnnknylawyqqkpgqppkllmywashresgvpdrfsgsgsgaeftltisslqaedvaiyycqqyys
tltfgggtkveik
SEQ ID NO: 172, VL amino acid sequence
divmtqspdslavslgeratinckssqsvlyaannknylawyqqkpgqppkllmywaseresgvpdrfsgsgsgaeftltisslqaedvaiyycqqyy
stltfgggtkveik
SEQ ID NO: 173, VL amino acid sequence
divmtqspdslavslgeratinckssqsvlyaynnknylawyqqkpgqppkllmywaseresgvpdrfsgsgsgaeftltisslqaedvaiyycqqyy
stltfgggtkveik
SEQ ID NO: 174, VL amino acid sequence
divmtqspdslavslgeratinckssqsvlysvnnknylawyqqkpgqppkllmnwastresgvpdrfsgsgsgaeftltisslqaedvaiyycqqyys
tltfgggtkveik
SEQ ID NO: 175, VL amino acid sequence
divmtqspdslavslgeratinckssqsvlysvnnknylawyqqkpgqppkllmnwaseresgvpdrfsgsgsgaeftltisslqaedvaiyycqqyy
stltfgggtkveik
SEQ ID NO: 176, VL amino acid sequence
divmtqspdslavslgeratinckssqsvlysvnnknylawyqqkpgqppkllmnwashresgvpdrfsgsgsgaeftltisslqaedvaiyycqqyy
stltfgggtkveik
SEQ ID NO: 177, VL amino acid sequence
divmtqspdslavslgeratinckssqsvlysynnknylawyqqkpgqppkllmewastresgvpdrfsgsgsgaeftltisslqaedvaiyycqqyys
tltfgggtkveik
SEQ ID NO: 178, VL amino acid sequence
divmtqspdslavslgeratinckssqsvlysynnknylawyqqkpgqppkllmewashresgvpdrfsgsgsgaeftltisslqaedvaiyycqqyy
stltfgggtkveik
SEQ ID NO: 179, VL amino acid sequence
divmtqspdslavslgeratinckssqsvlyrsnnlnylawyqqkpgqppkllmywastresgvpdrfsgsgsgaeftltisslqaedvaiyycqqyyst
ltfgggtkveik
SEQ ID NO: 180, VL amino acid sequence
divmtqspdslavslgeratinckssqsvlyrsnnlnylawyqqkpgqppkllmywaseresgvpdrfsgsgsgaeftltisslqaedvaiyycqqyyst
ltfgggtkveik
SEQ ID NO: 181, VL amino acid sequence
divmtqspdslavslgeratinckssqsvlysfnnknylawyqqkpgqppkllmywaseresgvpdrfsgsgsgaeftltisslqaedvaiyycqqyys
tltfgggtkveik
SEQ ID NO: 182, VL amino acid sequence
divmtqspdslavslgeratinckssqsvlysannknylawyqqkpgqppkllmywaseresgvpdrfsgsgsgaeftltisslqaedvaiyycqqyys
tltfgggtkveik
SEQ ID NO: 183, VL amino acid sequence
divmtqspdslavslgeratinckssqsvlyswnnknylawyqqkpgqppkllmywaseresgvpdrfsgsgsgaeftltisslqaedvaiyycqqyy
stltfgggtkveik
SEQ ID NO: 184, VL amino acid sequence
divmtqspdslavslgeratinckssqsvlysynnknylawyqqkpgqppkllmywaseresgvpdrfsgsgsgaeftltisslqaedvaiyycqqyy
stltfgggtkveik
SEQ ID NO: 185, VL amino acid sequence
divmtqspdslavslgeratinckssqsvlywannknylawyqqkpgqppkllmywastresgvpdrfsgsgsgaeftltisslqaedvaiyycqqyy
stltfgggtkveik
SEQ ID NO: 186, VL amino acid sequence
divmtqspdslavslgeratinckssqsvlywannknylawyqqkpgqppkllmywaseresgvpdrfsgsgsgaeftltisslqaedvaiyycqqyy
stltfgggtkveik
SEQ ID NO: 187, VL amino acid sequence
divmtqspdslavslgeratinckssqsvlywsnnknylawyqqkpgqppkllmywaseresgvpdrfsgsgsgaeftltisslqaedvaiyycqqyy
stltfgggtkveik
SEQ ID NO: 188, VL amino acid sequence
divmtqspdslavslgeratinckssqsvlyyvnnynylawyqqkpgqppkllmywastresgvpdrfsgsgsgaeftltisslqaedvaiyycqqyys
tltfgggtkveik
SEQ ID NO: 189, VL amino acid sequence
divmtqspdslavslgeratinckssqsvlyysnnynylawyqqkpgqppkllmywastresgvpdrfsgsgsgaeftltisslqaedvaiyycqqyys
tltfgggtkveik
SEQ ID NO: 190, VL amino acid sequence
divmtqspdslavslgeratinckssqsvlyyannynylawyqqkpgqppkllmywastresgvpdrfsgsgsgaeftltisslqaedvaiyycqqyys
tltfgggtkveik
SEQ ID NO: 191, VL amino acid sequence
divmtqspdslavslgeratinckssqsvlyyvnnynylawyqqkpgqppkllmywaseresgvpdrfsgsgsgaeftltisslqaedvaiyycqqyy
stltfgggtkveik
SEQ ID NO: 192, VL amino acid sequence
divmtqspdslavslgeratinckssqsvlyqynnknylawyqqkpgqppkllmywashresgvpdrfsgsgsgaeftltisslqaedvaiyycqqyy
stltfgggtkveik
SEQ ID NO: 193, VL amino acid sequence
divmtqspdslavslgeratinckssqsvlyqynnknylawyqqkpgqppkllmywaseresgvpdrfsgsgsgaeftltisslqaedvaiyycqqyy
stltfgggtkveik
SEQ ID NO: 194, VL amino acid sequence
divmtqspdslavslgeratinckssqsvlyswnnknylawyqqkpgqppkllmywastresgvpdrfsgsgsgaeftltisslqaedvaiyycqqyy
stltfgggtkveik
SEQ ID NO: 195, VL amino acid sequence
divmtqspdslavslgeratinckssqsvlysynnknylawyqqkpgqppkllmywastresgvpdrfsgsgsgaeftltisslqaedvaiyycqqyys
tltfgggtkveik
SEQ ID NO: 196 - CDRH1 amino acid sequence
gftfrdvw
SEQ ID NO: 197 - CDRH2 amino acid sequence
ikskEdggtt
SEQ ID NO: 198 - CDRH2 amino acid sequence
Ikskidggtt
SEQ ID NO: 199 - CDRH2 amino acid sequence
ikskWdggtt
SEQ ID NO: 200 - CDRH2 amino acid sequence
ikskDdggtt
SEQ ID NO: 201 - CDRH2 amino acid sequence
ikskHdggtt
SEQ ID NO: 202 - CDRH3 amino acid sequence
ttagsyyFdtvgpglpegkfdy
SEQ ID NO: 203 - CDRH3 amino acid sequence
ttagsyyydtRgpglpegkfdy
SEQ ID NO: 204 - CDRH3 amino acid sequence
ttagsyyydtvgpglpegkfdy
SEQ ID NO: 205 - CDRH3 amino acid sequence
ttagsyyydtvgpElpegkfdy
SEQ ID NO: 206 - CDRH3 amino acid sequence
ttagsyyDdtvgpglpegkIdy
SEQ ID NO: 207 - CDRH3 amino acid sequence
ttagsyyHdtvgpglpegkfdy
SEQ ID NO: 208 - CDRH3 amino acid sequence
ttagsyyydtvLpglpegkfdy
SEQ ID NO: 209 - CDRH3 amino acid sequence
ttagsyyydtRgpElpegkfdy
SEQ ID NO: 210 - CDRH3 amino acid sequence
ttagsyyydtRgpEVpegkfdy
SEQ ID NO: 211 - CDRH3 amino acid sequence
ttagsyyydtvgpEVpegkfdy
SEQ ID NO: 212 - CDRH3 amino acid sequence
ttagsyyDdtRgpglpegkIdy
SEQ ID NO: 213 - CDRH3 amino acid sequence
ttagsyyDdtvgpElpegkIdy
SEQ ID NO: 214 - CDRH3 amino acid sequence
ttagsyyDdtvgpgVpegkIdy
SEQ ID NO: 215 - CDRH3 amino acid sequence
ttagsyyydtRLpglpegkfdy
SEQ ID NO: 216 - CDRH3 amino acid sequence
ttagsyyydtvLpElpegkfdy
SEQ ID NO: 217 - CDRH3 amino acid sequence
ttagsyyydtvLpgVpegkfdy
SEQ ID NO: 218 - CDRH3 amino acid sequence
ttagsyyHdtRgpglpegkfdy
SEQ ID NO: 219 - CDRH3 amino acid sequence
ttagsyyHdtvgpElpegkfdy
SEQ ID NO: 220 - CDRH3 amino acid sequence
ttagsyyHdtRgpElpegkfdy
SEQ ID NO: 221 - CDRH3 amino acid sequence
ttagsyyydtvgpgVpegkfdy
SEQ ID NO: 222 - CDRH3 amino acid sequence
ttagsyyFdtRgpglpegkfdy
SEQ ID NO: 223 - CDRH3 amino acid sequence
ttagsyyFdtvgpElpegkfdy
SEQ ID NO: 224 - CDRH3 amino acid sequence
ttagsyyFdtvgpgVpegkfdy
SEQ ID NO: 225 - CDRH3 amino acid sequence
ttagsyyFdtRgpElpegkfdy
SEQ ID NO: 226 - CDRL1 amino acid sequence
qsvlyWAnnkny
SEQ ID NO: 227 - CDRLI amino acid sequence
qsvlyssnnkny
SEQ ID NO: 228 - CDRL1 amino acid sequence
qsvlysYnnkny
SEQ ID NO: 229 - CDRL1 amino acid sequence
qsvlysWnnkny
SEQ ID NO: 230 - CDRL1 amino acid sequence
qsvlyQYnnkny
SEQ ID NO: 231 - CDRL1 amino acid sequence
qsvlyWsnnkny
SEQ ID NO: 232 - CDRL1 amino acid sequence
qsvlysFnnkny
SEQ ID NO: 233 - CDRL1 amino acid sequence
qsvlyYVnnYny
SEQ ID NO: 234 - CDRLI amino acid sequence
qsvlyAAnnkny
SEQ ID NO: 235 - CDRL1 amino acid sequence
qsvlysVnnkny
SEQ ID NO: 236 - CDRLI amino acid sequence
qsvlyRsnnLny
SEQ ID NO: 237 - CDRLI amino acid sequence
qsvlyAsnnkny
SEQ ID NO: 238 - CDRLI amino acid sequence
qsvlysAnnkny
SEQ ID NO: 239 - CDRL1 amino acid sequence
qsvlyAYnnkny
SEQ ID NO: 240 - CDRL1 amino acid sequence
qsvlyYsnnYny
SEQ ID NO: 241 - CDRLI amino acid sequence
qsvlyYAnnYny
SEQ ID NO: 242 - CDRL2 amino acid sequence
was
SEQ ID NO: 243 - Extended CDRL2 amino acid sequence
ywastr
SEQ ID NO: 244 - Extended CDRL2 amino acid sequence
ywasHr
SEQ ID NO: 245 - Extended CDRL2 amino acid sequence
ywasEr
SEQ ID NO: 246 - Extended CDRL2 amino acid sequence
Nwastr
SEQ ID NO: 247 - Extended CDRL2 amino acid sequence
Ewastr
SEQ ID NO: 248 - Extended CDRL2 amino acid sequence
NwasEr
SEQ ID NO: 249 - Extended CDRL2 amino acid sequence
NwasHr
SEQ ID NO: 250 - Extended CDRL2 amino acid sequence
EwasHr
SEQ ID NO: 251 - CDRL3 amino acid sequence
qqyystlt
SEQ ID NO: 252 - Human IgD constant region
APTKAPDVFPIISGCRHPKDNSPVVLACLITGYHPTSVTVTWYMGTQSQPQRTFPEIQRRDSYYMTS
SQLSTPLQQWRQGEYKCVVQHTASKSKKEIFRWPESPKAQASSVPTAQPQAEGSLAKATTAPATTR
NTGRGGEEKKKEKEKEEQEERETKTPECPSHTQPLGVYLLTPAVQDLWLRDKATFTCFVVGSDLK
DAHLTWEVAGKVPTGGVEEGLLERHSNGSQSQHSRLTLPRSLWNAGTSVTCTLNHPSLPPQRLMA
LREPAAQAPVKLSLNLLASSDPPEAASWLLCEVSGFSPPNILLMWLEDQREVNTSGFAPARPPPQPG
STTFWAWSVLRVPAPPSPQPATYTCVVSHEDSRTLLNASRSLEVSYVTDHGPMK
SEQ ID NO: 253 - Human IgG1 constant region
ASTKGPSVFPLAPSSKSTSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEALHNHYTQKSLSLSPGK
SEQ ID NO: 254 - Human IgG2 constant region
ASTKGPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTLMISR
TPEVTCVVVDVSHEDPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLVKGFYPSDISVEWESNGQ
PENNYKTTPPMLDSDGSFFLYSKLTVDKSRWQQGNVFSCSVMHEALHNHYTQKSLSLSPGK
SEQ ID NO: 255 - Human IgG3 constant region
ASTKGPSVFPLAPCSRSTSGGTAALGCLVKDYFPEPVTVSWNSGALTSGVHTFPAVLQSSGLYSLSS
VVTVPSSSLGTQTYTCNVNHKPSNTKVDKRVELKTPLGDTTHTCPRCPEPKSCDTPPPCPRCPEPKS
CDTPPPCPRCPEPKSCDTPPPCPRCPAPELLGGPSVFLFPPKPKDTLMISRTPEVTCVVVDVSHEDPEV
QFKWYVDGVEVHNAKTKPREEQYNSTFRVVSVLTVLHQDWLNGKEYKCKVSNKALPAPIEKTISK
TKGQPREPQVYTLPPSREEMTKNQVSLTCLVKGFYPSDIAVEWESSGQPENNYNTTPPMLDSDGSFF
LYSKLTVDKSRWQQGNIFSCSVMHEALHNRFTQKSLSLSPGK
SEQ ID NO: 256 - Human IgM constant region
GSASAPTLFPLVSCENSPSDTSSVAVGCLAQDFLPDSITLSWKYKNNSDISSTRGFPSVLRGGKYAAT
SQVLLPSKDVMQGTDEHVVCKVQHPNGNKEKNVPLPVIAELPPKVSVFVPPRDGFFGNPRKSKLIC
QATGFSPRQIQVSWLREGKQVGSGVTTDQVQAEAKESGPTTYKVTSTLTIKESDWLGQSMFTCRV
DHRGLTFQQNASSMCVPDQDTAIRVFAIPPSFASIFLTKSTKLTCLVTDLTTYDSVTISWTRQNGEAV
KTHTNISESHPNATFSAVGEASICEDDWNSGERFTCTVTHTDLPSPLKQTISRPKGVALHRPDVYLLP
PAREQLNLRESATITCLVTGFSPADVFVQWMQRGQPLSPEKYVTSAPMPEPQAPGRYFAHSILTVSE
EEWNTGETYTCVAHEALPNRVTERTVDKSTGKPTLYNVSLVMSDTAGTCY
SEQ ID NO: 257 - Human IgG4 constant region
ASTKGPSVFPLAPCSRSTSESTAALGCLVKDYFPEPVTVSWNSGALTSGVHTFPAVLQSSGLYSLSS
VVTVPSSSLGTKTYTCNVDHKPSNTKVDKRVESKYGPPCPSCPAPEFLGGPSVFLFPPKPKDTLMIS
RTPEVTCVVVDVSQEDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCLVKGFYPSDIAVEWESNG
QPENNYKTTPPVLDSDGSFFLYSRLTVDKSRWQEGNVFSCSVMHEALHNHYTQKSLSLSLGK
SEQ ID NO: 258 - Human IgA1 constant region
ASPTSPKVFPLSLCSTQPDGNVVIACLVQGFFPQEPLSVTWSESGQGVTARNFPPSQDASGDLYTTSS
QLTLPATQCLAGKSVTCHVKHYTNPSQDVTVPCPVPSTPPTPSPSTPPTPSPSCCHPRLSLHRPALED
LLLGSEANLTCTLTGLRDASGVTFTWTPSSGKSAVQGPPERDLCGCYSVSSVLPGCAEPWNHGKTF
TCTAAYPESKTPLTATLSKSGNTFRPEVHLLPPPSEELALNELVTLTCLARGFSPKDVLVRWLQGSQ
ELPREKYLTWASRQEPSQGTTTFAVTSILRVAAEDWKKGDTFSCMVGHEALPLAFTQKTIDRLAGK
PTHVNVSVVMAEVDGTCY
SEQ ID NO: 259 - Human IgA2 constant region
ASPTSPKVFPLSLDSTPQDGNVVVACLVQGFFPQEPLSVTWSESGQNVTARNFPPSQDASGDLYTTS
SQLTLPATQCPDGKSVTCHVKHYTNPSQDVTVPCPVPPPPPCCHPRLSLHRPALEDLLLGSEANLTC
TLTGLRDASGATFTWTPSSGKSAVQGPPERDLCGCYSVSSVLPGCAQPWNHGETFTCTAAHPELKT
PLTANITKSGNTFRPEVHLLPPPSEELALNELVTLTCLARGFSPKDVLVRWLQGSQELPREKYLTWA
SRQEPSQGTTTFAVTSILRVAAEDWKKGDTFSCMVGHEALPLAFTQKTIDRMAGKPTHVNVSVVM
AEVDGTCY
SEQ ID NO: 260 - Human Ig kappa constant region
TVAAPSVFIFPPSDEQLKSGTASVVCLLNNFYPREAKVQWKVDNALQSGNSQESVTEQDSKDSTYS
LSSTLTLSKADYEKHKVYACEVTHQGLSSPVTKSFNRGEC
SEQ ID NO: 261 - Full Heavy Chain Sequence (VH + constant)
EVQLVESGGGLVKPGGSLRLSCAASGFTFRDVWMSWVRQAPGKGLEWVGRIKSKEDGGTTDYAA
PVKGRFTISRDDSKNTLYLQMNSLKTEDTAVYYCTTAGSYYFDTVGPGLPEGKFDYWGQGTLVTV
SSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPG
K*
SEQ ID NO: 262 - Full Heavy Chain Sequence (VH + constant)
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTRGPGLPEGKFDYWGQGTLVTV
SSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPG
K*
SEQ ID NO: 263 - Full Heavy Chain Sequence (VH + constant)
EVQLVESGGGLVKPGGSLRLSCAASGFTFRDVWMSWVRQAPGKGLEWVGRIKSKWDGGTTDYA
APVKGRFTISRDDSKNTLYLQMNSLKTEDTAVYYCTTAGSYYYDTVGPGLPEGKFDYWGQGTLVT
VSSASTKGPSVFPLAPSSKSTSGGTAALGCLVKDYFPEPVTVSWNSGALTSGVHTFPAVLQSSGLYS
LSSVVTVPSSSLGTQTYICNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHYTQKSLSLSP
GK*
SEQ ID NO: 264 - Full Heavy Chain Sequence (VH + constant)
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SSASTKGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYTQKSLSLSPG
K*
SEQ ID NO: 265 - Full Light Chain Sequence (VL + constant)
DIVMTQSPDSLAVSLGERATINCKSSQSVLYWANNKNYLAWYQQKPGQPPKLLMYWASTRESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 266 - Full Light Chain Sequence (VL + constant)
DIVMTQSPDSLAVSLGERATINCKSSQSVLYSSNNKNYLAWYQQKPGQPPKLLMYWASTRESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 267 - Full Light Chain Sequence (VL + constant)
DIVMTQSPDSLAVSLGERATINCKSSQSVLYSYNNKNYLAWYQQKPGQPPKLLMYWASTRESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 268 - Full Light Chain Sequence (VL + constant)
DIVMTQSPDSLAVSLGERATINCKSSQSVLYSWNNKNYLAWYQQKPGQPPKLLMYWASTRESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 269 - Full Light Chain Sequence (VL + constant)
DIVMTQSPDSLAVSLGERATINCKSSQSVLYQYNNKNYLAWYQQKPGQPPKLLMYWASHRESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 270 - Full Light Chain Sequence (VL + constant)
DIVMTQSPDSLAVSLGERATINCKSSQSVLYWSNNKNYLAWYQQKPGQPPKLLMYWASERESGVP
DRFSGSGSGAEFTLTISSLQAEDVAIYYCQQYYSTLTFGGGTKVEIKRTVAAPSVFIFPPSDEQLKSGT
ASVVCLLNNFYPREAKVQWKVDNALQSGNSQESVTEQDSKDSTYSLSSTLTLSKADYEKHKVYAC
EVTHQGLSSPVTKSFNRGEC*
SEQ ID NO: 271 - COV2-RBD (rpk9) Wuhan-Hu-1 (WT)
MGILPSPGMPALLSLVSLLSVLLMGCVAETGTRFPNITNLCPFGEVFNATRFASVYAWNRKRISNCV
ADFSVLYNSASFSTFKCYGVSPTKLNDLCWTNIYADSFVIRGDEVRQIAPGQTGKIADYNYKLPDDF
TGCVIAWNSNNLDSKVGGNYNYLYRLFRKSNLKPFERDISTEIYQAGSTPCNGVEGFNCYFPLQSY
GFQPTNGVGYQPYRVVVLSFELLHAPATVCGPKKSTGLNDIFEAQKIEWHEHHHHHHHH*
SEQ ID NO: 272 - COV2-RBD (rpk9) B.1.1.529 (BA.1)
MGILPSPGMPALLSLVSLLSVLLMGCVAETGTRFPNITNLCPFDEVFNATRFASVYAWNRKRISNCV
ADFSVLYNLAPFFTFKCYGVSPTKLNDLCWTNIYADSFVIRGDEVRQIAPGQTGNIADYNYKLPDDF
TGCVIAWNSNKLDSKVSGNYNYLYRLFRKSNLKPFERDISTEIYQAGNKPCNGVAGFNCYFPLRSY
SFRPTYGVGHQPYRVVVLSFELLHAPATVCGPKKSTGLNDIFEAQKIEWHEHHHHHHHH*
SEQ ID NO: 273 - COV2-RBD (rpk9) B.1.1.529 R346K (BA.1.1)
MGILPSPGMPALLSLVSLLSVLLMGCVAETGTRFPNITNLCPFDEVFNATKFASVYAWNRKRISNCV
ADFSVLYNLAPFFTFKCYGVSPTKLNDLCWTNIYADSFVIRGDEVRQIAPGQTGNIADYNYKLPDDF
TGCVIAWNSNKLDSKVSGNYNYLYRLFRKSNLKPFERDISTEIYQAGNKPCNGVAGFNCYFPLRSY
SFRPTYGVGHQPYRVVVLSFELLHAPATVCGPKKSTGLNDIFEAQKIEWHEHHHHHHHH*
SEQ ID NO: 274 - COV2-2130 Heavy chain variable domain
EVQLVESGGGLVKPGGSLRLSCAASGFTFRDVWMSWVRQAPGKGLEWVGRIKSKIDGGTTDYAA
PVKGRFTISRDDSKNTLYLQMNSLKTEDTAVYYCTTAGSYYYDTVGPGLPEGKFDYWGQGTLVTV
SS
SEQ ID NO: 275 - COV2-2130 Light chain variable domain
DIVMTQSPDSLAVSLGERATINCKSSQSVLYSSNNKNYLAWYQQKPGQPPKLLMYWASTRESGVP
DRFSGSGSGAEFTLTISSLQAEDVAIYYCQQYYSTLTFGGGTKVEIKR

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification. In some aspects, technical publications are referenced by name and date within parenthesis. The full bibliographic citations for these references are incorporated herein by reference.

### REFERENCES

[VanBlargan2022] VanBlargan, L. A., Errico, J. M., Halfmann, P. J. et al. An infectious SARS-CoV-2 B.1.1.529 Omicron virus escapes neutralization by therapeutic monoclonal antibodies. Nat Med 28, 490-495 (2022).
[WHO2020] WHO Coronavirus Disease (2020). (COVID-19) Dashboard. Available at: https://covid19.who.int (Accessed Feb. 28, 2022).
[Wolfel2020]Wölfel, R., Corman, V. M., Guggemos, W. et al. Virological assessment of hospitalized patients with COVID-2019. Nature 581, 465-469 (2020).
[Bai2020] Bai Y, Yao L, Wei T, et al. Presumed Asymptomatic Carrier Transmission of COVID-19. JAMA 2020; 323(14):1406-1407. doi:10.1001/jama.2020.2565
[Viana2022] Viana, R., Moyo, S., Amoako, D. G. et al. Rapid epidemic expansion of the SARS-CoV-2 Omicron variant in southern Africa. Nature 603, 679-686 (2022).
[Zost2020] Zost, S. J., Gilchuk, P., Case, J. B. et al. Potently neutralizing and protective human antibodies against SARS-CoV-2. Nature 584, 443-449 (2020). https://doi.org/10.1038/s41586-020-2548-6
[Dong2021] Dong, J., Zost, S. J., Greaney, A. J. et al. Genetic and structural basis for SARS-CoV-2 variant neutralization by a two-antibody cocktail. Nat Microbiol 6, 1233-1244 (2021).
[Cameroni2022] Cameroni, E., Bowen, J. E., Rosen, L. E. et al. Broadly neutralizing antibodies overcome SARS-CoV-2 Omicron antigenic shift. Nature 602, 664-670 (2022).
[Wang2022] Qian Wang, Yicheng Guo, View ORCID ProfileSho Iketani, Manoj S. Nair, Zhiteng Li, Hiroshi Mohri, Maple Wang, Jian Yu, Anthony D. Bowen, Jennifer Y. Chang, Jayesh G. Shah, Nadia Nguyen, Zhiwei Chen, Kathrine Meyers, Michael T. Yin, Magdalena E. Sobieszczyk, Sheng, Yaoxing Huang, Lihong Liu, David D. Ho. Antibody evasion by SARS-CoV-2 Omicron subvariants BA.2.12.1, BA.4, and BA.5. Biorxiv, 2022.
[Tuekprakhon2022] Tuekprakhon et al., 2022, Cell 185, 2422-2433. Jul. 7, 2022.
[Takashita2022] Takashita, Emi, et al. “Efficacy of Antibodies and Antiviral Drugs against Omicron BA. 2.12. 1, BA. 4, and BA. 5 Subvariants.” New England Journal of Medicine (2022).
[zem1a2005] Zemla A, Zhou C E, Slezak T, Kuczmarski T, Rama D, Tones C, Sawicka D, Barsky D, (2005), “AS2TS system for protein structure modeling and analysis” Nucleic Acids Research, 33:W111-W115.
[Mannar2022] Dhiraj Mannar, James W. Saville Xing Zhu Shanti S. Srivastava Alison M. Berezuk Katharine S. Tuttle, Ana Citlali Marquez, Inna Sekirov, Sriram Subramaniam. SARS-CoV-2 Omicron variant: Antibody evasion and cryo-EM structure of spike protein-ACE2 complex. Science, 20 Jan. 2022. Vol 375, Issue 6582, pp. 760-764. DOI: 10.1126/science.abn7760
[selaculang2013] Sela-Culang I, Kunik V, Ofran Y. The structural basis of antibody-antigen recognition. Front Immunol. 2013 Oct. 8; 4:302. doi: 10.3389/fimmu.2013.00302. PMID: 24115948; PMCID: PMC3792396.
[zem1a2022] Zemla A, Thomas Desautels, Edmond Y. Lau, Fangqiang Zhu, Kathryn T. Arrildt, Brent W. Segelke, Shankar Sundaram, Daniel Faissol. SARS-COV-2 Omicron variant predicted to exhibit higher affinity to ACE-2 receptor and lower affinity to a large range of neutralizing antibodies, using a rapid computational platform.
[barlow2018] Barlow K A, Conchuir S O, Thompson S, Suresh P, Lucas J E, Heinonen M, Kortemme T, (2018), “Flex ddG: Rosetta Ensemble-Based Estimation of Changes in Protein—Protein Binding Affinity upon Mutation”, The Journal of Physical Chemistry, B 122 (21), 5389-5399, DOI: 10.1021/acs.jpcb.7b11367.
[zhu2022] Fangqiang Zhu, Feliza A. Bourguet, William F. D. Bennett, Edmond Y. Lau, Kathryn T. Arrildt, Brent W. Segelke, Adam T. Zemla, Thomas A. Desautels, Daniel M. Faissol. “Large-scale application of free energy perturbation calculations for antibody design”. Scientific Reports, 12, 12489 (2022).
[Vashchenko2022] Vashchenko et al., “AbBERT: Learning Antibody Humanness via Masked. Language Modeling”, BioArxiv,
[Olsen2022] Olsen, Tobias H., Fergus Boyles, and Charlotte M. Deane. “Observed Antibody Space: A diverse database of cleaned, annotated, and translated unpaired and paired antibody sequences.” Protein Science 31.1 (2022): 141-146.
[ehrgott2005] Ehrgott, Matthias. Multicriteria optimization. Springer Berlin Heidelberg 2005. Chapter 2.
[pinto2020] Pinto, D., Park, Y J., Beltramello, M. et al. Cross-neutralization of SARS-CoV-2 by a human monoclonal SARS-CoV antibody. Nature 583, 290-295 (2020).
[Case2022] Case, J. B., Mackin, S., Errico, J. M., Chong, Z., Madden, E. A., Whitener, B., Guarino, B., Schmid, M. A., Rosenthal, K., Ren, K., et al. (2022). Resilience of 5309 and AZD7442 monoclonal antibody treatments against infection by SARS-CoV-2 Omicron lineage strains. Nat Commun 13, 3824. 10.1038/s41467-022-31615-7.
[Halfmann2022] Halfmann, P. J., Lida, S., Iwatsuki-Horimoto, K., Maemura, T., Kiso, M., Scheaffer, S. M., Darling, T. L., Joshi, A., Loeber, S., Singh, G., et al. (2022). SARS-CoV-2 Omicron virus causes attenuated disease in mice and hamsters. Nature 603, 687-692. 10.1038/s41586-022-04441-6.
[Uraki2022] Uraki, R., Kiso, M., Lida, S., Imai, M., Takashita, E., Kuroda, M., Halfmann, P. J., Loeber, S., Maemura, T., Yamayoshi, S., et al. (2022). Characterization and antiviral susceptibility of SARS-CoV-2 Omicron BA.2. Nature 607, 119-127. 10.1038/s41586-022-04856-1.
[Ying2022] Ying, B., Scheaffer, S. M., Whitener, B., Liang, C. Y., Dmytrenko, O., Mackin, S., Wu, K., Lee, D., Avena, L. E., Chong, Z., et al. (2022). Boosting with variant-matched or historical mRNA vaccines protects against Omicron infection in mice. Cell 185, 1572-1587 e1511. 10.1016/j.cell.2022.03.037.
[Chen2021] Chen, R. E., Zhang, X., Case, J. B., Winkler, E. S., Liu, Y., VanBlargan, L. A., Liu, J., Errico, J. M., Xie, X., Suryadevara, N., et al. (2021). Resistance of SARS-CoV-2 variants to neutralization by monoclonal and serum-derived polyclonal antibodies. Nat Med. 10.1038/s41591-021-01294-w.
[Laskowski2011] R. A. Laskowski, M. B. Swindells, LigPlot+: Multiple ligand-protein interaction diagrams for drug discovery. J. Chem. Inf. Model. 51, 2778-2786 (2011).
[Elnaggar2021] A. Elnaggar et al., “ProtTrans: Towards Cracking the Language of Lifes Code Through Self-Supervised Deep Learning and High Performance Computing,” in IEEE Transactions on Pattern Analysis and Machine Intelligence, doi: 10.1109/TPAMI.2021.3095381.
[gitMaestro] github.com/LLNL/maestrowf. Accessed 7/20/22.
[gitSina] github.com/LLNL/Sina. Accessed 7/20/22.
[Mockus1978] Mockus, Jonas, Vytautas Tiesis, and Antanas Zilinskas. “The application of Bayesian methods for seeking the extremum.” Towards global optimization 2, no. 117-129 (1978): 2.
[Gardner2018] Gardner, Jacob R., Geoff Pleiss, David Bindel, Kilian Q. Weinberger, and Andrew Gordon Wilson. “GPyTorch: Blackbox Matrix-Matrix Gaussian Process Inference with GPU Acceleration.” In NeurIPS (2018).
[Chng 2015] Chng, J. et al. Cleavage efficient 2A peptides for high level monoclonal antibody expression in CHO cells. MAbs 7, 403-412 (2015).
[Zost2020a] Zost, S. J. et al. Rapid isolation and profiling of a diverse panel of human monoclonal antibodies targeting the SARS-CoV-2 spike protein. Nat. Med. (2020).
[VPneut1] Crawford, K. H. D.; Eguia, R.; Dingens, A. S.; Loes, A. N.; Malone, K. D.; Wolf, C. R.; Chu, H. Y.; Tortorici, M. A.; Veesler, D.; Murphy, M.; Pettie, D.; King, N. P.; Balazs, A. B.; Bloom, J. D. Protocol and Reagents for Pseudotyping Lentiviral Particles with SARS-CoV-2 Spike Protein for Neutralization Assays. Viruses 2020, 12, 513.
[VanBlargan2021] VanBlargan, L. A., Adams, L. J., Liu, Z., Chen, R. E., Gilchuk, P., Raju, S., Smith, B. K., Zhao, H., Case, J. B., Winkler, E. S., et al. (2021). A potently neutralizing SARS-CoV-2 antibody inhibits variants of concern by utilizing unique binding residues in a highly conserved epitope. Immunity. 10.1016/j.immuni.2021.08.016.
[Plante2020] Plante, J. A., Liu, Y., Liu, J., Xia, H., Johnson, B. A., Lokugamage, K. G., Zhang, X., Muruato, A. E., Zou, J., Fontes-Garfias, C. R., et al. (2020). Spike mutation D614G alters SARS-CoV-2 fitness. Nature. 10.1038/s41586-020-2895-3.
[Ying2021] Ying, B., Whitener, B., VanBlargan, L. A., Hassan, A. O., Shrihari, S., Liang, C. Y., Karl, C. E., Mackin, S., Chen, R. E., Kafai, N. M., et al. (2021). Protective activity of mRNA vaccines against ancestral and variant SARS-CoV-2 strains. bioRxiv. 10.1101/2021.08.25.457693.
[Oliphant2005] Oliphant, T., Engle, M., Nybakken, G. E., Doane, C., Johnson, S., Huang, L., Gorlatov, S., Mehlhop, E., Marri, A., Chung, K. M., et al. (2005). Development of a humanized monoclonal antibody with therapeutic potential against West Nile virus. Nat Med 11, 522-530. 10.1038/nm1240.
[Case2020] James Brett Case, Adam L. Bailey, Arthur S. Kim, Rita E. Chen, Michael S. Diamond. Growth, detection, quantification, and inactivation of SARS-CoV-2. Virology, Volume 548, 2020, Pages 39-48. ISSN 0042-6822, doi.org/10.1016/j.virol.2020.05.015.
[Kimanius2021] D. Kimanius, L. Dong, G. Sharov, T. Nakane, S. H. W. Scheres, New tools for automated cryo-EM single-particle analysis in RELION-4.0. Biochem. J. 478, 4169-4185 (2021).
[Zheng2017] S. Q. Zheng, E. Palovcak, J.-P. Armache, K. A. Verba, Y. Cheng, D. A. Agard, MotionCor2: anisotropic correction of beam-induced motion for improved cryo-electron microscopy. Nat. Methods (2017), doi:10.1038/nmeth.4193.
[Rohou2015] Rohou, Alexis, and Nikolaus Grigorieff. “CTFFIND4: Fast and accurate defocus estimation from electron micrographs.” Journal of structural biology 192, no. 2 (2015): 216-221.
[Fernandez-Leiro2017] R. Fernandez-Leiro, S. H. W. Scheres, “A pipeline approach to single-particle processing in RELION” in Acta Crystallographica Section D: Structural Biology (2017), vol. 73, pp. 496-502.
[Bepler2020] T. Bepler, K. Kelley, A. J. Noble, B. Berger, Topaz-Denoise: general deep denoising models for cryoEM and cryoET. Nat. Commun. 2020 111. 11, 1-12 (2020).
[Kimanus2021] D. Kimanius, L. Dong, G. Sharov, T. Nakane, S. H. W. Scheres, New tools for automated cryo-EM single-particle analysis in RELION-4.0. Biochem. J. 478, 4169-4185 (2021).
[Zivanov2018] J. Zivanov, T. Nakane, B. O. Forsberg, D. Kimanius, W. J. Hagen, E. Lindahl, S. H. Scheres, New tools for automated high-resolution cryo-EM structure determination in RELION-3. Elife (2018), doi:10.7554/elife.42166.
[Scheres2018]S. H. W. Scheres, Single-particle processing in RELION. Manuals (2018).
[Pettersen2004] E. F. Pettersen, T. D. Goddard, C. C. Huang, G. S. Couch, D. M. Greenblatt, E. C. Meng, T. E. Ferrin, UCSF Chimera—A visualization system for exploratory research and analysis. J. Comput. Chem. 25, 1605-1612 (2004).
[Pettersen2021] E. F. Pettersen, T. D. Goddard, C. C. Huang, E. C. Meng, G. S. Couch, T. I. Croll, J. H. Morris, T. E. Ferrin, UCSF ChimeraX: Structure visualization for researchers, educators, and developers. Protein Sci. 30, 70-82 (2021).
[Emsley2004] P. Emsley, K. Cowtan, Coot: Model-building tools for molecular graphics. Acta Crystallogr. Sect. D Biol. Crystallogr. 60, 2126-2132 (2004).
[Adams2010] P. D. Adams, P. V. Afonine, G. Bunkóczi, V. B. Chen, I. W. Davis, N. Echols, J. J. Headd, L. W. Hung, G. J. Kapral, R. W. Grosse-Kunstleve, A. J. McCoy, N. W. Moriarty, R. Oeffner, R. J. Read, D. C. Richardson, J. S. Richardson, T. C. Terwilliger, P. H. Zwart, PHENIX: A comprehensive Python-based system for macromolecular structure solution. Acta Crystallogr. Sect. D Biol. Crystallogr. 66, 213-221 (2010).
[Afonine2018] P. Afonine, B. Poon, R. Read, O. Sobolev, T. Terwilliger, A. Urzhumtsev, P. Adams, Real-space refinement in Phenix for cryo-EM and crystallography. bioRxiv, 249607 (2018).
[Chen2010] V. B. Chen, W. B. Arendall, J. J. Headd, D. A. Keedy, R. M. Immormino, G. J. Kapral, L. W. Murray, J. S. Richardson, D. C. Richardson, MolProbity: All-atom structure validation for macromolecular crystallography. Acta Crystallogr. Sect. D Biol. Crystallogr. 66, 12-21 (2010).

1. An isolated antibody or antibody fragment that binds to a SARS-CoV-2 surface protein having enhanced binding and/or neutralization characteristics when compared to a monoclonal antibody selected from sotrovimab (S309), COV2-2381, cilgavimab (COV2-2130 or AZD1061), and/or tixagevimab (COV2-2196 or AZD8895); and wherein the SARS-CoV-2 surface protein is a spike (S) glycoprotein.

2. The antibody or antibody fragment of claim 1, wherein the antibody or antibody fragment is capable of neutralizing from about 80 to about 100% of the SARS-CoV-2 virus with a concentration of about 250 ng/ml to about 0.01 ng/ml.

3. The antibody or antibody fragment of claim 1, wherein the antibody or antibody fragment inhibits or blocks the binding of the SARS-CoV-2 virus to a human receptor angiotensin converting enzyme 2 (hACE2),
(a) wherein the antibody or antibody fragment inhibits or blocks the binding to hACE2 with an IC₅₀ of about 150 ng/ml to about 0.1 ng/ml; or
(b) wherein the antibody or antibody fragment binds to a target antigen with a binding affinity (K_{D}) of about 0.1 nM to about 0.05 nM or from about 10 nM to about 120 nM.

4. The antibody or antibody fragment of claim 1, wherein the antibody or antibody fragment binds:
(a) a trimeric spike protein ectodomain, and/or
(b) a monomeric spike protein binding domain (RBD),
wherein the binding affinity of the antibody or antibody fragment to the trimeric spike protein ectodomain and/or the monomeric spike protein RBD has an EC₅₀ of about 1.0 pg/ml to 0.1 ng/ml, or 1.0 pg/ml to about 10 ng/mL.

5.-7. (canceled)

8. The antibody or antibody fragment of claim 1, wherein the antibody or antibody fragment comprises:
(1) a heavy chain complementary determining region 1 (CDRH1) comprising the amino acid sequence of SEQ ID NO: 196, a CDRH2 comprising the amino acid sequence of SEQ ID NO: 197, and a CDRH3 comprising the amino acid sequence of SEQ ID NO: 202, and a light chain complementary determining region 1 (CDRL1) comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(2) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 203, and a CDRL1 comprising the amino acid sequence of SEQ ID NO: 227, a CDRL2 comprising the amino acid sequence of SEQ ID NO: 242 or 243, and a CDRL3 comprising the amino acid sequence of SEQ ID NO: 251;
(3) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 204, and a CDRL1 comprising the amino acid sequence of SEQ ID NO: 227, a CDRL2 comprising the amino acid sequence of SEQ ID NO: 242 or 243, and a CDRL3 comprising the amino acid sequence of SEQ ID NO: 251;
(4) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 228, a CDRL2 comprising the amino acid sequence of SEQ ID NO: 242 or 243, and a CDRL3 comprising the amino acid sequence of SEQ ID NO: 251;
(5) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 229, a CDRL2 comprising the amino acid sequence of SEQ ID NO: 242 or 243, and a CDRL3 comprising the amino acid sequence of SEQ ID NO: 251;
(6) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(7) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(8) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 204, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(9) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(10) a CDRH1 comprising the amino acid sequence of SEQ ID NO: 196, a CDRH2 comprising the amino acid sequence of SEQ ID NO: 198, and a CDRH3 comprising the amino acid sequence of S SEQ ID NO: 205, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(11) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 206, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(12) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 207, and a CDRL1 comprising the amino acid sequence of SEQ ID NO: 236, a CDRL2 comprising the amino acid sequence of SEQ ID NO: 242 or 243, and a CDRL3 comprising the amino acid sequence of SEQ ID NO: 251;
(13) a CDRH1 comprising the amino acid sequence of SEQ ID NO: 196, a CDRH2 comprising the amino acid sequence of SEQ ID NO: 201, and a CDRH3 comprising the amino acid sequence of SEQ ID NO: 208, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251; or
(14) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 209, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251.
(15) a heavy chain complementary determining region 1 (CDRH1) comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 227, a CDRL2 comprising the amino acid sequence of SEQ ID NO: 242 or 243, and a CDRL3 comprising the amino acid sequence of SEQ ID NO: 251;
(16) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 237, a CDRL2 comprising the amino acid sequence of SEQ ID NO: 242 or 243, and a CDRL3 comprising the amino acid sequence of SEQ ID NO: 251;
(17) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 238, a CDRL2 comprising the amino acid sequence of SEQ ID NO: 242 or 243, and a CDRL3 comprising the amino acid sequence of SEQ ID NO: 251;
(18) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 227, a CDRL2 comprising the amino acid sequence of SEQ ID NO: 242 or 245, and a CDRL3 comprising the amino acid sequence of SEQ ID NO: 251;
(19) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 227, a CDRL2 comprising the amino acid sequence of SEQ ID NO: 242 or 244, and a CDRL3 comprising the amino acid sequence of SEQ ID NO: 251;
(20) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 204, and a CDRL1 comprising the amino acid sequence of SEQ ID NO: 227, a CDRL2 comprising the amino acid sequence of SEQ ID NO: 242 or 243, and a CDRL3 comprising the amino acid sequence of SEQ ID NO: 251;
(21) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(22) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 209, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(23) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 210, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(24) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 209, and a CDRL1 comprising the amino acid sequence of SEQ ID NO: 239, a CDRL2 comprising the amino acid sequence of SEQ ID NO: 242 or 245, and a CDRL3 comprising the amino acid sequence of SEQ ID NO: 251;
(25) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 210, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(26) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 210, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(27) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 205, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(28) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 205, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(29) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 211, and a CDRL1 comprising the amino acid sequence of SEQ ID NO: 234, a CDRL2 comprising the amino acid sequence of SEQ ID NO: 242 or 245, and a CDRL3 comprising the amino acid sequence of SEQ ID NO: 251;
(30) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 239, a CDRL2 comprising the amino acid sequence of SEQ ID NO: 242 or 245, and a CDRL3 comprising the amino acid sequence of SEQ ID NO: 251;
(31) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 206, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(32) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 206, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(33) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 212, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(34) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 213, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(35) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 214, and a CDRL1 comprising the amino acid sequence of SEQ ID NO: 235, a CDRL2 comprising the amino acid sequence of SEQ ID NO: 242 or 246, and a CDRL3 comprising the amino acid sequence of SEQ ID NO: 251;
(36) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 206, and a CDRL1 comprising the amino acid sequence of SEQ ID NO: 235, a CDRL2 comprising the amino acid sequence of SEQ ID NO: 242 or 248, and a CDRL3 comprising the amino acid sequence of SEQ ID NO: 251;
(37) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 206, and a CDRL1 comprising the amino acid sequence of SEQ ID NO: 235, a CDRL2 comprising the amino acid sequence of SEQ ID NO: 242 or 249, and a CDRL3 comprising the amino acid sequence of SEQ ID NO: 251;
(38) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 208, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251;
(39) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 208, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251;
(40) a CDRH1 comprising the amino acid sequence of SEQ ID NO: 196, a CDRH2 comprising the amino acid sequence of SEQ ID NO: 201, and a CDRH3 comprising the amino acid sequence of SEQ ID NO: 215, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251;
(41) a CDRH1 comprising the amino acid sequence of SEQ ID NO: 196, a CDRH2 comprising the amino acid sequence of SEQ ID NO: 201, and a CDRH3 comprising the amino acid sequence of SEQ ID NO: 216, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251;
(42) a CDRH1 comprising the amino acid sequence of SEQ ID NO: 196, a CDRH2 comprising the amino acid sequence of SEQ ID NO: 201, and a CDRH3 comprising the amino acid sequence of SEQ ID NO: 217, and a CDRL1 comprising the amino acid sequence of SEQ ID NO: 228, a CDRL2 comprising the amino acid sequence of SEQ ID NO: 242 or 247, and a CDRL3 comprising the amino acid sequence of SEQ ID NO: 251;
(43) a CDRH1 comprising the amino acid sequence of SEQ ID NO: 196, a CDRH2 comprising the amino acid sequence of SEQ ID NO: 201, and a CDRH3 comprising the amino acid sequence of SEQ ID NO: 208, and a CDRL1 comprising the amino acid sequence of SEQ ID NO: 228, a CDRL2 comprising the amino acid sequence of SEQ ID NO: 242 or 250, and a CDRL3 comprising the amino acid sequence of SEQ ID NO: 251;
(44) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 218, and a CDRL1 comprising the amino acid sequence of SEQ ID NO: 236, a CDRL2 comprising the amino acid sequence of SEQ ID NO: 242 or 243, and a CDRL3 comprising the amino acid sequence of SEQ ID NO: 251;
(45) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 219, and a CDRL1 comprising the amino acid sequence of SEQ ID NO: 236, a CDRL2 comprising the amino acid sequence of SEQ ID NO: 242 or 243, and a CDRL3 comprising the amino acid sequence of SEQ ID NO: 251;
(46) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 220, and a CDRL1 comprising the amino acid sequence of SEQ ID NO: 236, a CDRL2 comprising the amino acid sequence of SEQ ID NO: 242 or 243, and a CDRL3 comprising the amino acid sequence of SEQ ID NO: 251;
(47) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 218, and a CDRL1 comprising the amino acid sequence of SEQ ID NO: 236, a CDRL2 comprising the amino acid sequence of SEQ ID NO: 242 or 245, and a CDRL3 comprising the amino acid sequence of SEQ ID NO: 251;
(48) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 207, and a CDRL1 comprising the amino acid sequence of SEQ ID NO: 236, a CDRL2 comprising the amino acid sequence of SEQ ID NO: 242 or 245, and a CDRL3 comprising the amino acid sequence of SEQ ID NO: 251;
(49) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 207, and a CDRL1 comprising the amino acid sequence of SEQ ID NO: 236, a CDRL2 comprising the amino acid sequence of SEQ ID NO: 242 or 243, and a CDRL3 comprising the amino acid sequence of SEQ ID NO: 251;
(50) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 204, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(51) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 204, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(52) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 203, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(53) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 205, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(54) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 221, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(55) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 209, and a CDRL1 comprising the amino acid sequence of SEQ ID NO: 232, a CDRL2 comprising the amino acid sequence of SEQ ID NO: 242 or 245, and a CDRL3 comprising the amino acid sequence of SEQ ID NO: 251;
(56) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 204, and a CDRL1 comprising the amino acid sequence of SEQ ID NO: 238, a CDRL2 comprising the amino acid sequence of SEQ ID NO: 242 or 245, and a CDRL3 comprising the amino acid sequence of SEQ ID NO: 251;
(57) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 204, and a CDRL1 comprising the amino acid sequence of SEQ ID NO: 229, a CDRL2 comprising the amino acid sequence of SEQ ID NO: 242 or 245, and a CDRL3 comprising the amino acid sequence of SEQ ID NO: 251;
(58) a CDRH1 comprising the amino acid sequence of SEQ ID NO: 196, a CDRH2 comprising the amino acid sequence of SEQ ID NO: 200, and a CDRH3 comprising the amino acid sequence of SEQ ID NO: 204, and a CDRL1 comprising the amino acid sequence of SEQ ID NO: 228, a CDRL2 comprising the amino acid sequence of SEQ ID NO: 242 or 245, and a CDRL3 comprising the amino acid sequence of SEQ ID NO: 251;
(59) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 202, and a CDRL1 comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(60) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 222, and a CDRL1 comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(61) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 223, and a CDRL1 comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(62) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 224, and a CDRL1 comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(63) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 225, and a CDRL1 comprising the amino acid sequence of SEQ ID NO: 226, a CDRL2 comprising the amino acid sequence of SEQ ID NO: 242 or 243, and a CDRL3 comprising the amino acid sequence of SEQ ID NO: 251;
(64) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 202, and a CDRL1 comprising the amino acid sequence of SEQ ID NO: 226, a CDRL2 comprising the amino acid sequence of SEQ ID NO: 242 or 245, and a CDRL3 comprising the amino acid sequence of SEQ ID NO: 251;
(65) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 204, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(66) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 204, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(67) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 203, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(68) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(69) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 221, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(70) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 203, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(71) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 205, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(72) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 203, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(73) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 205, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(74) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 204, and a CDRL1 comprising the amino acid sequence of SEQ ID NO: 226, a CDRL2 comprising the amino acid sequence of SEQ ID NO: 242 or 245, and a CDRL3 comprising the amino acid sequence of SEQ ID NO: 251;
(75) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 209, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(76) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(77) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 209, and a CDRL1 comprising the amino acid sequence of SEQ ID NO: 231, a CDRL2 comprising the amino acid sequence of SEQ ID NO: 242 or 245, and a CDRL3 comprising the amino acid sequence of SEQ ID NO: 251;
(78) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 205, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(79) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 205, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(80) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 209, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(81) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 211, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(82) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 240, a CDRL2 comprising the amino acid sequence of SEQ ID NO: 242 or 243, and a CDRL3 comprising the amino acid sequence of SEQ ID NO: 251;
(83) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 241, a CDRL2 comprising the amino acid sequence of SEQ ID NO: 242 or 243, and a CDRL3 comprising the amino acid sequence of SEQ ID NO: 251;
(84) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 233, a CDRL2 comprising the amino acid sequence of SEQ ID NO: 242 or 243, and a CDRL3 comprising the amino acid sequence of SEQ ID NO: 251;
(85) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 233, a CDRL2 comprising the amino acid sequence of SEQ ID NO: 242 or 245, and a CDRL3 comprising the amino acid sequence of SEQ ID NO: 251;
(86) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 204, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(87) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 203, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(88) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 205, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(89) a CDRH1 comprising the amino acid sequence of SEQ ID NO: 196, a CDRH2 comprising the amino acid sequence of SEQ ID NO: 198, and a CDRH3 comprising the amino acid sequence of SEQ ID NO: 221, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(90) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 203, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(91) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 203, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(92) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 205, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(93) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 205, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(94) a CDRH1 comprising the amino acid sequence of SEQ ID NO: 196, a CDRH2 comprising the amino acid sequence of SEQ ID NO: 197, and a CDRH3 comprising the amino acid sequence of SEQ ID NO: 209, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(95) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 230, a CDRL2 comprising the amino acid sequence of SEQ ID NO: 242 or 244, and a CDRL3 comprising the amino acid sequence of SEQ ID NO: 251;
(96) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 230, a CDRL2 comprising the amino acid sequence of SEQ ID NO: 242 or 245, and a CDRL3 comprising the amino acid sequence of SEQ ID NO: 251;
(97) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 229, a CDRL2 comprising the amino acid sequence of SEQ ID NO: 242 or 243, and a CDRL3 comprising the amino acid sequence of SEQ ID NO: 251;
(98) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 228, a CDRL2 comprising the amino acid sequence of SEQ ID NO: 242 or 243, and a CDRL3 comprising the amino acid sequence of SEQ ID NO: 251; or
(99) a CDRH1 comprising the amino acid sequence of SEQ ID NO: 196, a CDRH2 comprising the amino acid sequence of SEQ ID NO: 199, and a CDRH3 comprising the amino acid sequence of SEQ ID NO: 209, and a CDRL1 comprising the amino acid sequence of SEQ ID NO: 238, a CDRL2 comprising the amino acid sequence of SEQ ID NO: 242 or 243, and a CDRL3 comprising the amino acid sequence of SEQ ID NO: 251.

9. The antibody or antibody fragment of claim 1, wherein the antibody or antibody fragment comprises:
(a) a heavy chain variable sequence having at least 80% identity to the amino acid sequence of any one of SEQ ID NO: 100-154; and/or
(b) a light chain variable sequences having at least 80% identity to the amino acid sequence of any one of SEQ ID NO: 155-195.

10. The antibody or antibody fragment of claim 1, wherein the antibody or antibody fragment comprises a heavy chain variable sequence and a light chain variable sequence having at least 96% identity to an amino acid sequence selected from:
(1) the amino acid sequence of SEQ ID NO: 100 and the amino acid sequence of SEQ ID NO: 155;
(2) the amino acid sequence of SEQ ID NO: 101 and the amino acid sequence of SEQ ID NO: 156;
(3) the amino acid sequence of SEQ ID NO: 102 and the amino acid sequence of SEQ ID NO: 156;
(4) the amino acid sequence of SEQ ID NO: 103 and the amino acid sequence of SEQ ID NO: 157;
(5) the amino acid sequence of SEQ ID NO: 103 and the amino acid sequence of SEQ ID NO: 158;
(6) the amino acid sequence of SEQ ID NO: 103 and the amino acid sequence of SEQ ID NO: 159;
(7) the amino acid sequence of SEQ ID NO: 103 and the amino acid sequence of SEQ ID NO: 160;
(8) the amino acid sequence of SEQ ID NO: 104 and the amino acid sequence of SEQ ID NO: 161;
(9) the amino acid sequence of SEQ ID NO: 105 and the amino acid sequence of SEQ ID NO: 162;
(10) the amino acid sequence of SEQ ID NO: 105 and the amino acid sequence of SEQ ID NO: 163;
(11) the amino acid sequence of SEQ ID NO: 106 and the amino acid sequence of SEQ ID NO: 164;
(12) the amino acid sequence of SEQ ID NO: 107 and the amino acid sequence of SEQ ID NO: 165;
(13) the amino acid sequence of SEQ ID NO: 108 and the amino acid sequence of SEQ ID NO: 166; or
(14) the amino acid sequence of SEQ ID NO: 109 and the amino acid sequence of SEQ ID NO: 163.
(15) the amino acid sequence of SEQ ID NO: 110 and the amino acid sequence of SEQ ID NO: 167;
(16) the amino acid sequence of SEQ ID NO: 111 and the amino acid sequence of SEQ ID NO: 168;
(17) the amino acid sequence of SEQ ID NO: 111 and the amino acid sequence of SEQ ID NO: 169;
(18) the amino acid sequence of SEQ ID NO: 111 and the amino acid sequence of SEQ ID NO: 170;
(19) the amino acid sequence of SEQ ID NO: 111 and the amino acid sequence of SEQ ID NO: 171;
(20) the amino acid sequence of SEQ ID NO: 112 and the amino acid sequence of SEQ ID NO: 167;
(21) the amino acid sequence of SEQ ID NO: 113 and the amino acid sequence of SEQ ID NO: 172;
(22) the amino acid sequence of SEQ ID NO: 114 and the amino acid sequence of SEQ ID NO: 172;
(23) the amino acid sequence of SEQ ID NO: 115 and the amino acid sequence of SEQ ID NO: 172;
(24) the amino acid sequence of SEQ ID NO: 116 and the amino acid sequence of SEQ ID NO: 173;
(25) the amino acid sequence of SEQ ID NO: 117 and the amino acid sequence of SEQ ID NO: 172;
(26) the amino acid sequence of SEQ ID NO: 118 and the amino acid sequence of SEQ ID NO: 172;
(27) the amino acid sequence of SEQ ID NO: 119 and the amino acid sequence of SEQ ID NO: 172;
(28) the amino acid sequence of SEQ ID NO: 120 and the amino acid sequence of SEQ ID NO: 172;
(29) the amino acid sequence of SEQ ID NO: 121 and the amino acid sequence of SEQ ID NO: 172;
(30) the amino acid sequence of SEQ ID NO: 110 and the amino acid sequence of SEQ ID NO: 173;
(31) the amino acid sequence of SEQ ID NO: 122 and the amino acid sequence of SEQ ID NO: 174;
(32) the amino acid sequence of SEQ ID NO: 123 and the amino acid sequence of SEQ ID NO: 174;
(33) the amino acid sequence of SEQ ID NO: 124 and the amino acid sequence of SEQ ID NO: 174;
(34) the amino acid sequence of SEQ ID NO: 125 and the amino acid sequence of SEQ ID NO: 174;
(35) the amino acid sequence of SEQ ID NO: 126 and the amino acid sequence of SEQ ID NO: 174;
(36) the amino acid sequence of SEQ ID NO: 127 and the amino acid sequence of SEQ ID NO: 175;
(37) the amino acid sequence of SEQ ID NO: 127 and the amino acid sequence of SEQ ID NO: 176;
(38) the amino acid sequence of SEQ ID NO: 128 and the amino acid sequence of SEQ ID NO: 177;
(39) the amino acid sequence of SEQ ID NO: 129 and the amino acid sequence of SEQ ID NO: 177;
(40) the amino acid sequence of SEQ ID NO: 130 and the amino acid sequence of SEQ ID NO: 177;
(41) the amino acid sequence of SEQ ID NO: 131 and the amino acid sequence of SEQ ID NO: 177;
(42) the amino acid sequence of SEQ ID NO: 132 and the amino acid sequence of SEQ ID NO: 177;
(43) the amino acid sequence of SEQ ID NO: 133 and the amino acid sequence of SEQ ID NO: 178;
(44) the amino acid sequence of SEQ ID NO: 134 and the amino acid sequence of SEQ ID NO: 179;
(45) the amino acid sequence of SEQ ID NO: 135 and the amino acid sequence of SEQ ID NO: 179;
(46) the amino acid sequence of SEQ ID NO: 136 and the amino acid sequence of SEQ ID NO: 179;
(47) the amino acid sequence of SEQ ID NO: 134 and the amino acid sequence of SEQ ID NO: 180;
(48) the amino acid sequence of SEQ ID NO: 137 and the amino acid sequence of SEQ ID NO: 180;
(49) the amino acid sequence of SEQ ID NO: 138 and the amino acid sequence of SEQ ID NO: 179;
(50) the amino acid sequence of SEQ ID NO: 139 and the amino acid sequence of SEQ ID NO: 181;
(51) the amino acid sequence of SEQ ID NO: 112 and the amino acid sequence of SEQ ID NO: 181;
(52) the amino acid sequence of SEQ ID NO: 140 and the amino acid sequence of SEQ ID NO: 181;
(53) the amino acid sequence of SEQ ID NO: 141 and the amino acid sequence of SEQ ID NO: 181;
(54) the amino acid sequence of SEQ ID NO: 142 and the amino acid sequence of SEQ ID NO: 181;
(55) the amino acid sequence of SEQ ID NO: 143 and the amino acid sequence of SEQ ID NO: 181;
(56) the amino acid sequence of SEQ ID NO: 144 and the amino acid sequence of SEQ ID NO: 182;
(57) the amino acid sequence of SEQ ID NO: 144 and the amino acid sequence of SEQ ID NO: 183;
(58) the amino acid sequence of SEQ ID NO: 144 and the amino acid sequence of SEQ ID NO: 184;
(59) the amino acid sequence of SEQ ID NO: 145 and the amino acid sequence of SEQ ID NO: 185;
(60) the amino acid sequence of SEQ ID NO: 146 and the amino acid sequence of SEQ ID NO: 185;
(61) the amino acid sequence of SEQ ID NO: 147 and the amino acid sequence of SEQ ID NO: 185;
(62) the amino acid sequence of SEQ ID NO: 148 and the amino acid sequence of SEQ ID NO: 185;
(63) the amino acid sequence of SEQ ID NO: 149 and the amino acid sequence of SEQ ID NO: 185;
(64) the amino acid sequence of SEQ ID NO: 150 and the amino acid sequence of SEQ ID NO: 186;
(65) the amino acid sequence of SEQ ID NO: 139 and the amino acid sequence of SEQ ID NO: 187;
(66) the amino acid sequence of SEQ ID NO: 112 and the amino acid sequence of SEQ ID NO: 187;
(67) the amino acid sequence of SEQ ID NO: 151 and the amino acid sequence of SEQ ID NO: 187;
(68) the amino acid sequence of SEQ ID NO: 110 and the amino acid sequence of SEQ ID NO: 187;
(69) the amino acid sequence of SEQ ID NO: 152 and the amino acid sequence of SEQ ID NO: 187;
(70) the amino acid sequence of SEQ ID NO: 153 and the amino acid sequence of SEQ ID NO: 187;
(71) the amino acid sequence of SEQ ID NO: 119 and the amino acid sequence of SEQ ID NO: 187;
(72) the amino acid sequence of SEQ ID NO: 154 and the amino acid sequence of SEQ ID NO: 187;
(73) the amino acid sequence of SEQ ID NO: 120 and the amino acid sequence of SEQ ID NO: 187;
(74) the amino acid sequence of SEQ ID NO: 111 and the amino acid sequence of SEQ ID NO: 186;
(75) the amino acid sequence of SEQ ID NO: 116 and the amino acid sequence of SEQ ID NO: 187;
(76) the amino acid sequence of SEQ ID NO: 113 and the amino acid sequence of SEQ ID NO: 187;
(77) the amino acid sequence of SEQ ID NO: 114 and the amino acid sequence of SEQ ID NO: 187;
(78) the amino acid sequence of SEQ ID NO: 119 and the amino acid sequence of SEQ ID NO: 188;
(79) the amino acid sequence of SEQ ID NO: 120 and the amino acid sequence of SEQ ID NO: 188;
(80) the amino acid sequence of SEQ ID NO: 116 and the amino acid sequence of SEQ ID NO: 188;
(81) the amino acid sequence of SEQ ID NO: 121 and the amino acid sequence of SEQ ID NO: 188;
(82) the amino acid sequence of SEQ ID NO: 110 and the amino acid sequence of SEQ ID NO: 189;
(83) the amino acid sequence of SEQ ID NO: 110 and the amino acid sequence of SEQ ID NO: 190;
(84) the amino acid sequence of SEQ ID NO: 113 and the amino acid sequence of SEQ ID NO: 188;
(85) the amino acid sequence of SEQ ID NO: 113 and the amino acid sequence of SEQ ID NO: 191;
(86) the amino acid sequence of SEQ ID NO: 139 and the amino acid sequence of SEQ ID NO: 192;
(87) the amino acid sequence of SEQ ID NO: 151 and the amino acid sequence of SEQ ID NO: 192;
(88) the amino acid sequence of SEQ ID NO: 110 and the amino acid sequence of SEQ ID NO: 192;
(89) the amino acid sequence of SEQ ID NO: 152 and the amino acid sequence of SEQ ID NO: 192;
(90) the amino acid sequence of SEQ ID NO: 154 and the amino acid sequence of SEQ ID NO: 192;
(91) the amino acid sequence of SEQ ID NO: 153 and the amino acid sequence of SEQ ID NO: 192;
(92) the amino acid sequence of SEQ ID NO: 120 and the amino acid sequence of SEQ ID NO: 192;
(93) the amino acid sequence of SEQ ID NO: 119 and the amino acid sequence of SEQ ID NO: 192;
(94) the amino acid sequence of SEQ ID NO: 114 and the amino acid sequence of SEQ ID NO: 192;
(95) the amino acid sequence of SEQ ID NO: 113 and the amino acid sequence of SEQ ID NO: 192;
(96) the amino acid sequence of SEQ ID NO: 113 and the amino acid sequence of SEQ ID NO: 193;
(97) the amino acid sequence of SEQ ID NO: 113 and the amino acid sequence of SEQ ID NO: 194;
(98) the amino acid sequence of SEQ ID NO: 113 and the amino acid sequence of SEQ ID NO: 195; or
(99) the amino acid sequence of SEQ ID NO: 113 and the amino acid sequence of SEQ ID NO: 169.

11.-12. (canceled)

13. The antibody or antibody fragment of claim 1, wherein the antibody or antibody fragment is a monoclonal antibody, a human monoclonal antibody, a chimeric antibody, a bispecific antibody, humanized antibody, fully human antibody, or human antibody, a recombinant scFv (single chain fragment variable or single chain variable domain fragment) antibody, a Fab fragment, a F(ab′)2 fragment, or a variable domain fragment (Fv fragment).

14.-19. (canceled)

20. An isolated nucleic acid comprising a nucleotide sequence encoding the antibody or antibody fragment of claim 1.

21.-22. (canceled)

23. An engineered cell comprising the isolated nucleic acid of claim 20.

24. A composition comprising one or more antibodies or antibody fragments of claim 1.

25. A method of treating a subject infected with SARS-CoV-2 or reducing the likelihood of infection of a subject at risk of contracting SARS-CoV-2, comprising administering to the subject the antibody or antibody fragment of claim 1.

26.-27. (canceled)

28. A method of treating a subject infected with SARS-CoV-2 or reducing the likelihood of infection of a subject at risk of contracting SARS-CoV-2, comprising administering to the subject a first antibody or antibody fragment and a second antibody or antibody fragment, wherein the first and second antibodies or antibody fragments synergistically neutralize the SARS-CoV-2 virus.

29. The method of claim 28, wherein:
(a) the first antibody or antibody fragment and the second antibody or antibody fragment bind to non-overlapping sites on the S glycoprotein;
(b) the first antibody or antibody fragment and the second antibody or antibody fragment have a synergy score of about 10 or more, wherein the synergy score is based on experimental combination response in an focus reduction neutralization test (FRNT) and/or by synergy-scoring models.

30. The method of claim 28, wherein:
(a1) the first antibody or antibody fragment comprises a CDRH1 comprising the amino acid sequence of SEQ ID NO: 196, a CDRH2 comprising the amino acid sequence of SEQ ID NO: 198, a CDRH3 comprising the amino acid sequence of SEQ ID NO: 209, a CDRL1 comprising the amino acid sequence of SEQ ID NO: 234, a CDRL2 comprising the amino acid sequence of SEQ ID NO: 242 or 245, a CDRL3 comprising the amino acid sequence of SEQ ID NO: 251; and/or
(b1) wherein the second antibody or antibody fragment comprises a CDRH1 comprising the amino acid sequence of SEQ ID NO: 196, a CDRH2 comprising the amino acid sequence of SEQ ID NO: 198, a CDRH3 comprising the amino acid sequence of SEQ ID NO: 203, a CDRL1 comprising the amino acid sequence of SEQ ID NO: 227, a CDRL2 comprising the amino acid sequence of SEQ ID NO: 242 or 243, a CDRL3 comprising the amino acid sequence of SEQ ID NO: 251; or
(c1) wherein the second antibody or antibody fragment comprises a CDRH1 comprising the amino acid sequence of SEQ ID NO: 196, a CDRH2 comprising the amino acid sequence of SEQ ID NO: 198, a CDRH3 comprising the amino acid sequence of SEQ ID NO: 204, a CDRL1 comprising the amino acid sequence of SEQ ID NO: 229, a CDRL2 comprising the amino acid sequence of SEQ ID NO: 242 or 243, a CDRL3 comprising the amino acid sequence of SEQ ID NO: 251; or
(d1) wherein the second antibody is sotrovimab (S309), COV2-2381, cilgavimab (COV2-2130 or AZD1061), or tixagevimab (COV2-2196 or AZD8895), or
(a2) the first antibody or antibody fragment comprises a sequence selected from SEQ ID NOs: 15-99; and/or
(b2) wherein the second antibody or antibody fragment comprises a sequence selected from SEQ ID NOs: 15-99, wherein the first antibody or antibody fragment is different from the second antibody or antibody fragment; or
(c2) wherein the second antibody is sotrovimab (S309), COV2-2381, cilgavimab (COV2-2130 or AZD1061), or tixagevimab (COV2-2196 or AZD8895).

31. (canceled)

32. A vaccine formulation comprising (i) one or more antibodies or antibody fragments of claim 1, optionally further comprising a second antibody or antibody fragment that binds to a SARS-CoV-2 surface spike protein wherein the second antibody or antibody fragment is an antibody or antibody fragment of claim 8; or wherein the second antibody or antibody fragment is an antibody or antibody fragment selected from sotrovimab (S309), COV2-2381, cilgavimab (COV2-2130 or AZD1061), or tixagevimab (COV2-2196 or AZD8895) or (ii) one or more expression vectors, wherein the one or more expression vectors at least one nucleic acid encoding an antibody or antibody fragment according to claim 1, wherein the nucleic acid is DNA, RNA, or mRNA.

33.-37. (canceled)

38. A method of detecting infection with SARS-CoV-2 in a subject comprising:
(a) contacting a sample isolated from the subject with the antibody or antibody fragment of claim 1; and
(b) detecting SARS-CoV-2 in the sample by detecting the binding of the antibody or antibody fragment to a SARS-CoV-2 antigen in the sample.

39.-41. (canceled)

42. A method of determining a SARS-CoV-2 virus variant antigenic profile comprising:
(a) contacting a sample comprising the SARS-CoV-2 virus variant with the antibody or antibody fragment of claim 1; and
(b) determining the binding of the antibody or antibody fragment to the SARS-CoV-2 virus variant, wherein absence of binding indicates a conformational change and/or a mutation in the SARS-CoV-2 surface spike protein.

43.-46. (canceled)

47. A biosensor for detecting the presence of a SARS-CoV-2 antigen in a sample, the biosensor comprising
(a) a transducer component comprising an electrode operatively connected to a microprocessor, the microprocessor being adapted to receive, process and transmit a signal;
(b) a receptor component having a sensing element capable of detecting and binding to the SARS-CoV-2 antigen, wherein the sensing element comprises the antibody or antibody fragment of claim 1; and
(c) the transducer component and the receptor component are capable of being brought into direct contact with the sample in situ.

48. A kit comprising the antibody or antibody fragment of claim 1 and instructions for using the first antibody or antibody fragment.

49.-51. (canceled)
